SEQUENCE LISTING (31 sequences)

SEQ ID NO: 1 - protein, 221 aa, Homo sapiens
EDISEPPLHDFYCSRLLDLVFLLDGSSRLSEAEFEVLKAFVVDMMERLRISQKWVRVAVVEYHDGSHAYIGLKDRKRPSELRRIASQVKYAGSQVASTSEVLKYTLFQIFSKIDRPEASRIALLLMASQEPQRMSRNFVRYVQGLKKKKVIVIPVGIGPHANLKQIRLIEKQAPENKAFVLSSVDELEQQRDEIVSYLCDLAPEAPPPTLPPHMAQVTVGP

SEQ ID NO: 2 - protein, 221 aa, Mus musculus
EDTPEPPLHNFYCSKLLDLVFLLDGSSMLSEAEFEVLKAFVVGMMERLHISQKRIRVAVVEYHDGSRAYLELKARKRPSELRRITSQIKYTGSQVASTSEVLKYTLFQIFGKIDRPEASHITLLLTASQEPPRMARNLVRYVQGLKKKKVIVIPVGIGPHASLKQIRLIEKQAPENKAFLLSGVDELEQRRDEIVSYLCDLAPEAPAPTQPPQVAHVTVSP

SEQ ID NO: 3 - DNA, 663 bp, Homo sapiens
gaggacatctcggaaccgccgttgcacgatttctactgcagcaggctactggacctggtcttcctgctggatggctcctccaggctgtccgaggctgagtttgaagtgctgaaggcctttgtggtggacatgatggagcggctgcgcatctcccagaagtgggtccgcgtggccgtggtggagtaccacgacggctcccacgcctacatcgggctcaaggaccggaagcgaccgtcagagctgcggcgcattgccagccaggtgaagtatgcgggcagccaggtggcctccaccagcgaggtcttgaaatacacactgttccaaatcttcagcaagatcgaccgccctgaagcctcccgcatcgccctgctcctgatggccagccaggagccccaacggatgtcccggaactttgtccgctacgtccagggcctgaagaagaagaaggtcattgtgatcccggtgggcattgggccccatgccaacctcaagcagatccgcctcatcgagaagcaggcccctgagaacaaggccttcgtgctgagcagtgtggatgagctggagcagcaaagggacgagatcgttagctacctctgtgaccttgcccctgaagcccctcctcctactctgcccccccacatggcacaagtcactgtgggcccg

SEQ ID NO: 4 - DNA, 660 bp, Mus musculus
gaggatacccccgagccccccctgcacaacttctactgcagcaagctgctggatcttgtcttcctgctggatggctcctctatgttgtccgaggctgagtttgaagtgctcaaagcttttgtggtgggcatgatggagaggttacacatctctcagaagcgcatccgcgtggcagtggtagagtaccatgatggctcccgtgcctaccttgagctcaaggcccggaagcgaccctcagagcttcggcgcatcaccagccagattaagtatacaggcagccaggtggcctctaccagtgaggttttgaagtacacactgttccagatctttggcaaaattgaccgccctgaagcctcccatatcactctgctcctgactgctagccaggagcccccacggatggctaggaatttggtccgctatgtccaaggtctgaagaagaagaaggttatcgtgatccctgtgggcattgggccccacgccagcctcaaacagatccgcctcatcgagaagcaggcccctgaaaacaaggcttttctgctcagtggggtggatgagctggagcagagaagagatgagatagtcagctacctctgtgaccttgctcccgaggccccagccccaactcagcctccacaggtagcccacgtcaccgtgagt

SEQ ID NO: 5 - protein, 221 aa, Artificial
  Synthetic Construct
EDTPEPPLHNFYCSKLLDLVFLLDGSSMLSEAEFEVLKAFVVGMMERLHISQKRIRVAVVEYHDGSHAYLELKARKRPSELRRITSQIKYTGSQVASTSEVLKYTLFQIFGKIDRPEASHITLLLTASQEPPRMARNLVRYVQGLKKKKVIVIPVGIGPHASLKQIRLIEKQAPENKAFLLSGVDELEQRRDEIVSYLCDLAPEAPAPTQPPQVAHVTVSP

SEQ ID NO: 6 - protein, 2813 aa, Homo sapiens
MIPARFAGVLLALALILPGTLCAEGTRGRSSTARCSLFGSDFVNTFDGSMYSFAGYCSYLLAGGCQKRSFSIIGDFQNGKRVSLSVYLGEFFDIHLFVNGTVTQGDQRVSMPYASKGLYLETEAGYYKLSGEAYGFVARIDGSGNFQVLLSDRYFNKTCGLCGNFNIFAEDDFMTQEGTLTSDPYDFANSWALSSGEQWCERASPPSSSCNISSGEMQKGLWEQCQLLKSTSVFARCHPLVDPEPFVALCEKTLCECAGGLECACPALLEYARTCAQEGMVLYGWTDHSACSPVCPAGMEYRQCVSPCARTCQSLHINEMCQERCVDGCSCPEGQLLDEGLCVESTECPCVHSGKRYPPGTSLSRDCNTCICRNSQWICSNEECPGECLVTGQSHFKSFDNRYFTFSGICQYLLARDCQDHSFSIVIETVQCADDRDAVCTRSVTVRLPGLHNSLVKLKHGAGVAMDGQDIQLPLLKGDLRIQHTVTASVRLSYGEDLQMDWDGRGRLLVKLSPVYAGKTCGLCGNYNGNQGDDFLTPSGLAEPRVEDFGNAWKLHGDCQDLQKQHSDPCALNPRMTRFSEEACAVLTSPTFEACHRAVSPLPYLRNCRYDVCSCSDGRECLCGALASYAAACAGRGVRVAWREPGRCELNCPKGQVYLQCGTPCNLTCRSLSYPDEECNEACLEGCFCPPGLYMDERGDCVPKAQCPCYYDGEIFQPEDIFSDHHTMCYCEDGFMHCTMSGVPGSLLPDAVLSSPLSHRSKRSLSCRPPMVKLVCPADNLRAEGLECTKTCQNYDLECMSMGCVSGCLCPPGMVRHENRCVALERCPCFHQGKEYAPGETVKIGCNTCVCRDRKWNCTDHVCDATCSTIGMAHYLTFDGLKYLFPGECQYVLVQDYCGSNPGTFRILVGNKGCSHPSVKCKKRVTILVEGGEIELFDGEVNVKRPMKDETHFEVVESGRYIILLLGKALSVVWDRHLSISVVLKQTYQEKVCGLCGNFDGIQNNDLTSSNLQVEEDPVDFGNSWKVSSQCADTRKVPLDSSPATCHNNIMKQTMVDSSCRILTSDVFQDCNKLVDPEPYLDVCIYDTCSCESIGDCACFCDTIAAYAHVCAQHGKVVTWRTATLCPQSCEERNLRENGYECEWRYNSCAPACQVTCQHPEPLACPVQCVEGCHAHCPPGKILDELLQTCVDPEDCPVCEVAGRRFASGKKVTLNPSDPEHCQICHCDVVNLTCEACQEPGGLVVPPTDAPVSPTTLYVEDISEPPLHDFYCSRLLDLVFLLDGSSRLSEAEFEVLKAFVVDMMERLRISQKWVRVAVVEYHDGSHAYIGLKDRKRPSELRRIASQVKYAGSQVASTSEVLKYTLFQIFSKIDRPEASRIALLLMASQEPQRMSRNFVRYVQGLKKKKVIVIPVGIGPHANLKQIRLIEKQAPENKAFVLSSVDELEQQRDEIVSYLCDLAPEAPPPTLPPHMAQVTVGPGLLGVSTLGPKRNSMVLDVAFVLEGSDKIGEADFNRSKEFMEEVIQRMDVGQDSIHVTVLQYSYMVTVEYPFSEAQSKGDILQRVREIRYQGGNRTNTGLALRYLSDHSFLVSQGDREQAPNLVYMVTGNPASDEIKRLPGDIQVVPIGVGPNANVQELERIGWPNAPILIQDFETLPREAPDLVLQRCCSGEGLQIPTLSPAPDCSQPLDVILLLDGSSSFPASYFDEMKSFAKAFISKANIGPRLTQVSVLQYGSITTIDVPWNVVPEKAHLLSLVDVMQREGGPSQIGDALGFAVRYLTSEMHGARPGASKAVVILVTDVSVDSVDAAADAARSNRVTVFPIGIGDRYDAAQLRILAGPAGDSNVVKLQRIEDLPTMVTLGNSFLHKLCSGFVRICMDEDGNEKRPGDVWTLPDQCHTVTCQPDGQTLLKSHRVNCDRGLRPSCPNSQSPVKVEETCGCRWTCPCVCTGSSTRHIVTFDGQNFKLTGSCSYVLFQNKEQDLEVILHNGACSPGARQGCMKSIEVKHSALSVELHSDMEVTVNGRLVSVPYVGGNMEVNVYGAIMHEVRFNHLGHIFTFTPQNNEFQLQLSPKTFASKTYGLCGICDENGANDFMLRDGTVTTDWKTLVQEWTVQRPGQTCQPILEEQCLVPDSSHCQVLLLPLFAECHKVLAPATFYAICQQDSCHQEQVCEVIASYAHLCRTNGVCVDWRTPDFCAMSCPPSLVYNHCEHGCPRHCDGNVSSCGDHPSEGCFCPPDKVMLEGSCVPEEACTQCIGEDGVQHQFLEAWVPDHQPCQICTCLSGRKVNCTTQPCPTAKAPTCGLCEVARLRQNADQCCPEYECVCDPVSCDLPPVPHCERGLQPTLTNPGECRPNFTCACRKEECKRVSPPSCPPHRLPTLRKTQCCDEYECACNCVNSTVSCPLGYLASTATNDCGCTTTTCLPDKVCVHRSTIYPVGQFWEEGCDVCTCTDMEDAVMGLRVAQCSQKPCEDSCRSGFTYVLHEGECCGRCLPSACEVVTGSPRGDSQSSWKSVGSQWASPENPCLINECVRVKEEVFIQQRNVSCPQLEVPVCPSGFQLSCKTSACCPSCRCERMEACMLNGTVIGPGKTVMIDVCTTCRCMVQVGVISGFKLECRKTTCNPCPLGYKEENNTGECCGRCLPTACTIQLRGGQIMTLKRDETLQDGCDTHFCKVNERGEYFWEKRVTGCPPFDEHKCLAEGGKIMKIPGTCCDTCEEPECNDITARLQYVKVGSCKSEVEVDIHYCQGKCASKAMYSIDINDVQDQCSCCSPTRTEPMQVALHCTNGSVVYHEVLNAMECKCSPRKCSK

SEQ ID NO: 7 - DNA, 8923 bp, Homo sapiens
agctcacagctattgtggtgggaaagggagggtggttggtggatgtcacagcttgggctttatctcccccagcagtggggactccacagcccctgggctacataacagcaagacagtccggagctgtagcagacctgattgagcctttgcagcagctgagagcatggcctagggtgggcggcaccattgtccagcagctgagtttcccagggaccttggagatagccgcagccctcatttgcaggggaaggcaccattgtccagcagctgagtttcccagggaccttggagatagccgcagccctcatttatgattcctgccagatttgccggggtgctgcttgctctggccctcattttgccagggaccctttgtgcagaaggaactcgcggcaggtcatccacggcccgatgcagccttttcggaagtgacttcgtcaacacctttgatgggagcatgtacagctttgcgggatactgcagttacctcctggcagggggctgccagaaacgctccttctcgattattggggacttccagaatggcaagagagtgagcctctccgtgtatcttggggaattttttgacatccatttgtttgtcaatggtaccgtgacacagggggaccaaagagtctccatgccctatgcctccaaagggctgtatctagaaactgaggctgggtactacaagctgtccggtgaggcctatggctttgtggccaggatcgatggcagcggcaactttcaagtcctgctgtcagacagatacttcaacaagacctgcgggctgtgtggcaactttaacatctttgctgaagatgactttatgacccaagaagggaccttgacctcggacccttatgactttgccaactcatgggctctgagcagtggagaacagtggtgtgaacgggcatctcctcccagcagctcatgcaacatctcctctggggaaatgcagaagggcctgtgggagcagtgccagcttctgaagagcacctcggtgtttgcccgctgccaccctctggtggaccccgagccttttgtggccctgtgtgagaagactttgtgtgagtgtgctggggggctggagtgcgcctgccctgccctcctggagtacgcccggacctgtgcccaggagggaatggtgctgtacggctggaccgaccacagcgcgtgcagcccagtgtgccctgctggtatggagtataggcagtgtgtgtccccttgcgccaggacctgccagagcctgcacatcaatgaaatgtgtcaggagcgatgcgtggatggctgcagctgccctgagggacagctcctggatgaaggcctctgcgtggagagcaccgagtgtccctgcgtgcattccggaaagcgctaccctcccggcacctccctctctcgagactgcaacacctgcatttgccgaaacagccagtggatctgcagcaatgaagaatgtccaggggagtgccttgtcactggtcaatcccacttcaagagctttgacaacagatacttcaccttcagtgggatctgccagtacctgctggcccgggattgccaggaccactccttctccattgtcattgagactgtccagtgtgctgatgaccgcgacgctgtgtgcacccgctccgtcaccgtccggctgcctggcctgcacaacagccttgtgaaactgaagcatggggcaggagttgccatggatggccaggacatccagctccccctcctgaaaggtgacctccgcatccagcatacagtgacggcctccgtgcgcctcagctacggggaggacctgcagatggactgggatggccgcgggaggctgctggtgaagctgtcccccgtctacgccgggaagacctgcggcctgtgtgggaattacaatggcaaccagggcgacgacttccttaccccctctgggctggcagagccccgggtggaggacttcgggaacgcctggaagctgcacggggactgccaggacctgcagaagcagcacagcgatccctgcgccctcaacccgcgcatgaccaggttctccgaggaggcgtgcgcggtcctgacgtcccccacattcgaggcctgccatcgtgccgtcagcccgctgccctacctgcggaactgccgctacgacgtgtgctcctgctcggacggccgcgagtgcctgtgcggcgccctggccagctatgccgcggcctgcgcggggagaggcgtgcgcgtcgcgtggcgcgagccaggccgctgtgagctgaactgcccgaaaggccaggtgtacctgcagtgcgggaccccctgcaacctgacctgccgctctctctcttacccggatgaggaatgcaatgaggcctgcctggagggctgcttctgccccccagggctctacatggatgagaggggggactgcgtgcccaaggcccagtgcccctgttactatgacggtgagatcttccagccagaagacatcttctcagaccatcacaccatgtgctactgtgaggatggcttcatgcactgtaccatgagtggagtccccggaagcttgctgcctgacgctgtcctcagcagtcccctgtctcatcgcagcaaaaggagcctatcctgtcggccccccatggtcaagctggtgtgtcccgctgacaacctgcgggctgaagggctcgagtgtaccaaaacgtgccagaactatgacctggagtgcatgagcatgggctgtgtctctggctgcctctgccccccgggcatggtccggcatgagaacagatgtgtggccctggaaaggtgtccctgcttccatcagggcaaggagtatgcccctggagaaacagtgaagattggctgcaacacttgtgtctgtcgggaccggaagtggaactgcacagaccatgtgtgtgatgccacgtgctccacgatcggcatggcccactacctcaccttcgacgggctcaaatacctgttccccggggagtgccagtacgttctggtgcaggattactgcggcagtaaccctgggacctttcggatcctagtggggaataagggatgcagccacccctcagtgaaatgcaagaaacgggtcaccatcctggtggagggaggagagattgagctgtttgacggggaggtgaatgtgaagaggcccatgaaggatgagactcactttgaggtggtggagtctggccggtacatcattctgctgctgggcaaagccctctccgtggtctgggaccgccacctgagcatctccgtggtcctgaagcagacataccaggagaaagtgtgtggcctgtgtgggaattttgatggcatccagaacaatgacctcaccagcagcaacctccaagtggaggaagaccctgtggactttgggaactcctggaaagtgagctcgcagtgtgctgacaccagaaaagtgcctctggactcatcccctgccacctgccataacaacatcatgaagcagacgatggtggattcctcctgtagaatccttaccagtgacgtcttccaggactgcaacaagctggtggaccccgagccatatctggatgtctgcatttacgacacctgctcctgtgagtccattggggactgcgcctgcttctgcgacaccattgctgcctatgcccacgtgtgtgcccagcatggcaaggtggtgacctggaggacggccacattgtgcccccagagctgcgaggagaggaatctccgggagaacgggtatgagtgtgagtggcgctataacagctgtgcacctgcctgtcaagtcacgtgtcagcaccctgagccactggcctgccctgtgcagtgtgtggagggctgccatgcccactgccctccagggaaaatcctggatgagcttttgcagacctgcgttgaccctgaagactgtccagtgtgtgaggtggctggccggcgttttgcctcaggaaagaaagtcaccttgaatcccagtgaccctgagcactgccagatttgccactgtgatgttgtcaacctcacctgtgaagcctgccaggagccgggaggcctggtggtgcctcccacagatgccccggtgagccccaccactctgtatgtggaggacatctcggaaccgccgttgcacgatttctactgcagcaggctactggacctggtcttcctgctggatggctcctccaggctgtccgaggctgagtttgaagtgctgaaggcctttgtggtggacatgatggagcggctgcgcatctcccagaagtgggtccgcgtggccgtggtggagtaccacgacggctcccacgcctacatcgggctcaaggaccggaagcgaccgtcagagctgcggcgcattgccagccaggtgaagtatgcgggcagccaggtggcctccaccagcgaggtcttgaaatacacactgttccaaatcttcagcaagatcgaccgccctgaagcctcccgcatcgccctgctcctgatggccagccaggagccccaacggatgtcccggaactttgtccgctacgtccagggcctgaagaagaagaaggtcattgtgatcccggtgggcattgggccccatgccaacctcaagcagatccgcctcatcgagaagcaggcccctgagaacaaggccttcgtgctgagcagtgtggatgagctggagcagcaaagggacgagatcgttagctacctctgtgaccttgcccctgaagcccctcctcctactctgcccccccacatggcacaagtcactgtgggcccggggctcttgggggtttcgaccctggggcccaagaggaactccatggttctggatgtggcgttcgtcctggaaggatcggacaaaattggtgaagccgacttcaacaggagcaaggagttcatggaggaggtgattcagcggatggatgtgggccaggacagcatccacgtcacggtgctgcagtactcctacatggtgaccgtggagtaccccttcagcgaggcacagtccaaaggggacatcctgcagcgggtgcgagagatccgctaccagggcggcaacaggaccaacactgggctggccctgcggtacctctctgaccacagcttcttggtcagccagggtgaccgggagcaggcgcccaacctggtctacatggtcaccggaaatcctgcctctgatgagatcaagaggctgcctggagacatccaggtggtgcccattggagtgggccctaatgccaacgtgcaggagctggagaggattggctggcccaatgcccctatcctcatccaggactttgagacgctcccccgagaggctcctgacctggtgctgcagaggtgctgctccggagaggggctgcagatccccaccctctcccctgcacctgactgcagccagcccctggacgtgatccttctcctggatggctcctccagtttcccagcttcttattttgatgaaatgaagagtttcgccaaggctttcatttcaaaagccaatatagggcctcgtctcactcaggtgtcagtgctgcagtatggaagcatcaccaccattgacgtgccatggaacgtggtcccggagaaagcccatttgctgagccttgtggacgtcatgcagcgggagggaggccccagccaaatcggggatgccttgggctttgctgtgcgatacttgacttcagaaatgcatggtgccaggccgggagcctcaaaggcggtggtcatcctggtcacggacgtctctgtggattcagtggatgcagcagctgatgccgccaggtccaacagagtgacagtgttccctattggaattggagatcgctacgatgcagcccagctacggatcttggcaggcccagcaggcgactccaacgtggtgaagctccagcgaatcgaagacctccctaccatggtcaccttgggcaattccttcctccacaaactgtgctctggatttgttaggatttgcatggatgaggatgggaatgagaagaggcccggggacgtctggaccttgccagaccagtgccacaccgtgacttgccagccagatggccagaccttgctgaagagtcatcgggtcaactgtgaccgggggctgaggccttcgtgccctaacagccagtcccctgttaaagtggaagagacctgtggctgccgctggacctgcccctgcgtgtgcacaggcagctccactcggcacatcgtgacctttgatgggcagaatttcaagctgactggcagctgttcttatgtcctatttcaaaacaaggagcaggacctggaggtgattctccataatggtgcctgcagccctggagcaaggcagggctgcatgaaatccatcgaggtgaagcacagtgccctctccgtcgagctgcacagtgacatggaggtgacggtgaatgggagactggtctctgttccttacgtgggtgggaacatggaagtcaacgtttatggtgccatcatgcatgaggtcagattcaatcaccttggtcacatcttcacattcactccacaaaacaatgagttccaactgcagctcagccccaagacttttgcttcaaagacgtatggtctgtgtgggatctgtgatgagaacggagccaatgacttcatgctgagggatggcacagtcaccacagactggaaaacacttgttcaggaatggactgtgcagcggccagggcagacgtgccagcccatcctggaggagcagtgtcttgtccccgacagctcccactgccaggtcctcctcttaccactgtttgctgaatgccacaaggtcctggctccagccacattctatgccatctgccagcaggacagttgccaccaggagcaagtgtgtgaggtgatcgcctcttatgcccacctctgtcggaccaacggggtctgcgttgactggaggacacctgatttctgtgctatgtcatgcccaccatctctggtctacaaccactgtgagcatggctgtccccggcactgtgatggcaacgtgagctcctgtggggaccatccctccgaaggctgtttctgccctccagataaagtcatgttggaaggcagctgtgtccctgaagaggcctgcactcagtgcattggtgaggatggagtccagcaccagttcctggaagcctgggtcccggaccaccagccctgtcagatctgcacatgcctcagcgggcggaaggtcaactgcacaacgcagccctgccccacggccaaagctcccacgtgtggcctgtgtgaagtagcccgcctccgccagaatgcagaccagtgctgccccgagtatgagtgtgtgtgtgacccagtgagctgtgacctgcccccagtgcctcactgtgaacgtggcctccagcccacactgaccaaccctggcgagtgcagacccaacttcacctgcgcctgcaggaaggaggagtgcaaaagagtgtccccaccctcctgccccccgcaccgtttgcccacccttcggaagacccagtgctgtgatgagtatgagtgtgcctgcaactgtgtcaactccacagtgagctgtccccttgggtacttggcctcaaccgccaccaatgactgtggctgtaccacaaccacctgccttcccgacaaggtgtgtgtccaccgaagcaccatctaccctgtgggccagttctgggaggagggctgcgatgtgtgcacctgcaccgacatggaggatgccgtgatgggcctccgcgtggcccagtgctcccagaagccctgtgaggacagctgtcggtcgggcttcacttacgttctgcatgaaggcgagtgctgtggaaggtgcctgccatctgcctgtgaggtggtgactggctcaccgcggggggactcccagtcttcctggaagagtgtcggctcccagtgggcctccccggagaacccctgcctcatcaatgagtgtgtccgagtgaaggaggaggtctttatacaacaaaggaacgtctcctgcccccagctggaggtccctgtctgcccctcgggctttcagctgagctgtaagacctcagcgtgctgcccaagctgtcgctgtgagcgcatggaggcctgcatgctcaatggcactgtcattgggcccgggaagactgtgatgatcgatgtgtgcacgacctgccgctgcatggtgcaggtgggggtcatctctggattcaagctggagtgcaggaagaccacctgcaacccctgccccctgggttacaaggaagaaaataacacaggtgaatgttgtgggagatgtttgcctacggcttgcaccattcagctaagaggaggacagatcatgacactgaagcgtgatgagacgctccaggatggctgtgatactcacttctgcaaggtcaatgagagaggagagtacttctgggagaagagggtcacaggctgcccaccctttgatgaacacaagtgtctggctgagggaggtaaaattatgaaaattccaggcacctgctgtgacacatgtgaggagcctgagtgcaacgacatcactgccaggctgcagtatgtcaaggtgggaagctgtaagtctgaagtagaggtggatatccactactgccagggcaaatgtgccagcaaagccatgtactccattgacatcaacgatgtgcaggaccagtgctcctgctgctctccgacacggacggagcccatgcaggtggccctgcactgcaccaatggctctgttgtgtaccatgaggttctcaatgccatggagtgcaaatgctcccccaggaagtgcagcaagtgaggctgctgcagctgcatgggtgcctgctgctgcctgccttggcctgatggccaggccagagtgctgccagtcctctgcatgttctgctcttgtgcccttctgagcccacaataaaggctgagctcttatcttgctgcatgttctgctcttgtgcccttctgagcccacaat

SEQ ID NO: 8 - protein, 2813 aa, Mus musculus
MNPFRYEICLLVLALTWPGTLCTEKPRDRPSTARCSLFGDDFINTFDETMYSFAGGCSYLLAGDCQKRSFSILGNFQDGKRMSLSVYLGEFFDIHLFANGTVTQGDQSISMPYASQGLYLEREAGYYKLSSETFGFAARIDGNGNFQVLMSDRHFNKTCGLCGDFNIFAEDDFRTQEGTLTSDPYDFANSWALSSEEQRCKRASPPSRNCESSSGDMHQAMWEQCQLLKTASVFARCHPLVDPESFVALCEKILCTCATGPECACPVLLEYARTCAQEGMVLYGWTDHSACRPACPAGMEYKECVSPCPRTCQSLSINEVCQQQCVDGCSCPEGELLDEDRCVQSSDCPCVHAGKRYPPGTSLSQDCNTCICRNSLWICSNEECPGECLVTGQSHFKSFDNRYFTFSGICQYLLARDCEDHTFSIVIETMQCADDPDAVCTRSVSVRLSALHNSLVKLKHGGAVGIDGQDVQLPFLQGDLRIQHTVMASVRLSYAEDLQMDWDGRGRLLVKLSPVYSGKTCGLCGNYNGNKGDDFLTPAGLVEPLVVDFGNAWKLQGDCSDLRRQHSDPCSLNPRLTRFAEEACALLTSSKFEACHHAVSPLPYLQNCRYDVCSCSDSRDCLCNAVANYAAECARKGVHIGWREPGFCALGCPQGQVYLQCGNSCNLTCRSLSLPDEECSEVCLEGCYCPPGLYQDERGDCVPKAQCPCYYDGELFQPADIFSDHHTMCYCEDGFMHCTTSGTLGSLLPDTVLSSPLSHRSKRSLSCRPPMVKLVCPADNPRAQGLECAKTCQNYDLECMSLGCVSGCLCPPGMVRHENKCVALERCPCFHQGAEYAPGDTVKIGCNTCVCRERKWNCTNHVCDATCSAIGMAHYLTFDGLKYLFPGECQYVLVQDYCGSNPGTFQILVGNEGCSYPSVKCRKRVTILVDGGELELFDGEVNVKRPLRDESHFEVVESGRYVILLLGQALSVVWDHHLSISVVLKHTYQEQVCGLCGNFDGIQNNDFTTSSLQVEEDPVNFGNSWKVSSQCADTRKLSLDVSPATCHNNIMKQTMVDSACRILTSDVFQGCNRLVDPEPYLDICIYDTCSCESIGDCACFCDTIAAYAHVCAQHGQVVAWRTPTLCPQSCEEKNVRENGYECEWRYNSCAPACPVTCQHPEPLACPVQCVEGCHAHCPPGRILDELLQTCVDPQDCPVCEVAGRRLAPGKKITLSPDDPAHCQNCHCDGVNLTCEACQEPGGLVAPPTDAPVSSTTPYVEDTPEPPLHNFYCSKLLDLVFLLDGSSMLSEAEFEVLKAFVVGMMERLHISQKRIRVAVVEYHDGSRAYLELKARKRPSELRRITSQIKYTGSQVASTSEVLKYTLFQIFGKIDRPEASHITLLLTASQEPPRMARNLVRYVQGLKKKKVIVIPVGIGPHASLKQIRLIEKQAPENKAFLLSGVDELEQRRDEIVSYLCDLAPEAPAPTQPPQVAHVTVSPGIAGISSPGPKRKSMVLDVVFVLEGSDEVGEANFNKSKEFVEEVIQRMDVSPDATRISVLQYSYTVTMEYAFNGAQSKEEVLRHVREIRYQGGNRTNTGQALQYLSEHSFSPSQGDRVEAPNLVYMVTGNPASDEIKRLPGDIQVVPIGVGPHANMQELERISRPIAPIFIRDFETLPREAPDLVLQTCCSKEGLQLPTLPPLPDCSQPLDVVLLLDGSSSLPESSFDKMKSFAKAFISKANIGPHLTQVSVIQYGSINTIDVPWNVVQEKAHLQSLVDLMQQEGGPSQIGDALAFAVRYVTSQIHGARPGASKAVVIIIMDTSLDPVDTAADAARSNRVAVFPVGVGDRYDEAQLRILAGPGASSNVVKLQQVEDLSTMATLGNSFFHKLCSGFSGVCVDEDGNEKRPGDVWTLPDQCHTVTCLANGQTLLQSHRVNCDHGPRPSCANSQSPVRVEETCGCRWTCPCVCTGSSTRHIVTFDGQNFKLTGSCSYVIFQNKEQDLEVLLHNGACSPGAKQACMKSIEIKHAGVSAELHSNMEMAVDGRLVLAPYVGENMEVSIYGAIMYEVRFTHLGHILTYTPQNNEFQLQLSPKTFASKMHGLCGICDENGANDFTLRDGTVTTDWKRLVQEWTVQQPGYTCQAVPEEQCPVSDSSHCQVLLSASFAECHKVIAPATFHTICQQDSCHQERVCEVIASYAHLCRTSGVCVDWRTTDFCAMSCPPSLVYNHCERGCPRHCDGNTSFCGDHPSEGCFCPQHQVFLEGSCVPEEACTQCVGEDGVRHQFLETWVPDHQPCQICMCLSGRKINCTAQPCPTARAPTCGPCEVARLKQSTNLCCPEYECVCDLFNCNLPPVPPCEGGLQPTLTNPGECRPTFTCACRKEECKRVSPPSCPPHRTPTLRKTQCCDEYECACSCVNSTLSCPLGYLASATTNDCGCTTTTCLPDKVCVHRGTVYPVGQFWEEGCDTCTCTDMEDTVVGLRVVQCSQRPCEDSCQPGFSYVLHEGECCGRCLPSACKVVAGSLRGDSHSSWKSVGSRWAVPENPCLVNECVRVEDAVFVQQRNISCPQLAVPTCPTGFQLNCETSECCPSCHCEPVEACLLNGTIIGPGKSVMVDLCTTCRCIVQTDAISRFKLECRKTTCEACPMGYREEKSQGECCGRCLPTACTIQLRGGRIMTLKQDETFQDGCDSHLCRVNERGEYIWEKRVTGCPPFDEHKCLAEGGKIVKIPGTCCDTCEEPDCKDITAKVQYIKVGDCKSQEEVDIHYCQGKCASKAVYSIDIEDVQEQCSCCLPSRTEPMRVPLHCTNGSVVYHEVINAMQCRCSPRNCSK

SEQ ID NO: 9 - DNA, 8537 bp, Mus musculus
agtagcggctgggtttcctcaagggaccttggagatacagcccctgtttgtatgggcaagatgaaccctttcaggtatgagatctgcctgcttgttctggccctcacctggccagggaccctctgcacagaaaagccccgtgacaggccgtcgacggcccgatgcagcctctttggggacgacttcatcaacacgtttgatgagaccatgtacagctttgcagggggctgcagttatctcctggctggggactgccagaaacgttccttctccattctcgggaacttccaagatggcaagagaatgagcctgtctgtgtatcttggggagttttttgacatccatttgtttgccaatggcaccgtaacgcagggtgaccaaagcatctccatgccctacgcctcccaaggactctacctagaacgcgaggctgggtactataagctctccagtgagacctttggctttgcggccagaatcgatggcaatggcaacttccaagtcctgatgtcagacagacacttcaacaagacctgtgggctgtgcggtgattttaacatcttcgcggaagatgattttaggacgcaggaggggaccttgacctcagacccctatgattttgccaactcctgggccctgagcagtgaggaacagcggtgtaaacgggcatctcctcccagcaggaactgcgagagctcttctggggacatgcatcaggccatgtgggagcaatgccagctactgaagacggcatcggtgtttgcccgctgccaccctctggtggatcccgagtcctttgtggctctgtgtgagaagattttgtgtacgtgtgctacggggccagagtgcgcatgtcctgtactccttgagtatgcccgaacctgcgcccaggaagggatggtgctgtacggctggactgaccacagtgcctgtcgtccagcttgcccagctggcatggaatataaggagtgtgtgtctccttgccccagaacctgccagagcctgtctatcaatgaagtgtgtcagcagcaatgtgtagacggctgtagctgccctgagggagagctcttggatgaagaccgatgtgtgcagagctccgactgtccttgcgtgcacgctgggaagcggtaccctcctggcacctccctctctcaggactgcaacacttgtatctgcagaaacagcctatggatctgcagcaatgaggaatgcccaggggagtgtcttgtcacaggccaatcgcacttcaagagcttcgacaacaggtacttcaccttcagtgggatctgccaatatctgctggcccgggactgcgaggatcacactttctccattgtcatagagaccatgcagtgtgccgatgaccctgatgctgtctgcacccgctcggtcagtgtgcggctctctgccctgcacaacagcctggtgaaactgaagcacgggggagcagtgggcatcgatggtcaggatgtccagctccccttcctgcaaggtgacctccgcatccagcacacagtgatggcttctgtacgcctcagctatgcggaggacctgcagatggactgggatggccgtgggcggctactggttaagctgtccccagtctattctgggaagacctgtggcttgtgtgggaattacaacggcaacaagggagacgacttcctcacgccggccggcttggtggagcccctggtggtagacttcggaaacgcctggaagcttcaaggggactgttcggacctgcgcaggcaacacagcgacccctgcagcctgaatccacgcttgaccaggtttgcagaggaggcttgtgcgctcctgacgtcctccaagttcgaggcctgccaccacgcagtcagccctctgccctatctgcagaactgccgttatgatgtttgctcctgctccgacagccgggattgcctgtgtaacgcagtagctaactatgctgccgagtgtgcccgaaaaggcgtgcacatcgggtggcgggagcctggcttctgtgctctgggctgtccacagggccaggtgtacctgcagtgtgggaattcctgcaacctgacctgccgctccctctccctcccggatgaagaatgcagtgaagtctgtcttgaaggctgctactgcccaccagggctctaccaggatgaaagaggggactgtgtgcccaaggcccagtgcccctgctactacgatggtgagctcttccagcctgcggacattttctcagaccaccataccatgtgttactgtgaagatggcttcatgcactgtaccacaagtggcaccctggggagcctgttgcctgacactgtcctcagcagtcccctgtctcaccgtagcaaaaggagcctttcctgccggccacccatggtcaagctggtgtgtcctgctgacaacccacgggctcaagggctggagtgtgctaagacgtgccagaactacgacctggagtgtatgagcctgggctgtgtgtctggctgcctctgtcccccaggcatggtccggcacgaaaacaagtgtgtggccttggagcggtgtccctgcttccatcagggtgcagagtacgccccgggagacacagtgaagattggctgcaacacctgtgtctgccgggagcggaagtggaactgcacgaaccatgtgtgtgacgccacttgctctgccattggtatggcccactacctcaccttcgatggactcaagtacctgttcccgggggagtgccagtatgttctggtgcaggattactgtggcagtaaccctgggacctttcagatcctggtgggaaatgagggttgcagctatccctcggtgaagtgcaggaagcgggtgaccatcctggtggatggaggggagcttgaactgtttgacggagaggtgaacgttaagaggcccctgagagatgaatctcactttgaggtggtggagtcgggccggtacgtcatcctgctgctgggtcaggccctttctgtggtctgggaccaccacctcagcatctctgtggtcctgaagcacacataccaggaacaggtgtgtggcctctgcgggaactttgatggcatccagaacaatgacttcaccactagcagcctccaggtggaggaagaccccgtcaactttgggaactcctggaaagtgagctcacagtgtgctgacacgagaaagctgtcactagatgtttcccctgccacttgccacaacaacatcatgaaacagacgatggtggactcagcctgcagaatccttaccagtgacgtcttccagggctgcaacaggctggtggaccctgagccatacctggacatctgtatttatgacacttgctcctgtgagtccatcggggactgcgcctgtttctgtgacaccattgctgcctatgcccacgtgtgtgcccagcatggccaggtggtagcctggaggacacccacactgtgcccccagagctgtgaagaaaagaatgttcgggaaaatggctatgagtgtgagtggcgttataacagctgtgcgcctgcttgcccagtcacgtgtcagcaccctgagcctctggcttgccctgtgcagtgtgtggagggttgtcatgcacattgccctccagggagaatcctggatgaacttctgcagacctgcgtagacccccaagactgccccgtgtgtgaggtggctggtcggcgcttggctcctggaaagaaaatcaccttgagtcctgatgaccctgcacactgtcagaattgtcactgtgatggtgtgaaccttacgtgtgaagcctgccaagagcccggaggcctggtggcacccccaactgatgccccagtcagctctaccaccccatatgttgaggatacccccgagccccccctgcacaacttctactgcagcaagctgctggatcttgtcttcctgctggatggctcctctatgttgtccgaggctgagtttgaagtgctcaaagcttttgtggtgggcatgatggagaggttacacatctctcagaagcgcatccgcgtggcagtggtagagtaccatgatggctcccgtgcctaccttgagctcaaggcccggaagcgaccctcagagcttcggcgcatcaccagccagattaagtatacaggcagccaggtggcctctaccagtgaggttttgaagtacacactgttccagatctttggcaaaattgaccgccctgaagcctcccatatcactctgctcctgactgctagccaggagcccccacggatggctaggaatttggtccgctatgtccaaggtctgaagaagaagaaggttatcgtgatccctgtgggcattgggccccacgccagcctcaaacagatccgcctcatcgagaagcaggcccctgaaaacaaggcttttctgctcagtggggtggatgagctggagcagagaagagatgagatagtcagctacctctgtgaccttgctcccgaggccccagccccaactcagcctccacaggtagcccacgtcaccgtgagtccagggatcgctgggatctcgtcaccgggaccaaaacggaagtccatggttctggatgtggtgtttgtcctggaggggtcagacgaagttggtgaagccaacttcaataagagcaaggagttcgtggaggaggtaatccagcgcatggacgtgagcccggatgcaacgcgcatctcagtactgcagtattcctacacggtaaccatggagtatgccttcaatggggcccagtccaaggaggaggtgctgcggcacgtgcgagagatccgctaccagggcggcaataggacaaacactgggcaggccctgcagtacctttctgagcacagcttctctcccagccaaggggaccgggtagaggcacctaacctggtctacatggtcacggggaaccccgcctctgatgagatcaagaggttgcctggagacatccaggtggtacccattggggtgggcccccatgccaacatgcaggaactggagaggatcagcaggcccatcgctcccatcttcatccgggactttgagacacttccccgagaggctcctgacctggtcctgcagacatgttgctccaaggagggtctgcaactgcccaccctcccccctctccctgactgcagccaacccctggatgtggtcctgctcctggatggctcctctagcttgccagagtcttcctttgataaaatgaagagttttgccaaggctttcatttcaaaggccaacattgggccccacctcacacaggtgtccgtgatacagtatggaagcatcaataccattgatgtaccatggaatgtggttcaggagaaagcccatctacagagtttggtggacctcatgcagcaggagggtggccccagccagattggggatgctctggcctttgccgtgcgctatgtaacttcacaaatccacggagccaggcctggggcctccaaagcagtggtcatcatcatcatggatacctccttggatcccgtggacacagcagcagatgctgccagatccaaccgagtggcagtgtttcccgttggggttggggatcggtatgatgaagcccagctgaggatcttggcaggccctggggccagctccaatgtggtaaagctccagcaagttgaagacctctccaccatggccaccctgggcaactccttcttccacaaactgtgttctgggttttctggagtttgtgtggatgaagatgggaatgagaagaggcctggggatgtctggaccttgccggatcagtgccacacagtgacttgcttggcaaatggccagaccttgctgcagagtcatcgtgtcaattgtgaccatggaccccggccttcatgtgccaacagccagtctcctgttcgggtggaggagacgtgtggctgccgctggacctgcccttgtgtgtgcacgggcagttccactcggcacatcgtcaccttcgatgggcagaatttcaagcttactggtagctgctcctatgtcatctttcaaaacaaggagcaggacctggaagtgctcctccacaatggggcctgcagccccggggcaaaacaagcctgcatgaagtccattgagattaagcatgctggcgtctctgctgagctgcacagtaacatggagatggcagtggatgggagactggtccttgccccgtacgttggtgaaaacatggaagtcagcatctacggcgctatcatgtatgaagtcaggtttacccatcttggccacatcctcacatacacgccacaaaacaacgagttccaactgcagcttagccccaagacctttgcttcgaagatgcatggtctttgcggaatctgtgatgaaaacggggccaatgacttcacgttgcgagatggcacggtcaccacagactggaaaaggcttgtccaggaatggacggtgcagcagccagggtacacatgccaggctgttcccgaggagcagtgtcccgtctctgacagctcccactgccaggtcctcctctcagcgtcgtttgctgaatgccacaaggtcatcgctccagccacattccataccatctgccagcaagacagttgccaccaggagcgagtgtgtgaggtgattgcttcttacgcccatctctgtcggaccagtggggtctgtgttgattggaggacaactgatttctgtgctatgtcatgcccaccgtccctggtgtataaccactgtgagcgtggctgccctcggcactgcgatgggaacactagcttctgtggggaccatccctcagaaggctgcttctgtccccaacaccaagtttttctggaaggcagctgtgtccccgaggaggcctgcactcagtgtgttggcgaggatggagttcgacatcagttcctggagacctgggtcccagaccatcagccctgtcagatctgtatgtgcctcagtgggagaaagattaactgcactgcccagccgtgtcccacagcccgagctcccacgtgtggcccatgtgaagtggctcgcctcaagcagagcacaaacctgtgctgcccagagtatgagtgtgtgtgtgacctgttcaactgcaacttgcctccagtgcctccgtgtgaaggagggctccagccaaccctgaccaaccctggagaatgcagacccacctttacctgtgcctgcaggaaagaagagtgcaaaagagtgtccccaccctcctgcccccctcaccggacacccactctccggaagacccagtgctgtgatgaatacgagtgtgcttgcagctgtgtcaactccacgctgagctgcccacttggctacctggcctcagccactaccaatgactgtggctgcaccacgaccacctgtctccctgacaaggtttgtgtccaccgaggcaccgtctaccctgtgggccagttctgggaggagggctgtgacacgtgcacctgtacggacatggaggatactgtcgtgggcctgcgtgtggtccagtgctctcaaaggccctgtgaagacagctgtcagccaggtttttcttatgttctccacgaaggcgagtgctgtggaaggtgcctgccctctgcttgcaaggtggtggctggctcactgcggggcgattcccactcttcctggaaaagtgttggatctcggtgggctgttcctgagaacccctgcctcgtcaacgagtgtgtccgcgtggaggatgcagtgtttgtgcagcagaggaacatctcctgcccacagctggctgtccctacctgtcccacaggcttccaactgaactgtgagacctcagagtgctgtcctagctgccactgtgagcctgtggaggcctgcctgctcaatggcaccatcattgggcccgggaagagtgtgatggttgacctatgcacgacctgccgctgcatcgtgcagacagacgccatctccagattcaagctggagtgcaggaagactacctgtgaggcctgccccatgggctatcgggaagagaagagccagggtgaatgctgtgggagatgcttgcctacagcttgcactattcagctaagaggaggacggatcatgaccctgaagcaagatgagacattccaggatggctgtgacagtcatttgtgcagggtcaacgagagaggagagtacatctgggagaagagggtcacgggctgcccaccatttgatgaacacaagtgtctggctgaaggaggcaaaatcgtgaaaattccaggcacctgctgtgacacatgtgaggagcctgattgcaaagacatcacagccaaggtgcagtacatcaaagtgggagattgtaagtcccaagaggaagtggacattcattactgccagggaaagtgtgccagcaaagctgtgtactccattgacatcgaggatgtgcaggagcaatgctcctgctgcctgccctcgaggacggagcccatgcgcgtgcccttgcactgcaccaatggctctgtcgtgtaccacgaggtcatcaacgccatgcagtgcaggtgttctccccggaactgcagcaagtgaggcctgtgcagctacagcggattcctactgatacc

SEQ ID NO: 10 - protein, 208 aa, Mus musculus
DISEPPLHDFYCSRLLDLVFLLDGSSRLSEAEFEVLKAFVVDMMERLRISQKWVRVAVVEYHDGSHAYIGLKDRKRPSELRRIASQVKYAGSQVASTSEVLKYTLFQIFSKIDRPEASRIALLLMASQEPQRMSRNFVRYVQGLKKKKVIVIPVGIGPHANLKQIRLIEKQAPENKAFVLSSVDELEQQRDEIVSYLCDLAPEAPPPT

SEQ ID NO: 11 - DNA, 7939 bp, Artificial
  Synthetic Construct
gatcccagcatggggtagtgaacaattctacctcactctaactgggcatcacatacaatctgaggatcagtatctatgaatattaaagatctgcccccccccccccgatctgtgaatgtaggaaggaaagggatatgggattcttggccctcaaatataacatcttggggctgtggtccaggtcacagctttcagaacccggtgggaagacattcactactgtcaccctaggctcttgctggaacaaactctgagaacctggctttgggaaggtgaggggtcctgagggtggttcctggactttttgaagtcctgcccttaggtagggaacatttattcaaatgttgacatagtcagcaccactccctgacaccaatcgagcctaatgggaacttgggttcactggggacgggctgtggctgcagctgccacagccagagcctgccgccaccatctgtacaaacaagagtgaacactgcccagggtgagttttctagcaaacacaggaaaccaggaagtatgtcactcatgcaccattaacggaggattgaggccataaattgtgggattatgaggaagtaggaattaacttcccagtaagatcctacccaccttccttgtgctgaaacagacatgaggaataagggagggggggggtaatgttcactgcaagcttacttagggcatccagctcaaagggaatggaaactctaagtctcttgtcccctcagctggcctggagaaaggtacatgataatctacattagtgtttcaaggcaggttggggacattggctagcagtgaggtcatgggacatgctcagggtttagggcactgtcaccttcttatatttactgtaaagatggaagaccaagggagcagaatggaggaggcgcattctgtgtggatccccaccacagcccatcttgatttcccttatggtctggctatgggtggctttctggaacatgacatggttgaagggtacagtgattacagagggctggaaacttgtctggtcctagagaaacctgtgggctgggcaatgactcaacacaggcttgtgggcatcatggagccggctgagggctagccactccttggtagacagcatgtctaccatctgcctgttggcagaatgggcaatggagccatcgacacttcctagatgcaacacgtctagtcctcccacaccctgtgcctgccagatggagcagtgattttacacttttatccgtcttcctgacgtcttcttactctcctttgtcttctttcccgtgaggtccagcatcattaaacaccggttcagaatccatacggcttaagttctcataacatttgtaatggtttctaatgtaaaccattcatctccagtggaacatttacaagtcccagcgtgggccaacattttagaatgctatctgtcctcagagacacttataagagggtttgaattataattaaatattaatgtggctagagacatagattggaaggaaatcaatgttacatggctatggagggcttagaacaacagattacgtttcgggttctaaaatggattaaagcttgaggaagcctctgcttcctctttaccccatctctctctctcttgcttgctctctctctctctctctctctctctctctctctctctctctctgtattcaagactcatcttgggaaaacatcaacaatgacatcccactcaggtgagacctgcttgatgggagcaggcaggacgttccatgggggacctgcgaggcagtcctgcctgcccataggtcctccctgaatcattgtggtttctttcccttctgctacagtcactgtgatggtgtgaaccttacgtgtgaagcctgccaagagcccgggggcctggtggtgcctcccacagatgccccggtgagccccaccactctgtatgtggaggacatctcggaaccgccgttgcacgatttctactgcagcaggctactggacctggtcttcctgctggatggctcctccaggctgtccgaggctgagtttgaagtgctgaaggcctttgtggtggacatgatggagcggctgcgcatctcccagaagtgggtccgcgtggccgtggtggagtaccacgacggctcccacgcctacatcgggctcaaggaccggaagcgaccgtcagagctgcggcgcattgccagccaggtgaagtatgcgggcagccaggtggcctccaccagcgaggtcttgaaatacacactgttccaaatcttcagcaagatcgaccgccctgaagcctcccgcatcgccctgctcctgatggccagccaggagccccaacggatgtcccggaactttgtccgctacgtccagggcctgaagaagaagaaggtcattgtgatcccggtgggcattgggccccatgccaacctcaagcagatccgcctcatcgagaagcaggcccctgagaacaaggccttcgtgctgagcagtgtggatgagctggagcagcaaagggacgagatcgttagctacctctgtgaccttgcccctgaagcccctcctcctactctgcccccccacatggcacaagtcactgtgggccccgggatcgctgggatctcgtcaccgggaccaaaacggaagtccatggttctggatgtggtgtttgtcctggaggggtcagacgaagttggtgaagccaacttcaataagagcaaggagttcgtggaggaggtaatccagcgcatggacgtgagcccggatgcaacgcgcatctcagtactgcagtattcctacacggtaaccatggagtatgccttcaatggggcccagtccaaggaggaggtgctgcggcacgtgcgagagatccgctaccagggcggcaataggacaaacactgggcaggccctgcagtacctttctgagcacagcttctctcccagccaaggggaccgggtagaggcacctaacctggtctacatggtcacggggaaccccgcctctgatgagatcaagaggttgcctggagacatccaggtggtacccattggggtgggcccccatgccaacatgcaggaactggagaggatcagcaggcccatcgctcccatcttcatccgggactttgagacacttccccgagaggctcctgacctggtcctgcagacatgttgctccaaggagggtctgcaactgcccaccctcccccctctccctggtatgctggaacccagtgtgtggtgatcttgcatgggacattatctgatgatgctctaacccctaggctctcatgctaacttcctggctctcaaagggcatgtgctcaaagcccggcatcagcccactgatatttgtcccacctccaggcagagtgtcactgtcattcttttgatgggaagggtctcctcttattgcctctgctcataaattcagtggtggtaggagcccatttctaaaatttaaacagtacagaggacatacagaaaacgtacaagtcctattaacccccgcttccctacccaggctagatcaccttctgcttggcaggcaacttctgctcgtttgatgattgttaactgtcctttgggccatgctggcacacggaattattccaagcagcttatgttctcagaagcaaggctaggagaccaggcggcatgtgcaggctaaacacacagatccagaagccagagtctagaactgccactcgctagctgatataattttggcctttgctaatctcctcatctagtggataaacctcgagataacttcgtataatgtatgctatacgaagttatggcgcgccataacttcgtataatgtatgctatacgaagttatgaatctaccggcagtacttttcccaaggcagtctggagcatgcgctttagcagccccgctgggcacttggcgctacacaagtggcctctggcctcgcacacattccacatccaccggtaggcgccaaccggctccgttctttggtggccccttcgcgccaccttctactcctcccctagtcaggaagttcccccccgccccgcagctcgcgtcgtgcaggacgtgacaaatggaagtagcacgtctcactagtctcgtgcagatggacagcaccgctgagcaatggaagcgggtaggcctttggggcagcggccaatagcagctttgctccttcgctttctgggctcagaggctgggaaggggtgggtccgggggcgggctcaggggcgggctcaggggcggggcgggcgcccgaaggtcctccggaggcccggcattctgcacgcttcaaaagcgcacgtctgccgcgctgttctcctcttcctcatctccgggcctttcgacctgcagccaatatgggatcggccattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgaggggatcaattctctagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctcgagatccactagttctagcctcgaggctagagcggccttataacttcgtataatgtatgctatacgaagttatctcgaggcggccatatcagcaactacttgtgaaaagcttgaaacagcatctggcataccatggtggcttaaagagtgcagctgttactgttgcttgcttgcttatttatttatttgctttgttttgagtcaggatcttattatgtgaccctgaaattctctgtgtagaccaggcatgcctcaaactcacagagatcctactgcctctgccttctgagtgctaggattaaaccattgccttactaatctatactgggatcaaatcagtaacttgtctgtctgtctgtctgtctgtctgtttctataatgagacagactaagatttgaaactctggcttgatccaagctacgcaagtacccaatcactgagtcacccagccctctttccctttctttctgagacaggatcttactaagttccccagactggccttaaaattactctgtaggccagttgagccttataacttacagtacacccccatagctaggttggcaagctgatgtccttaaagcagaagaaccagctactttcagtatctctccatccctgcgctctgtcttttttgtttatagccagcttcacagattggttaatatcgctttgatttttctgctagtaagaaaagtacctgggttgtcagcatgtgttctggctgggtgttttcttcttatggttgcagctgtggccagcttgaaccagagtgcctgccatgtgggactggcatgggatggaggaactctacctgccctagatactcaaaaacacacaagctcattttctttgctttttaagttaatgttttagttagcccataaaataatgggtttcattatattgttgttcaaacgcttcagatactttgctcacatttattactttgtcccattagtcttctctgtccctcttgctggccccttccttccttcaaatagcttcctttctattttcatcacacacacgcgcacacacacacacgcatgtgcacgcacacatggtttatgtatagatcttagattcagcatatgagaaaggatgtgatgtttgcatgtgatgtttgtgagttatttcacttaacgcagtgatctcagttctgtttacttctctgtgaatgacacaacttccttctctcccaggcactgatgccacattttcttgactccttcaaaggtcacacacagcctgtttcccagttacatgctgtactgtctctttgcccttgtttcaaccagtcttagacccaagaacacagaagcagattttctttctcttattaattgtttagctaattctcagaattatgagtcttagaatgacattttcatatatatacatatacacatacacatacacatacatatacatatatatttttttcctggcccccttcttccccacaaacaacctcagttacttttcctgttatttagagagggcagcctcttgctctcatttgcagctatttgcactgtctgtgggtagagctccagtcttttcgatgactgtcaatctagtgagcccatagattcaggaactgtctcctctgtccttctacctgacccattcccatgccctgccctccctggcaaacacgtgctcagtggtgcactgaagaccactggctgttgtgggggctgacggctggccttccattagcacctgtgacttgtgtacccatgctcttgtttctctctgcagactgcagccaacccctggatgtggtcctgctcctggatggctcctctagcttgccagagtcttcctttgataaaatgaagagttttgccaaggctttcatttcaaaggccaacattggtgagtgatacccttgaacctgcaggtgagggagtggctcttcctggttcattgattctaaatgtctcccttctccttttcctgttagggccccacctcacacaggtgtccgtgatacagtatggaagcatcaataccattgatgtaccatggaatgtggttcaggagaaagcccatctacagagtttggtggacctcatgcagcaggagggtggccccagccagattggtaatgcttggagccacgagctagatgtagaacttgtgttctgatcctcactcttgtgttctgattgagtgatcttgaccagtaactttactccttggtctgagtttctcttttgattggcgagaagctagatggtcctttgtgtcattttccagtcccaccaagtattgttctgagtcataactgctcatcttttgaatgtacctgagtcagcccttaagcccattgctcagagggtctagaatactccatcccattctttcctctcaggggatgctctggcctttgccgtgcgctatgtaacttcacaaatccacggagccaggcctggggcctccaaagcagtggtcatcatcatcatggatacctccttggatcca

SEQ ID NO: 12 - DNA, 3930 bp, Artificial
  Synthetic Construct
gatcccagcatggggtagtgaacaattctacctcactctaactgggcatcacatacaatctgaggatcagtatctatgaatattaaagatctgcccccccccccccgatctgtgaatgtaggaaggaaagggatatgggattcttggccctcaaatataacatcttggggctgtggtccaggtcacagctttcagaacccggtgggaagacattcactactgtcaccctaggctcttgctggaacaaactctgagaacctggctttgggaaggtgaggggtcctgagggtggttcctggactttttgaagtcctgcccttaggtagggaacatttattcaaatgttgacatagtcagcaccactccctgacaccaatcgagcctaatgggaacttgggttcactggggacgggctgtggctgcagctgccacagccagagcctgccgccaccatctgtacaaacaagagtgaacactgcccagggtgagttttctagcaaacacaggaaaccaggaagtatgtcactcatgcaccattaacggaggattgaggccataaattgtgggattatgaggaagtaggaattaacttcccagtaagatcctacccaccttccttgtgctgaaacagacatgaggaataagggagggggggggtaatgttcactgcaagcttacttagggcatccagctcaaagggaatggaaactctaagtctcttgtcccctcagctggcctggagaaaggtacatgataatctacattagtgtttcaaggcaggttggggacattggctagcagtgaggtcatgggacatgctcagggtttagggcactgtcaccttcttatatttactgtaaagatggaagaccaagggagcagaatggaggaggcgcattctgtgtggatccccaccacagcccatcttgatttcccttatggtctggctatgggtggctttctggaacatgacatggttgaagggtacagtgattacagagggctggaaacttgtctggtcctagagaaacctgtgggctgggcaatgactcaacacaggcttgtgggcatcatggagccggctgagggctagccactccttggtagacagcatgtctaccatctgcctgttggcagaatgggcaatggagccatcgacacttcctagatgcaacacgtctagtcctcccacaccctgtgcctgccagatggagcagtgattttacacttttatccgtcttcctgacgtcttcttactctcctttgtcttctttcccgtgaggtccagcatcattaaacaccggttcagaatccatacggcttaagttctcataacatttgtaatggtttctaatgtaaaccattcatctccagtggaacatttacaagtcccagcgtgggccaacattttagaatgctatctgtcctcagagacacttataagagggtttgaattataattaaatattaatgtggctagagacatagattggaaggaaatcaatgttacatggctatggagggcttagaacaacagattacgtttcgggttctaaaatggattaaagcttgaggaagcctctgcttcctctttaccccatctctctctctcttgcttgctctctctctctctctctctctctctctctctctctctctctctgtattcaagactcatcttgggaaaacatcaacaatgacatcccactcaggtgagacctgcttgatgggagcaggcaggacgttccatgggggacctgcgaggcagtcctgcctgcccataggtcctccctgaatcattgtggtttctttcccttctgctacagtcactgtgatggtgtgaaccttacgtgtgaagcctgccaagagcccgggggcctggtggtgcctcccacagatgccccggtgagccccaccactctgtatgtggaggacatctcggaaccgccgttgcacgatttctactgcagcaggctactggacctggtcttcctgctggatggctcctccaggctgtccgaggctgagtttgaagtgctgaaggcctttgtggtggacatgatggagcggctgcgcatctcccagaagtgggtccgcgtggccgtggtggagtaccacgacggctcccacgcctacatcgggctcaaggaccggaagcgaccgtcagagctgcggcgcattgccagccaggtgaagtatgcgggcagccaggtggcctccaccagcgaggtcttgaaatacacactgttccaaatcttcagcaagatcgaccgccctgaagcctcccgcatcgccctgctcctgatggccagccaggagccccaacggatgtcccggaactttgtccgctacgtccagggcctgaagaagaagaaggtcattgtgatcccggtgggcattgggccccatgccaacctcaagcagatccgcctcatcgagaagcaggcccctgagaacaaggccttcgtgctgagcagtgtggatgagctggagcagcaaagggacgagatcgttagctacctctgtgaccttgcccctgaagcccctcctcctactctgcccccccacatggcacaagtcactgtgggccccgggatcgctgggatctcgtcaccgggaccaaaacggaagtccatggttctggatgtggtgtttgtcctggaggggtcagacgaagttggtgaagccaacttcaataagagcaaggagttcgtggaggaggtaatccagcgcatggacgtgagcccggatgcaacgcgcatctcagtactgcagtattcctacacggtaaccatggagtatgccttcaatggggcccagtccaaggaggaggtgctgcggcacgtgcgagagatccgctaccagggcggcaataggacaaacactgggcaggccctgcagtacctttctgagcacagcttctctcccagccaaggggaccgggtagaggcacctaacctggtctacatggtcacggggaaccccgcctctgatgagatcaagaggttgcctggagacatccaggtggtacccattggggtgggcccccatgccaacatgcaggaactggagaggatcagcaggcccatcgctcccatcttcatccgggactttgagacacttccccgagaggctcctgacctggtcctgcagacatgttgctccaaggagggtctgcaactgcccaccctcccccctctccctggtatgctggaacccagtgtgtggtgatcttgcatgggacattatctgatgatgctctaacccctaggctctcatgctaacttcctggctctcaaagggcatgtgctcaaagcccggcatcagcccactgatatttgtcccacctccaggcagagtgtcactgtcattcttttgatgggaagggtctcctcttattgcctctgctcataaattcagtggtggtaggagcccatttctaaaatttaaacagtacagaggacatacagaaaacgtacaagtcctattaacccccgcttccctacccaggctagatcaccttctgcttggcaggcaacttctgctcgtttgatgattgttaactgtcctttgggccatgctggcacacggaattattccaagcagcttatgttctcagaagcaaggctaggagaccaggcggcatgtgcaggctaaacacacagatccagaagccagagtctagaactgccactcgctagctgatataattttggcctttgctaatctcctcatctagtggataaacctcgagataacttcgtataatgtatgctatacgaagttatggcgcgccataacttcgtataatgtatgctatacgaagttatgaatctaccggcagtacttttcccaaggcagtctggagcatgcgctttagcagccccgctgggcacttggcgctacacaagtggcctctggcctcgcaca

SEQ ID NO: 13 - DNA, 732 bp, Artificial
  Synthetic Construct
cccgggggcctggtggtgcctcccacagatgccccggtgagccccaccactctgtatgtggaggacatctcggaaccgccgttgcacgatttctactgcagcaggctactggacctggtcttcctgctggatggctcctccaggctgtccgaggctgagtttgaagtgctgaaggcctttgtggtggacatgatggagcggctgcgcatctcccagaagtgggtccgcgtggccgtggtggagtaccacgacggctcccacgcctacatcgggctcaaggaccggaagcgaccgtcagagctgcggcgcattgccagccaggtgaagtatgcgggcagccaggtggcctccaccagcgaggtcttgaaatacacactgttccaaatcttcagcaagatcgaccgccctgaagcctcccgcatcgccctgctcctgatggccagccaggagccccaacggatgtcccggaactttgtccgctacgtccagggcctgaagaagaagaaggtcattgtgatcccggtgggcattgggccccatgccaacctcaagcagatccgcctcatcgagaagcaggcccctgagaacaaggccttcgtgctgagcagtgtggatgagctggagcagcaaagggacgagatcgttagctacctctgtgaccttgcccctgaagcccctcctcctactctgcccccccacatggcacaagtcactgtgggccccgggcccggg

SEQ ID NO: 14 - DNA, 38 bp, Artificial
  Synthetic Construct
cgagataacttcgtataatgtatgctatacgaagttat

SEQ ID NO: 15 - DNA, 34 bp, Artificial
  Synthetic Construct
ataacttcgtataatgtatgctatacgaagttat

SEQ ID NO: 16 - DNA, 35 bp, Artificial
  Synthetic Construct
tataacttcgtataatgtatgctatacgaagttat

SEQ ID NO: 17 - DNA, 1650 bp, Artificial
  Synthetic Construct
gaatctaccggcagtacttttcccaaggcagtctggagcatgcgctttagcagccccgctgggcacttggcgctacacaagtggcctctggcctcgcacacattccacatccaccggtaggcgccaaccggctccgttctttggtggccccttcgcgccaccttctactcctcccctagtcaggaagttcccccccgccccgcagctcgcgtcgtgcaggacgtgacaaatggaagtagcacgtctcactagtctcgtgcagatggacagcaccgctgagcaatggaagcgggtaggcctttggggcagcggccaatagcagctttgctccttcgctttctgggctcagaggctgggaaggggtgggtccgggggcgggctcaggggcgggctcaggggcggggcgggcgcccgaaggtcctccggaggcccggcattctgcacgcttcaaaagcgcacgtctgccgcgctgttctcctcttcctcatctccgggcctttcgacctgcagccaatatgggatcggccattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgaggggatcaattctctagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctcgagatccactagttctagcctcgaggctagagcggcct

SEQ ID NO: 18 - DNA, 2359 bp, Artificial
  Synthetic Construct
gcttaaagagtgcagctgttactgttgcttgcttgcttatttatttatttgctttgttttgagtcaggatcttattatgtgaccctgaaattctctgtgtagaccaggcatgcctcaaactcacagagatcctactgcctctgccttctgagtgctaggattaaaccattgccttactaatctatactgggatcaaatcagtaacttgtctgtctgtctgtctgtctgtctgtttctataatgagacagactaagatttgaaactctggcttgatccaagctacgcaagtacccaatcactgagtcacccagccctctttccctttctttctgagacaggatcttactaagttccccagactggccttaaaattactctgtaggccagttgagccttataacttacagtacacccccatagctaggttggcaagctgatgtccttaaagcagaagaaccagctactttcagtatctctccatccctgcgctctgtcttttttgtttatagccagcttcacagattggttaatatcgctttgatttttctgctagtaagaaaagtacctgggttgtcagcatgtgttctggctgggtgttttcttcttatggttgcagctgtggccagcttgaaccagagtgcctgccatgtgggactggcatgggatggaggaactctacctgccctagatactcaaaaacacacaagctcattttctttgctttttaagttaatgttttagttagcccataaaataatgggtttcattatattgttgttcaaacgcttcagatactttgctcacatttattactttgtcccattagtcttctctgtccctcttgctggccccttccttccttcaaatagcttcctttctattttcatcacacacacgcgcacacacacacacgcatgtgcacgcacacatggtttatgtatagatcttagattcagcatatgagaaaggatgtgatgtttgcatgtgatgtttgtgagttatttcacttaacgcagtgatctcagttctgtttacttctctgtgaatgacacaacttccttctctcccaggcactgatgccacattttcttgactccttcaaaggtcacacacagcctgtttcccagttacatgctgtactgtctctttgcccttgtttcaaccagtcttagacccaagaacacagaagcagattttctttctcttattaattgtttagctaattctcagaattatgagtcttagaatgacattttcatatatatacatatacacatacacatacacatacatatacatatatatttttttcctggcccccttcttccccacaaacaacctcagttacttttcctgttatttagagagggcagcctcttgctctcatttgcagctatttgcactgtctgtgggtagagctccagtcttttcgatgactgtcaatctagtgagcccatagattcaggaactgtctcctctgtccttctacctgacccattcccatgccctgccctccctggcaaacacgtgctcagtggtgcactgaagaccactggctgttgtgggggctgacggctggccttccattagcacctgtgacttgtgtacccatgctcttgtttctctctgcagactgcagccaacccctggatgtggtcctgctcctggatggctcctctagcttgccagagtcttcctttgataaaatgaagagttttgccaaggctttcatttcaaaggccaacattggtgagtgatacccttgaacctgcaggtgagggagtggctcttcctggttcattgattctaaatgtctcccttctccttttcctgttagggccccacctcacacaggtgtccgtgatacagtatggaagcatcaataccattgatgtaccatggaatgtggttcaggagaaagcccatctacagagtttggtggacctcatgcagcaggagggtggccccagccagattggtaatgcttggagccacgagctagatgtagaacttgtgttctgatcctcactcttgtgttctgattgagtgatcttgaccagtaactttactccttggtctgagtttctcttttgattggcgagaagctagatggtcctttgtgtcattttccagtcccaccaagtattgttctgagtcataactgctcatcttttgaatgtacctgagtcagcccttaagcccattgctcagagggtctagaatactccatcccattctttcctctcaggggatgctctggcctttgccgtgcgctatgtaacttcacaaatccacggagccaggcctggggcctccaaagcagtggtcatcatcatcatggatacctccttggatcca

SEQ ID NO: 19 - DNA, 1453 bp, Artificial
  Synthetic Construct
gcatgctccagaccttccttcccctcttgcatgttctgtcttctccagcttccattctgggcttttatacttgctgtgctcactgtgtggaacattctttccccagctgtatttgtggctccttcatccttatctcaggtctttctccatgaaacgattctcactctctgtgcattttccatccttcccatctcctagtttcttgcaagcatctctctgcatctgaaaatgccatgcatttgccttttatgtttgctatttatgtcctgctggtgttggctatgaggacaggagacagggactttacctgctcagcatccccattgcttagacctgtgcctgatacacagcagtagctcattagcacctgctgcctgcttgggcatctccctcaaaggcttgctggtgtgtggccagcttggattgcatgaggctctgtttggctgcctgatgtagtgttatgtgttgtgctcacctgaccatagaccctttcttgttgcctggctaatgtaggagtggcagtgtttcccgttggggttggggatcggtatgatgaagcccagctgaggatcttggcaggccctggggccagctccaatgtggtaaagctccagcaagttgaagacctctccaccatggccaccctgggcaactccttcttccacaaactgtgttctggtgagtcttctgatatgtttcctttttcagttctcaagcaaccactaccaccctggccagcaccaaaccagaaaaccccttaaaaacaaagttaccattccgaagactcaaagatacctgtatataaaatttcgtcttagatgaagagtgaagactgtcacgtggaccagaactttcacatttctgttggtgaaggtgtgcgctgccacctttaggggtctctggagagtttcctgtgtacaagcctgcttctgcttaaactttccagctgactttttagggagaggaagtcccggcttgtttgtactgaggggattttgaatgaaagacttgagagacataagcaaaaacatcccagcatcctaatgagccatggtcctctgcttactctcagtatacatgtaaaggactttagaaatacaccatcgaatccaacattgaaggaccacaggctaaattgactttaggttagaaacgtgttttgttttctggcctggttttatatattaatatgattagttttaaactaaaaccttctatggagttatggacctttcctgatggatggtagctatcttggcagcggtcgttaagcagatctctttcaagttttatgtgatctggtttcacaccccccacacccactcctggaaggcatggaaggagaaaaatgaaagctactgccatgtgatagctgatgttatcacaattattgactgtcataaagtacgtctgaagggccacaggatggtcatctccttgtgtcctcggatcc

SEQ ID NO: 20 - DNA, 1398 bp, Artificial
  Synthetic Construct
ggacactggcttcttgccttgctgagagtcacatggtcaccaccttttcttttgtctccacagcccagagctgtgaagaaaagaatgttcgggaaaatggctatgagtgtgagtggcgttataacagctgtgcgcctgcttgcccagtcacgtgtcagcaccctgagcctctggcttgccctgtgcagtgtgtggagggttgtcatgcacattgccctccaggtgaggcctctttgactggaacaggctggagggatggagggatgggttcttagacaggtgtgcttcttcttcatctgttctccactgtccaagggtgcctccctccaacctccagcccccaaaaatgtctgtttgacaggatctctttgtcaaagccagacagctctgatgccacattggggctgtatcatctgtcctttgatttttcagtagtgaggaaagaacttgccccctccctgagctctcccatgtagtcacaccctattgcttcttaaacagttttttgtgggcctatttgtggatttatcatatagaacagtccgtcttctttccttccttccttccttccttccttcctcccttccttccttccttccttccttccttccttccttccttccttctttcctttccttccctcctttcctttcctttcctttcctttcctttcctttcctttcctttcctttcctttcctttcctttcctttcctttcctttcctttcctttcctttccttccttcctttctcctctcctctctccctcactcctttcctccctctctttctcctttcctctttccctctccccgtagagtcttgctgtgtatctcaggctgtcttcaaactcaccatccttctgcctcaacaaagtgtcagcaccctgtgtgagttagagaagcaatgtgctccagcagaatttgcagtgatggctatggtccattcaatgcaatgatgcctagtagatgtgaggaaatggcttcctcatctcactgaattatgcttagtttacatgagcagcctcatggcaggacatgttggatagcccagctctaaagtattgagttcatgctcgggaagagtgagctgagccaagctgttccttgcctccttggggataagatttcatttgtcctagggctccggggaagggagggttgtggtataggaagctccatctcagcttctcagaggggcagacaatgcaccgagaaagaaggatgctccctgtgtgctgaggtgatcactgaggagtctggagagtgagtttctgggaaggttcccctagtggcaggtgggtggagctacctggtatgagaggtgactggtgggctcctaatgaggtgtcttgtgcagggagaatcctggatgaacttctgcagacctgcgtagacc

SEQ ID NO: 21 - DNA, 24 bp, Artificial
  Synthetic construct
acatcccactcaggtgagacctgc

SEQ ID NO: 22 - DNA, 30 bp, Artificial
  Synthetic Construct
ctggctagcagtcaggagcagagtgatatg

SEQ ID NO: 23 - DNA, 26 bp, Artificial
  Synthetic Construct
gccttgcttctgagaacataagctgc

SEQ ID NO: 24 - DNA, 33 bp, Artificial
  Synthetic Construct
agaacaaggccttcgtgctgagcagtgtggatg

SEQ ID NO: 25 - protein, 2813 aa, Artificial
  Synthetic construct
MNPFRYEICLLVLALTWPGTLCTEKPRDRPSTARCSLFGDDFINTFDETMYSFAGGCSYLLAGDCQKRSFSILGNFQDGKRMSLSVYLGEFFDIHLFANGTVTQGDQSISMPYASQGLYLEREAGYYKLSSETFGFAARIDGNGNFQVLMSDRHFNKTCGLCGDFNIFAEDDFRTQEGTLTSDPYDFANSWALSSEEQRCKRASPPSRNCESSSGDMHQAMWEQCQLLKTASVFARCHPLVDPESFVALCEKILCTCATGPECACPVLLEYARTCAQEGMVLYGWTDHSACRPACPAGMEYKECVSPCPRTCQSLSINEVCQQQCVDGCSCPEGELLDEDRCVQSSDCPCVHAGKRYPPGTSLSQDCNTCICRNSLWICSNEECPGECLVTGQSHFKSFDNRYFTFSGICQYLLARDCEDHTFSIVIETMQCADDPDAVCTRSVSVRLSALHNSLVKLKHGGAVGIDGQDVQLPFLQGDLRIQHTVMASVRLSYAEDLQMDWDGRGRLLVKLSPVYSGKTCGLCGNYNGNKGDDFLTPAGLVEPLVVDFGNAWKLQGDCSDLRRQHSDPCSLNPRLTRFAEEACALLTSSKFEACHHAVSPLPYLQNCRYDVCSCSDSRDCLCNAVANYAAECARKGVHIGWREPGFCALGCPQGQVYLQCGNSCNLTCRSLSLPDEECSEVCLEGCYCPPGLYQDERGDCVPKAQCPCYYDGELFQPADIFSDHHTMCYCEDGFMHCTTSGTLGSLLPDTVLSSPLSHRSKRSLSCRPPMVKLVCPADNPRAQGLECAKTCQNYDLECMSLGCVSGCLCPPGMVRHENKCVALERCPCFHQGAEYAPGDTVKIGCNTCVCRERKWNCTNHVCDATCSAIGMAHYLTFDGLKYLFPGECQYVLVQDYCGSNPGTFQILVGNEGCSYPSVKCRKRVTILVDGGELELFDGEVNVKRPLRDESHFEVVESGRYVILLLGQALSVVWDHHLSISVVLKHTYQEQVCGLCGNFDGIQNNDFTTSSLQVEEDPVNFGNSWKVSSQCADTRKLSLDVSPATCHNNIMKQTMVDSACRILTSDVFQGCNRLVDPEPYLDICIYDTCSCESIGDCACFCDTIAAYAHVCAQHGQVVAWRTPTLCPQSCEEKNVRENGYECEWRYNSCAPACPVTCQHPEPLACPVQCVEGCHAHCPPGRILDELLQTCVDPQDCPVCEVAGRRLAPGKKITLSPDDPAHCQNCHCDGVNLTCEACQEPGGLVVPPTDAPVSPTTLYVEDISEPPLHDFYCSRLLDLVFLLDGSSRLSEAEFEVLKAFVVDMMERLRISQKWVRVAVVEYHDGSHAYIGLKDRKRPSELRRIASQVKYAGSQVASTSEVLKYTLFQIFSKIDRPEASRIALLLMASQEPQRMSRNFVRYVQGLKKKKVIVIPVGIGPHANLKQIRLIEKQAPENKAFVLSSVDELEQQRDEIVSYLCDLAPEAPPPTLPPHMAQVTVGPGIAGISSPGPKRKSMVLDVVFVLEGSDEVGEANFNKSKEFVEEVIQRMDVSPDATRISVLQYSYTVTMEYAFNGAQSKEEVLRHVREIRYQGGNRTNTGQALQYLSEHSFSPSQGDRVEAPNLVYMVTGNPASDEIKRLPGDIQVVPIGVGPHANMQELERISRPIAPIFIRDFETLPREAPDLVLQTCCSKEGLQLPTLPPLPDCSQPLDVVLLLDGSSSLPESSFDKMKSFAKAFISKANIGPHLTQVSVIQYGSINTIDVPWNVVQEKAHLQSLVDLMQQEGGPSQIGDALAFAVRYVTSQIHGARPGASKAVVIIIMDTSLDPVDTAADAARSNRVAVFPVGVGDRYDEAQLRILAGPGASSNVVKLQQVEDLSTMATLGNSFFHKLCSGFSGVCVDEDGNEKRPGDVWTLPDQCHTVTCLANGQTLLQSHRVNCDHGPRPSCANSQSPVRVEETCGCRWTCPCVCTGSSTRHIVTFDGQNFKLTGSCSYVIFQNKEQDLEVLLHNGACSPGAKQACMKSIEIKHAGVSAELHSNMEMAVDGRLVLAPYVGENMEVSIYGAIMYEVRFTHLGHILTYTPQNNEFQLQLSPKTFASKMHGLCGICDENGANDFTLRDGTVTTDWKRLVQEWTVQQPGYTCQAVPEEQCPVSDSSHCQVLLSASFAECHKVIAPATFHTICQQDSCHQERVCEVIASYAHLCRTSGVCVDWRTTDFCAMSCPPSLVYNHCERGCPRHCDGNTSFCGDHPSEGCFCPQHQVFLEGSCVPEEACTQCVGEDGVRHQFLETWVPDHQPCQICMCLSGRKINCTAQPCPTARAPTCGPCEVARLKQSTNLCCPEYECVCDLFNCNLPPVPPCEGGLQPTLTNPGECRPTFTCACRKEECKRVSPPSCPPHRTPTLRKTQCCDEYECACSCVNSTLSCPLGYLASATTNDCGCTTTTCLPDKVCVHRGTVYPVGQFWEEGCDTCTCTDMEDTVVGLRVVQCSQRPCEDSCQPGFSYVLHEGECCGRCLPSACKVVAGSLRGDSHSSWKSVGSRWAVPENPCLVNECVRVEDAVFVQQRNISCPQLAVPTCPTGFQLNCETSECCPSCHCEPVEACLLNGTIIGPGKSVMVDLCTTCRCIVQTDAISRFKLECRKTTCEACPMGYREEKSQGECCGRCLPTACTIQLRGGRIMTLKQDETFQDGCDSHLCRVNERGEYIWEKRVTGCPPFDEHKCLAEGGKIVKIPGTCCDTCEEPDCKDITAKVQYIKVGDCKSQEEVDIHYCQGKCASKAVYSIDIEDVQEQCSCCLPSRTEPMRVPLHCTNGSVVYHEVINAMQCRCSPRNCSK

SEQ ID NO: 26 - protein, 242 aa, Mus musculus
PGGLVAPPTDAPVSSTTPYVEDTPEPPLHNFYCSKLLDLVFLLDGSSMLSEAEFEVLKAFVVGMMERLHISQKRIRVAVVEYHDGSRAYLELKARKRPSELRRITSQIKYTGSQVASTSEVLKYTLFQIFGKIDRPEASHITLLLTASQEPPRMARNLVRYVQGLKKKKVIVIPVGIGPHASLKQIRLIEKQAPENKAFLLSGVDELEQRRDEIVSYLCDLAPEAPAPTQPPQVAHVTVSPG

SEQ ID NO: 27 - protein, 242 aa, Homo sapiens
PGGLVVPPTDAPVSPTTLYVEDISEPPLHDFYCSRLLDLVFLLDGSSRLSEAEFEVLKAFVVDMMERLRISQKWVRVAVVEYHDGSHAYIGLKDRKRPSELRRIASQVKYAGSQVASTSEVLKYTLFQIFSKIDRPEASRIALLLMASQEPQRMSRNFVRYVQGLKKKKVIVIPVGIGPHANLKQIRLIEKQAPENKAFVLSSVDELEQQRDEIVSYLCDLAPEAPPPTLPPHMAQVTVGPG

SEQ ID NO: 28 - DNA, 22 bp, Artificial
  Synthetic Construct
gctgtgctcgacgttgtcactg

SEQ ID NO: 29 - DNA, 23 bp, Artificial
  Synthetic Construct
ggaagagagcttgggaacctagc

SEQ ID NO: 30 - DNA, 23 bp, Artificial
  Synthetic Construct
cccacccactcatctctctgaag

SEQ ID NO: 31 - DNA, 21 bp, Artificial
  Synthetic Construct
aagcgcatgctccagactgcc

(19) United States
(12) Patent Application Publication
(10) Pub. No.: US 2013/0347134 A1
(43) Pub. Date: Dec. 26, 2013

(54) TRANSGENIC VON WILLEBRAND FACTOR ANIMALS AND USES THEREOF

(71) Applicants: Thomas Diacovo, Larchmont, NY (US); Jianchun Chen, Fort Lee, NJ (US)

(72) Inventors: Thomas Diacovo, Larchmont, NY (US); Jianchun Chen, Fort Lee, NJ (US)

(21) Appl. No.: 13/923,088

(22) Filed: Jun. 20, 2013

Related U.S. Application Data
(60) Provisional application No. 61/662,896, filed on Jun. 21, 2012.

(51) Int. Cl. A61K 49/00

(52) U.S. Cl. CPC A61K 49/0008
USPC 800/3; 435/7.9; 435/13; 435/320.1; 436/501; 530/381; 536/23.5; 800/4; 800/14; 800/18

(57) ABSTRACT

The present invention provides, inter alia, transgenic non-human animals, such as transgenic mice. The animals contain in their genome a polynucleotide encoding a von Willebrand factor (VWF) polypeptide, which polypeptide forms a thrombus when in the presence of human platelets. Nucleic acid sequences and vectors for generating the transgenic non-human animals, and methods for using the transgenic non-human animals are provided as well. Chimeric VWF proteins are also provided.

### CROSS-REFERENCE TO RELATED APPLICATIONS

The present invention claims benefit to U.S. provisional application Ser. No. 61/662,896 filed Jun. 21, 2012, the entire contents of which are incorporated by reference.

### FIELD OF INVENTION

The present invention provides, inter alia, a transgenic non-human animal, such as a transgenic mouse, containing in its genome a polynucleotide encoding a von Willebrand factor (VWF) polypeptide. Nucleic acid sequences and vectors for generating the transgenic non-human animals, and methods using the transgenic non-human animals are provided as well.

### INCORPORATION BY REFERENCE OF SEQUENCE LISTING

This application contains references to amino acids and/or nucleic acid sequences that have been filed concurrently herewith as sequence listing text file 0352923.txt, file size of 137 KB, created on Jun. 20, 2013. The aforementioned sequence listing is hereby incorporated by reference in its entirety pursuant to 37 C.F.R. §1.52(e)(5).

### BACKGROUND OF THE INVENTION

The ability of platelets to rapidly stick to the damaged wall of arterial blood vessels is critical for preventing blood loss (hemorrhage). Inappropriate deposition of these hemostatic cells in arterial blood vessels due to pathological disease processes such as atherosclerosis can result in lack of blood flow to vital organs such as the heart and brain. Thus a delicate balance exists between providing adequate hemostasis without causing blockage of blood vessels by excessive platelet deposition (a.k.a. thrombus formation).

von Willebrand Factor (VWF) is a multidomain, plasma glycoprotein of complex multimeric structure which is synthesized by vascular endothelial cells and megakaryocytes (Jaffe et al., 1973; Nachman et al., 1977; Sporn et al., 1985). Its presence in the blood is vital to maintaining the integrity of the vasculature. To accomplish this task, VWF forms a “bridge” between the injured vessel wall and platelets by virtue of its ability to interact with extracellular matrix components, such as collagen, and receptors expressed on platelets, such as glycoprotein Ib alpha (Sakariassen et al., 1979; Meyer et al., 1983; Cruz et al., 1995; Handa et al., 1986; Murata et al., 1991; Fressinaud et al., 1988). It also binds to and confers stability to factor VIII (Wiss et al., 1977). The importance of this glycoprotein in hemostasis is underscored by the occurrence of clinical bleeding when the plasma VWF levels fall below 50 IU/dL (type I von Willebrand's disease, abbreviated as “VWD”), or when functional defects in the protein occur (Type 2 VWD, which includes 4 subtypes: Type 2A, Type 2B, Type 2M, and Type 2N) (Ewenstein et al., 1997; Sadler et al., 1995).

Upon surface immobilization of VWF at sites of vascular injury, it is the role of the A1 domain of VWF (which includes e.g., residues 1240-1481, such as residues 1260-1480) to initiate the process of platelet deposition at sites of vascular injury and under conditions of high rates of shear flow (>1,000 s⁻¹) (Ruggeri et al., 2006). The critical nature of this interaction is exemplified by the bleeding disorder, termed type 2M VWD, which results from the incorporation of loss-of-function mutations within this domain that perturb interactions with GPIb alpha (Sadler et al., 2006; Rabinowitz et al., 1992; Cruz et al., 2000). In addition, recombinant VWF multimers lacking the A1 domain cannot support platelet adhesion at high rates of flow despite retaining the ability to interact with collagen (Sixma et al., 1991).

The structure of the A1 domain includes the α/β fold with a central β-sheet flanked by α-helices on each side as well as one intra-disulfide bond (Cys1272-Cys 1458), but no MIDAS motif (Emsley et al., 1998). Its overall shape is cuboid, with the top and bottom faces forming the major and minor binding sites, respectively, that interact with the concave surface of GPIb α. The most extensive contact site buries about 1700 Å² of surface area, interacting with leucine-rich repeat (LRR) five to eight and the C-terminal flank of the GPIb α (Huizing a et al., 2002). For this to occur, the β-switch region of this platelet receptor undergoes a conformation change so that it aligns itself with the central beta sheet of the A1 domain. The smaller site (about 900 Å²) accommodates the binding of the β-finger and the first LRR of GPIb α, an event that appears to require the displacement of the amino-terminal extension of the A1 domain. Based on these findings as well as the preferential localization of mutations in humans within this region, which enhance GPIb α binding, it is speculated that the amino-terminal extension regulates the adhesive properties of this domain. This is also supported by the fact that recombinant A1 proteins lacking this extension have a higher affinity for this platelet receptor (Sugimoto et al., 1993). Despite these observations, the physiological relevance of such structural changes in this receptor-ligand pair remains to be determined as well as the contribution of other domains to this process.

In addition to its role in hemostasis, VWF also contributes to pathological thrombus formation on the arterial side of the circulation. This may be the consequence of injury to the blood vessel wall from inflammatory disease states and/or medical/surgical interventions. Pathological thrombus formation is the leading cause of death in the Western world. Thus, pharmaceutical companies have committed considerable resources towards the research and design of drugs to prevent or treat thrombosis. However, there remains an urgent need to develop new and improved therapies such as those aimed at reducing platelet and/or VWF interactions with the injured arterial wall. One major hurdle hindering drug development in this field is the lack of an appropriate small animal model of thrombosis to test promising therapies. For instance, differences in the structure or isoform of protein receptors or ligands on mouse vs. human platelets that are critical for the activation and/or binding of these cells to the injured vessel wall preclude testing of drugs developed against human platelets in a mouse model of thrombosis. Moreover, this issue cannot be overcome by simply transfusing mice with human platelets because mouse VWF does not support significant interactions with human cells (see below). Thus, the development of “humanized” mouse models of hemostasis and thrombosis would potentially expedite drug discovery and testing.

Biophysical and molecular approaches are essential for understanding the structure-function relationship between a receptor and its ligand. Thus, the ability to study such interactions in an appropriate physiological and/or pathological setting is desirable. To do so, one requires an animal model that is amenable to genetic manipulation and has receptor-ligand interactions that closely resemble those found in humans. Thrombosis models in hamsters and guinea pigs have proven useful in pharmacological studies, but a mouse model would prove to be more beneficial based on the ability to insert or delete genes of interest, accessibility of tissues for study, and cost and ease of handling (Yamamoto, et al., 1998, Azzam et al., 1995). Regarding GPIb α-VWF interactions, two groups have significantly advanced the understanding of the importance of these interactions in mediating thrombosis by generating mice deficient in these proteins (Denis, et al., 1998, Ware, et al., 2000). Yet, no information regarding the role of the biophysical properties of the GPIb α-VWF-A1 in regulating the processes of thrombosis and hemostasis are obtained.

Thus, there is a need for a biological platform for testing of drugs to be used in human beings. The present invention addresses these and other needs.

### SUMMARY OF THE INVENTION

One embodiment of the present invention is a transgenic non-human animal. This animal comprises in its genome a polynucleotide encoding a von Willebrand factor (VWF) polypeptide, wherein the transgenic non-human animal expresses the VWF and forms a thrombus when in the presence of human platelets.

Another embodiment of the present invention is a transgenic mouse. This mouse comprises in its genome a polynucleotide encoding a von Willebrand factor (VWF) polypeptide, wherein the transgenic mouse expresses the VWF and forms a thrombus when in the presence of human platelets.

A further embodiment of the present invention is a nucleic acid sequence. This nucleic acid sequence comprises SEQ ID NO:13.

An additional embodiment of the present invention is a vector. This vector comprises a nucleic acid sequence comprising SEQ ID NO:13.

A further embodiment of the present invention is a mouse-human chimeric polypeptide sequence. This sequence comprises the amino acid sequence of SEQ ID NO:25.

An additional embodiment of the present invention is a method for identifying a candidate agent that modulates human platelet mediated thrombosis. This method comprises:
(a) providing a candidate agent;
(b) providing a non-human transgenic animal disclosed herein;
(c) administering the candidate agent to the non-human transgenic animal or to VWF produced by the non-human transgenic animal; and
(d) evaluating an effect, if any, of the candidate agent on human platelet mediated thrombosis in the non-human transgenic animal or the VWF produced by the non-human transgenic animal by detecting an alteration in interactions between the VWF and human platelets.

Yet another embodiment of the present invention is a method for identifying a candidate agent that modulates human platelet mediated thrombosis. This method comprises:
(a) providing a candidate agent;
(b) providing a transgenic mouse disclosed herein;
(c) administering the candidate agent to the transgenic mouse or to VWF produced by the transgenic mouse; and
(d) evaluating an effect, if any, of the candidate agent on human platelet mediated thrombosis in the transgenic mouse or the VWF produced by the transgenic mouse by detecting an alteration in interactions between the VWF and human platelets.

Another embodiment of the present invention is a method for determining whether platelet function or morphology in a subject is abnormal. This method comprises:

a) affixing a protein comprising a VWF-A1 domain obtained from a transgenic non-human animal disclosed herein to a surface of a flow chamber;

b) perfusing through the flow chamber a volume of blood or plasma from a subject at a shear flow rate of at least about 100 s⁻¹;

c) perfusing a targeted molecular imaging agent into the flow chamber; and

d) comparing the flow rate of the blood or plasma from the subject as compared to a normal flow rate, so as to determine whether the subject's platelet function or morphology is abnormal.

Yet another embodiment of the present invention is a method for producing chimeric von Willebrand Factor A1 protein that specifically binds to human platelets. This method comprises:

(a) providing a non-human animal expressing a chimeric von Willebrand Factor A1 protein, wherein the chimeric protein causes the platelet binding specificity of the non-human animal von Willebrand Factor A1 protein to change to be specific for human platelets; and

(b) harvesting the chimeric von Willebrand Factor A1 from the non-human animal, which specifically binds human platelets.

Another embodiment of the present invention is a method for calibrating an aggregometry device or a device for measuring clot formation or retraction. This method comprises:

a) providing hematologic data obtained from a subject, wherein blood or platelets from the subject is assessed by the device;

b) determining whether or not a thrombotic event occurs in a transgenic non-human animal disclosed herein, wherein the animal is perfused with a sample of blood or platelets from the subject; and

c) correlating data obtained from step (b) with the data obtained in step (a) so as to calibrate the device, wherein a certain data obtained from the device is indicative of the corresponding thrombotic outcome determined in the transgenic non-human animal.

### BRIEF DESCRIPTION OF THE DRAWINGS

This patent or application file contains at least one drawing executed in color. Copies of this patent or patent application publication with color drawing(s) will be provided by the Office upon request and payment of the necessary fee.

FIG. 1A is a schematic representation of the prepro form of VWF. From top to bottom: repeated homologous regions; A1 and A3 disulfide loops and functional domains of the mature VWF subunit.

FIG. 1B is an illustration depicting the sequential adhesive and activation events that promote platelet deposition at sites of vascular injury.

FIG. 2 is a model depicting the location of residues in the human VWF-A1 domain that, if mutated, diminish GPIb alpha-mediated platelet binding under flow conditions.

FIG. 3A is a structure model depicting residues associated with type 2M or type 2B VWD. FIG. 3B shows a silver stained gel that clinically depicts a type 2B VWD disease state individual, which is characterized by a loss of circulating high molecular weight VWF multimers (HMWM, FIG. 3B, Lane 2).

FIG. 4 shows structure models of the human VWF-A1 domain. FIG. 4A shows the location of the Ile1309 mutation and its proposed effects on residues critical for GPIb binding. FIG. 4B shows the loss of the isoleucine methyl group allows a water molecule to enter, which ultimately results in changes in orientation of the G1324 peptide plane and the side chain of H1326 as depicted, residues critical for GPIb binding.

FIG. 5 is a space-filling model of the botrocetin-A1 complex with sites involved in GPIb alpha binding and location of type 2B mutations indicated (FIG. 5A), wherein botrocetin does not alter the conformation of VWF-A1. In FIG. 5B, minor conformational changes in the A1 domain are represented. Uncomplexed and complexed mutant domains are superimposed onto the WT structure.

FIG. 6 is schematic wherein the uncomplexed A1 domain, an amino-terminal extension appears to block a binding site for the amino-terminal β-hairpin (arrows) of GPIb alpha. Binding requires the amino-terminal extension of A1 to move, and also induces the β-switch (loop) of GPIb alpha to form a β-strand motif.

FIG. 7 depicts microscope images wherein the use of platelets in lieu of recombinant proteins or transfected cells as the immobilized substrate enables evaluation of GPIb alpha in its native form (i.e. correct orientation and proper post-translational modification). Platelet coverage of <10% can be bound in this manner and can remain relatively unactivated for up to 30 minutes as evident by morphology on light microscopic examination (FIG. 7A) and lack of expression of P-selectin by fluorescence microscopy (FIG. 7B).

FIG. 8 shows an assay for quantitating bead-platelet interactions under flow conditions. FIG. 8A demonstrates the direct visualization of bead-platelet interaction under flow conditions (60×DIC microscopy). An approaching bead moving at a velocity of 609±97 μm/sec (wall shear stress of 1.5 dyn cm⁻²) is captured by a surface-immobilized platelet at (t=12.8 msec), pivots a distance of less than 3 μm in under 40 msec, and is then released after a pause time of tₚ=228.2 msec into the flow stream (escape velocity=288±90.4 μm/sec). FIG. 8B is a plot of distance vs. time based on the visualization. FIG. 8C depicts representative experiments of k_{off} values for WT human VWF-A1 coated beads based on a distribution of interaction (pause) times. FIG. 8D shows that the kinetics of the GPIb alpha tether bond are identical whether platelets are metabolically inactivated or fixed in paraformaldehyde to prevent activation upon surface-immobilization.

FIG. 9 shows the deduced single-letter amino acid sequence of portions of mouse VWF-A1 domain (M VWF) compared to its human counterpart (H VWF) from amino acid 1260 to 1480. The locations of cysteines forming the loop structure are numbered (1272 and 1458). Conversion of the arginine (R) residue 1326 in the mouse A1 domain to histidine (H) as found in its human counterpart (x) enables mouse VWF to bind human platelets.

FIG. 10 represents graphs of ristocetin-induced platelet aggregation assays (RIPA). Concentrations of the ristocetin modulator known to cause agglutination of human platelets (about 1.0 mg/ml) had no effect using murine platelet rich plasma (FIG. 10B and FIG. 10D). Incubation of murine platelet rich plasma (PRP) with thrombin resulted in >90% platelet aggregation (FIG. 10A). Concentrations of ≧2.5 mg/ml of modulator resulted in murine platelet aggregation (30%, FIG. 10C).

FIG. 11 depicts a multimer gel analysis of purified VWF from human (lane 1, FIG. 11A) and mouse (lane 2, FIG. 11A) plasma. The ability of human and mouse VWF to mediate platelet adhesion in flow was determined in order to evaluate platelet interactions between human and murine VWF with GPIb alpha, as depicted in the bar graph of FIG. 11B. Surface-immobilized murine VWF supports adhesion of syngeneic platelets (1×10⁸/ml) at a shear rate encountered in the arterial circulation (1600 s⁻¹) as observed for the human plasma protein (FIG. 11B, first panel). In contrast, murine VWF did not support significant interactions with human platelets and vice versa.

FIG. 12 is an image of a gel of mouse and human VWF-A1 highly purified protein, which was dialyzed against 25 mM Tris-HCl, 150 mM NaCl, 0.05% Tween 20, pH 7.8. SDS-PAGE analysis revealed a prominent protein band of 34,000 Da for mouse VWF-A1 under non-reducing conditions.

FIG. 13 depicts bar graphs of a series of in vitro flow chamber assays performed to assess platelet adhesion, in which human or murine platelets (5×10⁷/ml) were infused through a parallel plate flow chamber containing glass cover slips coated with either human (H) VWF-A1 or murine (M) VWF-A1 protein (100 μg/ml final concentration) at a shear rate of 800 s⁻¹. M VWF-A1 protein supported platelet adhesion as efficiently as its human counterpart under physiological flow conditions (FIG. 13A). The translocation of mouse platelets occurred to a similar degree as its human counterpart under physiological flow conditions (FIG. 13B). However, human platelets had a reduced capacity to interact with M VWF-A1 protein and mouse platelets had a reduced capacity to interact with H VWF-A1 protein in flow.

FIG. 14A shows purified bacterial His-tagged VWF-A1 protein and non-His tagged VWF-A1 protein that was analyzed by SDS-PAGE (12.5%) under non-reducing and reducing conditions. FIG. 14B depicts a bar graph of a human platelet adhesion assay to recombinant VWF proteins with and without the presence of a His-tag at a shear rate of 800 s⁻¹.

FIG. 15 shows models of the crystal structure of VWF-A1 domains solved using a recombinant protein. The main chain schematic of the mouse VWF-A1 domain, with β-strands (arrows) and helices (coils), is shown in FIG. 15A. FIG. 15B demonstrates that the C-alpha atoms of human and mouse VWF-A1 domains closely overlap. FIG. 15C shows the model of the murine VWF-A 1 domain and the residues that purportedly interact with GPIb alpha.

FIG. 16 shows graphs that depict platelet adhesion assays (FIG. 16A) and platelet translocation measurements (FIG. 16B). The ability of murine and human platelets to interact with a mutant protein substrate (human VWF-A1 domain wherein amino acid residue 1326 was mutated from H is to Arg and mouse VWF-A1 domain wherein amino acid residue 1326 was mutated from Arg to His) was evaluated at a wall shear rate of 800 s⁻¹.

FIG. 17 shows data from an ELISA assay. Following several injections of mouse (M) VWF-A1, serum was collected from rats and screened by ELISA for anti-VWF-A1 antibodies. Spleens from animals with the highest antibody titers were harvested and splenocytes fused with Sp2/0 mouse myeloma cells (Alon et al., 1998). Supernatants of hybridomas were screened for reactivity to murine (M) VWF-A1 by ELISA. Pre-immune rat serum was used as control. Mabs to M VWF-A1 not only reacted with WT and mutant proteins (1324G>S) but also recognized native VWF purified from mouse plasma.

FIG. 18 shows representative graphs depicting the distribution of interaction times for more than 35 individual transient attachment events at various times. Analysis of the distribution of interaction times between human or murine VWF-A1 coated beads and their respective platelet substrates, as measured by high temporal resolution video microscopy, indicate that >95% of all transient tether bond events fit a straight line, the regressed slope of which corresponded to a single k_{off}, wherein the cellular off-rates of these quantal units of adhesion for the wild type human (H) and murine (M) proteins are found in FIGS. 18A and B and M VWF-A1 protein containing the type 2B mutation 11309V (1309I>V) corresponds to FIG. 18C.

FIG. 19 shows graphs that represent an assessment of transient tether events (FIG. 19A) and analysis of the distribution of interaction times (FIG. 19B) between human VWF-A1 coated microspheres and human immobilized platelets. The type 2B mutation Ile1309 Val (1309I>V) was incorporated into recombinant human (H) VWF-A1 containing either the type 2M mutation Gly1324Ser (1324G>S) or the function reducing mutation His1326Arg (1326H>R).

FIG. 20 is a scheme for generating transgenic mice with mutant VWF-A1 domains. FIG. 20A is a diagram of a knock-in construct for proposed mutations in the VWF-A1 domain of mice. FIG. 20B represents Southern blot hybridization with probe “a” or “b”, respectively, to determine if the construct was appropriately targeted.

FIG. 21 shows Southern blot analysis wherein heterozygous and homozygous mice for the amino acid substitution at residue 1326 (R1326H; 1326R>H) display the Arg1326His mutation (lanes 2 and 3 respectively) while wild-type animals did not (lane 1).

FIGS. 22A-C show sequence analysis of purified PCR products of WT, heterozygous, or homozygous VWF-A1 domains, respectively, wherein the boxed area denotes the conversion of Arg to His (CGT in FIG. 22A wherein the codon corresponds to Arg and CAT in FIG. 22C wherein the codon corresponds to the amino acid His).

FIG. 23 is a graph of an ELISA assay which demonstrated that conversion of 1326 Arg to His in the mouse A1 domain did not alter plasma protein levels of VWF in mutant mice nor its ability to form multimers. The ELISA assay detected mouse VWF in plasma obtained from WT and homozygous R1326H (KI) animals, but not from plasma obtained from animals deficient in VWF (VWF KO).

FIG. 24 is a gel image of multimer gel analysis of plasma VWF that revealed an identical banding pattern between mouse and human VWF. Incorporation of His at position 1326 in the mouse A1 domain had no effect on multimerization of VWF in mutant mice.

FIG. 25 is a graphical representation of the bleeding times (s) observed in the mutant VWF-A1 mice that are either heterozygous or homozygous for the 1326R>H mutation. Results are compared to normal counterparts and VWF-deficient mice. Tail cut=1 cm.

FIG. 26 is a bar graph depicting thrombus formation induced by perfusion of whole blood from either wild type (WT) or homozygous mutant mice (for the 1326R>H mutation) over surface-immobilized collagen in vitro wherein an 80% reduction in thrombus formation was observed compared to WT controls.

FIG. 27 shows micrographs that demonstrate reduced thrombus formation occurring when whole blood from either the knock-in animals (homozygous for the R1326H mutation) or WT is perfused over collagen-coated cover slips at a shear rate of 1600 s⁻¹ indicating a 70% reduction in thrombi formed on collagen as compared to WT controls.

FIG. 28 demonstrates a platelet adhesion assay under flow conditions. R1326H mutant mouse VWF promotes interactions with human platelets under physiologic flow conditions, wherein anticoagulated human blood was infused over surface-immobilized WT or mutant mouse plasma VWF at 1600 s⁻¹ as shown in the micrographs of FIG. 28A. FIG. 28B is a graph that depicts the amount of human platelets that bound to WT murine VWF or R1326H mutant murine VWF.

FIG. 29 shows transmitted light micrographs demonstrating that homozygous R1326H mutant mice infused with human (FIG. 29A) but not mouse platelets (FIG. 29B) were able to generate an arterial thrombus that occludes the vessel lumen in response to laser-induced vascular injury as depicted by intravital microscopy.

FIG. 30 is a bar graph that depicts the average bleeding time for mice receiving blood-banked human platelets (about 3 minutes for a 1 cm tail cut) or given an intravenous infusion of a physiological buffered saline solution (10 minutes (end point)).

FIG. 31 is a schematic representing the isolation of the cremaster muscle and the catheter set-up used in intravital microscopy assays to assess thrombus formation.

FIG. 32 is a schematic of an intravital microscopy method.

FIG. 33 demonstrates images of mouse platelet interactions and a bar graph of such interactions in a wild type animal. FIG. 33A are representative intravital photomicrographs that depict the range of platelet interactions that occur at a site of vascular injury (60×). Platelets were observed to either transiently pause (*) or rapidly tether to and translocate (TP) on damaged arterial endothelium. A composite image demonstrates translocation of two platelets over a 3 second interval of time (panel 6). FIG. 33B depicts interacting platelets at the site of arterial injury that were classified as either undergoing translocation or firm adhesion (sticking) during an observation period of 1 minute.

FIG. 34 shows representative photomicrographs that depict the vessel wall in a wild type mouse in the (A) absence of injury or (B) post-laser-induced injury as visualized under transillumination (40× objective). Thrombus is indicated by the arrows.

FIG. 35 is a graphical representation of the bleeding phenotype observed in the mutant VWF-A1 1326R>H heterozygous or homozygous mouse compared to its WT counterpart or mice without VWF (VWF KO) when tails were cut either 5 mm (FIG. 35A) or 15 mm (FIG. 35B) from the tip of the tail.

FIGS. 36A-B are graphs that depict ex vivo analysis of human platelet interactions with plasma VWF or recombinant VWF-A1 proteins. Accumulation of human platelets on surface-immobilized plasma human or mouse VWF (FIG. 36A) or recombinant human or mouse A1 domain proteins (FIG. 36B) after 4 minutes of perfusion with whole blood (shear rate of 1600 s⁻¹) is shown. Data are representative of three separate experiments performed in triplicate (mean±s.e.m.).

FIGS. 36C-D are graphs that depict ex vivo analysis of mouse platelet interactions with plasma VWF or recombinant VWF-A1 proteins. Accumulation of murine platelets on surface-immobilized human or mouse plasma VWF (FIG. 36C) or recombinant human or mouse A1 domain proteins (FIG. 36D) after 4 minutes of perfusion with whole blood (shear rate of 1600 s⁻¹) is shown. Data are representative of three separate experiments performed in triplicate (mean±s.e.m.).

FIGS. 37A-B are structural representations of human and murine VWF-A1 domains. FIG. 37A depicts the alignment of Ca atoms for human and murine A1 domains. Key residues are shown as spheres or as ball-and-stick side-chains. FIG. 37B shows a view rotated 90° about a horizontal axis to reveal the packing of residue 1397 (Phe in human, Leu in mouse) that results in a 3 Å shift (arrow) of helix α4.

FIGS. 37C-D are structural representations of human and murine GPIbα-VWF-A1 complexes. FIG. 37C depicts the model of the murine-murine complex. FIG. 37D depicts the crystal structure of the human-human complex. Salt bridges are circled and key residue differences are boxed. Zooms reveal details of the electrostatic interactions at the β-switch contact region. The region of contact involving helix α3 of the A1 domain and one face of the LRR repeats of GPIbα is highly conserved between species, except for two residue changes that do not participate in bond formation: GPIbα E151K and VWF-A1 G1370S (human:mouse). Thus, minor differences in this region are unlikely to contribute to a reduction in binding between the murine and human proteins. This is also the case with the contact area located at the bottom of the A1 domain, which is invariant in both species and participates in salt-bridge formation (circle).

FIG. 37E is a model of the human GPIbα-murine A1 complex, showing the loss (arrow) and gain (circle) of salt-bridges. The upper zoom shows the interspecies interface at the β-switch region, revealing the electrostatic clash. The lower zoom shows the murine VWF-A1 point mutant 1326R>H, which removes the electrostatic clash and now closely resembles the human-human complex.

FIG. 37F is a model of the murine GPIbα-human VWF-A1 complex. Two salt-bridges are lost as compared to the murine complex; murine GPIbα D238 with residue 1326 due to the R>H change in human VWF-A1, and murine GPIbα K237 with residue 1330 owing to the E>G change in the human protein. Moreover, neither the chimeric nor murine complex forms a salt-bridge between residues 225 and 1395 on GPIbα and VWF-A1, respectively, as compared to its human counterpart (circle). The upper zoom shows the interspecies interface at the β-switch region; there is no electrostatic clash but no salt-bridge can form with H1326. The lower zoom shows the human point mutant 1326H>R, which adds a salt-bridge and now closely resembles the murine-murine complex.

FIG. 37G is a graph that shows the accumulation of human platelets on surface-immobilized recombinant WT murine VWF-A1 domain proteins, those containing the selected mutations 1326R>H, 1330E>G and 1370S>G, or WT human VWF-A1 protein (shear rate of 1600 s⁻¹). Data are representative of three separate experiments performed in triplicate (mean±s.e.m.).

FIG. 38A is schematic for the generation of the VWF^{1326R>H} mouse that represents the targeting strategy for insertion of exon 28 containing adenine in lieu of guanine at position 3977 of the cDNA for murine VWF. Abbreviations: R1, EcoRI; RV, EcoRV; B, BamHI; X, XhoI; pGK-TK, pGK-Neo, thymidine kinase/neomycin resistance cassette; [symbol] , loxP sites.

FIG. 38B is a blot of a Southern analysis of tailed DNA digested with EcoR1. Wild-type (WT) allele, 14 kb; mutant allele, 6 kb using Probe A. FIG. 38C represents the DNA sequencing of the tailed DNA demonstrating successful incorporation of adenine at position 3977 in heterozygous and homozygous animals (CGT>CAT). Sequence analysis of genomic DNA from these animals, 2 kb upstream and 6 kb downstream of exon 28, did not reveal any other alterations in nucleotide base pairs that would affect VWF production and/or function.

FIGS. 39A-B show the analysis of VWF gene transcription and translation. FIG. 39A is a gel of RT-PCR of lung tissue from WT or mutant VWF mice to detect for A1, A2, and/or A3 domain message. β-actin was analyzed to demonstrate use of equivalent amounts of mRNA. FIG. 39B is a graph demonstrating VWF antigen levels in plasma obtained from WT littermates (pooled) or six individual mice homozygous for 1326R>H mutation as detected by ELISA. Data are representative of two independent experiments performed in triplicate.

FIG. 39C is a gel showing the analysis of VWF multimers in plasma from WT or homozygous VWF^{1326R>H} animals. Normal human plasma as well as that obtained from a patient with type 2B VWD is shown for comparison.

FIG. 39D shows an analysis of factor VIII activity in homozygous VWF^{1326R>H} animals as compared to WT littermate controls (n=2; performed in triplicate).

FIG. 39E shows an analysis of collagen binding of VWF activity in homozygous VWF^{1326R>H} animals as compared to WT littermate controls and mice without VWF (VWF^{-/-}) (n=2; performed in triplicate). OD, optical density.

FIG. 40 depicts representative photomicrographs showing murine platelet accumulation at sites of laser-induced arteriolar injury in WT or homozygous mutant animals 20 seconds and 2 minutes post-injury. White lines demarcate the extent of the thrombus.

FIG. 41A is a graphical representation of the tail bleeding times (in seconds) for heterozygous and homozygous VWF^{1326R>H} and WT mice when tails were cut 1 cm from the tip of the tail. Each point represents one individual mouse and experiments were performed on five separate days.

FIG. 41B is an ex vivo analysis of human platelet interactions with surface-immobilized plasma VWF^{1326R>H} at a shear rate of 1,600 s⁻¹. A role for GPIb alpha on human platelets is demonstrated by the function-blocking antibodies to this platelet receptor (mAb 6D1 and mAb AP-1) to prevent adhesion in flow.

FIG. 41C shows microscopy images of in vivo analysis of human platelet interactions with murine plasma VWF^{1326R>H} using infused fluorescently labeled human platelets into the vasculature of the cremaster muscle of mice. Human platelet accumulation was examined at sites of laser-induced arteriolar injury in WT (n=10) or homozygous mutant animals (n=12) using 2 channel confocal microscopy with excitation wavelengths of 488 nm (BCECF) and 561 nm (rhodamine 6G). Representative composite images of fluorescent images depicting human thrombus formation in homozygous mutant (upper panels) or WT (lower panels) mice (V=venule; A=arteriole).

FIG. 41D is a bar graph depicting the composition of thrombi (% of total area) in WT or homozygous VWF^{1326R>H} animals.

FIG. 41E is a bar graph measuring thrombus size during an in vivo study of human platelet interactions with plasma VWF^{1326R>H} to determine the effect of GPIbα or αIIb β3 blockade on human platelet adhesion in vivo. The requirement for GPIb alpha-mediated adhesion is shown by the ability of a function-blocking antibody (mAB 6D1, mAB AP-1, or mAb 7E3) to GPIb alpha to prevent human platelet thrombus formation in vivo. Fluorescently labeled human platelet accumulation was examined at sites of laser-induced arteriolar injury in WT (n=6) or homozygous mutant animals (n=8). Data represent the mean±s.e.m.

FIG. 41F is a graphical representation of tail bleeding times (in seconds) for homozygous VWF^{1326R>H} that received an infusion of either normal saline or human platelets prior to severing 10 mm of distal tail, wherein the ability of human platelets to restore hemostasis in homozygous VWF^{1326R>H} was examined. Each point represents one individual mouse and experiments were performed on 3 separate days.

FIGS. 41G and H show the effect of Plavix or ReoPro on laser-induced human platelet thrombus formation (n=4 per genotype with minimum of 4 arterioles per animal) (FIG. 41G) or hemostasis (FIG. 41H).

FIG. 42 is a schematic depicting a perfluorocarbon nanoparticle capable of incorporating imaging agents (Gd⁺³, ⁹⁹ mTc) and chemotherapeutics into the outer layers. Antibodies complexed to the surface of the particle can target the agent to specific sites within the body.

FIG. 43 is a photographic image depicting the accumulation of fluorescent PNP, coupled to an antibody that recognizes human alphaIIb beta 3 on the surface of human platelets, at a site of vascular injury in homozygous 1326R>H mutant mice infused with human platelets.

FIG. 44 is a graphical representation of the structure of the VWF-A1-GPIb alpha-botrocetin ternary complex. FIG. 44A is a ribbon representation. FIG. 44B demonstrates the location of the previously unknown interface between GPIb alpha and botrocetin.

FIG. 45A is a schematic representation showing that recombinant GPIb alpha is surface-immobilized in a 96 well format. After blocking potential non-specific binding sites, recombinant VWF-A1 containing a His tag is added to the wells and allowed to interact with GPIb alpha for a specified period of time. The unbound material is removed by washing the wells and the complex formed between the 2 proteins detected by the addition of a HRP-conjugated antibody that binds to the His tag on A1. The amount of bound A1 can then be quantified by either fluorescence (addition of LumiGlow) or by color change.

FIG. 45B is an image representing that the specificity of the interaction can be determined by the addition of the GPIb function blocking antibody 6D1 prior to the addition of recombinant VWF-A1. DMSO (0.5%) was added to illustrate that this reagent does not interfere with the assay.

FIG. 46 shows graphs depicting the effect of Plavix (FIG. 46A) or ReoPro (FIG. 46B) on human platelet-induced hemostasis in homozygous VWF^{1326R>H} mice.

FIG. 47 is a graph showing the efficacy of anti-platelet drugs administered to patients by studying the ability of platelets harvested from patients on platelet adhesion in the VWF^{1326R>H} mouse.

FIG. 48 shows a schematic of the main chain of the human VWF A1 domain depicting residues that differ from murine VWF A1 domain.

FIG. 49 shows a graph demonstrating the inability of mAb AvW3 to detect mouse plasma VWF or recombinant mouse VWF A1 domain by ELISA, as measured by optical density (OD).

FIG. 50 is a graph showing that MAb AvW3 does not reduce the ability of mouse platelets to accumulate on surface-immobilized mouse plasma VWF in flow (SR 1600 s⁻¹)

FIG. 51 shows a schematic of the targeting vectors used in the generation of a VWF transgenic mouse. FIG. 51A shows the original construct, and FIG. 51B shows the modified human Alknock-in construct. Only the construct shown in FIG. 51B was successful in generating a mouse containing a region of the human VWF A1 domain (from amino acid 1240P through 1481G).

FIG. 52A is a Southern blot identifying mice bearing the human VWF A1 domain. FIG. 52B shows the translated results of genomic DNA sequencing of the indicated WT mouse, human and mouse-human chimeric DNA.

FIG. 53 is a graph showing evaluation of mice homozygous for the VWF-HA1 substitution (VWF HA1), which revealed evidence of a significant bleeding diathesis as manifested by tail bleeding times of greater than 10 minutes.

FIG. 54 is a graph showing that platelet counts in VWF HA1 mice were similar to WT littermate controls.

FIG. 55 is a graph showing that plasma levels in VWF HA1 mice were similar to WT littermate controls.

FIG. 56 is a bar graph showing that circulating mouse platelets were unable to participate in thrombus formation in injured arterioles of VWF HA1 mice.

FIG. 57 is a bar graph showing that human platelets could support thrombus formation in injured arterioles of VWF HA1 mice.

FIG. 58 is a bar graph showing that GPIbα on mouse platelets could not support any significant interactions with plasma VWF containing the human A1 domain.

FIG. 59 is a bar graph showing that human platelets could interact at levels observed for human plasma VWF.

FIG. 60 is a line graph showing optical aggregometry data for human platelets resuspended in platelet-poor plasma from VWF^{R1326H} and VWF^{HA1} mice and subsequently treated with ristocetin. A trace generated from human plasma treated with ristocetin is shown as a control. The left axis corresponds to percent aggregration.

### DETAILED DESCRIPTION OF THE INVENTION

One embodiment of the present invention is a transgenic non-human animal. This animal comprises in its genome a polynucleotide encoding a von Willebrand factor (VWF) polypeptide, wherein the transgenic non-human animal expresses the VWF and forms a thrombus when in the presence of human platelets.

Assays for thrombus formation are known in the art (See e.g., Harrison's Principle of Internal Medicine, 15th ed. (Chapter 116) 2001, McGraw Hill, Columbus, Ohio) and include the in vivo (the tail bleeding assay, for example) and in vitro (ex vivo platelet adhesion study, for example) assays disclosed in the Example section. More details with respect to methods for assessing thrombotic events in vivo are set forth below.

In one aspect of this embodiment, the VWF polypeptide comprises the A1 domain of human VWF polypeptide, preferably amino acids 1240P through 1481G of the full length human VWF polypeptide, the amino acid sequence of which is depicted in SEQ ID NO:6. The literature appears to loosely define the bounds of the A1 domain of human VWF to include amino acids 1240-1481, 1260-1480, and other ranges (see, e.g., Emsley et al., 1998 (residues 1238-1472); Bonnefoy et al., 2006 (residues 1262-1492); Huang et al., 2009 (residues 1260-1468)).

In another aspect of this embodiment, the VWF polypeptide is at least 85%, preferably at least 90%, identical to the amino acid sequence depicted in SEQ ID NO:25, which is a chimeric VWF protein in which the human VWF A1 domain replaces the non-human, e.g., mouse, VWF A1 domain. For example, the VWF polypeptide is at least 91%, 92%, 93%, 94%, 95%, 96%, 97%, 98%, 99%, 99.5%, 99.7%, or 99.9% identical to the amino acid sequence depicted in SEQ ID NO:25.

In an additional aspect of this embodiment, the polynucleotide encodes a VWF to which AvW3 specifically binds. AvW3 is a monoclonal antibody that specifically recognizes the human VWF-A1 domain and blocks VWF's interaction with GPIb. (See e.g., Mancuso et al., 1996; Kroner et al., 1992; Rathore et al., 2003). AvW3 is available from commercial vendors such as Linscott's USA, catalog #GTI-V3A, Mill Valley, Calif.).

In yet another aspect of this embodiment, the animal may be any useful non-human laboratory or agricultural animal. For example, the animal may be selected from the group consisting of mouse, rat, hamster, guinea pig, rabbit, dog, goat, horse, and monkey. Preferably, the animal is a mouse.

Another embodiment of the present invention is a transgenic mouse. This mouse comprises in its genome a polynucleotide encoding a von Willebrand factor (VWF) polypeptide, wherein the transgenic mouse expresses the VWF and forms a thrombus when in the presence of human platelets.

In one aspect of this embodiment, the VWF polypeptide comprises amino acids 1240P through 1481G of SEQ ID NO:6.

In another aspect of this embodiment, the VWF polypeptide is at least 85%, preferably at least 90%, identical to the amino acid sequence depicted in SEQ ID NO:25. For example, the VWF polypeptide is at least 91%, 92%, 93%, 94%, 95%, 96%, 97%, 98%, 99%, 99.5%, 99.7%, or 99.9% identical to the amino acid sequence depicted in SEQ ID NO:25.

In an additional aspect of this embodiment, the polynucleotide encodes a VWF to which the monoclonal antibody AvW3 specifically binds.

A further embodiment of the present invention is a nucleic acid sequence. This nucleic acid sequence comprises, consists essentially of, or consists of, SEQ ID NO:13, which depicts the nucleic acid sequence encoding the human VWF A1 domain. We note that SEQ ID NO:13 is not a naturally occurring nucleotide sequence, because while greater than 85% of the human A1 domain sequence was substituted for its murine counterpart, a small portion of the murine A1 domain sequence still remains.

An additional embodiment of the present invention is a vector. This vector comprises, consists essentially of, or consists of, the nucleic acid sequence depicted in SEQ ID NO:13. For example, the nucleic acid sequence of one such vector is depicted in SEQ ID NO:11.

A further embodiment of the present invention is a mouse-human chimeric polypeptide sequence. This sequence comprises, consists essentially of, or consists of, the amino acid sequence of SEQ ID NO:25.

In another aspect of this embodiment, the VWF polypeptide is at least 85%, preferably at least 90%, identical to the amino acid sequence depicted in SEQ ID NO:25. For example, the VWF polypeptide is at least 91%, 92%, 93%, 94%, 95%, 96%, 97%, 98%, 99%, 99.5%, 99.7%, or 99.9% identical to the amino acid sequence depicted in SEQ ID NO:25.

An additional embodiment of the present invention is a method for identifying a candidate agent that modulates human platelet mediated thrombosis. This method comprises:
(a) providing a candidate agent;
(b) providing a non-human transgenic animal disclosed herein;
(c) administering the candidate agent to the non-human transgenic animal or to VWF produced by the non-human transgenic animal; and
(d) evaluating an effect, if any, of the candidate agent on human platelet mediated thrombosis in the non-human transgenic animal or the VWF produced by the non-human transgenic animal by detecting an alteration in interactions between the VWF and human platelets.

In one aspect of this embodiment, the VWF polypeptide comprises amino acids 1240P through 1481G of SEQ ID NO:6.

In another aspect of this embodiment, the VWF polypeptide is at least 85%, preferably at least 90%, identical to the amino acid sequence depicted in SEQ ID NO:25. For example, the VWF polypeptide is at least 91%, 92%, 93%, 94%, 95%, 96%, 97%, 98%, 99%, 99.5%, 99.7%, or 99.9% identical to the amino acid sequence depicted in SEQ ID NO:25.

In an additional aspect of this embodiment, the polynucleotide encodes a VWF to which the monoclonal antibody AvW3 specifically binds.

In a further aspect of this embodiment, the animal is as defined above. The animal may be selected from the group consisting of mouse, rat, hamster, guinea pig, rabbit, dog, goat, horse, and monkey. Preferably, the animal is a mouse.

Yet another embodiment of the present invention is a method for identifying a candidate agent that modulates human platelet mediated thrombosis. This method comprises:
(a) providing a candidate agent;
(b) providing a transgenic mouse disclosed herein;
(c) administering the candidate agent to the transgenic mouse or to VWF produced by the transgenic mouse; and
(d) evaluating an effect, if any, of the candidate agent on human platelet mediated thrombosis in the transgenic mouse or the VWF produced by the transgenic mouse by detecting an alteration in interactions between the VWF and human platelets.

In one aspect of this embodiment, the VWF polypeptide comprises amino acids 1240P through 1481G of SEQ ID NO:6.

In another aspect of this embodiment, the VWF polypeptide is at least 85%, preferably at least 90%, identical to the amino acid sequence depicted in SEQ ID NO:25. For example, the VWF polypeptide is at least 91%, 92%, 93%, 94%, 95%, 96%, 97%, 98%, 99%, 99.5%, 99.7%, or 99.9% identical to the amino acid sequence depicted in SEQ ID NO:25.

In an additional aspect of this embodiment, the polynucleotide encodes a VWF to which the monoclonal antibody AvW3 specifically binds.

In a further aspect of this embodiment, the evaluating step (i.e., step (d)) comprises the use of a diagnostic assay for determining GPIb-alpha-VWF-A1 protein interaction. As used herein, “GPIb-alpha-VWF-A1 protein interaction” means the binding or other association between the platelet GPIb-alpha and the A1 domain of a chimeric VWF protein according to this embodiment.

In another aspect of this embodiment, the diagnostic assay comprises perfusing platelets into a flow chamber at a shear flow rate of at least 100s⁻¹, wherein the VWF protein is immobilized on a bottom surface of the chamber. In the present invention, other shear flow rates may be used, particularly those set forth in more detail in the Examples below, or as may be determined by one skilled in this art. In one aspect of this embodiment, the administration of the candidate agent and the perfusion of the platelets occur sequentially. For example, the perfusion of platelets may occur prior to administration of the candidate agent. Perfusion of platelets may be followed by perfusion of a labeled agent, as defined in more detail below. The labeled agent may target a platelet receptor, a VWF protein, or a portion thereof. The platelets used in this embodiment preferably are not murine platelets; preferably, they are human platelets.

In another preferred embodiment, the diagnostic assay comprises perfusing platelets into the transgenic mouse. In this embodiment, the administration of the candidate agent and the perfusion of the platelets may occur sequentially. For example, the perfusion of platelets may occur prior to administration of the candidate agent. Perfusion of platelets may be followed by perfusion of a labeled agent. In the present invention, the labeled agent may be any agent capable of providing a detectable signal and that does not substantially interfere with the evaluating step. For example, the labeled agent may comprise one or more of a nanoparticle, a fluorophore, a quantum dot, a microcrystal, a radiolabel, a dye, a gold biolabel, an antibody, or a small molecule ligand. The labeled agent may target a platelet receptor, a VWF protein, or a portion thereof. The platelets used in this embodiment preferably are not murine platelets; preferably, they are human platelets.

In an additional aspect of this embodiment, the evaluating step comprises detecting an increase or decrease in the dissociation rate between the VWF produced by the transgenic mouse and GPIb-alpha protein by at least two-fold.

Certain candidate agents may slow the on-rate, and/or increase the off-rate (k_{off}) binding kinetics, and/or reduce bond strength of the interaction between VWF-A1, e.g., the VWF produced by a non-human transgenic animal, preferably a transgenic mouse according to the present invention, and GPIb-alpha by at least two-fold, thus resulting in a decreased lifetime of the bond(s). Such candidate agents could reduce thrombosis formation. Other candidate agents may abbreviate off-rate (k_{off}) binding kinetics between VWF-A1, e.g., the VWF produced by a non-human transgenic animal, preferably a transgenic mouse according to the present invention, and GPIb-alpha by at least two-fold, thus resulting in a prolongation in the lifetime of the bond(s). Such candidate agents could promote platelet adhesion due to the compound stabilizing an interaction between VWF-A1, e.g., the VWF produced by a non-human transgenic animal, preferably a transgenic mouse according to the present invention, and GPIb-alpha. To assess binding efficiency between VWF-A1, e.g., the VWF produced by a non-human transgenic animal, preferably a transgenic mouse according to the present invention, and GPIb-alpha, binding kinetics can be determined by measuring translocation velocity, tethering frequency, and bond strength (Fukuda, K., et al., (2005) Nat. Struct. Mol. Biol. 12:152-159; Doggett, et al., (2003) Blood 102(10): 152-60; Doggett, T. A. et al. (2002) Biophys. J. 83, 194-205; Schmidtke and Diamond (2000) J Cell Bio 149(3): 719-29; Mody et al., (2005) Biophys. J. 88: 1432-43, all of which are incorporated by reference in their entirety).

The candidate agents, including compounds identified and tested using the methods described above can be anti-platelet drugs. In one embodiment, the anti-platelet drug can be a cyclooxygenase inhibitor, a phosphodiesterase inhibitor, an adenosine diphosphate receptor inhibitor, a PI3K inhibitor, an adenosine reuptake inhibitors, thrombin receptor inhibitor or inhibitor of any intracellular signaling pathway in platelets, an alphaIIb beta3 inhibitor, an alpha2 beta1 inhibitor, a glycoprotein V inhibitor, a glycoprotein VI inhibitor, a PECAM-1 inhibitor or any adhesion molecule and/or activation pathway critical for human platelet function.

In a further aspect of this embodiment, the evaluating step comprises detecting an increase or decrease of platelet adhesion to a surface expressing VWF produced by the transgenic mouse of the present invention.

In another aspect of this embodiment, the evaluating step comprises detecting an increase or decrease in a stabilization of an interaction between VWF produced by the transgenic mouse of the present invention and GPIb-alpha protein.

In an additional aspect of this embodiment, the evaluating step comprises detecting thrombosis formation.

In yet another aspect of this embodiment, the evaluating step comprises identifying an occurrence of an abnormal thrombotic event in the transgenic mouse. Non-limiting examples of an abnormal thrombotic event may comprise abnormal bleeding, abnormal clotting, death, or a combination thereof. As used herein, “abnormal” refers to clinical abnormality, which is readily determined by a physician.

In a further aspect of this embodiment, the evaluating step comprises any suitable method or assay such as, e.g., dynamic force microscopy, a coagulation factor assay, a platelet adhesion assay, thrombus imaging, a bleeding time assay, aggregometry, review of real-time video of blood flow, a Doppler ultrasound vessel occlusion assay, or a combination thereof. These assays are known in the art. For example, Merkel et al. (1999) discloses using dynamic force microscopy to detect and measure receptor-ligand bonds. These assays are disclosed below in the Examples section.

Another embodiment of the present invention is a method for determining whether platelet function or morphology in a subject is abnormal. This method comprises:

a) affixing a protein comprising a VWF-A1 domain, preferably including amino acids 1240P-1481G of SEQ ID NO:6, obtained from a transgenic non-human animal disclosed herein to a surface of a flow chamber;

b) perfusing through the flow chamber a volume of blood or plasma from a subject at a shear flow rate of at least about 100 s⁻¹;

c) perfusing a targeted molecular imaging agent into the flow chamber; and

d) comparing the flow rate of the blood or plasma from the subject as compared to a normal flow rate, so as to determine whether the subject's platelet function or morphology is abnormal. In this assay method, the lack of platelet binding may suggest functional defects in the subject's platelets.

As used herein, a “subject” is a mammal, preferably, a human. In addition to humans, categories of mammals within the scope of the present invention include, for example, agricultural animals, domestic animals, laboratory animals, etc. Some examples of agricultural animals include bovines, porcines, equines, goats, etc. Some examples of domestic animals include canines, felines, etc. Some examples of laboratory animals include murines, rabbits, guinea pigs, hamsters, etc. In one aspect of this embodiment, the subject is selected from the group consisting of a human, a canine, a feline, a murine, a porcine, an equine, or a bovine.

In another aspect of this embodiment, the affixing comprises:

(i) coating a surface of the chamber with an antibody that specifically binds VWF-A1 domain, preferably including amino acids 1240P-1481G of SEQ ID NO:6 and

(ii) perfusing the VWF-A1 protein, preferably including amino acids 1240P-1481G of SEQ ID NO:6 produced by the transgenic mouse in the flow chamber at a shear flow rate of at least 100 s⁻¹.

In yet another aspect of this embodiment, the targeted molecular imaging agent includes any agent capable of providing a detectable signal and that does not substantially interfere with the method. Preferably, the imaging agent comprises a nanoparticle, a fluorophore, a quantum dot, a microcrystal, a radiolabel, a dye, a gold biolabel, an antibody, a peptide, a small molecule ligand, or a combination thereof.

In a further aspect of this embodiment, the targeted molecular imaging agent binds to a platelet receptor, a platelet ligand, or any region of a VWF protein or a portion thereof.

In an additional aspect of this embodiment, the targeted molecular imaging agent comprises horseradish peroxidase (HRP) coupled to an antibody that specifically binds to VWF-A1 or a fragment thereof, preferably amino acids 1240P-1481G of SEQ ID NO:6. Following binding, a reaction with diaminobenzidine (DAB) can be performed where DAB is reduced by HRP to produce a brown precipitate at the site of binding. This technique allows for enzymatic, calorimetric detection of binding that can be visualized by transmitted light microscopy. For example, if the antibody is directed at a platelet receptor, and calorimetric detection represents whether the antibody bound to the platelet-VWF-A1 complex, the absence of color would denote the lack of a complex formation, thus suggesting that platelets were unable to bind to VWF-A1.

In another aspect of this embodiment, the comparing step comprises a platelet adhesion assay, fluorescence imaging, a chromogenic indicator assay, a microscopy morphology analysis, or any combination thereof.

In a further aspect of this embodiment, platelets bound to VWF-A1 are less than about 500 cells/mm².

In yet another aspect of this embodiment, the platelets are substantially spherical.

The normal platelet morphology is discoid with some spherical shaping, but some are substantially spherical in shape. To further analyze platelet morphology, gross platelet histology can be assessed via light microscopy or electron microscopy. In another embodiment, platelets having an abnormal morphology are greater than about 2 μm in diameter. (Ross M H, Histology: A text and atlas 3rd edition, Williams and Wilkins, 1995: Chapter 9). Various assays can be used to assess whether platelet function is normal, such as a platelet adhesion assay, fluorescence imaging, a chromogenic indication, microscopy morphology analysis, or those listed in Harrison's Principle of Internal Medicine, 15th ed. ((Chapter 116) 2001, McGraw Hill, Columbus, Ohio), which are hereby incorporated by reference.

In an additional aspect of this embodiment, the protein comprising the VWF-A1 is affixed to the chamber with any appropriate agent. Representative, non-limiting examples of such an agent include an antibody, a peptide, and a Fab fragment that specifically binds to a VWF polypeptide or a portion thereof.

Yet another embodiment of the present invention is a method for producing chimeric von Willebrand Factor A1 protein that specifically binds to human platelets. This method comprises:

(a) providing a non-human animal expressing a chimeric von Willebrand Factor A1 protein, wherein the chimeric protein causes the platelet binding specificity of the non-human animal von Willebrand Factor A1 protein to change to be specific for human platelets; and

(b) harvesting the chimeric von Willebrand Factor A1 from the non-human animal, which specifically binds human platelets.

In one aspect of this embodiment, the chimeric von Willebrand Factor A1 protein comprises amino acids 1240P through 1481G of SEQ ID NO:6.

In another aspect of this embodiment, the chimeric von Willebrand Factor A1 protein is at least 85%, preferably at least 90%, identical to the amino acid sequence depicted in SEQ ID NO:25. For example, the chimeric von Willebrand Factor A1 protein is at least 91%, 92%, 93%, 94%, 95%, 96%, 97%, 98%, 99%, 99.5%, 99.7%, or 99.9% identical to the amino acid sequence depicted in SEQ ID NO:25.

In an additional aspect of this embodiment, monoclonal antibody AvW3 specifically binds to the chimeric von Willebrand Factor A1 protein.

In yet another aspect of this embodiment, the animal is selected from the group consisting of mouse, rat, hamster, guinea pig, rabbit, dog, goat, horse, and monkey. Preferably, the animal is a mouse.

Another embodiment of the present invention is a method for calibrating an aggregometry device or a device for measuring clot formation or retraction. This method comprises:

a) providing hematologic data obtained from a subject, wherein blood or platelets from the subject is assessed by the device;

b) determining whether or not a thrombotic event occurs in a transgenic non-human animal disclosed herein, wherein the animal is perfused with a sample of blood or platelets from the subject; and

c) correlating data obtained from step (b) with the data obtained in step (a) so as to calibrate the device, wherein a certain data obtained from the device is indicative of the corresponding thrombotic outcome determined in the transgenic non-human animal.

Suitable subjects are as disclosed above.

In one aspect of this embodiment, the thrombotic event comprises blood clotting, abnormal bleeding, abnormal clotting, death, or a combination thereof.

### ADDITIONAL DEFINITIONS

The terminology used herein is for the purpose of describing particular embodiments only and is not intended to be limiting. As used in the specification and the appended claims, the singular forms “a,” “an,” and “the” include plural referents unless the context clearly dictates otherwise.

For recitation of numeric ranges herein, each intervening number there between with the same degree of precision is explicitly contemplated. For example, for the range of 6-9, the numbers 7 and 8 are contemplated in addition to 6 and 9, and for the range 6.0-7.0, the numbers 6.0, 6.1, 6.2, 6.3, 6.4, 6.5, 6.6, 6.7, 6.8, 6,9, and 7.0 are explicitly contemplated.

### Transgenic Non-Human Animals

The transgenic non-human animals of the current invention are produced by experimental manipulation of the genome of the germline of the non-human animal. These genetically engineered non-human animals may be produced by several methods well known in the art which include the introduction of a “transgene” that comprises a nucleic acid (for example, DNA such as the A1 domain of VWF) integrated into a chromosome of the somatic and/or germ line cells of a non-human animal via methods known to one skilled in the art or into an embryonal target cell. As used herein, a “transgenic animal” is an animal whose genome has been altered by the introduction of a transgene.

The term “transgene” as used herein refers to a foreign gene that is placed into an organism by introducing the foreign gene into newly fertilized eggs, embryonic stem (ES) cells, or early embryos. The term “foreign gene” refers to any nucleic acid (for example, a gene sequence) that is introduced into the genome of an animal by experimental manipulations. These nucleic acids may include gene sequences found in that animal so long as the introduced gene contains some modification (for example, the presence of a selectable marker gene, a point mutation—such as the base pair substitution mutant that contributes to the amino acid change at amino acid residue 1326 of the A1 domain of VWF, the replacement of mouse VWF A1 domain with the human VWF A1 domain (e.g., 1240P-1481G of SEQ ID NO:6), the presence of a loxP site, and the like) relative to the naturally-occurring gene.

The term “loxP site” refers to a short (34 bp) DNA sequence that is recognized by the Cre recombinase of the E. coli bacteriophage P1. In the presence of Cre recombinase, placement of two loxP sites in the same orientation on either side of a DNA segment can result in efficient excision of the intervening DNA segment, leaving behind only a single copy of the loxP site (Sauer et al., 1988).

The embryonic stem (ES) cells suitable for generating transgenic animals are those that harbor introduced expression vectors (constructs), such as plasmids and the like. Such ES cells are known in the art. The expression vector constructs can be introduced via transfection, lipofection, transformation, injection, electroporation, or infection, or other techniques known in the art. The expression vectors can contain coding sequences, or portions thereof, encoding proteins for expression. Such expression vectors can include the required components for the transcription and translation of the inserted coding sequence.

Introducing targeting vectors (as disclosed below in more detail) into ES cells can generate the VWF transgenic animals of the present invention, in particular those encoding amino acids 1240P-1481G of SEQ ID NO:6. ES cells are obtained by culturing pre-implantation embryos in vitro under appropriate conditions (Evans, et al. (1981) Nature 292:154-156; Bradley, et al. (1984) Nature 309:255-258; Gossler, et al. (1986) Proc. Acad. Sci. USA 83:9065-9069; and Robertson, et al. (1986) Nature 322:445-448). Using a variety of methods known to those skilled in the art, transgenes can be efficiently introduced into the ES cells via DNA transfection methods, which include (but are not limited to), protoplast or spheroplast fusion, electroporation, retrovirus-mediated transduction, calcium phosphate co-precipitation, lipofection, microinjection, and DEAE-dextran-mediated transfection. Following the introduction into the blastocoel of a blastocyst-stage embryo, transfected ES cells can thereafter colonize an embryo and contribute to the germ line of the resulting chimeric animal (see Jaenisch, (1988) Science 240:1468-1474). Assuming that the transgene provides a means for selection, the transfected ES cells may be subjected to various selection protocols to enrich for ES cells that have integrated the transgene prior to the introduction of transfected ES cells into the blastocoel. Alternatively, the polymerase chain reaction (PCR) may be used to screen for ES cells that have integrated the transgene and precludes the need for growth of the transfected ES cells under appropriate selective conditions prior to transfer into the blastocoel.

Alternative methods for the generation of transgenic animals (such as transgenic mice) containing an altered A1 domain of the VWF gene encoding, e.g., amino acids 1240P-1481G of SEQ ID NO:6, are established in the art. For example, embryonic cells at various stages of development can be used to introduce transgenes for the production of transgenic animals and different methods are used that depend on the stage of embryonic cell development. For microinjection methods, the zygote is best suited. In the mouse, the male pronucleus reaches the size of approximately 20 microns in diameter, which allows for reproducible injection of 1-2 picoliters (pl) of suspended DNA solution. A major advantage in using zygotes as a gene transfer target is that in most cases, the injected DNA will be incorporated into the host genome before the first cleavage (Brinster, et al. (1985) Proc. Natl. Acad. Sci. USA 82:4438-4442). Thus, all cells of the transgenic non-human animal (such as a mouse) will carry the incorporated transgene (encoding, e.g., amino acids 1240P-1481G of SEQ ID NO:6), which can result in the efficient transmission of the transgene to the offspring of the founder since 50% of the germ cells will harbor the transgene (see e.g., U.S. Pat. No. 4,873,191).

Yet another method known in the art that can be used to introduce transgenes into a non-human animal is retroviral infection. The developing non-human embryo can be cultured in vitro to the blastocyst stage wherein during this time, the blastomeres can be targets for retroviral infection (Janenich (1976) Proc. Natl. Acad. Sci. USA 73:1260-1264). Enzymatic treatment to remove the zona pellucida can increase infection efficiency of the blastomeres (Hogan et al. (1986) in Manipulating the Mouse Embryo, Cold Spring Harbor Laboratory Press, Plainview, N.Y.). The viral vector system used by one skilled in the art in order to introduce the transgene is usually a replication-defective retrovirus that harbors the transgene (Jahner, D. et al. (1985) Proc. Natl. Acad. Sci. USA 82:6927-6931; Van der Putten, et al. (1985) Proc. Natl. Acad. Sci. USA 82:6148-6152). Transfection can be easily and efficiently obtained via culturing blastomeres on a monolayer of virus-producing cells (Van der Putten, supra; Stewart, et al. (1987) EMBO J. 6:383-388). Infection can also be performed at a later stage whereby virus or virus-producing cells are injected into the blastocoele (Jahner, D. et al. (1982) Nature 298:623-628). Most of the founder non-human animals will be mosaic for the transgene since incorporation occurs only in a subset of cells that form the transgenic animal and the founder may additionally contain various retroviral insertions of the transgene at different positions in the genome that generally will segregate in the offspring. Additional methods of using retroviruses or retroviral vectors to create transgenic animals known to those skilled in the art involves microinjecting mitomycin C-treated cells or retroviral particles producing retrovirus into the perivitelline space of fertilized eggs or early embryos (see Haskell and Bowen (1995) Mol. Reprod. Dev. 40:386).

### von Willebrand Factor (VWF), the A1 Domain, and Related Diseases

As used herein, von Willebrand factor is abbreviated “VWF”. VWF polypeptides are known in the art. For example, pre-pro-human VWF was assigned the Genbank GI accession number of 401413. VWF polypeptide sequences in other animals, such as, e.g., dog (GI accession number 1478046), cat (GI accession number 974579), pig (GI accession number 243984), Norway rat (GI accession number 1256375), and Equus asinus (GI accession number 974573), are also known. (Jenkins et al., 1998).

The VWF sequences from mouse and human have been aligned as shown in Jenkins et al. (1998). The cDNA sequence encoding pre-pro-human VWF (SEQ ID NO: 7) is readily available to those skilled in the art, under Genbank Accession No. X04385. The translated polypeptide sequence of human VWF is listed in SEQ ID NO: 6. The A1 domain of VWF runs from, e.g., amino acid residue number 1240 (proline) to amino acid residue number 1481 (glycine) in both human and mouse VWF. The amino acid sequence of mouse A1 domain is listed as SEQ ID NO:26, and the amino acid sequence of human A1 domain is listed as SEQ ID NO:27. A portion of the human VWF A1 domain, amino acid residue number 1260 to amino acid residue number 1480 of the amino acid sequence of SEQ ID NO:6, is shown in SEQ ID NO: 1. Additionally, a portion of the mouse VWF A1 domain, amino acid residue number 1260 to amino acid residue number 1480 of the of SEQ ID NO:8, is shown in SEQ ID NO: 2.

VWF is one of the key players in arterial thrombosis, which is a pathological consequence of disease states such as atherosclerosis and which remains a major cause of morbidity and mortality in the Western world, with healthcare cost ranging in the billions of dollars in the USA alone (Circulation 2006; 113:e85). Central to this process is the inappropriate deposition and activation of platelets in diseased vessels that can ultimately occlude the lumen, thus impeding blood flow to vital organ such as the heart and brain.

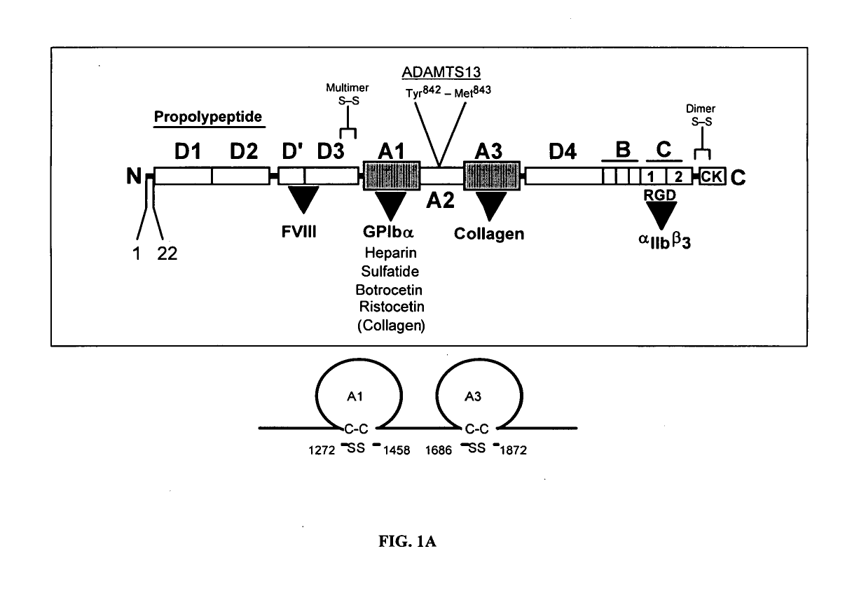

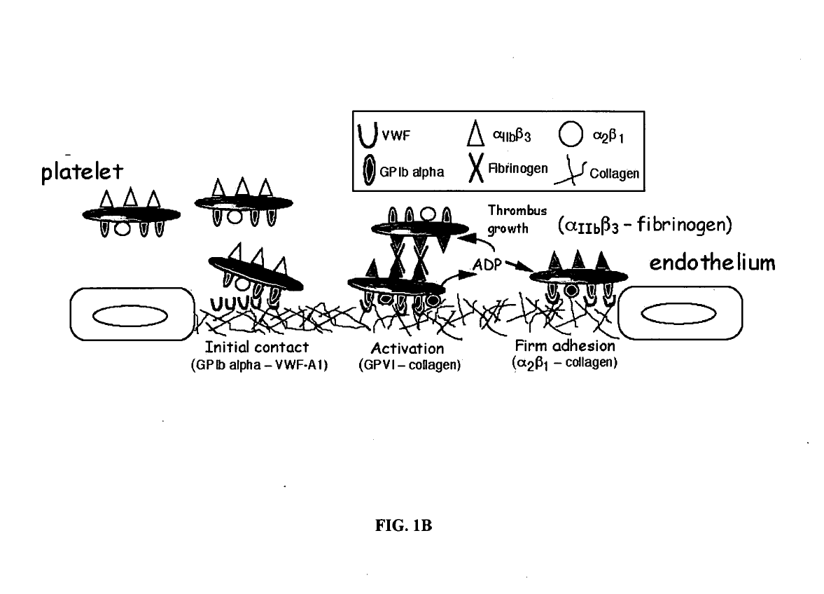

VWF is a large plasma glycoprotein of complex multimeric structure, which under normal physiological conditions prevents excessive bleeding by promoting platelet deposition at sites of vascular injury, thus “sealing off” leaky blood vessels. In order for this event to occur, VWF must form a “bridge” between receptors expressed on circulating platelets and exposed components of the injured vessel wall. This is the function of the A1 and A3 domains of this plasma protein, respectively. Each is folded into a disulfide-bonded loop structure that is critical for optimal biological activity (FIG. 1A).

It is the A1 domain that contains residues that compose the binding site for its receptor on platelets known as GPIb alpha, an adhesive event essential for the ability of these cells to rapidly attach to the injured vessel wall. The critical nature of this interaction is exemplified by the bleeding disorder, termed type 2M von Willebrand Disease (VWD), which results from the incorporation of loss-of-function point mutations within this domain that reduce the interaction between VWF-A1 and GPIb alpha (Sadler J E et al., (2006) J. Thromb. Haemost. 4: 2103-14). The A3 domain, on the other hand, is believed to be important in anchoring plasma VWF to sites where extracellular matrix components (i.e. collagen) are exposed as a result of disruption of the overlying vasculature endothelium (Wu D. et al. Blood 2002). Once in contact with exposed elements of the damaged vessel wall, platelets become “activated” through various signaling pathways (i.e. GPVI) enabling other adhesion molecules, such as α2β1 (collagen receptor) and aIIbβ3 (fibrinogen and VWF receptor) integrins, to firmly anchor these cells at the site of injury and to each other (FIG. 1B). In addition, ADP released from adherent platelets serves to amplify the activation of integrin receptors as well as other platelets leading to thrombus growth. Considerable emphasis has been placed on understanding the mechanism(s) that govern the interaction between GPIb alpha and the A1 domain of VWF and how it can be perturbed by point mutations associated with von Willebrand Disease, information relevant to the development of anti-thrombotic therapies.

During the past two decades, there has been considerable progress in understanding how VWF mediates platelet adhesion. Both the VWF cDNA and gene have been cloned and the primary structure of the VWF subunit (FIG. 1A) has been determined (Bonthron et al., 1986; Shelton-Inloes et al., 1986; Verweij et al., 1986; Mancuso et al., 1989). It has been reported that about 59% of the mature VWF consists of repeated segments which are 29% to 43% homologous (Sadler et al., 1985). These regions consist of domains that are triplicated (domains “A” and “B”), duplicated (domain “C”) or quadruplicated (domain “D”). The triplicated A repeats encode for the central region of each VWF subunit. The A1 and A3 domains contain the sequences that mediate VWF's interaction with receptors on platelets or components of subendothelial extracellular matrix, respectively. Each is folded into a disulfide-bonded loop structure that is critical for optimal biological activity. The sequences of the amino terminal halves of each loop and the location of the cysteines forming the loop structure of each domain are highly conserved. It is the VWF-A1 domain (1240-1481) that contains sequences that provide binding sites for the platelet glycoprotein receptor Ib alpha, an interaction critical for the ability of these cells to rapidly attach and translocate at sites vascular injury (Cruz et al., 2000; Savage et al., 1996). The role of the A3 domain, however, is believed to be in anchoring plasma VWF at sites where extracellular matrix components (i.e. collagen) are exposed as a result of disruption of the endothelium (Kalafatis et al., 1987; Pareti et al., 1987; Roth et al., 1986; Lankhof et al., 1996; Pareti et al., 1986; Pietu et al., 1989).

With regard to mediating adhesive interactions with platelets, it has become increasingly evident that the VWF-A1 domain plays a crucial role in this process based on molecular genetic studies of individuals with type 2M or 2B VWD (Meyer et al, 1997; Ginsburg et al, 1993; Hillery et al., 1998; Mancuso et al., 1996; Ruggeri et al., 1980; Cooney et al., 1996). VWD is a common hereditary coagulation abnormality that arises from a quantitative or qualitative deficiency of VWF). VWD affects humans, in addition to dogs and cats. There are three types of VWD: type 1, type 2, and type 3. Type 1 VWD is a quantitative defect, wherein decreased levels of VWF are detected but subjects may not have clearly impaired clotting, Type 2 VWD is a qualitative defect, wherein subjects have normal VWF levels but VWF multimers are structurally abnormal, or subgroups of large or small multimers are absent. Four subtypes exist: Type 2A, Type 2B, Type 2M, and Type 2N. Type 3 is rare and the most severe form of VWD (homozygous for the defective gene). (Braunwald et al., Harrison's Principle of Internal Medicine, 15th ed., (Chapter 116) 2001, McGraw Hill, Columbus, Ohio).

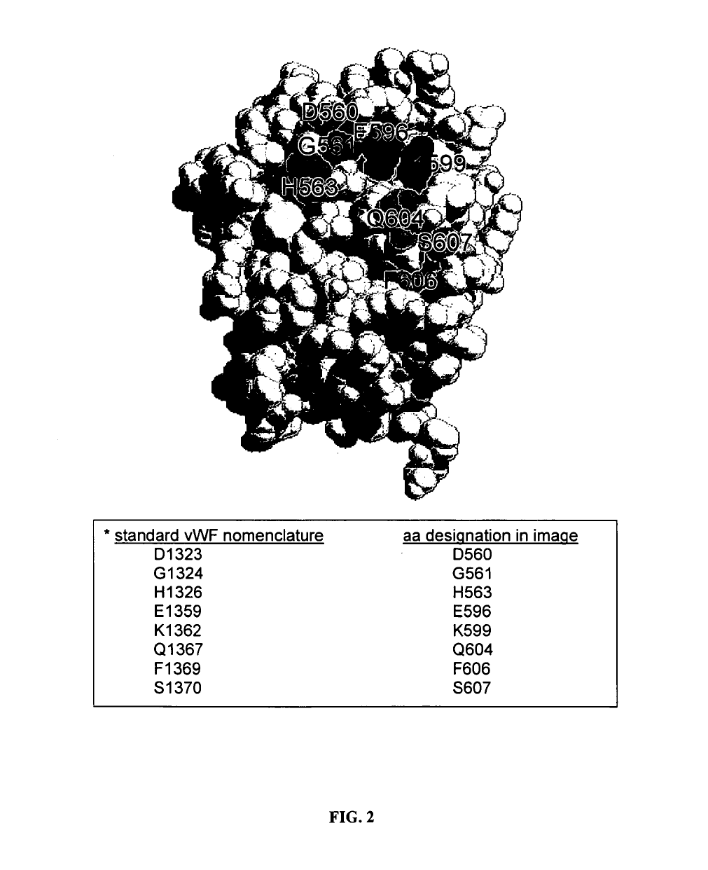

In the majority of cases with type 2M or type 2B VWD, patients with these designated genotypes have single point mutations contained within the disulfide loop (between Cys 1272 and Cys 1458) of this domain. With regard to type 2M VWD, afflicted individuals have significant impairments in hemostasis that appears to result from a lack of or reduced adhesive interactions between GPIb alpha and VWF at sites of vascular injury, and not from an alteration in VWF multimer structure. Structural and functional evidence has been provided in that type 2M mutations, such as 1324G>S, are localized within a region of the A1 domain (FIG. 5B), which is critical for supporting GPIb alpha-mediated platelet adhesion at physiological flow rates. Confirmation that this residue, as well as others predicted by our analysis of the crystal structure of the A1 domain, does contribute to GPIb alpha binding is suggested by studies evaluating the structure of the complex formed between this receptor-ligand pair (Cruz et al., 2000; Huizing a et al., 2002). Thus, it is possible to make accurate predictions about protein function from the three-dimensional protein structure and to confirm these postulates by site-specific mutagenesis and analysis under physiologically relevant flow conditions. The localization of some of the residues within the A1 domain that when mutated disrupt GPIb alpha binding is shown below (FIG. 2). The present invention provides methods for evaluating the effect that loss-of-function mutations have on hemostasis and thrombus formation.

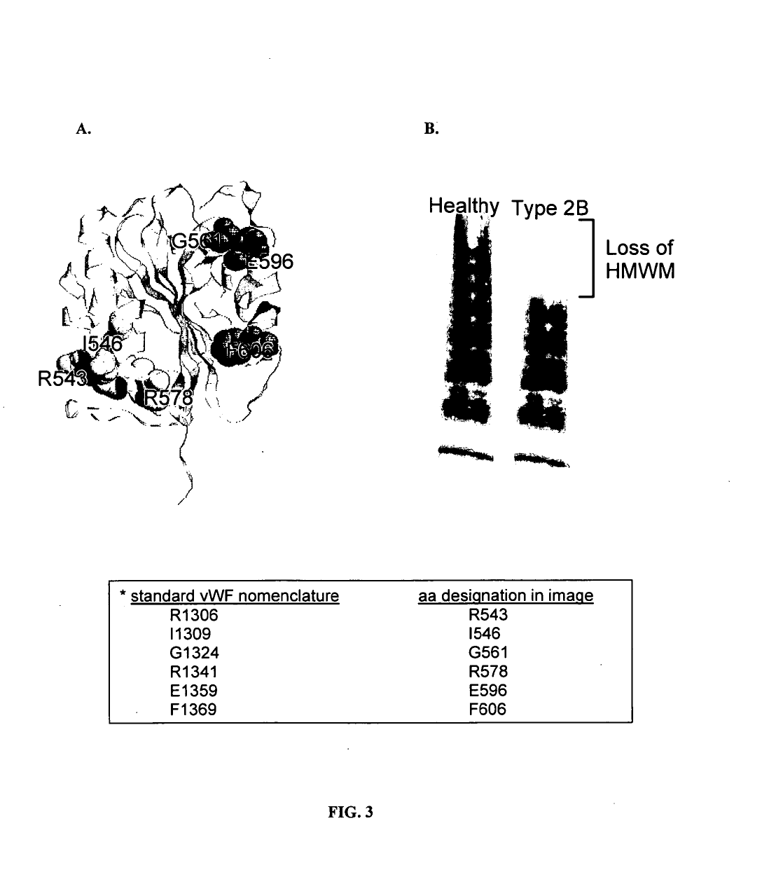

In contrast to type 2M VWD, mutations associated with type 2B VWD are known to enhance the interaction between VWF-A1 and GPIb alpha, that is, they mitigate the requirement for exogenous modulators such as ristocetin or botrocetin to induce platelet agglutination (Ruggeri et al., 1980). Moreover, these altered residues are localized in a region remote from the major GPIb alpha binding site that has been identified by mutagenesis (Meyer et al., 1997; FIG. 3A). Clinically, this disease state is characterized by a loss of circulating high molecular weight VWF multimers (HMWM, FIG. 3B) together with a mild to moderate thrombocytopenia, which ultimately results in bleeding but not thrombosis (Ruggeri et al., 1980; Cooney et al., 1996). It is the clearance of the HMWM of VWF from plasma that is believed to be responsible for the increased bleeding tendencies in patients with this disorder as they contribute to the majority of the hemostatic function associated with this plasma glycoprotein (Federici et al., 1989). The present invention provides methods for evaluating the effect that gain-of-function mutations have on hemostasis, thrombus formation, and plasma levels of VWF.

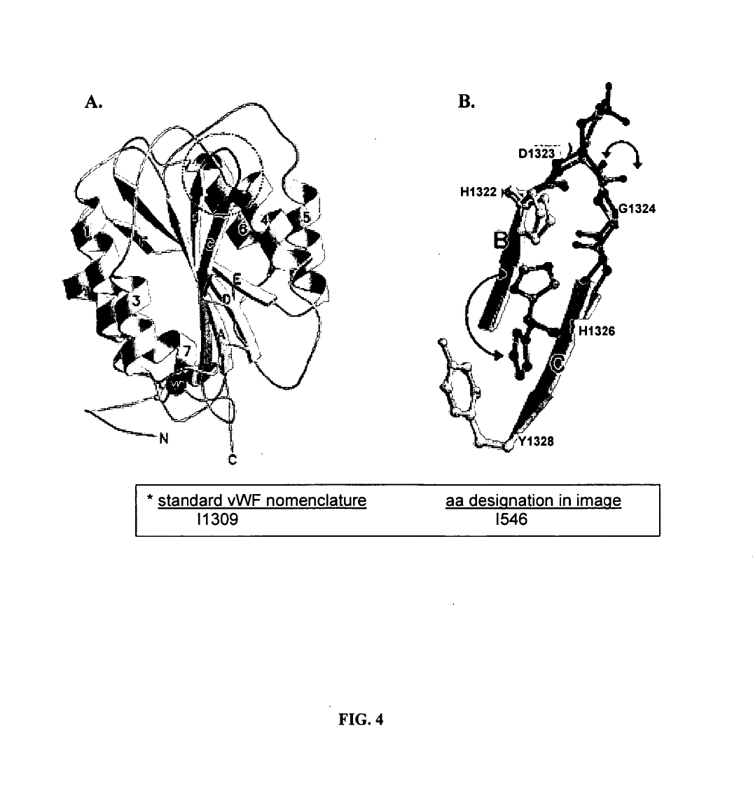

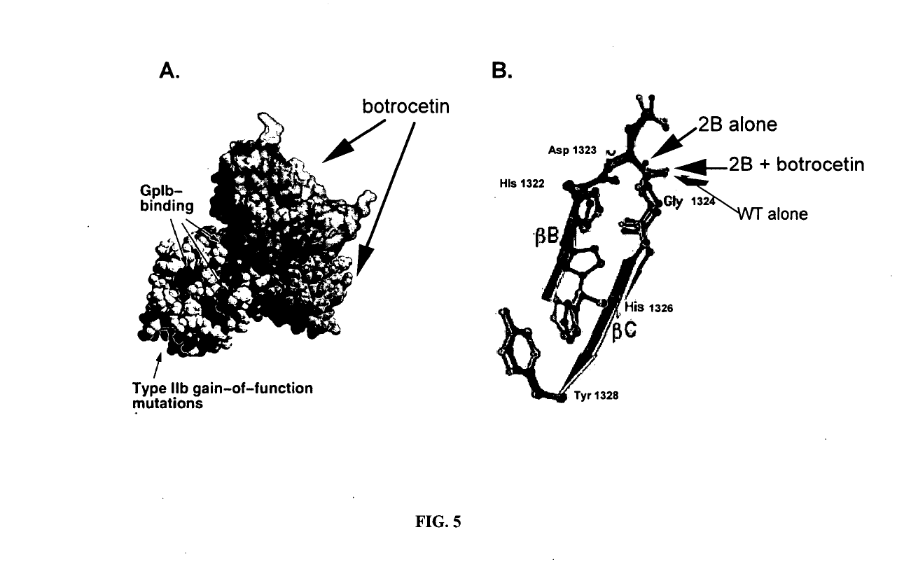

Surface-immobilization of VWF and subsequent exposure to physiologically relevant shear forces appears to be a prerequisite for its ability to support interactions with platelets as this multimeric protein does not bind appreciably to these cells in the circulation. These hydrodynamic conditions are believed to promote structural changes within the A1 domain that in turn increases its affinity for GPIb alpha (Roth et al., 1991; Siedlecki et al., 1996; Ruggeri et al., 1992). Evidence suggested to support the existence of such an alteration in structure includes the ability of non-physiologic modulators such as the antibiotic ristocetin or the snake venom protein botrocetin to promote platelet agglutination in solution-based assays (Howard et al., 1981; Read et al., 1989). Moreover, this “on” and “off” conformation is exemplified by type 2B VWD. For instance, it was initially hypothesized that incorporation of type 2B mutations into the A1 domain shifted the equilibrium between two distinct tertiary conformations, analogous to those seen in crystal structures of the integrin 1 domain in ligand-free and collagen-bound states (Emsley et al., 2000). The location of the type 2B mutants at sites distinct from the GPIb alpha binding site suggest that they disrupt a region responsible for regulation of binding affinity, thus affecting ligand binding allosterically. The crystal structure of a type 2B mutant A1 domain, 1309I>V, was determined and compared to its wild-type counterpart (Celikel et al., 2000). A change was discovered in the structure of a loop, thought to be involved in GPIb alpha binding, lying on the surface distal to the mutation site. A similar finding has been observed for the VWF-A1 crystal structure containing the identical mutation (FIG. 4).

This altered conformation represents a high affinity binding state of the A1 domain. However, the pathway of allosteric change proposed previously, involving the burial of a water molecule, cannot be a general feature of type 2B mutants, and the structural rearrangements appear too subtle to explain the altered kinetics. Interestingly, complex formation between botrocetin and VWF-A1 in which the type 2B mutation 1309I>V has been incorporated, and has demonstrated that most of these structural differences are reversed including: (1) loss of the buried water molecule at the mutation site; (2) the peptide plan between Asp 1323 and Gly 1324 flips back to a conformation similar to that in the WT structure; and (3) the side chain of His 1326 remains in the “mutant” position, although there is some evidence from electron density of an alternative conformation similar to WT. However, this reversion in structure does not correlate with a loss in the function-enhancing activity associated with the type 2B mutation. In fact, the addition of botrocetin further augments the interaction between the mutant A1 domain and platelets in flow (Fukuda et al., 2002). Thus, an alternative mechanism must account for function-enhancing nature of type 2B mutations. Moreover, these subtle alterations in structure did not compare to the large conformational changes in homologous integrin I-type domains that occur on ligand binding.

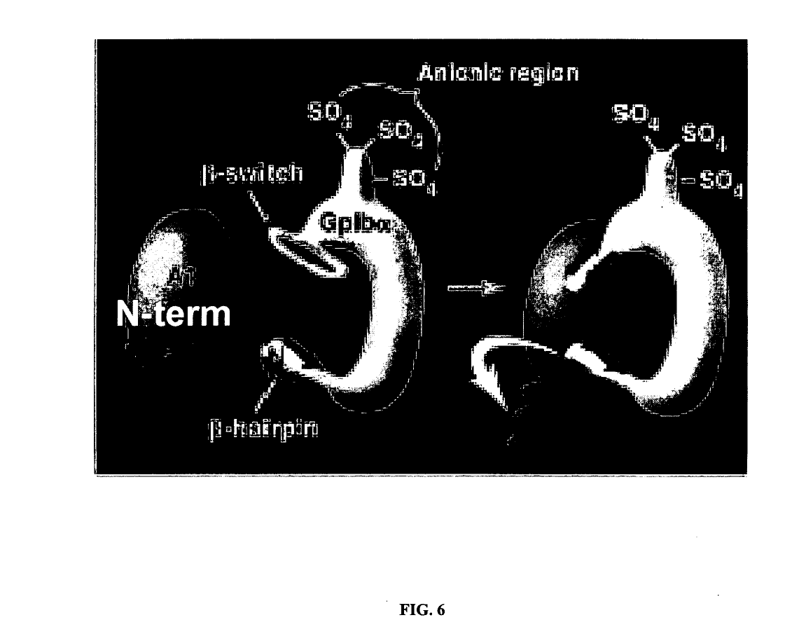

Recent findings provide support that type 2B mutations may stabilize the binding of a region of GPIb alpha known as the β-hairpin to an area near the location of these altered residues, distinct from that identified by site-directed mutagenesis. Type 2B mutations have been suggested to destabilize a network of interactions observed between the bottom face of the A1 domain and its terminal peptides in the wild type A1 structure, thereby making the binding site accessible (FIG. 6).

Another disease of interest is Bernard-Soulier Syndrome, which is a rare disorder caused by a deficiency of the surface platelet receptor GPIb alpha. As a result, platelets fail to stick and clump together at the site of the injury. Functional abnormalities have also been observed in some hereditary platelet disorders wherein the platelets are of abnormal size or shape, such as in May-Hegglin Anomaly and Chediak Higashi syndrome. (Braunwald et al., Harrison's Principle of Internal Medicine, 15th ed. (Chapter 116) 2001, McGraw Hill, Columbus, Ohio).

Progress has been made in understanding the structure of VWF and GPIb alpha proteins and potential alterations in conformation that may regulate this protein-protein interaction. This model is non-limiting. However, the model permits determination of the kinetic and biomechanical basis for 1) the regulation of VWF-A1 domain activity in response to hydrodynamic forces, 2) the alterations in bond kinetics that result from incorporation of type 2B mutations into the VWF-A1 domain, and 3) the susceptibility of the kinetics of the GPIb alpha-VWF-A1 bond to an applied force.

Nucleic acids and Vectors

“Nucleic acid” or “oligonucleotide” or “polynucleotide” used herein mean at least two nucleotides covalently linked together.

Nucleic acids may be synthesized chemically or isolated by one of several approaches established in art. The basic strategies for identifying, amplifying, and isolated desired DNA sequences as well as assembling them into larger DNA molecules containing the desired sequence domains in the desired order, are well known to those of ordinary skill in the art. See, e.g., Sambrook, et al., (1989) Nature November 16; 342(6247):224-5; Perbal, B. et al., (1983) J. Virol. March; 45(3):925-40. DNA segments corresponding to all or a portion of the VWF sequence may be isolated individually using the polymerase chain reaction (M. A. Innis, et al., “PCR Protocols: A Guide To Methods and Applications,” Academic Press, 1990). A complete sequence may be assembled from overlapping oligonucleotides prepared by standard methods and assembled into a complete coding sequence. See, e.g., Edge (1981), Nature, 292:756; Nambiar, et al. (1984), Science, 223:1299; Jay et al. (1984), J. Biol. Chem., 259:6311. Thus, procedures for construction and expression of mutant proteins of defined sequence are well known in the art.

The assembled nucleotide sequence can be cloned into a suitable vector. As used herein, a “vector” means a vehicle to carry a nucleic acid into a cell. Vectors include, without limitation, cloning vectors, targeting vectors, and expression vectors.

Vectors generally contain a selectable marker. A selectable marker can include a gene which encodes an enzymatic activity that confers resistance to an antibiotic or drug upon the cell in which the selectable marker is expressed. Selectable markers may be positive. A positive selectable marker is usually a dominant selectable marker wherein the genes encode an enzymatic activity that can be detected in a mammalian cell or a cell line (including ES cells). Some non-limiting examples of dominant selectable markers include the bacterial xanthine-guanine phosphoribosyl transferase gene (also referred to as the gpt gene) which confers the ability to grow in the presence of mycophenolic acid, the bacterial hygromycin G phosphotransferase (hyg) gene which confers resistance to the antibiotic hygromycin, and the bacterial aminoglycoside 3′ phosphotransferase gene (also referred to as the neo gene) which confers resistance to the drug G418 in mammalian cells. Selectable markers may also be negative. Negative selectable markers encode an enzymatic activity whose expression is toxic to the cell when grown in an appropriate selective medium. One non-limiting example of a negative selectable marker is the HSV-tk gene wherein HSV-tk expression in cells grown in the presence of gancyclovir or acyclovir is catatonic. Growth of cells in selective medium containing acyclovir or gancyclovir therefore selects against cells capable of expressing a functional HSV TK enzyme.

Those of ordinary skill in the art are familiar with numerous cloning vectors, and the selection of an appropriate cloning vector is a matter of choice. The construction of vectors containing desired nucleotide sequences linked by appropriate DNA sequences is accomplished by discussed above. These vectors may be constructed to contain additional DNA sequences, such as bacterial origins of replication to make shuttle vectors in order to shuttle between prokaryotic hosts and mammalian hosts.

A suitable targeting vector contains the modified A1 domain of VWF gene sequence, containing, e.g., a VWF gene sequence modified to encode amino acids 1240P-1481G of SEQ ID NO:6, sufficient to permit the homologous recombination of the targeting vector into at least one allele of the A1 domain of the VWF gene resident in the chromosomes of the target or recipient cell (for example, ES cells). The targeting vector will usually harbor 10 to 15 kb of DNA homologous to the A1 domain of the VWF gene, wherein this 10 to 15 kb of DNA will be divided more or less equally on each side of the selectable marker gene. One non-limiting exemplary targeting vector is shown in SEQ ID NO:11.

Targeting vectors can also be of the replacement-type wherein the integration of a replacement-type vector results in the insertion of a selectable marker into the target gene. Replacement-type targeting vectors may be employed to disrupt a gene (such as the VWF gene or the A1 domain of the VWF gene). This can result in the generation of a null allele; for example, an allele not capable of expressing a functional protein wherein the null alleles may be generated by deleting a portion of the coding region, deleting the entire gene, introducing an insertion and/or a frameshift mutation, and the like. Expression vectors containing sequences encoding the produced proteins and polypeptides, as well as the appropriate transcriptional and translational control elements, can be generated using methods well known to and practiced by those skilled in the art. These methods include in vitro recombinant DNA techniques, synthetic techniques, and in vivo genetic recombination which are described in J. Sambrook et al., 1989, Molecular Cloning, A Laboratory Manual, Cold Spring Harbor Press, Plainview, N.Y. and in F. M. Ausubel et al., 1989, Current Protocols in Molecular Biology, John Wiley & Sons, New York, N.Y. In one embodiment, loxP expressing targeting vectors are used for transfection methods (such as pDNR-1r vector, pACD4K-C vector, and the like). In other embodiments, Cre-recombinase-expressing plasmids are also utilized (for example, crAVE cre recombinase vectors).

An expression vector containing a nucleotide sequence encoding a protein of interest, such as a VWF-A1 molecule, encoding, e.g., amino acids 1240P-1481G of SEQ ID NO:6, is transfected into a host cell, either eukaryotic (for example, yeast, mammalian, or insect cells) or prokaryotic, by conventional techniques well established in the art. Transfection techniques carried out depend on the host cell used. For example, mammalian cell transfection can be accomplished using lipofection, protoplast fusion, DEAE-dextran mediated transfection, CaPO₄ co-precipitation, electroporation, direct microinjection, as well as other methods known in the art which can comprise: scraping, direct uptake, osmotic or sucrose shock, lysozyme fusion or erythrocyte fusion, indirect microinjection such as via erythrocyte-mediated techniques, and/or by subjecting host cells to electric currents. Some of the techniques mentioned above are also applicable to unicellular organisms, such as bacteria or yeast. As other techniques for introducing genetic information into host cells will be developed, the above-mentioned list of transfection methods is not considered to be exhaustive. The transfected cells are then cultured by conventional techniques to produce a VWF-A1 molecule harboring at least one of the mutations previously described, particularly a VWF-A1 molecule encoding amino acids 1240P-1481G of SEQ ID NO:6.

One skilled in the art understands that expression of desired protein products in prokaryotes is most often carried out in E. coli with vectors that contain constitutive or inducible promoters. Some non-limiting examples of bacterial cells for transformation include the bacterial cell line E. coli strains DH5a or MC1061/p3 (Invitrogen Corp., San Diego, Calif.), which can be transformed using standard procedures practiced in the art, and colonies can then be screened for the appropriate plasmid expression. Some E. coli expression vectors (also known in the art as fusion-vectors) are designed to add a number of amino acid residues, usually to the N-terminus of the expressed recombinant protein. Such fusion vectors can serve three functions: 1) to increase the solubility of the desired recombinant protein; 2) to increase expression of the recombinant protein of interest; and 3) to aid in recombinant protein purification by acting as a ligand in affinity purification. In some instances, vectors, which direct the expression of high levels of fusion protein products that are readily purified, may also be used. Some non-limiting examples of fusion expression vectors include pGEX, which fuse glutathione S-tranferase to desired protein; pcDNA 3.1V5-His A B & C (Invitrogen Corp, Carlsbad, Calif.) which fuse 6×-His to the recombinant proteins of interest; pMAL (New England Biolabs, MA) which fuse maltose E binding protein to the target recombinant protein; the E. coli expression vector pUR278 (Ruther et al., (1983) EMBO 12:1791), wherein the coding sequence may be ligated individually into the vector in frame with the lac Z coding region in order to generate a fusion protein; and pIN vectors (Inouye et al., (1985) Nucleic Acids Res. 13:3101-3109; Van Heeke et al., (1989) J. Biol. Chem. 24:5503-5509. Fusion proteins generated by the likes of the above-mentioned vectors are generally soluble and can be purified easily from lysed cells via adsorption and binding to matrix glutathione agarose beads subsequently followed by elution in the presence of free glutathione. For example, the pGEX vectors are designed to include thrombin or factor Xa protease cleavage sites so that the cloned target can be released from the GST moiety.

Other suitable cell lines, in addition to microorganisms such as bacteria (e.g., E. coli and B. subtilis) transformed with recombinant bacteriophage DNA, plasmid DNA or cosmid DNA expression vectors containing coding sequences for a VWF-A1 molecule described above, such as, e.g., one encoding amino acid 1240P-1481G of SEQ ID NO:6, may alternatively be used to produce the molecule of interest. Non-limiting examples include plant cell systems infected with recombinant virus expression vectors (for example, tobacco mosaic virus, TMV; cauliflower mosaic virus, CaMV) or transformed with recombinant plasmid expression vectors (e.g., Ti plasmid) containing coding sequences for a VWF-A1 molecule described above, such as, e.g., one encoding amino acid 1240P-1481G of SEQ ID NO:6; insect cell systems infected with recombinant virus expression vectors (e.g., baculovirus) containing coding sequences for a VWF-A1 molecule described above, such as, e.g., one encoding amino acid 1240P-1481G of SEQ ID NO:6; yeast (for example, Saccharomyces sp., Pichia sp.) transformed with recombinant yeast expression vectors containing coding sequences for a VWF-A1 molecule described above, such as, e.g., one encoding amino acid 1240P-1481G of SEQ ID NO:6; or mammalian cell lines harboring a vector that contains coding sequences for a VWF-A1 molecule described above, such as, e.g., one encoding amino acid 1240P-1481G of SEQ ID NO:6.

Mammalian cells (such as BHK cells, VERO cells, CHO cells and the like) can also contain an expression vector (for example, one that harbors a nucleotide sequence encoding a VWF-A1 molecule described above, such as, e.g., one encoding amino acid 1240P-1481G of SEQ ID NO:6) for expression of a desired product. Expression vectors containing such a nucleic acid sequence linked to at least one regulatory sequence in a manner that allows expression of the nucleotide sequence in a host cell can be introduced via methods known in the art, as described above. To those skilled in the art, regulatory sequences are well known and can be selected to direct the expression of a protein of interest in an appropriate host cell as described in Goeddel (Gene Expression Technology (1990) Methods in Enzymology 185, Academic Press, San Diego, Calif.). Regulatory sequences can comprise the following: enhancers, promoters, polyadenylation signals, and other expression control elements. Practitioners in the art understand that designing an expression vector can depend on factors, such as the choice of host cell to be transfected and/or the type and/or amount of desired protein to be expressed.

Animal or mammalian host cells capable of harboring, expressing, and secreting large quantities of a VWF-A1 molecule (described above, such as, e.g., one encoding amino acid 1240P-1481G of SEQ ID NO:6) of interest into the culture medium for subsequent isolation and/or purification include, but are not limited to, Chinese hamster ovary cells (CHO), such as CHO-K1 (ATCC CCL-61), DG44 (Chasin et al., (1986) Som. Cell Molec. Genet, 12:555-556; Kolkekar et al., (1997) Biochemistry, 36:10901-10909; and WO 01/92337 A2), dihydrofolate reductase negative CHO cells (CHO/dhfr-, Urlaub et al., (1980)

Proc. Natl. Acad. Sci. U.S.A., 77:4216), and dp12.CHO cells (U.S. Pat. No. 5,721,121); monkey kidney CV1 cells transformed by SV40 (COS cells, COS-7, ATCC CRL-1651); human embryonic kidney cells (e.g., 293 cells, or 293 cells subcloned for growth in suspension culture, Graham et al., (1977) J. Gen. Virol., 36:59); baby hamster kidney cells (BHK, ATCC CCL-10); monkey kidney cells (CV1, ATCC CCL-70); African green monkey kidney cells (VERO-76, ATCC CRL-1587; VERO, ATCC CCL-81); mouse sertoli cells (TM4; Mather (1980) Biol. Reprod., 23:243-251); human cervical carcinoma cells (HELA, ATCC CCL-2); canine kidney cells (MDCK, ATCC CCL-34); human lung cells (W138, ATCC CCL-75); human hepatoma cells (HEP-G2, HB 8065); mouse mammary tumor cells (MMT 060562, ATCC CCL-51); buffalo rat liver cells (BRL 3A, ATCC CRL-1442); TR1 cells (Mather (1982) Annals NY Acad. Sci., 383:44-68); MCR 5 cells; FS4 cells. A cell line transformed to produce a VWF-A1 molecule described above, such as, e.g., one encoding amino acid 1240P-1481G of SEQ ID NO:6, can also be an immortalized mammalian cell line of lymphoid origin, which include but are not limited to, a myeloma, hybridoma, trioma or quadroma cell line. The cell line can also comprise a normal lymphoid cell, such as a B cell, which has been immortalized by transformation with a virus, such as the Epstein Barr virus (such as a myeloma cell line or a derivative thereof).

A host cell strain, which modulates the expression of the inserted sequences, or modifies and processes the nucleic acid in a specific fashion desired also may be chosen. Such modifications (for example, glycosylation and other post-translational modifications) and processing (for example, cleavage) of protein products may be important for the function of the protein. Different host cell strains have characteristic and specific mechanisms for the post-translational processing and modification of proteins and gene products. As such, appropriate host systems or cell lines can be chosen to ensure the correct modification and processing of the foreign protein expressed, which includes, for example, a VWF-A1 molecule described above, such as, e.g., one encoding amino acid 1240P-1481G of SEQ ID NO:6. Thus, eukaryotic host cells possessing the cellular machinery for proper processing of the primary transcript, glycosylation, and phosphorylation of the gene product may be used. Non-limiting examples of mammalian host cells include 3T3, W138, BT483, Hs578T, CHO, VERY, BHK, Hela, COS, BT2O, T47D, NSO (a murine myeloma cell line that does not endogenously produce any immunoglobulin chains), CRL7O3O, MDCK, 293, HTB2, and HsS78Bst cells.

For protein recovery, isolation and/or purification, the cell culture medium or cell lysate is centrifuged to remove particulate cells and cell debris. The desired polypeptide molecule (for example, a VWF-A1 protein such as, e.g., one encoding amino acid 1240P-1481G of SEQ ID NO:6) is isolated or purified away from contaminating soluble proteins and polypeptides by suitable purification techniques. Non-limiting purification methods for proteins include: separation or fractionation on immunoaffinity or ion-exchange columns; ethanol precipitation; reverse phase HPLC; chromatography on a resin, such as silica, or cation exchange resin, e.g., DEAE; chromatofocusing; SDS-PAGE; ammonium sulfate precipitation; gel filtration using, e.g., Sephadex G-75, Sepharose; protein A sepharose chromatography for removal of immunoglobulin contaminants; and the like. Other additives, such as protease inhibitors (e.g., PMSF or proteinase K) can be used to inhibit proteolytic degradation during purification. Purification procedures that can select for carbohydrates can also be used, e.g., ion-exchange soft gel chromatography, or HPLC using cation- or anion-exchange resins, in which the more acidic fraction(s) is/are collected.

### Peptide, Polypeptide, Protein

The terms “peptide,” “polypeptide,” and “protein” are used interchangeably herein. In the present invention, these terms mean a linked sequence of amino acids, which may be natural, synthetic, or a modification, or combination of natural and synthetic. The term includes antibodies, antibody mimetics, domain antibodies, lipocalins, targeted proteases, and polypeptide mimetics. The term also includes vaccines containing a peptide or peptide fragment intended to raise antibodies against the peptide or peptide fragment.

In the present invention, the term “antibody” means full immunoglobulin molecules, as well as to parts of such immunoglobulin molecules except Fab fragments, and encompasses naturally occurring antibodies as well as non-naturally occurring antibodies, including antibody-like molecules. The term “a Fab fragment” means a Fab fragment of an antibody. Full immunoglobulin molecules include IgMs, IgDs, IgEs, IgAs or IgGs, such as IgG1, IgG2a, IgG2b, IgG3 or IgG4. Antigen-binding fragments of full immunoglobulin include, for example, Fab′, F(ab′)₂, Fv and rIgG. The term antibody further include single chain antibodies, chimeric, bifunctional and humanized antibodies. See also, e.g., Pierce Catalog and Handbook, 1994-1995 (Pierce Chemical Co., Rockford, Ill.; Kuby, J., Immunology, 3rd Ed., W.H. Freeman & Co., New York (1998). Non-naturally occurring antibodies can be constructed using solid phase peptide synthesis, can be produced recombinantly or can be obtained, for example, by screening combinatorial libraries consisting of variable heavy chains and variable light chains as described by Huse et al., Science 246:1275-1281 (1989), which is incorporated herein by reference. These and other methods of making, for example, chimeric, humanized, CDR-grafted, single chain, and bifunctional antibodies are well known to those skilled in the art (Winter and Harris, Immunol. Today 14:243-246 (1993); Ward et al., Nature 341:544-546 (1989); Harlow and Lane, supra, 1988; Hilyard et al., Protein Engineering: A practical approach (IRL Press 1992); Borrabeck, Antibody Engineering, 2d ed. (Oxford University Press 1995); each of which is incorporated herein by reference). Antibody-like molecule include affibody, affilin molecule, adnectin, anticalin, designed ankyrin repeat protein (DARPin), domain antibody, evibody, a knottin, Kunitz-type domain, maxibody, nanobody, tetranectin, trans-body, or a V(NAR).

The term “antibody” includes both polyclonal and monoclonal antibodies. The term also includes genetically engineered forms such as chimeric antibodies (e.g., humanized murine antibodies) and heteroconjugate antibodies (e.g., bispecific antibodies). The term also refers to recombinant single chain Fv fragments (scFv). As set forth above, the term antibody also includes bivalent or bispecific molecules, diabodies, triabodies, and tetrabodies. Bivalent and bispecific molecules are described in, e.g., Kostelny et al. (1992) J Immunol 148:1547, Pack and Pluckthun (1992) Biochemistry 31:1579, Hollinger et al., 1993, supra, Gruber et al. (1994) J Immunol:5368, Zhu et al. (1997) Protein Sci 6:781, Hu et al. (1996) Cancer Res. 56:3055, Adams et al. (1993) Cancer Res. 53:4026, and McCartney, et al. (1995) Protein Eng. 8:301.

Typically, an antibody has a heavy and light chain. Each heavy and light chain contains a constant region and a variable region, (the regions are also known as “domains”). Light and heavy chain variable regions contain four “framework” regions interrupted by three hypervariable regions, also called “complementarity-determining regions” or “CDRs”. The extent of the framework regions and CDRs have been defined. The sequences of the framework regions of different light or heavy chains are relatively conserved within a species. The framework region of an antibody, that is the combined framework regions of the constituent light and heavy chains, serves to position and align the CDRs in three dimensional space.

The CDRs are primarily responsible for binding to an epitope of an antigen. The CDRs of each chain are typically referred to as CDR1, CDR2, and CDR3, numbered sequentially starting from the N-terminus, and are also typically identified by the chain in which the particular CDR is located. Thus, a V_{H} CDR3 is located in the variable domain of the heavy chain of the antibody in which it is found, whereas a V_{L} CDR1 is the CDR1 from the variable domain of the light chain of the antibody in which it is found.

“V_{H}” refer to the variable region of an immunoglobulin heavy chain of an antibody, including the heavy chain of an Fv, scFv, or Fab. “V_{L}” refer to the variable region of an immunoglobulin light chain, including the light chain of an Fv, scFv, dsFv or Fab.

The phrase “single chain Fv” or “scFv” refers to an antibody in which the variable domains of the heavy chain and of the light chain of a traditional two chain antibody have been joined to form one chain. Typically, a linker peptide is inserted between the two chains to allow for proper folding and creation of an active binding site.

A “chimeric antibody” is an immunoglobulin molecule in which (a) the constant region, or a portion thereof; is altered, replaced or exchanged so that the antigen binding site (variable region) is linked to a constant region of a different or altered class, effector function and/or species, or an entirely different molecule which confers new properties to the chimeric antibody, e.g., an enzyme, toxin, hormone, growth factor, drug, etc.; or (b) the variable region, or a portion thereof, is altered, replaced or exchanged with a variable region having a different or altered antigen specificity.

A “humanized antibody” is an immunoglobulin molecule that contains minimal sequence derived from non-human immunoglobulin. Humanized antibodies include human immunoglobulins (recipient antibody) in which residues from a complementary determining region (CDR) of the recipient are replaced by residues from a CDR of a non-human species (donor antibody) such as mouse, rat or rabbit having the desired specificity, affinity and capacity. In some instances, Fv framework residues of the human immunoglobulin are replaced by corresponding non-human residues. Humanized antibodies may also comprise residues which are found neither in the recipient antibody nor in the imported CDR or framework sequences. In general, a humanized antibody will comprise substantially all of at least one, and typically two, variable domains, in which all or substantially all of the CDR regions correspond to those of a non-human immunoglobulin and all or substantially all of the framework (FR) regions are those of a human immunoglobulin consensus sequence. The humanized antibody optimally also will comprise at least a portion of an immunoglobulin constant region (Fc), typically that of a human immunoglobulin (Jones et al., Nature 321:522-525 (1986); Riechmann et al., Nature 332:323-329 (1988); and Presta, Curr. Op. Struct. Biol 2:593-596 (1992)). Humanization can be essentially performed following the method of Winter and co-workers (Jones et al., Nature 321:522-525 (1986); Riechmann et al., Nature 332:323-3′27 (1988); Verhoeyen et al., Science 239:1534-1536 (1988)), by substituting rodent CDRs or CDR sequences for the corresponding sequences of a human antibody. Accordingly, such humanized antibodies are chimeric antibodies (U.S. Pat. No. 4,816,567), wherein substantially less than an intact human variable domain has been substituted by the corresponding sequence from a non-human species.

“Epitope” or “antigenic determinant” refers to a site on an antigen to which an antibody binds. Epitopes can be formed both from contiguous amino acids or noncontiguous amino acids juxtaposed by tertiary folding of a protein. Epitopes formed from contiguous amino acids are typically retained on exposure to denaturing solvents whereas epitopes formed by tertiary folding are typically lost on treatment with denaturing solvents. An epitope typically includes at least 3, and more usually, at least 5 or 8-10 amino acids in a unique spatial conformation. Methods of determining spatial conformation of epitopes include, for example, x-ray crystallography and 2-dimensional nuclear magnetic resonance. See, e.g., Epitope Mapping Protocols in Methods in Molecular Biology, Vol. 66, Glenn E. Morris, Ed (1996). A preferred method for epitope mapping is surface plasmon resonance, which has been used to identify preferred granulation inhibitors recognizing the same epitope region as the IIA1 antibody disclosed herein.

The phrase “specifically (or selectively) binds” or when referring to protein-protein interaction, refers to a binding reaction between two molecules that is at least two times the background and more typically more than 10 to 100 times background molecular associations under physiological conditions. When using one or more detectable binding agents that are proteins, specific binding is determinative of the presence of the protein, in a heterogeneous population of proteins and other biologics. Thus, under designated immunoassay conditions, the specified antibodies bind to a particular protein sequence, thereby identifying its presence.

Peptides binding agents include receptor traps. A receptor trap is a decoy receptor that can comprise fusions between two distinct receptor components and the Fc region of an antibody molecule, which can result in the generation of a molecule with an increased affinity over single component reagents. This technology is available from Regeneron (Tarrytown, N.Y.) and is described in Wachsberger et al., (2007) Int J Radiat Oncol Biol Phys. 67(5):1526-37; Holash et al., (2002) Proc Natl Acad Sci USA. 2002 99(17):11393-8; Davis et al., (1996) Cell. 87(7):1161-9; U.S. Pat. No. 7,087,411; and in United States Publication Applications 2004/0014667, 2005/0175610, 2005/0260203, 2006/0030529, 2006/0058234, which are all hereby incorporated by reference in their entirety.

Specific binding to an antibody under such conditions requires an antibody that is selected for its specificity for a particular protein. For example, antibodies raised against a particular protein, polymorphic variants, alleles, orthologs, and conservatively modified variants, or splice variants, or portions thereof; can be selected to obtain only those polyclonal antibodies that are specifically immunoreactive with, e.g., a VWF protein (or a portion thereof, such as the A1 domain) and not with other proteins. This selection may be achieved by subtracting out antibodies that cross-react with other molecules. A variety of immunoassay formats may be used to select antibodies specifically immunoreactive with a particular protein. For example, solid-phase ELISA immunoassays are routinely used to select antibodies specifically immunoreactive with a protein (see, e.g., Harlow & Lane, Antibodies, A Laboratory Manual (1988) for a description of immunoassay formats and conditions that can be used to determine specific immunoreactivity). Methods for determining whether two molecules specifically interact are disclosed herein, and methods of determining binding affinity and specificity are well known in the art (see, for example, Harlow and Lane, Antibodies: A laboratory manual (Cold Spring Harbor Laboratory Press, 1988); Friefelder, “Physical Biochemistry: Applications to biochemistry and molecular biology” (W.H. Freeman and Co. 1976)).

Furthermore, VWF binding agent (or a VWF-A1 domain binding agent) can interfere with the specific binding of a VWF and a platelet (or a protein in the platelet, such as, e.g., GPIb-alpha protein) by various mechanism. For purposes of the methods disclosed herein, an understanding of the mechanism by which the interference occurs is not required and no mechanism of action is proposed. An VWF binding agent (or a VWF-A1 domain binding agent), such as an anti-VWF antibody, an anti-VWF-A1 domain antibody, or Fab fragments thereof, is characterized by having specific binding activity (Kₐ) for a VWF protein, e.g., a polypeptide that includes amino acid 1240P-1481G of SEQ ID NO:6 or a functional equivalent thereof, or the VWF-A1 domain antibody, as appropriate, of at least about 10⁵ mol⁻¹, 10⁶ mol⁻¹, or greater, preferably 10⁷ mol⁻¹, or greater, more preferably 10⁸ mol⁻¹, or greater, and most preferably 10⁹ mol⁻¹, or greater. The binding affinity of an antibody can be readily determined by one of ordinary skill in the art, for example, by Scatchard analysis (Scatchard, Ann. NY Acad. Sci. 51: 660-72, 1949).

### Candidate Agents

Candidate agents include compounds that may be obtained from large libraries of synthetic or natural compounds. Numerous means are currently used for random and directed synthesis of saccharide, peptide, and nucleic acid based compounds. Synthetic compound libraries are commercially available from Maybridge Chemical Co. (Trevillet, Cornwall, UK), Comgenex (Princeton, N.J.), Brandon Associates (Merrimack, N.H.), and Microsource (New Milford, Conn.). A rare chemical library is available from Aldrich (Milwaukee, Wis.). Alternatively, libraries of natural compounds in the form of bacterial, fungal, plant and animal extracts are available from e.g. Pan Laboratories (Bothell, Wash.) or MycoSearch (N.C.), or are readily producible. Additionally, natural and synthetically produced libraries and compounds are readily modified through conventional chemical, physical, and biochemical means (Blondelle et al., (1996) Tib Tech 14:60).

Methods for preparing libraries of molecules are well known in the art and many libraries are commercially available. Libraries of interest in the invention include peptide libraries, randomized oligonucleotide libraries, synthetic organic combinatorial libraries, and the like. Degenerate peptide libraries can be readily prepared in solution, in immobilized form as bacterial flagella peptide display libraries or as phage display libraries. Peptide ligands can be selected from combinatorial libraries of peptides containing at least one amino acid. Libraries can be synthesized of peptoids and non-peptide synthetic moieties. Such libraries can further be synthesized which contain non-peptide synthetic moieties, which are less subject to enzymatic degradation compared to their naturally-occurring counterparts. Libraries are also meant to include for example but are not limited to peptide-on-plasmid libraries, polysome libraries, aptamer libraries, synthetic peptide libraries, synthetic small molecule libraries and chemical libraries. The libraries can also comprise cyclic carbon or heterocyclic structure and/or aromatic or polyaromatic structures substituted with one or more of the above-identified functional groups. Screening compound libraries listed above [also see Examples below and U.S. Patent Application Publication No. 2005/0009163, which is hereby incorporated by reference], in combination with dynamic force microscopy, a coagulation factor assay, a platelet adhesion assay, thrombus imaging, a bleeding time assay, aggregometry, review of real-time video of blood flow, a Doppler ultrasound vessel occlusion assay, or a combination of these assays (for example, those assays described in EXAMPLES 1-5) can be used to identify modulators of VWF-A1 binding to GPIb-alpha, wherein the compound abbreviates or increases off-rate (k_{off}) binding kinetics between VWF-A1 and GPIb-alpha by at least two-fold (Lew et al., (2000) Curr. Med. Chem. 7(6):663-72; Werner et al., (2006) Brief Funct. Genomic Proteomic 5(1):32-6).

Small molecule combinatorial libraries may also be generated. A combinatorial library of small organic compounds is a collection of closely related analogs that differ from each other in one or more points of diversity and are synthesized by organic techniques using multi-step processes. Combinatorial libraries include a vast number of small organic compounds. One type of combinatorial library is prepared by means of parallel synthesis methods to produce a compound array. A compound array can be a collection of compounds identifiable by their spatial addresses in Cartesian coordinates and arranged such that each compound has a common molecular core and one or more variable structural diversity elements. The compounds in such a compound array are produced in parallel in separate reaction vessels, with each compound identified and tracked by its spatial address. Examples of parallel synthesis mixtures and parallel synthesis methods are provided in U.S. Ser. No. 08/177,497, filed Jan. 5, 1994 and its corresponding PCT published patent application WO95/18972, published Jul. 13, 1995 and U.S. Pat. No. 5,712,171 granted Jan. 27, 1998 and its corresponding PCT published patent application WO96/22529, which are hereby incorporated by reference.

Examples of chemically synthesized libraries are described in Fodor et al., (1991) Science 251:767-773; Houghten et al., (1991) Nature 354:84-86; Lam et al., (1991) Nature 354:82-84; Medynski, (1994) BioTechnology 12:709-710; Gallop et al., (1994) J. Medicinal Chemistry 37(9):1233-1251; Ohlmeyer et al., (1993) Proc. Natl. Acad. Sci. USA 90:10922-10926; Erb et al., (1994) Proc. Natl. Acad. Sci. USA 91:11422-11426; Houghten et al., (1992) Biotechniques 13:412; Jayawickreme et al., (1994) Proc. Natl. Acad. Sci. USA 91:1614-1618; Salmon et al., (1993) Proc. Natl. Acad. Sci. USA 90:11708-11712; PCT Publication No. WO 93/20242, dated Oct. 14, 1993; and Brenner et al., (1992) Proc. Natl. Acad. Sci. USA 89:5381-5383.

Examples of phage display libraries are described in Scott et al., (1990) Science 249:386-390; Devlin et al., (1990) Science, 249:404-406; Christian, et al., (1992) J. Mol. Biol. 227:711-718; Lenstra, (1992) J. Immunol. Meth. 152:149-157; Kay et al., (1993) Gene 128:59-65; and PCT Publication No. WO 94/18318.

In vitro translation-based libraries include but are not limited to those described in PCT Publication No. WO 91/05058; and Mattheakis et al., (1994) Proc. Natl. Acad. Sci. USA 91:9022-9026.

In one non-limiting example, non-peptide libraries, such as a benzodiazepine library (see e.g., Bunin et al., (1994) Proc. Natl. Acad. Sci. USA 91:4708-4712), can be screened. Peptoid libraries, such as that described by Simon et al., (1992) Proc. Natl. Acad. Sci. USA 89:9367-9371, can also be used. Another example of a library that can be used, in which the amide functionalities in peptides have been permethylated to generate a chemically transformed combinatorial library, is described by Ostresh et al. (1994), Proc. Natl. Acad. Sci. USA 91:11138-11142.

Preferred candidate agents are small molecules. Small molecules can include any number of therapeutic agents presently known and used, or can be synthesized in a library of such molecules for the purpose of screening for biological function(s). Small molecules are distinguished from macromolecules by size. The small molecules of this invention usually have a molecular weight less than about 5,000 Daltons (Da), preferably less than about 2,500 Da, more preferably less than 1,000 Da, most preferably less than about 500 Da.

Preferred small molecules are relatively easier and less expensively manufactured, formulated or otherwise prepared. Preferred small molecules are stable under a variety of storage conditions. Preferred small molecules may be placed in tight association with macromolecules to form molecules that are biologically active and that have improved pharmaceutical properties. Improved pharmaceutical properties include changes in circulation time, distribution, metabolism, modification, excretion, secretion, elimination, and stability that are favorable to the desired biological activity. Improved pharmaceutical properties include changes in the toxicological and efficacy characteristics of the chemical entity.

Diversity libraries, such as random or combinatorial peptide or non-peptide libraries can be screened for small molecules and compounds that specifically bind to a VWF-A1 protein. Many libraries are known in the art that can be used such as, e.g., chemically synthesized libraries, recombinant (e.g., phage display) libraries, and in vitro translation-based libraries.

Any screening technique known in the art can be used to screen for agonist (i.e., compounds that promote platelet adhesion) or antagonist molecules (such as anti-thrombotics) directed at a target of interest (e.g. VWF-A1). The present invention contemplates screens for small molecule ligands or ligand analogs and mimics, as well as screens for natural ligands that bind to and modulate VWF-A1 binding to GPIb-alpha, such as via examining the degree of thrombus formation, platelet adhesion, coagulation, blood flow, vessel occlusion, or bleeding times. For example, natural products libraries can be screened using assays of the invention for molecules that modulate the activity of a molecule of interest, such as a VWF-A1 binding to GPIb-alpha.

Knowledge of the primary sequence of a molecule of interest, such as a VWF-A1, can provide an initial clue as to proteins that can modulate VWF-A1 binding to GPIb-alpha. Identification and screening of modulators is further facilitated by determining structural features of the protein, e.g., using X-ray crystallography, neutron diffraction, nuclear magnetic resonance spectrometry, and other techniques for structure determination. These techniques provide for the rational design or identification of such modulators.

Screening the libraries can be accomplished by any variety of commonly known methods. See, for example, the following references, which disclose screening of peptide libraries: Parmley and Smith, (1989) Adv. Exp. Med. Biol. 251:215-218; Scott and Smith, (1990) Science 249:386-390; Fowlkes et al., (1992) BioTechniques 13:422-427; Oldenburg et al., (1992) Proc. Natl. Acad. Sci. USA 89:5393-5397; Yu et al., (1994) Cell 76:933-945; Staudt et al., (1988) Science 241:577-580; Bock et al., (1992) Nature 355:564-566; Tuerk et al., (1992) Proc. Natl. Acad. Sci. USA 89:6988-6992; Ellington et al., (1992) Nature 355:850-852; U.S. Pat. Nos. 5,096,815; 5,223,409; and 5,198,346, all to Ladner et al.; Rebar et al., (1993) Science 263:671-673; and PCT Pub. WO 94/18318.

The present invention provides methods for evaluating potential anti-thrombotic reagents in pre-clinical testing using a non-human transgenic animal. The animal may be any useful non-human laboratory or agricultural animal. For example, the animal may be selected from the group consisting of mouse, rat, hamster, guinea pig, rabbit, dog, goat, horse, and monkey.

There are at least three classes of antithrombotic drugs that can be screened using the transgenic mouse model of the invention: Anticoagulant drugs (such as Heparins; Vitamin K antagonists, which are currently the only anticoagulants that can be administered orally; and direct thrombin inhibitors), Antiplatelet drugs (such as cyclooxygenase inhibitors like aspirin; phosphodiesterase inhibitors like ticlopidine (Ticlid); adenosine diphosphate receptor inhibitors like clopidogrel (Plavix); tirofiban (Aggrastat); adenosine reuptake inhibitors, and inhibitors of integrins on platelets (for example, alpha IIb Beta3) like eptifibatide (Integrilin)), and Thrombolytic or fibrinolytic drugs (such as t-PA (alteplase Activase); reteplase (Retavase); urokinase (Abbokinase); streptokinase (Kabikinase, Streptase); tenectaplase; lanoteplase; and anistreplase (Eminase)).

The invention provides an in vivo model to test the efficacy of potential anti-thrombotic drugs against human platelets prior to FDA approval. To date, in vitro models of thrombosis do not accurately recapitulate the hemodynamic conditions, cell-cell interactions, or cell-protein interactions that occur at sites of vascular injury in a living animal. Thus, anti-thrombotics can be identified and their potential therapeutic effects can be assessed for treatment of abnormal thrombotic events associated with atherothrombotic arterial diseases and venous thrombotic diseases (such as abnormal bleeding and/or abnormal clotting).

Atherothrombotic arterial diseases can include, but is not limited to, coronary artery disease, (for example, acute myocardial infarction, acute coronary syndromes (such as unstable angina pectoris) and stable angina pectoris); mesenteric ischemia, “abdominal angina,” and mesenteric infarction; cerebral vascular disease, including acute stroke and transient ischemic attack; mesenteric arterial disease; as well as peripheral arterial disease, including acute peripheral arterial occlusion and intermittent claudication. Anti-thrombotic compounds identified by the pre-clinical testing method of the present invention can also be useful for the treatment of coronary artery disease (which includes, but is not limited to anti-thrombotic therapy during coronary angioplasty, anti-thrombotic therapy during cardiopulmonary bypass, and limiting of platelet activation during ischemia reperfusion) as well as venous thrombotic diseases (which include, but are not limited to deep venous thrombosis and pulmonary thromboembolism). Anti-thrombotic compounds identified by the pre-clinical testing method of the present invention can also be useful in anti-thrombotic therapy for pulmonary hypertension.

### Administering Candidate Agents

The candidate agents may be administered by a topical, oral, rectal, parenteral (such as subcutaneously, intramuscularly, intravenously, intraperitoneally, intrapleurally, intravesicularly or intrathecally), or nasal route. These compounds may also be applied topically or locally, in liposomes, solutions, gels, ointments, biodegradable microcapsules, or impregnated bandages. Compositions or dosage forms for topical application may include suspensions, dusting powder, solutions, lotions, suppositories, sprays, aerosols, biodegradable polymers, ointments, creams, gels, impregnated bandages and dressings, liposomes, and artificial skin.

The candidate agents may also be formulated in a pharmaceutical composition prior to administration. A “pharmaceutical composition” refers to a mixture of one or more of the compounds, or pharmaceutically acceptable salts, hydrates, polymorphs, or pro-drugs thereof, with other chemical components, such as physiologically acceptable carriers and excipients. The purpose of a pharmaceutical composition is to facilitate administration of a compound to an organism. The pharmaceutical composition may contain other components so long as the other components do not reduce the effectiveness of the compound according to this invention so much that the therapy is negated. Some other components may have independent therapeutic effects. Pharmaceutically acceptable carriers are well known, and one skilled in the pharmaceutical art can easily select carriers suitable for particular routes of administration (see, e.g., Remington's Pharmaceutical Sciences, Mack Publishing Co., Easton, Pa., 1985).

Pharmaceutical carriers utilized by one skilled in the art which make up the foregoing compositions include petrolatum, polyethylene glycol, alginates, carboxymethylcellulose, methylcellulose, agarose, pectins, gelatins, collagen, vegetable oils, phospholipids, stearic acid, stearyl alcohol, polysorbate, mineral oils, polylactate, polyglycolate, polyanhydrides, polyvinylpyrrolidone, and the like.

A “pro-drug” refers to an agent which is converted into the parent drug in vivo. Pro-drugs are often useful because, in some situations, they are easier to administer than the parent drug. They are bioavailable, for instance, by oral administration whereas the parent drug is not. The pro-drug also has improved solubility in pharmaceutical compositions over the parent drug. For example, the compound carries protective groups which are split off by hydrolysis in body fluids, e.g., in the bloodstream, thus releasing active compound or is oxidized or reduced in body fluids to release the compound.

A compound of the present invention also can be formulated as a pharmaceutically acceptable salt, e.g., acid addition salt, and complexes thereof. The preparation of such salts can facilitate the pharmacological use by altering the physical characteristics of the agent without preventing its physiological effect. Examples of useful alterations in physical properties include, but are not limited to, lowering the melting point to facilitate transmucosal administration and increasing the solubility to facilitate administering higher concentrations of the drug.

The term “pharmaceutically acceptable salt” means a salt, which is suitable for or compatible with the treatment of a patient or a subject such as a human patient or an animal.

The term “pharmaceutically acceptable acid addition salt” as used herein means any non-toxic organic or inorganic salt of any base compounds of the invention or any of their intermediates. Illustrative inorganic acids which form suitable acid addition salts include hydrochloric, hydrobromic, sulfuric and phosphoric acids, as well as metal salts such as sodium monohydrogen orthophosphate and potassium hydrogen sulfate. Illustrative organic acids that form suitable acid addition salts include mono-, di-, and tricarboxylic acids such as glycolic, lactic, pyruvic, malonic, succinic, glutaric, fumaric, malic, tartaric, citric, ascorbic, maleic, benzoic, phenylacetic, cinnamic and salicylic acids, as well as sulfonic acids such as p-toluene sulfonic and methanesulfonic acids. Either the mono or di-acid salts can be formed and such salts exist in either a hydrated, solvated or substantially anhydrous form. In general, the acid addition salts of compounds of the invention are more soluble in water and various hydrophilic organic solvents, and generally demonstrate higher melting points in comparison to their free base forms. The selection of the appropriate salt will be known to one skilled in the art. Other non-pharmaceutically acceptable salts, e.g., oxalates, are used, for example, in the isolation of compounds of the invention for laboratory use or for subsequent conversion to a pharmaceutically acceptable acid addition salt.

The candidate agent that alters interactions between a VWF, such as the VWF which includes amino acids 1240P-1481G of SEQ ID NO:6, and a human platelet may be used to treat diseases disclosed herein. As used herein and as well understood in the art, “treatment” is an approach for obtaining beneficial or desired results, including clinical results. Beneficial or desired clinical results can include, but are not limited to, alleviation or amelioration of one or more symptoms or conditions, diminishment of extent of disease, stabilized (i.e., not worsening) state of disease, preventing spread of disease, delay or slowing of disease progression, amelioration or palliation of the disease state and remission (whether partial or total), whether detectable or undetectable. “Treatment” can also mean prolonging survival as compared to expected survival if not receiving treatment.

Therapy dose and duration will depend on a variety of factors, such as the disease type, patient age, therapeutic index of the drugs, patient weight, and tolerance of toxicity. Initial dose levels will be selected based on their ability to achieve ambient concentrations shown to be effective in in vitro models (for example, a dose level used to determine therapeutic index), in vivo models, and in clinical trials. The skilled clinician using standard pharmacological approaches can determine the dose of a particular drug and duration of therapy for a particular patient in view of the above stated factors. The response to treatment can be monitored by analysis of body fluid or blood levels of the compound and the skilled clinician will adjust the dose and duration of therapy based on the response to treatment revealed by these measurements.

### Labeled Agent

A “labeled” agent means an agent that has an attachment which renders the agent detectable. In the present invention, the labeled agent would aid in the detection of either the presence or absence of a thrombus. As set forth above, the labeled agent may comprise a nanoparticle, a fluorophore, a quantum dot, a microcrystal, a radiolabel, a dye, a gold biolabel, an antibody, a peptide, a small molecule ligand, or a combination thereof. A fluorophore, for example green fluorescent protein, (such as GFP, RFP, YFP and the like; see Johnson and Johnson, (2007) ACS Chem. Biol. 2(1):31-8) can be used as a biomarker. A quantum dot is a semiconductor nanocrystal, that can be as small as 2 to 10 nm or can 15-20 nm (for example, Q-dot nanocrystals; also see Kaji et al., (2007) Anal Sci. 23(1):21-4). Quantum dot fluorescence can be induced by exposure to ultraviolet light. Both a fluorescent protein and a quantum dot can be obtained commercially (for example, Molecular Probes—Invitrogen, Carlsbad, Calif. or Evident Technologies, Troy N.Y.). A fluorophore can also be generated in the laboratory according to molecular biology methods practiced in the art. A radiolabel is a radioactive isotope that can be used as a tracer. Non-limiting examples of radiolabels include: Technetium-99m, Iodine-123 and 131, Thallium-201, Gallium-67, Fluorine-18, -19, Indium-111, Xenon-I 33, and Krypton-81m. Radiolabels can be obtained commercially, for example, from SRI International (Menlo Park, Calif.). As set forth above, certain methods include the use of a nanoparticle, which may comprise a perfluorocarbon (PFC). Non-limiting examples of perfluorocarbons include perfluorobutane, perfluorohexane, perfluorooctane, perfluorodecal in, perfluoromethyldecalin, and perfluoroperhydrophenanthrene. These can be synthesized according to the method described in EXAMPLE 5 or according to Partlow et al. (FASEB J (2007) February 6 on-line publication). The perfluorocarbon molecules can also be obtained commercially (F2 Chemicals Ltd.; Lancashire, UK). The PFC nanoparticle can be coupled to a platelet receptor antibody (such as platelet receptor alpha-IIb beta₃). In some embodiments, imaging can comprise a PET scan, a CT scan, an MRI, an IR scan, an ultrasound, nuclear imaging, or a combination thereof.

### Methods for Assessing Thrombotic Events In Vivo

The invention provides methods for detecting an internal vascular injury site (occult bleeding) in a subject. This could be useful in emergency room (ER) settings or on the battlefield in order to quickly identify sites of internal bleeding. For instance, the method can entail: administering to a subject a targeted molecular imaging agent, wherein the molecule circulates for an effective period of time in order to bind to the injury site within the subject; tracking a deposition of the labeled thrombosis-indicating-molecule in the subject; and identifying the site of a thrombus formation in the subject by imaging the labeled targeted molecular imaging agent. Thus, the deposition of the targeted molecular imaging agent at the internal vascular injury site can be indicative of internal bleeding within a subject. For example, a targeted molecular imaging agent can recognize constituents of thrombi that comprise a lipid, a protein, a cellular molecule, or a combination thereof.

The invention also provides a method to test contrast agents for imaging of human platelets at sites of thrombosis. For instance, one could test the ability of nanoparticle contrast agents targeted to human platelets to identify areas of thrombosis or occult bleeding. In some embodiments, the prevention or reduction of thrombus formation at site of injury upon administration of a compound can be visually examined via tracking the localization of labeled platelets (such as with high resolution in vivo microscopy or MRI). In further embodiments, the platelets can be labeled with a nanoparticle, fluorophore, quantum dot, microcrystal, radiolabel, dye, or gold biolabel. The prevention or reduction of thrombus formation also can be readily determined by methods known to one skilled in the art, which include but are not limited to aggregometry, review of real-time video of blood flow in the animal, and determination of vessel occlusion, as well as by the examples provided below.

The invention also provides a method to correlate results obtained with an in vitro assay designed to measure the effects of antithrombotics or biomarkers of platelet activation in patients. For example, a biomarker is an indicator of a particular disease state or a particular state of an organism, such as when the subject experiences vascular vessel wall injury. Upon injury to the vessel wall and subsequent damage to the endothelial lining, exposure of the subendothelial matrix to blood flow results in deposition of platelets at the site of injury via binding to the collagen with the surface collagen-specific glycoprotein Ia/IIa receptor. This adhesion is strengthened further by the large multimeric circulating protein VWF, which forms links between the platelet glycoprotein Ib/IX/V and collagen fibrils. The platelets are then activated and release the contents of their granules into the plasma, in turn activating other platelets. For example, Glycoprotein VI (GP6) is a 58-kD platelet membrane glycoprotein that plays a crucial role in the collagen-induced activation and aggregation of platelets. The shedding of GP6 can act as a marker representing that a person is at risk of myocardial infarction. In one embodiment, platelets obtained from a subject determined to have an elevated biomarker level (for example, GP6) can be infused into the non-human transgenic animal described above according to previously described methods, wherein the occurrence of a thrombotic event can be evaluated. In another embodiment, platelets obtained from a subject undergoing an anti-thrombotic treatment can be infused into the non-human transgenic animal described above according to previously described methods, wherein the occurrence of a thrombotic event can then be evaluated.

The following examples are provided to further illustrate the methods of the present invention. These examples are illustrative only and are not intended to limit the scope of the invention in any way.

### EXAMPLES

### Example 1

### VWF Characterization

### VWF Microsphere Studies

The association and dissociation kinetics of the GPIb α-VWF-A1 bond and the impact of fluid shear and particle size on these parameters can be determined by measuring the frequency and duration of transient adhesive events, known as transient tethers, that represent the smallest unit of interaction observable in a parallel-platelet flow chamber.

The generation of recombinant VWF-A1 protein (residues 1238 to 1472 of the mature, recombinant VWF) and its subsequent coupling to microspheres is performed as previously described (Doggett, et al., 2002). Proper size, purity, and disulfide bonding of all proteins is assessed by coomasie-blue staining of SDS-PAGE gels run under reducing and non-reducing conditions. Mass spectrometry is also employed to evaluate size and disulfide bonding pattern.

The resulting recombinant proteins are bound to polystyrene microspheres (goat anti-mouse IgG (FC); Bangs Lab, Inc., Fishers, Ind.) that were initially coated with a saturating concentration of mouse anti-6-HIS antibody as previously described. This coating method was found to be superior to direct covalent coupling of the VWF-A1 to the beads because it prevents significant loss in protein function. Estimation of the amount of VWF-A1 bound to the beads is determined using monoclonal antibodies generated in the inventors' laboratory against the human and murine A1 domains, mAb AMD-1 and mAb AMD-2, respectively, and a calibrated microbead system (Quantum Simply Cellular; Flow Cytometry Standards Corp., San Juan, P.R.) following the manufacturer's instructions.

### Laminar Flow Assays.

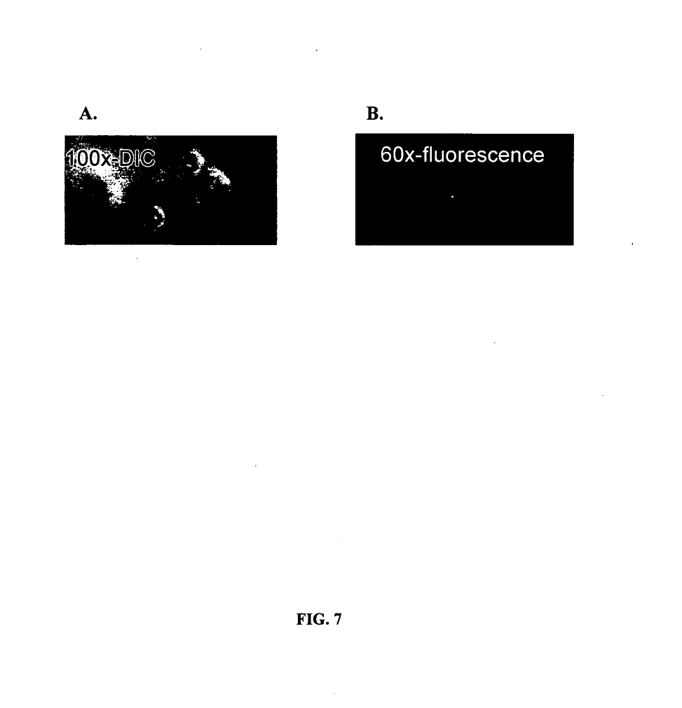

In flow assays involving protein-coated microspheres, human or murine platelets purified by gel filtration are incubated with 10 mM sodium azide (NaN₃), 50 ng/ml prostaglandin E₁, and 10 μm indomethacin (Sigma Immunochemicals, St. Louis, Mo.) to reduce the possibility of activation and potential alterations in expression and/or distribution of GPIb α on their surface. Platelets are subsequently allowed to settle in stasis on Fab fragments of monoclonal antibodies that recognize either human (i.e., mAb 7E3) or murine (i.e., mAb NAD-1) αIIb/β₃ in order to form a reactive substrate. The use of platelets in lieu of recombinant proteins or transfected cells as the immobilized substrate enables evaluation of GPIb α in its native form (i.e. correct orientation and proper post-translational modification). Platelet coverage of <10% bound in this manner can remain relatively unactivated for up to 30 minutes as evident by morphology on light microscopic examination (FIG. 7A) and lack of expression of P-selectin by fluorescence microscopy (FIG. 7B).

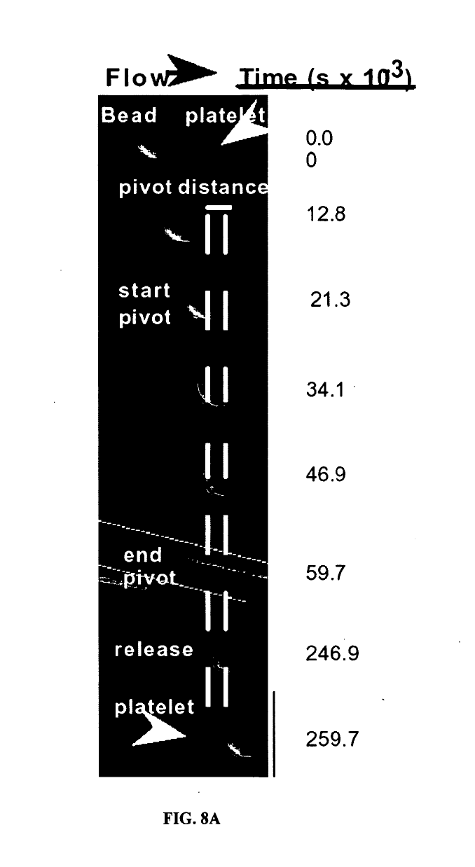

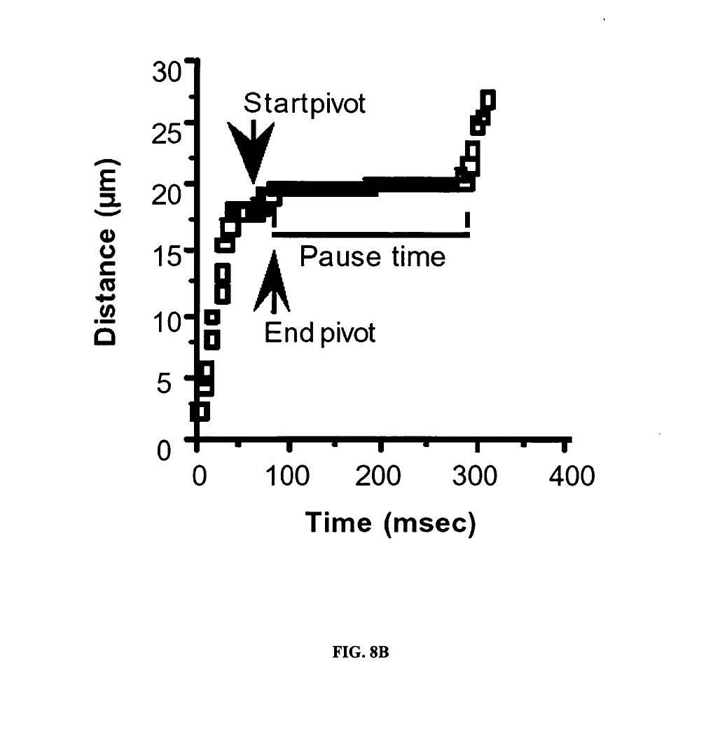

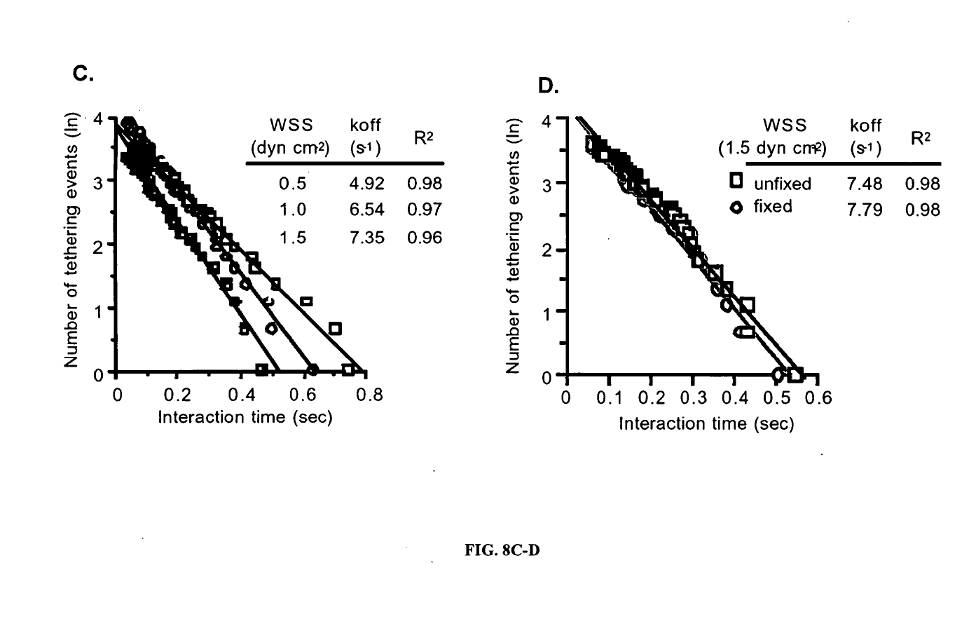

To reduce the possibility of multiple bond formation that would result in a prolongation in interaction times between the beads and immobilized platelets, the lowest site densities of VWF-A1 capable of supporting these brief interactions is used, a value that corresponds to about 30 molecules μm². At this site density, the formation of transient tethers between this receptor-ligand pair has distribution of bond lifetimes that obey first order dissociation kinetics. The duration of these interactions are measured by recording images from a Nikon X60 DIC objective (oil immersion) viewed at a frame rate of 235 fps (Speed Vision Technologies, San Diego, Calif.) and subjected to wall shear stresses (WSS) ranging from 0.5 to 3.0 dyn cm⁻². The cellular off-rates are determined by plotting the natural log of the number of VWF-A1 coated microspheres that interacted as a function of time after the initiation of tethering, the slope of the line=−k_{off} (s⁻¹) which is the inverse of the bond lifetime. The force acting on the i tether bond was calculated from force balance equations and k_{off} plotted as a function of these forces. An example of the measurement of the duration of a transient tether and estimation of off-rates as a function of WSS is demonstrated for WT human VWF-A1 (FIG. 8, A-C). To demonstrate that this method for surface immobilization of platelets does not result in an alteration in the kinetics of the GPIb α-VWF-A1 tether bond, resting platelets were first fixed in paraformaldehyde prior to immobilization. Because these platelets cannot activate, the kinetics should be reflective of GPIb α in the resting state. Indeed, analysis of the k_{off} for this interaction using fixed platelets was identical to that observed for platelets treated with metabolic inhibitors (FIG. 8D).

### The Structure-Function of Murine VWF-A1

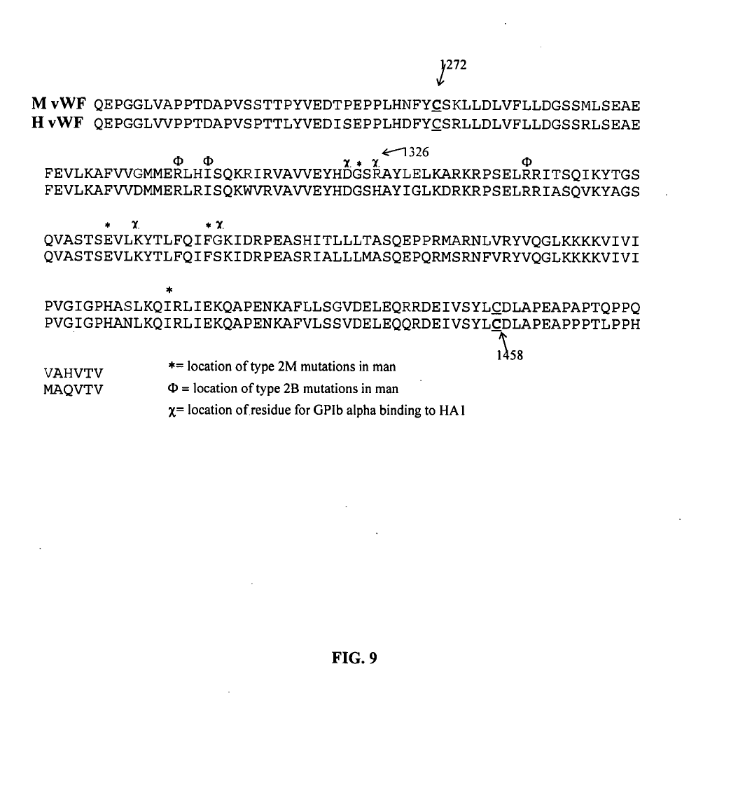

To determine the structure and function of murine VWF-A1, its adhesive interactions with murine and human GPIb α, and whether the kinetics of this interaction mimic those reported in studies of its human counterpart, the domain was initially cloned by PCR from purified mouse genomic DNA. For the purpose of generating a mouse with a genetically modified VWF-A1 domain, a 100-kb P1 clone was obtained from screening a 129/Svj DNA genomic library (Genomic Systems, St. Louis, Mo.) by polymerase chain reaction (PCR) using primers directed against a 200 by region of exon 28. Sequence analysis of flanking regions (10 kb in size) as well as the A1 domain itself was performed and compared to those obtained from a BLAST search to confirm the fidelity of the clone. The deduced single-letter amino acid sequence of mouse VWF-A1 domain (M VWF) is shown compared to its human counterpart (H VWF) and encompasses amino acids 1260 to 1480 (FIG. 9). The locations of cysteines forming the loop structure are numbered (1272 and 1458). Conversion of the arginine (R) in the mouse A1 domain to histidine (H) as found in its human counterpart (x) has been shown to enable mouse VWF to bind human platelets and simultaneously reduce the binding of mouse platelets. Locations of some, but not all, mutations known to affect human VWF-A1 function are also depicted.

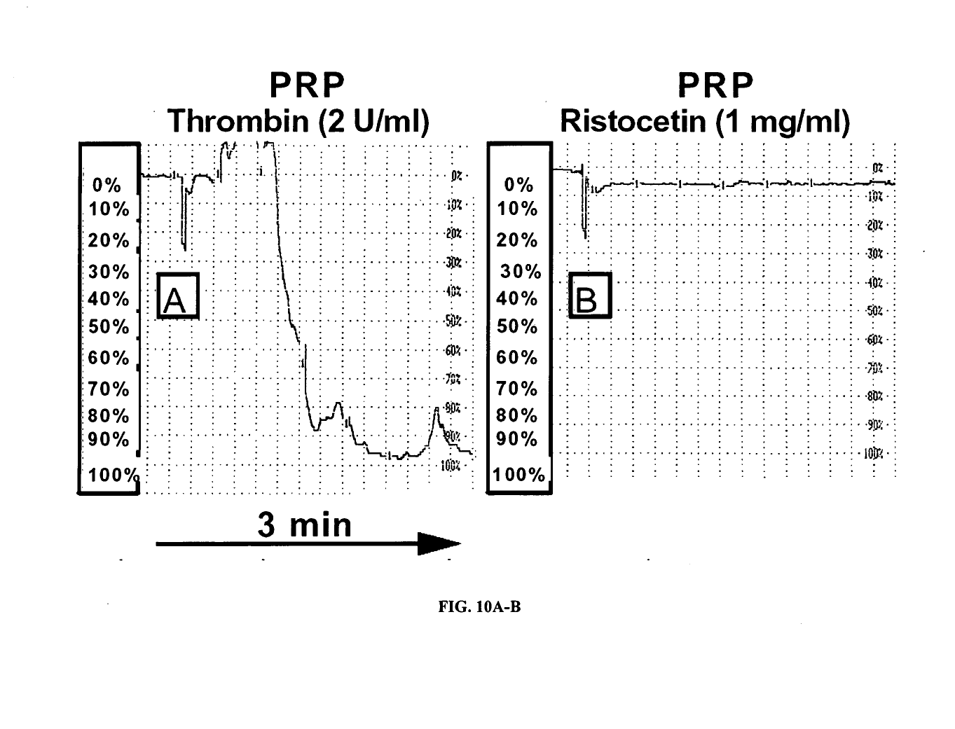

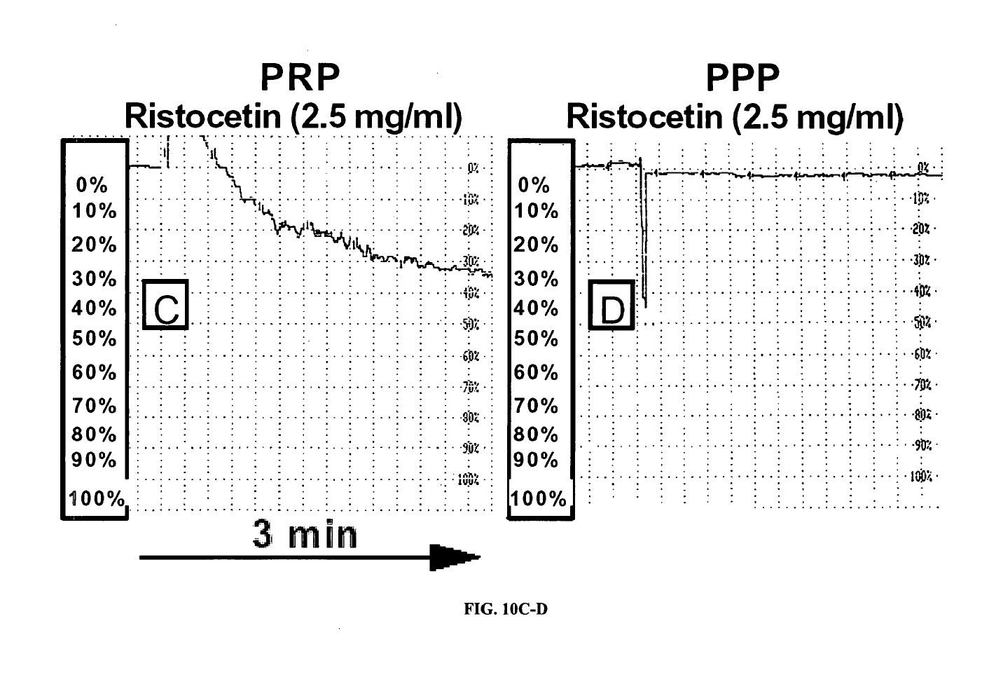

Although the amino acid sequence homology between the human and mouse VWF-A1 domains is high (about 85% identity), preliminary studies suggest that functional differences do exist between human and murine VWF-A1 domains. In Ristocetin-induced platelet aggregation assays (RIPA), platelet GPIb a binding to wild type human VWF or mouse VWF was analyzed in the absence or presence of ristocetin as described previously (Inbal, et al., 1993). In this method, platelet rich plasma (PRP) is placed in a clear cuvette containing a stir bar and inserted into the aggregometer. Platelet aggregation is induced by the addition of ristocetin. In this RIPA, it was observed that concentrations of this modulator that are known to cause agglutination of human platelets (about 1.0 mg/ml) had no such effect using murine PRP (FIG. 10B). In fact, only at concentrations of 2.5 mg/ml was there any evidence of murine platelet aggregation observed (about 30%, FIG. 10C). In comparison, incubation of murine PRP with thrombin resulted in >90% platelet aggregation (FIG. 10A).

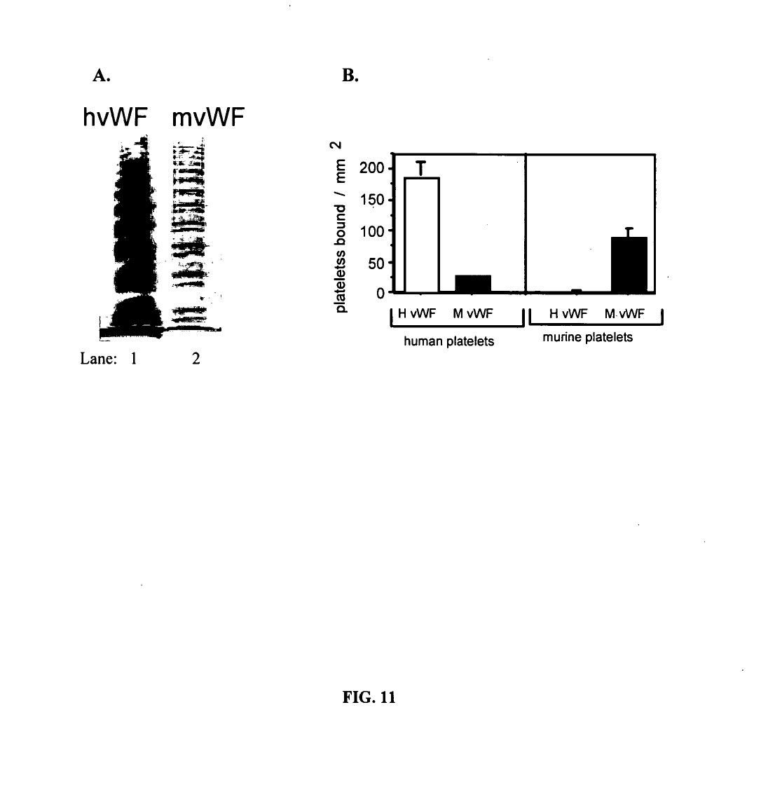

To better evaluate the above interactions and to compare functional relationships between human and murine VWF with GPIb α, VWF from human and mouse plasma was purified and its ability to mediate platelet adhesion in flow was determined. Multimer gel analysis did not reveal any differences between the two species, especially with regard to high molecular weight components (FIG. 11A). Moreover, surface-immobilized murine VWF could support adhesion of syngeneic platelets (1×10⁸/ml) at a shear rate encountered in the arterial circulation (1600 s⁻¹) as observed for the human plasma protein (FIG. 11B). In contrast, murine VWF did not support significant interactions with human platelets and vice versa. These results suggest that functional and possibly significant structural differences do exist between the A1 domains of murine and human VWF as primary attachment of platelets at this wall shear rate is dependent on its function. Thus, generation of a recombinant murine VWF-A1 domain is required to fully evaluate similarities and/or differences from its human counterpart.

Recombinant protein was expressed using a bacterial expression vector under the control of an inducible promoter (pQE9, Qiagen). Insertion of the murine fragment containing the majority of the VWF A1-domain (encoding for amino acids 1233 to 1471) into pQE9 produces an amino-terminal fusion protein containing 10 amino acids (including 6Xhistidine) contributed by the vector. After induction, inclusion bodies were harvested, washed, and solubilized according to previously published methods (Cruz et al., 2000). The solubilized protein was diluted 40-fold in 50 mM Tris-HCl, 500 mM NaCl, 0.2% Tween 20, pH 7.8 and initially purified over a Ni²⁺-chelated Sepharose (Pharmacia) column. To increase the yield of functional protein, the material purified from the Ni²⁺ column was absorbed to and eluted from a Heparin-Sepharose column (Amersham Pharmacia Biotech).

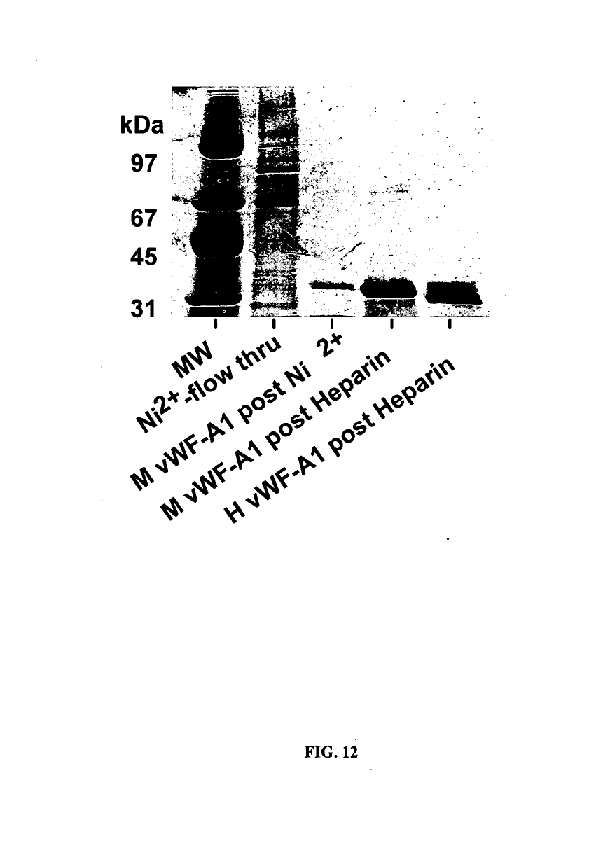

The highly purified protein was dialyzed against 25 mM Tris-HCl, 150 mM NaCl, 0.05% Tween 20, pH 7.8. SDS-PAGE analysis revealed a prominent protein band of about 34,000 Da under non-reducing conditions (FIG. 12). The overall yield of protein obtained using the purification methods described above is about 2 mg/l of bacterial cells.

The protein was subsequently used in a series of in vitro flow chamber assays to assess function. Washed human or murine platelets (5×10⁷/ml) were infused through a parallel plate flow chamber containing glass cover slips coated with either human VWF-A1 or mouse VWF-A1 protein (100 μg/ml final concentration) at a shear rate of 800 s⁻¹. After 5 minutes of continuous flow, adherent platelets were quantified.

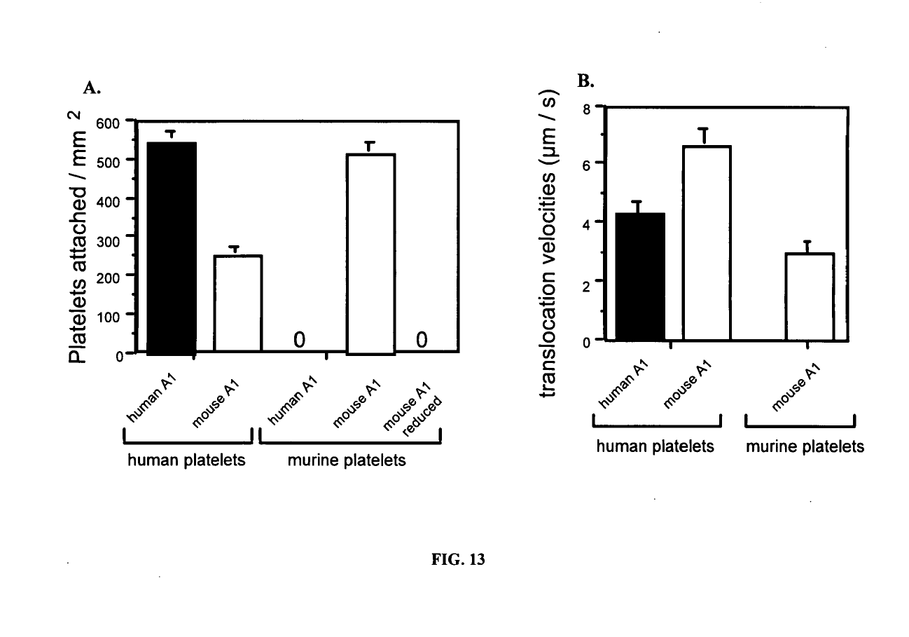

As shown in FIG. 13A, mouse VWF-A1 protein supported platelet adhesion as efficiently as its human counterpart under physiological flow conditions. To demonstrate the importance of the single disulfide bond formed by C1272 and C1458, reduced (DTT) and alkylated (iodoacetamide) mouse VWF-A1 was prepared and tested in flow. Reduction and alkylation of the protein abrogated attachment of murine platelets in flow. In addition, the limited ability of the native form of the protein to mediate adhesion of human platelets and lack of interaction between human VWF-A1 and mouse platelets suggests that structural/conformational differences exist between the species. However, this does not preclude the study of GPIb α-VWF-A1 interactions in mice as both proteins must share common kinetic attributes because they support rapid attachment and translocation of platelets to a similar degree under physiological flow conditions (FIG. 13B).

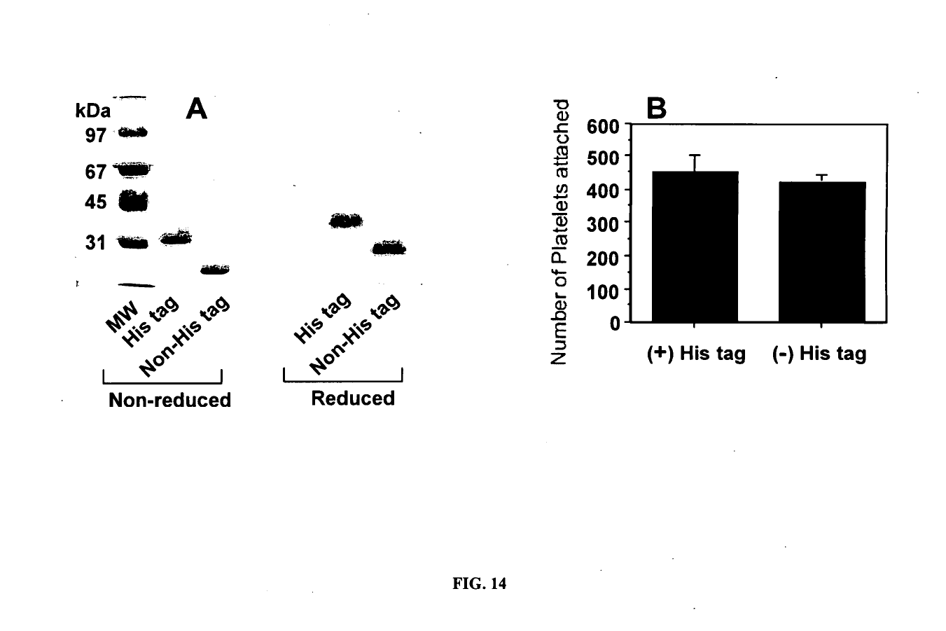

To demonstrate that the presence of the N-terminus His tag does not appear to affect the function of the recombinant protein, the ability of a tagged vs. a non-tagged M VWF-A1 to support mouse platelet adhesion in flow was compared. In the case of the latter, the murine A1 fragment was inserted into pET-11b (Stratagene) and purified as previously described (Miura et al., 2000). The purified bacterial non-His tag protein was analyzed by SDS-PAGE (12.5%) and found to migrate in an analogous manner to its tagged counterpart under non-reducing and reducing conditions (FIG. 14A). In addition, no differences were observed in the number of platelets that adhered to and translocated on either protein (449±53 platelets/mm² His-tag vs. 423±17 platelets/mm² non-His tag) at a wall shear rate of 800 s⁻¹ (FIG. 14B).

### Characterization of the M VWF-A1 Domain.

The A1 domains of human and mouse serve an identical purpose: to mediate primary attachment and translocation of platelets in flow. The crystal structure of the mouse A1 domain was solved using recombinant proteins (Fukuda, et al., 2005). The main chain schematic of this domain, with β-strands (arrows) and helices (coils), is shown in FIG. 15A. The model was built from residues 1270 to 1463 of the murine VWF-A1 crystal. The two cysteines involved in the disulfide bridge are shown as spheres (involving residues 1272 and 1458). The mouse and human A1 domains appear to overlap very closely, which suggests that only minor structural differences may account for the preferential binding of platelets from mice or man to their respective VWF-A1 proteins (FIG. 15B). In fact the β-sheets of both species are identical within experimental error (a root mean square difference of 0.33 Å for C α atoms). Thus, minor differences in residues, but not structure, most probably account for the inability of human platelets to interact with mouse VWF-A1 and vice versa.

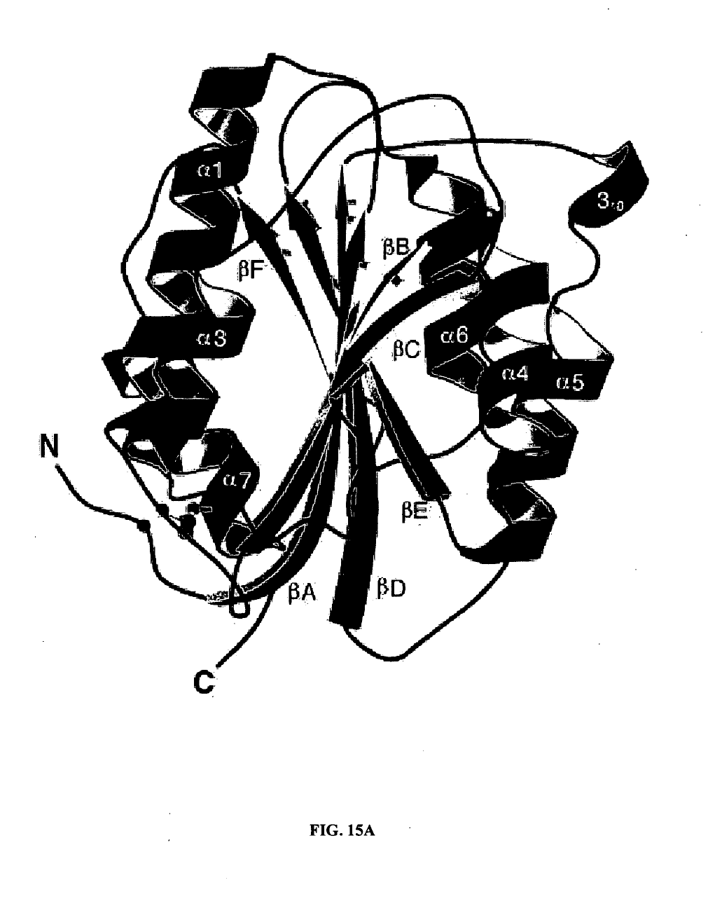

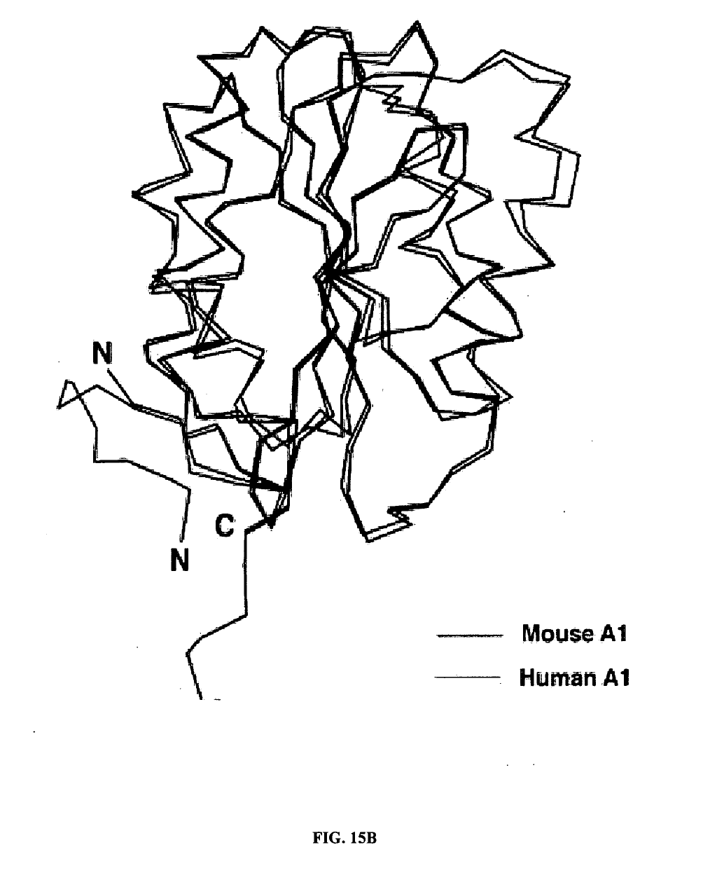

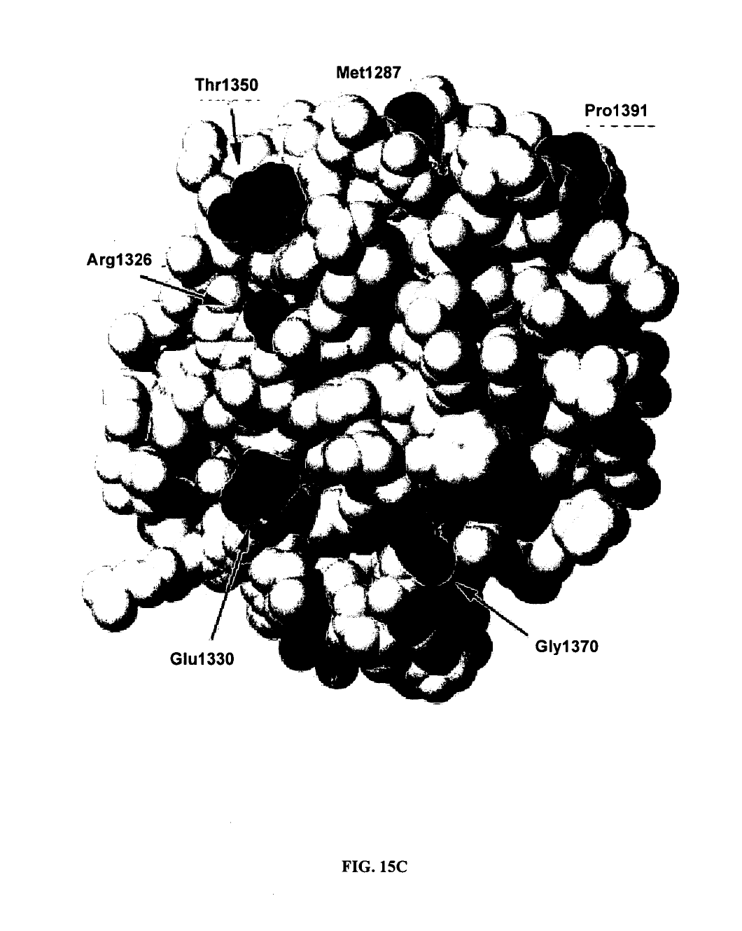

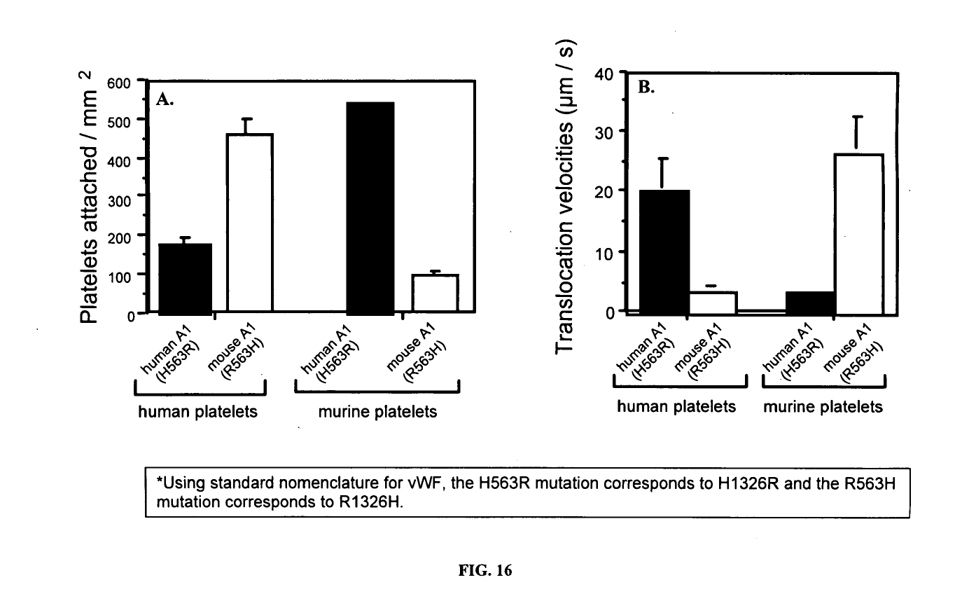

Support for this hypothesis is provided by mutagenesis studies. By analyzing the data obtained from the crystal structure of the murine VWF-A1 domain, the inventors have identified several residues that may participate in interactions with GPIb α (FIG. 15C). Residue 1326 was initially chosen for study and was mutated to the corresponding amino acid at the identical location in its human counterpart (from Arg to His). Subsequently, the ability of murine and human platelets to interact with this mutant protein substrate was evaluated at a wall shear rate of 800 s⁻¹. Incorporation of a histidine for arginine at position 1326 in murine VWF-A1 reduced murine platelet adhesion by about 5-fold and increased translocation velocities of cells by about 7-fold as compared to the WT mouse protein (FIGS. 13 and 16). Interestingly, human platelet interactions with the mutated murine protein were comparable to that of WT human VWF-A1. Conversely, substitution of Arg for His in the human VWF-A1 protein resulted in an increased ability of murine platelets to attach and translocate in a manner similar to that observed for WT murine VWF-A1. These studies support the hypothesis that from a structural and functional standpoint, mouse and human VWF-A1 are very similar.

Thus, all that remains is to demonstrate that the kinetics of the interaction between the murine GPIb α and murine VWF-A1 are similar to its human counterpart and that mutations in man that cause functional alterations in platelet adhesion with VWF have the identical impact on the biophysical properties of the murine receptor-ligand pair.

### The Kinetics of Murine VWF-A1 and its Mutants.

To determine whether the kinetics of the murine GPIb α interactions with the murine VWF-A1 domain is similar to that of the human receptor-ligand pair, the dissociation of transient tethering events was measured using VWF-A1 coated beads (7 μm diameter) interacting with surface-immobilized platelets. The use of beads with one uniform size and shape permits determination of the relationship between wall shear stress and the force directly acting on the GPIb α-VWF-A1 tether bond (F_{b}), a parameter difficult to estimate for discoid-shaped objects such as platelets. A coating concentration of VWF-A1 was chosen (5 μg/ml corresponding to 30 molecules/μm²) that supported tether bond formation at wall shear stresses ranging from 0.5 to 3 dyn cm⁻². Estimation of the site density of murine VWF-A1 on beads was performed using a monoclonal antibody generated in the inventors' laboratory designated as AMD-2. This antibody was made by immunizing Fischer 344 rats (3-4 months old) with recombinant WT protein. Following several injections of murine VWF-A1, serum was collected and screened by ELISA for anti-VWF-A1 antibodies. Spleens from animals with the highest antibody titers were harvested and splenocytes fused with Sp2/0 mouse myeloma cells (Kohler, et al., 1975).

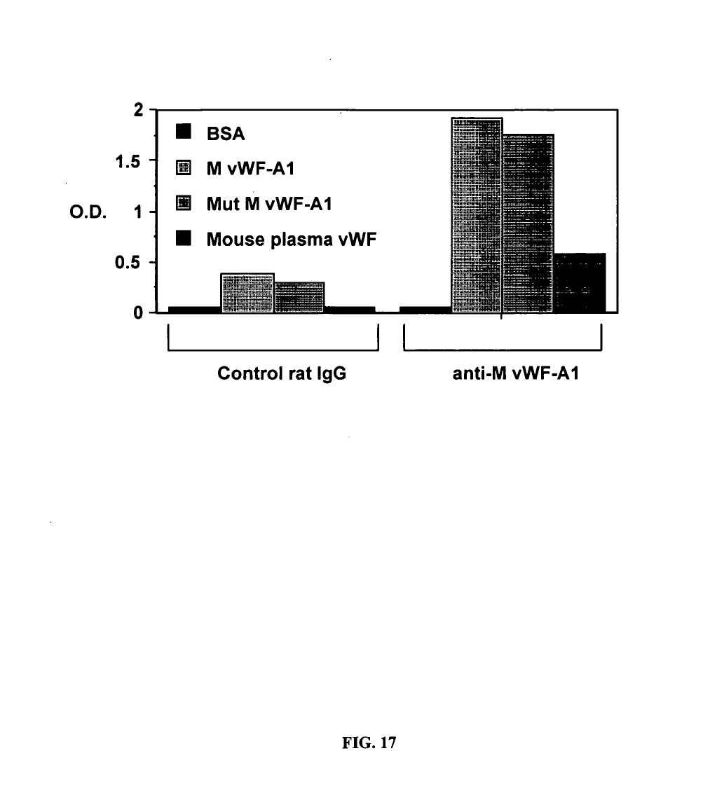

Supernatants of hybridomas were screened for reactivity to mouse VWF-A1 by ELISA (FIG. 17). Pre-immune rat serum was used as control. Monoclonal antibodies (Mabs) to murine VWF-A1 not only reacted with WT and mutant proteins (1324G>S) but also recognized native VWF purified from mouse plasma. Antibodies are tested for function blocking capabilities to use in both in vitro and in vivo assays. Antibodies will also be used for epitope mapping.

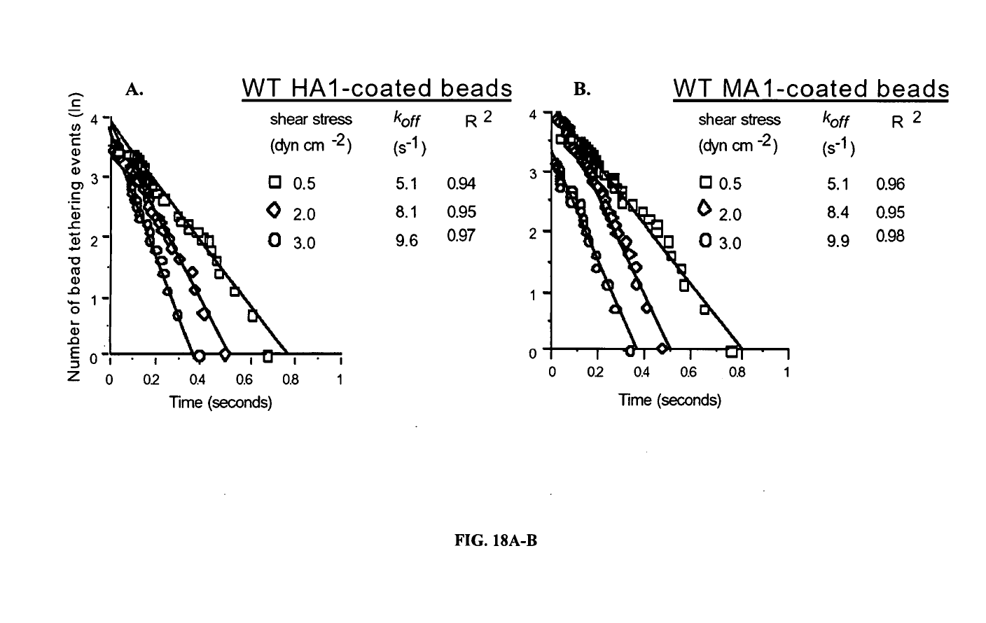

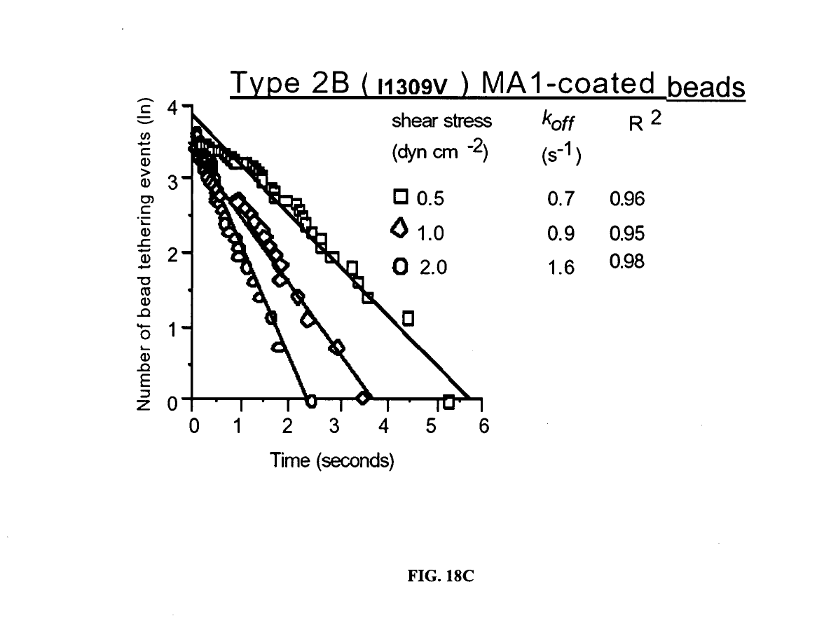

Analysis of the distribution of interaction times between human or murine VWF-A1 coated beads and their respective platelet substrates, as measured by high temporal resolution video microscopy, indicates that >95% of all transient tether bonds events fit a straight line, the regressed slope of which corresponded to a single k_{off} (FIGS. 18A-C). Notably, the cellular off-rates of these quantal units of adhesion for the WT human and mouse proteins (FIGS. 18A and B) were quite similar.

Based on these results, it appears that the dissociation kinetics of murine GPIb α interactions with murine VWF-A1 are nearly identical to its human counterpart.

### Example 2

### VWF-A1 Mutagenesis

It is possible that minor differences may exist between murine and human VWF that would preclude one from studying human platelet behavior in a mouse model of thrombosis. However, the findings above that the estimated off-rate values and structure of these domains are similar suggest that one can investigate the role of the biophysical properties of the GPIb α-VWF-A1 bond in regulating platelet-VWF interactions in vivo using a mouse model. Both murine and human A1 crystal structures can be exploited to 1) identify candidate residues involved in the binding site for murine GPIb α and to determine their impact on the kinetic properties of this receptor-ligand pair, 2) identify residues that confer species specificity, and 3) ascertain whether insertion of known point mutations that cause type 2M and 2B VWD in man alter the kinetic properties of the murine A1 domain in a similar manner. Critical residues can be classified in terms of their impact on the cellular association and dissociation rate constants. Information obtained from these studies can be used to generate mice with mutant A1 domains in order to establish the degree in alteration in the kinetics of the GPIb α-VWF-A1 bond that is necessary to perturb hemostasis.

Similar critical structural elements exist in murine A1 domain to those identified in its human counter-part that contribute to the biophysical properties of the bond formed with GPIb α. Thus, to identify structural elements within the murine VWF-A1 domain that impact on the kinetics of interaction with GPIb α, the hypothesis that only minor structural alterations in this domain are responsible for its reduced ability to support interactions with GPIb α receptor on human platelets will be tested.

### Site-Specific Mutagenesis of Murine VWF-A1 Domain.

Site-specific mutagenesis of murine VWF-A1 domain may be performed to define residues that contribute to GPIb α binding as well as those that regulate this interaction. Studies will initially focus on amino acids that differ between human and mouse A1 that lie within the vicinity of the proposed GPIb α binding pocket.

To better define residues within murine VWF-A1 that are critical for binding of GPIb α on mouse platelets, mutations into VWF-A1 cDNA using a PCR-based strategy will be introduced and the resulting DNA will be sequenced to confirm the presence of the desired mutation(s). Mutations will be based on the murine A1 crystal structure and amino acid substitutions known to affect human VWF function such as those associated with Type 2M or 2B vWD (Tables 1-3). Several surface exposed residues have been identified within the murine A1 domain likely to participate in GPIb α binding. These are non-conserved residues in comparison to the human domain. Thus, these residues will be converted at first singly (then doubly and triply), into the murine VWF-A1 to those found in human VWF-A1. Residues are chosen based on surface-exposure on the front and upper surfaces of the domain as understood by modeling and crystal structure analysis.

TABLE 1

| | | | | | | | |
|---|---|---|---|---|---|---|---|
| SINGLE | R1326 H* | E1330G* | M1287R* | P1391Q | T1350A* | G1370S* | A1333D* |

| | |
|---|---|
| DOUBLE | M1287R* + P1391Q* |
| TRIPLE | R1326H* + E1330G* + A1333D* |

TABLE 2

| | | | | |
|---|---|---|---|---|
| SINGLE | S1289R* | D1323R* | K1348E* | R1392E* |

Residues that perturb but do not abrogate platelet binding in the human VWF-A1 protein are shown in Table 2 and FIG. 5B.

TABLE 3

| | | | | |
|---|---|---|---|---|
| TYPE 2M | G1324S* | Q1367R* | I1369F* | I1425F* |
| TYPE 2B | R1306L* | I1309V* | V1316M* | R1341L* |

Residues chosen based on their ability to abrogate or enhance interactions between human VWF and GPIb α are shown in Table 3.

Type 2B mutations will also be combined with those that dramatically shorten the bond lifetime to determine (increase k_{off}) if these function-enhancing mutations can restore adhesion to that observed for WT VWF-A1.

Laminar flow assays will be performed to assess the impact of various mutations on platelet adhesion as well as the degree in alteration in the kinetic properties of the GPIb α-VWF-A1 bond.

Murine platelets will be purified as described above and stored in Tyrode's buffer containing 0.25% BSA, pH 7.4. For studies requiring human platelets, blood will be collected by venopuncture from healthy donors and cells obtained from centrifugation of PRP. All platelets will be used within 2 hours of purification.

To evaluate the impact of mutations on platelet adhesion, both human and murine platelets are perfused over high concentrations of murine VWF-A1 proteins (100 μg per ml) absorbed to glass cover slips in a parallel plate flow chamber at wall shear rates ranging from 20 to 1600 s⁻¹. An enzyme-linked immunosorbent assay is utilized to ensure that an equivalent amount of recombinant WT or mutant protein is immobilized. The number of platelets that attach per unit area per minute and their velocity of forward motion (μm/s), termed translocation, is recorded on Hi-8 videotape using an inverted Nikon microscope with a plan 10× or 40× objective, respectively. In addition, whether the incorporated mutations alter the requirement for a critical level of hydrodynamic flow, termed the shear threshold phenomenon, to support interactions of platelets with the reactive substrate can be determined. It has been previously demonstrated that human attachment to immobilized VWF-A1 requires a minimum of >85 s⁻¹ of WSR to initiate and sustain this interaction. This phenomenon has also been well described for selectin-dependent adhesion and is believed to rely on a balance between the number of times a receptor encounters the ligand over a defined period of time and the rate at which a bond can form, parameters affected by shear rate, association rate constant, and receptor-ligand concentrations (Chen, et al., 2001, Greenberg, et al., 2002). Once attached, however, the bond lifetime influences the velocity at which the cell will move on the reactive substrate in response to shear-induced force (Chen, et al., 1999). Thus, it is likely that several mutations may perturb platelet accumulation on surface-bound VWF-A1 by altering the level of shear flow required to promote platelet attachment as well as the translocation velocities of these cells. For example, it has been shown that the type 2B mutation, Ile1309Val (1309I>V), promotes greater platelet attachment at low shear rates and reduces their translocation velocities as compared to the WT substrate. Similar results were observed upon incorporation of the identical mutation into murine VWF-A1. Demonstration that GPIb α on murine platelets is responsible for mediating interactions with recombinant A1 domains can be confirmed by antibody blocking experiments.

### Determination of Tethering Frequencies, Translocation Velocities, Detachment Profiles, and Dissociation Rate Constants Using VWF-A1 Coated Microspheres.

To better ascertain the alteration in kinetics associated with the proposed mutations, the tethering frequency, translocation velocities, detachment profile, and off-rates of VWF-A1 coated beads (7 μm diameter) interacting with surface-immobilized platelets can be measured. As stated before, the use of beads with one uniform size and shape will permit determination of the relationship between wall shear stress and the force directly acting on the GPIb α-VWF-A1 tether bond (F_{b}), a parameter difficult to estimate for discoid shaped objects such as platelets. Platelet coverage >90% of the glass surface area is used in determining the tethering frequency (on rate driven phenomenon), translocation velocities (correlates with off-rate) and resistance to detachment forces (measure of bond strength) of VWF-A1 coated beads in flow. It was demonstrated that comparison of cellular on-rates and apparent bond strengths between WT and mutant forms of VWF-A1 can be achieved by limiting the concentration of these molecules to prevent multiple bond formation, a process that can mimic an enhancement in either of these kinetic parameters. By using a similar strategy, whether the proposed mutations will alter the apparent on-rate of the GPIb α-VWF-A1 bond may be determined by evaluating the frequency of transient tethering events between microspheres coated with low site densities of VWF-A1 proteins and a platelet substrate. Results are expressed as the percentage of beads (per 10× field) that paused, but did not translocate, over a range of wall shear rates that support such interactions (20 to 400 s⁻¹). Tethers per minute are divided by the flux of beads near the wall per minute to obtain the frequency of this adhesive interaction. Only one tethering event per bead is counted during the observation period.

For determining translocation velocities, beads (1×10⁶/ml), coated with a saturating concentration of VWF-A1 protein are infused into the parallel-plate flow chamber at 1.0 dyn cm⁻² and allowed to accumulate for 5 minutes. Subsequently, the wall shear stress is increased every 10 seconds to a maximum 36 dyn cm⁻² and the velocities of the beads determined.

For detachment assays, beads (1×10⁶/ml), coated with the minimum but equal amounts of VWF-A1 required to support translocation, are infused into the parallel-plate flow chamber at 1.0 dyn cm⁻² and allowed to accumulate for 5 minutes. Subsequently, the wall shear stress is increased every 10 seconds to a maximum 36 dyn cm⁻². The number of beads remaining bound at the end of each incremental increase in wall shear stress is determined and expressed as the percentage of the total number of beads originally bound. Using this strategy, it was found that type 2B mutations do not strengthen, and in fact may even weaken the interaction between GPIb α and VWF-A1 as suggested by the increase in reactive compliance as compared to the native complex (Table 1). In all studies, video images are recorded using a Hi8 VCR (Sony, Boston, Mass.) and analysis performed using a PC-based image analysis system (Image Pro Plus).

For determining the kinetics of dissociation, the duration of transient tethers between murine VWF-A1 coated microspheres and immobilized murine platelets is measured as described herein. MC simulations are run and estimates of k_{off} fit to the Bell model by standard linear regression to obtain the intrinsic off-rate (k⁰_{off}) and the reactive compliance σ. Results are compared for all mutations to determine their impact on these kinetic parameters (i.e.—increase or shorten the bond lifetime (k⁰_{off}) and/or increase or decrease the susceptibility of the bond to hydrodynamic forces (σ).

These experiments complement recent work on identifying residues in human VWF-A1 domain critical for interacting with the GPIb α. Moreover, they allow for delineation of its binding site in murine VWF-A1. This work elucidates the role of the biophysical properties of this receptor-ligand pair in regulating platelet-VWF interactions in vivo. Furthermore, it will pave the way for the generation of mice with comparable types of human VWD (i.e. type 2B) and may even permit the study of human platelets in a mouse model of thrombosis.

Although there is no guarantee that the introduction of mutations will not significantly perturb protein structure and thus function, the ability of murine VWF-A1 specific mAbs to recognize mutant proteins should clarify this matter. Similarly, the gain/loss of function experiments involving swapping of residues between human and mouse VWF-A1 will also prove useful in avoiding this pitfall.

To know whether the regions flanking the mouse A1 domain are important in mediating interactions with GPIb α, full-length mouse VWF will be inserted into a mammalian expression vector and expressed in COS-7 cells (Cooney, et al., 1996). Mutations found to be critical for binding will be inserted into the full-length construct. A recombinant protein containing the A1-A2-A3 domains will be generated initially. This will be accomplished using a baculovirus expression system as demonstrated for GPIb α.

### The Relationship Between the Major and Minor Binding Sites for GPIb α.

The recent results on the structure of GPIb α and its complex with VWF-A1 domain has not only confirmed the major binding site for this platelet receptor, but has shed new light into the mechanism by which type 2B mutations may enhance this critical interaction. As shown in FIG. 6, the concave face of GPIb α embraces the A1 domain in two distinct regions. The C-terminal loop of this receptor binds near the top of the domain (major binding site) and the N-terminal region known as the β-finger, at the bottom face (minor binding site) adjacent to the site where type 2B mutations are clustered. Based on these results that type 2B mutations appear to enhance the on-rate (reduced shear rate needed for formation of transient tethers in flow) and prolong the lifetime (5-6 fold) of the interaction between VWF-A1 and GPIb α, it is interesting to speculate whether similar alterations in bond kinetics would be observed with type 2B mutations if one interfered with the primary site. For instance, would inclusion of a type 2B with a type 2M mutation reconstitute adhesion, or is some finite interaction time required in the primary binding pocket for GPIb α before the effects of these mutations can be observed? These are important questions as they will guide the development of reagents that can either enhance or reduce the interaction between GPIb α and VWF-A1.

### The Kinetics of Murine VWF-A1 Mutants.

Notably, the cellular off-rates of these quantal units of adhesion for the WT human and mouse proteins (FIGS. 18A and B) were quite similar, but were significantly higher than those observed for the murine VWF-A1 containing the type 2B mutation 11309V (1309I>V) (FIG. 18C). This is consistent with previous results obtained using the same mutation in the human protein (See Table 4 for a list of mutations).

TABLE 4

| | | | | | | | |
|---|---|---|---|---|---|---|---|
| SINGLE | H1326R* | G1330E* | R1287M* | Q1391P* | A1350T* | S1370G* | D1333A* |

Based on these results and the results in Example 1 above, it appears that the dissociation kinetics of murine GPIb α interactions with murine VWF-A1 are nearly identical to its human counterpart and that type 2B mutations also prolong the bond lifetime of this interaction as seen in man.

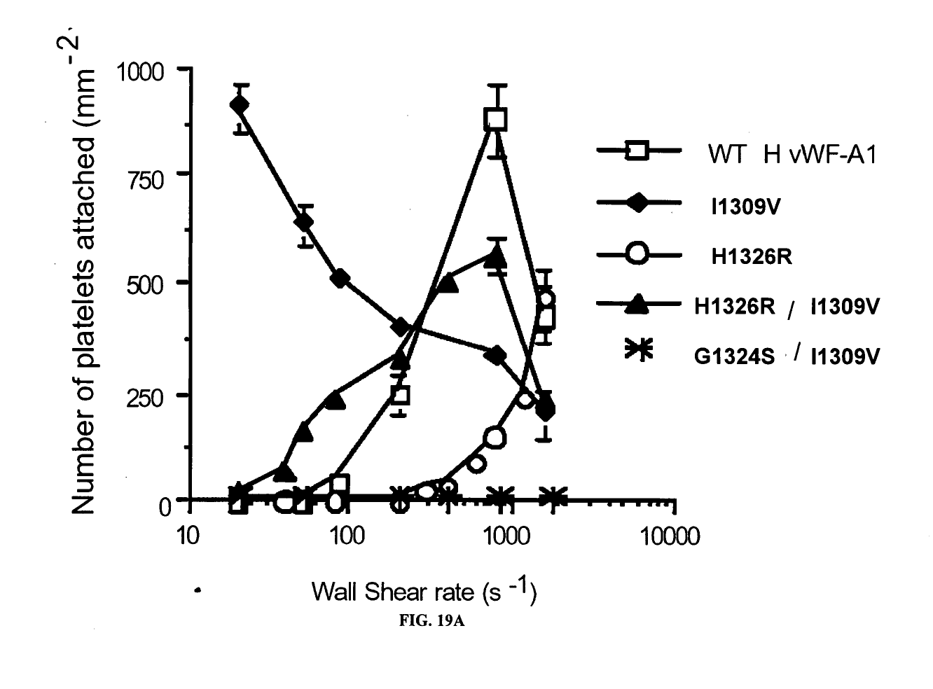

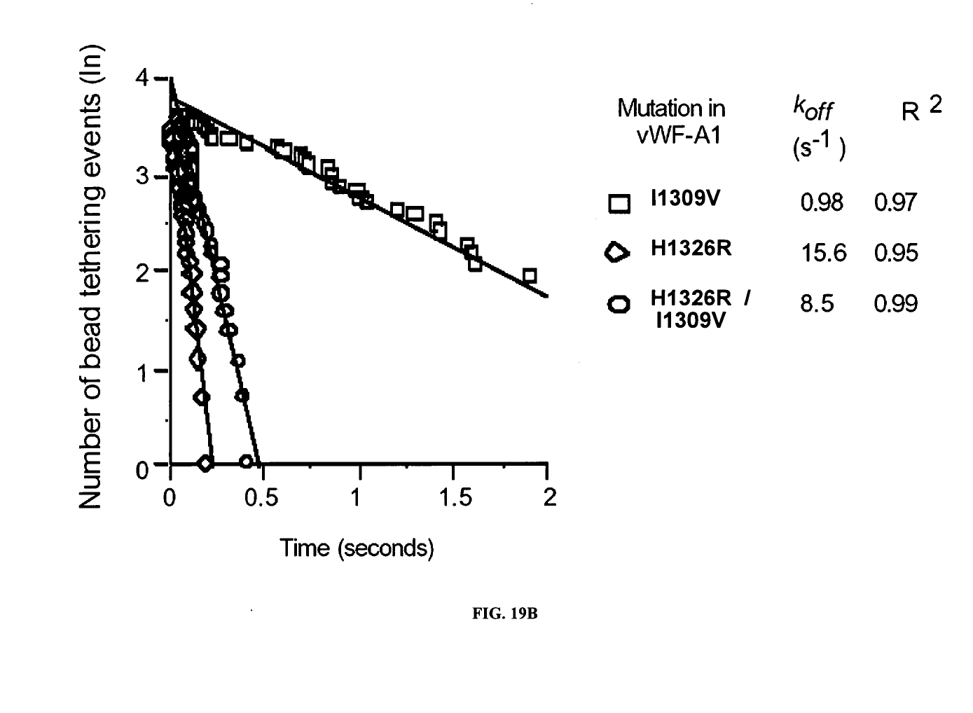

The type 2B mutation 1309I>V was incorporated into recombinant human VWF-A1 containing either the type 2M mutation Gly1324Ser (1324G>S) that completely abolishes adhesion or the function reducing mutation His1326Arg (1326H>R) and the ability of these doubly mutated proteins to support human platelet adhesion in flow was determined. In comparison to WT, an about 3-fold increase in wall shear rate is required to promote platelet attachment to human VWF-A1 containing Arg at 1326 (FIG. 19A). Incorporation of the type 2B mutation, however, appeared to enhance the on-rate of this interaction as manifested by an increase in platelet binding at lower levels of shear flow, but not to levels observed for the type 2B mutation alone.

To determine whether the 1309 mutation would also prolong the lifetime of the interaction with GPIb α, the distribution of interaction times was analyzed between VWF-A1 coated beads and surface-immobilized platelets at a wall shear stress of 1 dyn cm⁻². Remarkably, a 2-fold reduction in k_{off} was noted (from 15.6 to 8.5 s⁻¹) as compared to the single, function-diminishing mutation (FIG. 19B). This value is similar to that of the native receptor-ligand interaction with a k_{off} of 6.5 s⁻¹ under identical flow conditions. By contrast, incorporation of Val for Ile at residue 1309 in an A1 domain containing the type 2M mutation 1324G>S did not reconstitute platelet adhesion. Thus, these results suggest that it is essential for bond formation to occur in the primary GPIb α binding site (top face of the A1 domain). Moreover, the region of the A1 domain where type 2B mutations are clustered appears to be critical for stabilizing interactions with GPIb α. Similar findings were observed for murine VWF-A1 containing the identical type 2B mutation but with a change in Arg to His at residue 1326.

### Example 3

### Defining the In Vivo Role of the von Willebrand Factor A1 Domain by Modifying a Species-Divergent Bond

### The VWF^{1326R>H} Mice

VWF contributes to human health and disease by promoting adhesive interactions between cells (Whittaker, C. A., et al., 2002). The VWF-A1 domain is thought to play a critical role in hemostasis by initiating the rapid deposition of platelets at sites of vascular damage by binding to the platelet receptor glycoprotein Ib α (GPIbα) at high shear rates (Roth, G. J., et al., 1991, Cruz, M. A., et al., 1993, Sugimoto, M. et al., 1991, Pietu, G. et al., 1989). Although congenital absence of VWF in humans has established a role for this plasma glycoprotein in hemostasis (Sadler, J. E. et al. 2006), the contribution of its A1 domain in clot formation has been questioned in a mouse model of vascular injury (Denis, C. et al., 1998).

Murine plasma VWF or its A1 domain fails to support significant interactions with human platelets (and likewise human VWF with murine platelets) under flow conditions. Atomic models of GPIb α-VWF-A1 complexes suggest that the structural basis for this behavior arises primarily from an electrostatic “hot-spot” at the binding interface. Introduction of a single point mutation within this region of murine VWF-A1 is sufficient to switch its binding specificity from murine to human platelets. In addition, introduction of a single point mutation within the electrostatic “hot-spot” region of human VWF-A1 is sufficient to switch its binding specificity from human to murine platelets. Moreover, mice possessing the 1326R>H mutation in their VWF have a bleeding phenotype distinct from VWF-deficient animals, and can be corrected by the administration of human platelets. Mechanistically, mutant animals can generate but not maintain thrombi at sites of vascular injury, whereas those infused with human platelets can form stable thrombi, a process that relies on GPIb α-VWF-A1 interaction. Thus, interspecies differences at protein interfaces can provide insight into the biological importance of a receptor-ligand bond, and aid in the development of an animal model to study human platelet behavior and drug therapies.

### Methods.

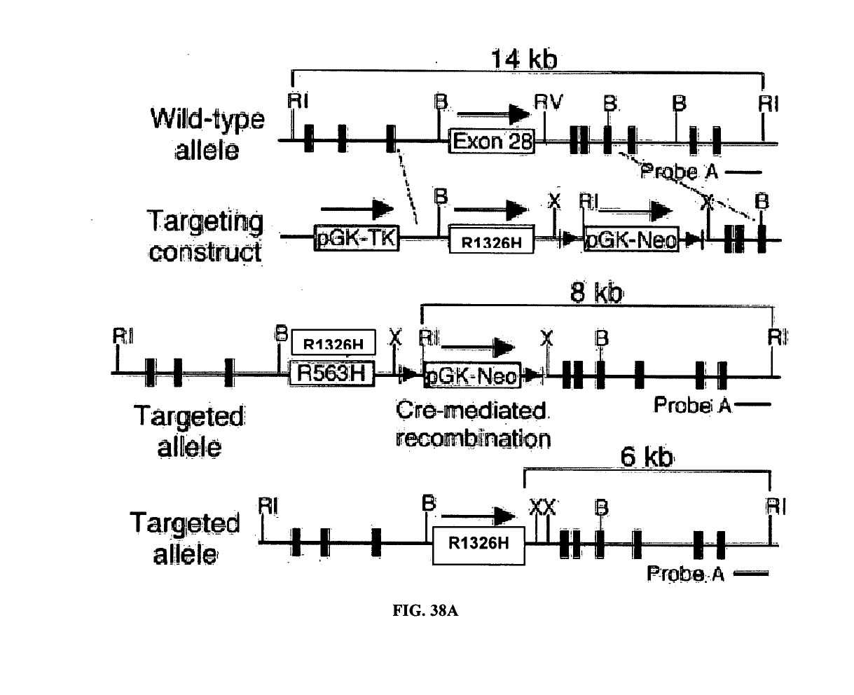

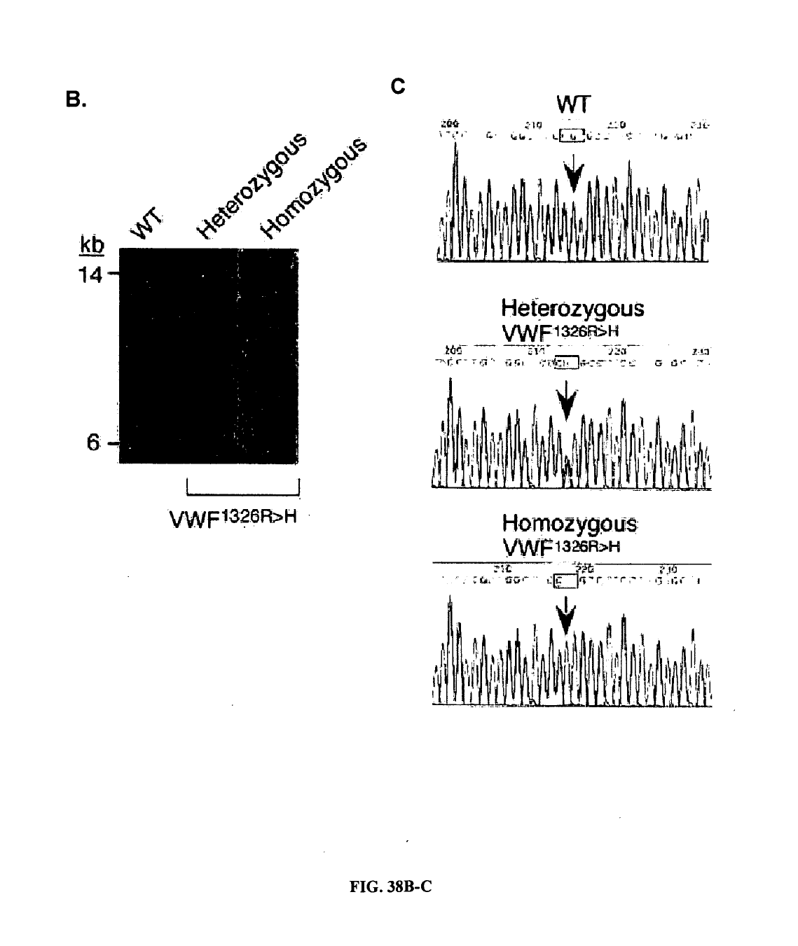

Generation of VWF^{1326R>H} mice. The VWF^{1326R>H} targeting vector (FIG. 38A) was prepared from a 129/SvJ mouse genomic library. The clone was identified by PCR using primers specific for exon 28 of the mouse VWF gene and sequence fidelity of the region to be targeted validated by comparison to published sequence for chromosome 6 (GenBank accession number NW_{—}001030811). The targeting vector is identical to the corresponding region in the mouse genome, except the 1326R>H mutation was created in exon 28 and the Neo cassette flanked by loxP sites was inserted into intron 28. This resulted in the loss of an EcoRV site and the introduction of a new EcoRi and two new XhoI sites. The construct was electroporated into an embryonic stem (ES) cell line, and potential clones identified by continued growth of cells in G418 and Gancyclovir supplemented media. DNA was isolated from surviving colonies, digested with EcoRI, and screened by Southern analysis using a 1.5 kb probe (A) corresponding to a DNA sequence downstream of the targeting construct. Chimeric mice generated from VWF^{1326R>H} targeted ES cell lines were subsequently bred to a Cre transgenic mouse (C57BL/6 background) and animals containing the 1326R>H mutation, but without the Neo cassette, subsequently identified by both PCR and Southern analysis. WT and homozygous animals were the product of matings between heterozygous mice.

Analysis of VWF transcripts, antigen levels, multimers, and collagen binding. Detection of transcripts from the A1-A2-A3 domains of murine VWF was performed by RT-PCR. Briefly, mRNA was isolated from lung tissue harvested from either homozygous VWF-A1^{1326R>H} mice or aged-mated WT littermate controls (Oligotex™, Qiagen, Germantown, Md.). Generation of cDNA and PCR-amplification of desired transcripts was performed using SuperScript™. One-Step RT-PCR (Invitrogen Corp., Carlsbad, Calif.) and oligos specific for the A domains of VWF.

Functional factor VIII levels were determined by a mechanical clot detection method using the STA automated coagulation analyzer (Diagnostica Stago, Parsippany, N.J.). A log-log calibration curve was established by measuring the activated partial Thromboplastin time (aPTT) of varying dilutions of reference plasma. The aPTT of a 1:10 dilution of sample plasma mixed with factor VIII deficient plasma was determined, compared to the calibration curve, and the activity expressed as a percent of normal.

Evaluation of VWF antigen levels was performed as previously described (Denis, C. et al., 1998). For multimer analysis, plasma from sodium citrate treated whole blood was diluted 1:5 in electrophoresis sample buffer (final concentration 10 mM Tris-HCl pH 8.0, 2% SDS, 1 mM EDTA) and heated at 56° C. for 30 minutes. Electrophoresis was carried out overnight (64 volts, 15° C.) through a horizontal SDS-agarose gel in 1.2% agarose (Ruggeri, Z. M., et al., 1981). The gel was then electrophoretically transferred (150 mA, 90 minutes) to Immobilon (Millipore, Billerica, Mass.) followed by blocking (2 hours) with 5% powdered milk in TBST (Tris HCl pH 8.0, 0.15M NaCl, 0.05% Tween-20). The membrane was incubated with a 1:500 dilution of rabbit anti-human VWF antiserum (Dako, Fort Collins, Colo.) for 1 hour, washed in TBST, and then incubated with a 1:10,000 dilution of HRP-conjugated mouse anti-rabbit IgG (Calbiochem, Merck KGaA, Darmstadt, Germany). Bands were subsequently detected by a chemiluminescence system (GE Healthcare, Waukesha, Wis.). For comparison, a sample containing pooled human plasma from healthy individuals or patients with type 2B VWD was also loaded on the gel. Binding of VWF to surface-immobilized collagen was performed as previously described (Smith, C. et al., 2000). Briefly, 100 μg/ml of acid soluble type I collagen from human placenta (Sigma, St. Louis, Mo.) was added to a 96 well microtiter plate and allowed to incubate overnight (4° C.). After washing and blocking with TBS containing 3% BSA and 0.05% Tween 20, varying concentrations of platelet poor plasma harvested and pooled from WT, homozygous VWF^{1326R>H}, and VWF deficient mice was added to the wells and incubated for 1 hour (37° C.). Wells were then washed and bound VWF detected by an ELISA as described above.

Ex vivo platelet adhesion studies. Experiments were performed in a parallel-plate flow chamber as previously described (Offermanns, S., et al., 2006). For studies involving plasma VWF, a polyclonal anti-VWF antibody (Dako) was absorbed overnight (4° C.) to a six well tissue culture plate. Subsequently, the plate was washed and non-specific interactions blocked by the addition of TBS containing 3% BSA, pH 7.4 (1 hour, 37° C.). Human or murine plasma obtained from heparinized whole blood was added and the plates placed at 37° C. for an additional 2 hours. Generation, purification, and surface-immobilization of recombinant VWF-A1 proteins was performed as previously described (Doggett, T. A. et al., 2002). Both human and murine VWF-A1 constructs consist of amino acid residues 1238 to 1471, with a single intra-disulfide bond formed between residues 1272 and 1458 and were generated in bacteria. Citrated whole blood (150 μl) collected via cardiac puncture from anesthetized homozygous VWF^{1326R>H} or WT mice or from venopuncture from human volunteers was perfused over the immobilized substrates at a wall shear rate of 1600 s⁻¹ for 4 minutes, followed by washing with Tyrode's buffer under the identical flow conditions. The number of platelets attached per unit area (0.07 mm²) and translocation velocities were determined by off-line analysis (Image-Pro Plus, Media Cybernetics). For GPIb α inhibition studies, the function-blocking mAb 6D1 (20 μg/ml) or mAb SZ2 (20 μg/ml; Beckman Coulter, Brea, Calif.) was added to anticoagulated human blood for 30 minutes prior to use. Experiments were performed in triplicate on two separate days. An ELISA was used to ensure equivalent coating concentration of plasma and recombinant proteins (Denis, C. et al., 1998).

In vivo thrombus formation. Administration of anesthesia, insertion of venous and arterial catheters, fluorescent labeling and administration of human platelets (5×10⁸/ml), and surgical preparation of the cremaster muscle in mice have been previously described (Doggett, T. A. et al., 2002, Diacovo, T. G., et al., 1996). Injury to the vessel wall of arterioles (about 40-65 μm diameter) was performed using a pulsed nitrogen dye laser (440 nm, Photonic Instruments) applied through a 20× water-immersion Olympus objective (LUMPIanFI, 0.5 NA) of a Zeiss Axiotech vario microscope. Mouse platelet- and human platelet-vessel wall interactions were visualized using either bright field or fluorescence microscopy. The latter utilized a fluorescent microscope system equipped with a Yokogawa CSU-22 spinning disk confocal scanner and 488 nm laser line (Revolution XD, Andor™ Technology). The extent of thrombus formation was assessed for 2 minutes post injury and the area (μm²) of coverage determined (Image IQ, Andor™ Technology). For GPIb α or αIIb β3 inhibition studies, the function-blocking mAb 6D1 or 7E3 (20 μg/ml), respectively (from B. Coller, Rockefeller University), was added to purified human platelets for 30 min prior to administration.

Tail bleeding assay. Bleeding times were measured in 7-week old mice after amputating 1 cm of the tail tip as previously described (Denis, C. et al., 1998). In studies involving human platelets, platelet concentrates were obtained from Columbia Presbyterian Hospital Blood Bank, washed and resuspended in normal saline (1.5×10⁹/300 μl) before administering through a catheter inserted into the right internal jugular vein. Tail cuts were performed 5 minutes after completion of the infusion of platelets. PLAVIX and ReoPro™ were obtained from the research pharmacy at Columbia University Medical Center. For studies involving PLAVIX, animals received a 50 mg/kg oral dose of the drug the day before and 2 hours prior to the administration of human platelets. ReoPro™ was given initially as an intravenous bolus (0.25 mg/kg) 5 minutes after the administration of human platelets, followed by a continuous infusion (0.125 μg/kg/min) as per the manufacturer's recommendations.

Structural Modeling. There are three crystal structures of the GPIb α-VWF-A1 complex: two are WT except for mutated N-glycosylation sites in GPIb a (Fukuda, K. et al., 2005), and one is a gain-of-function mutant (Huizing a, E. G. et al., 2002). The structures have only small differences that are not the result of the presence of mutations or botrocetin binding (Fukuda, K., et al., 2005). Both N-glycosylation sites in human GPIb α lie on the well-ordered upper ridge of the LRR, 18 Å and 27 Å (Cα-Cα) from the nearest VWF-A1 residue, so their absence is unlikely to affect the structure of the complex. Murine GPIb α has no predicted N-glycosylation sites.

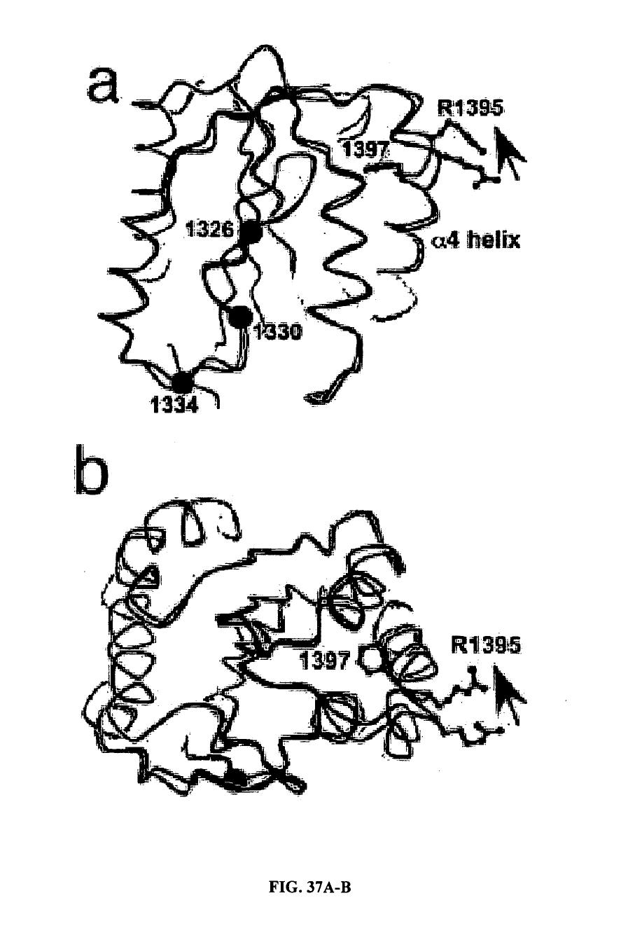

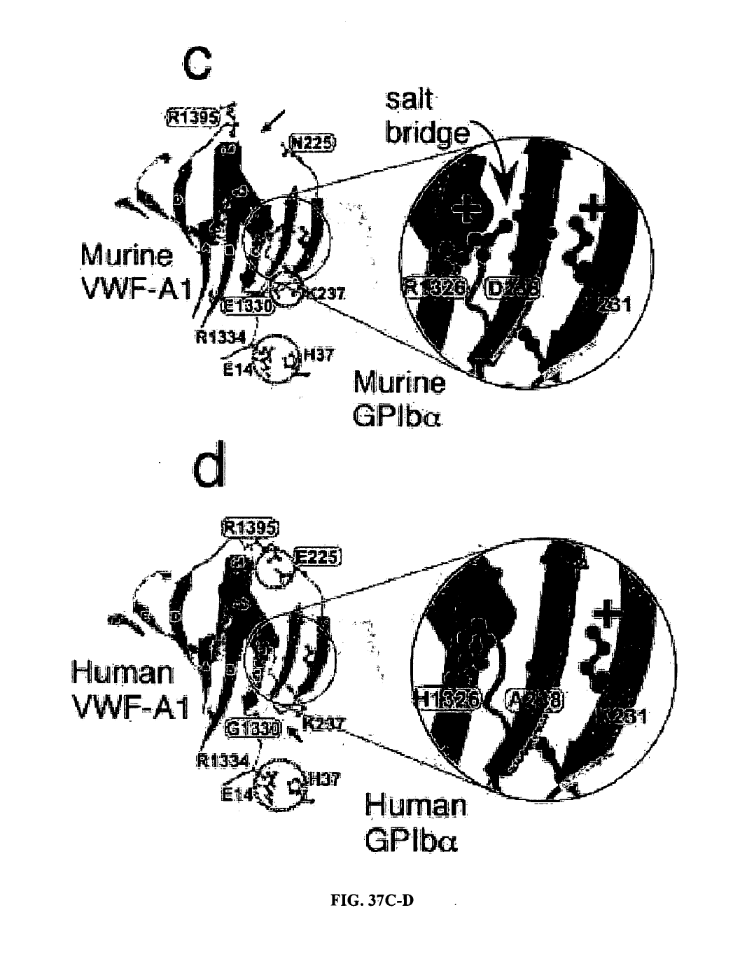

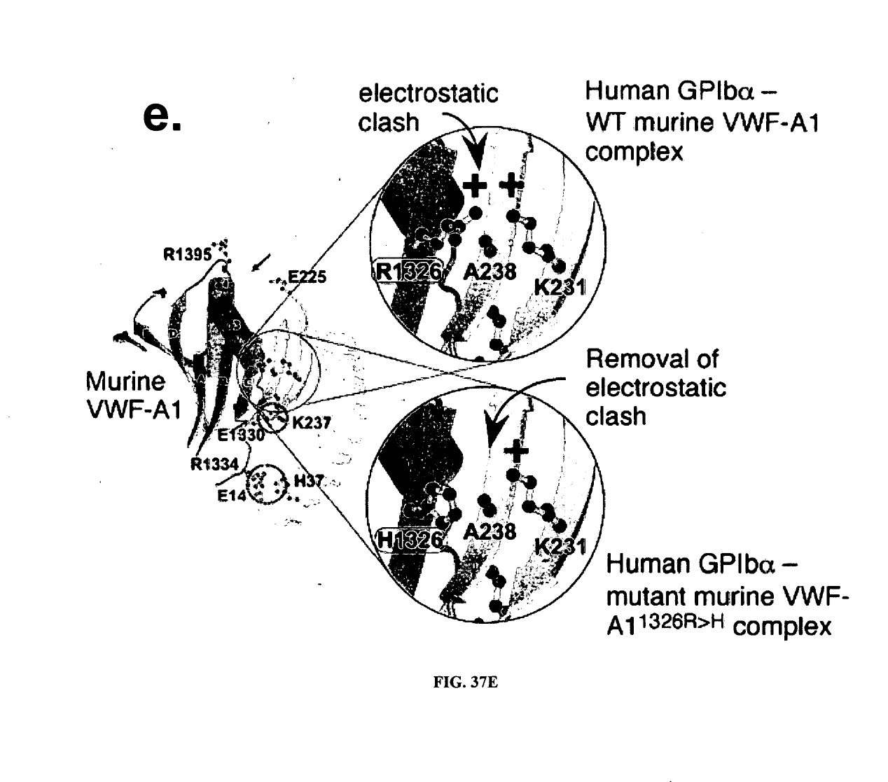

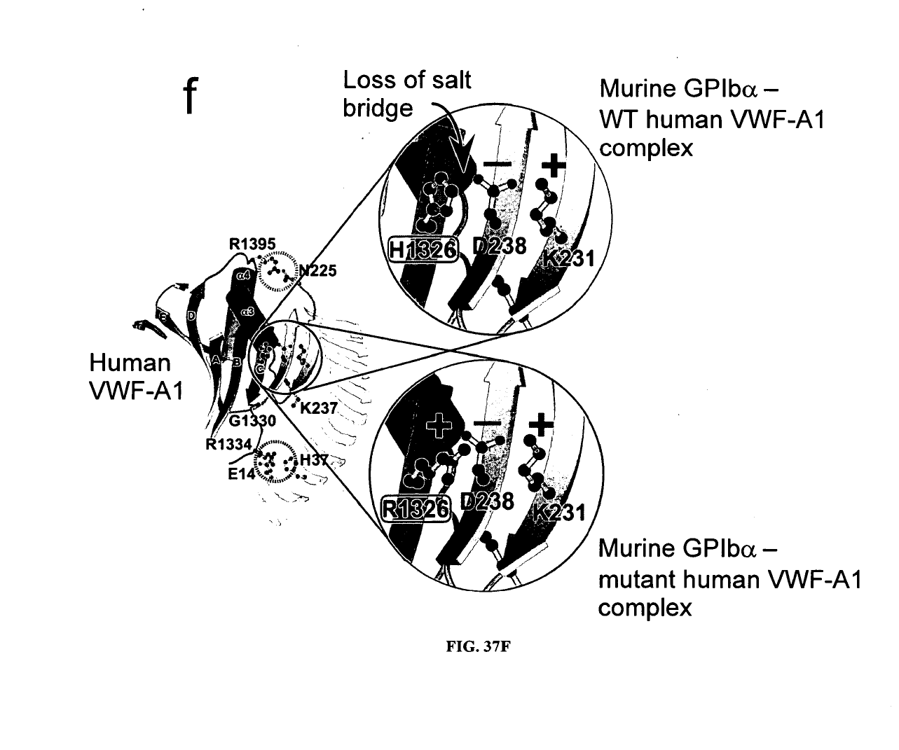

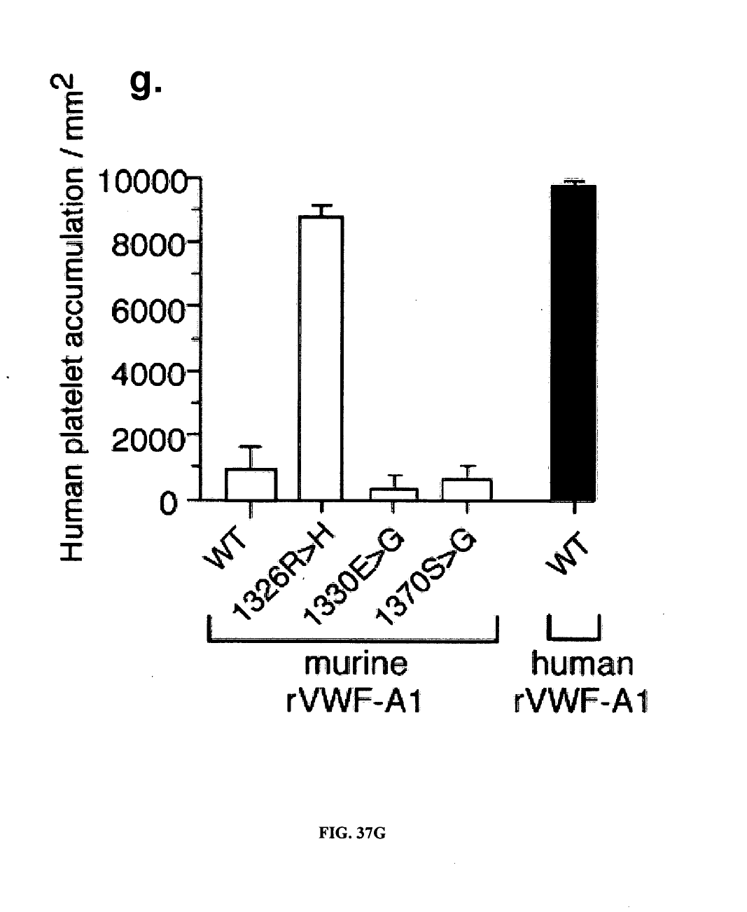

Human GPIb α contains sulfated tyrosines implicated in binding VWF within an acidic loop just C-terminal to the sequence included in the crystal structures. Murine GPIb α has a predicted sulfation site in the same loop, so that the differential binding of human vs. murine GPIb α to VWF-A1 is also likely to be small. The interfacial regions are otherwise highly conserved between species, with the exception of three salt bridges (See FIGS. 37C-G). The conformation of the β-switch region is highly constrained. The only change to a buried interfacial residue is M239T (human to mouse), which lies in an invariant pocket. Notably, the crystal structure of the human “gain-of-function” mutant, M239V, shows no perturbations in this region, and given that valine is isosteric with threonine, this species difference is unlikely to affect either the complex structure or interspecies binding.

A consensus model of the human complex was used to build the murine model. Murine A1 onto human A1 was first overlaid by fitting the central β-sheets (RMSD 0.3 Å; within experimental error); the only notable difference is the location of helix α 4, which is shifted by 2-3 Å away from the GPIb α interface in the mouse owing to a larger residue on the buried face of this helix. For the GPIb α model, only the side-chains were altered, since the human and murine LRRs have identical lengths. Consensus rotamers with minimal steric clashes were chosen, followed by manual adjustment where necessary to create sensible van der waals interactions and H-bonding, using TURBOFRODO (Bio-Graphics, Marseille, France). Molecular overlays were optimized using LSQKAB (Collaborative Computational Project, 1994); molecular figures were created using MOLSCRIPT (Esnouf, R. M., 1997)) and OPENGL (Khronos Group, Beaverton, Oreg.)

Statistics. An unpaired, two-tailed Student t test was used for multiple comparisons.

### Results.

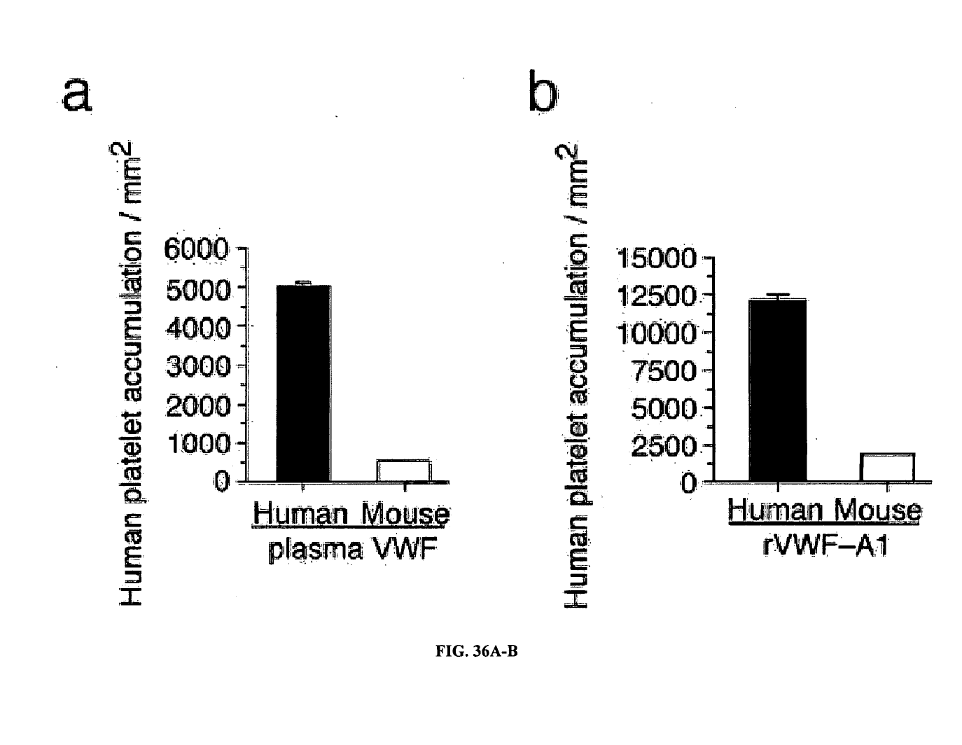

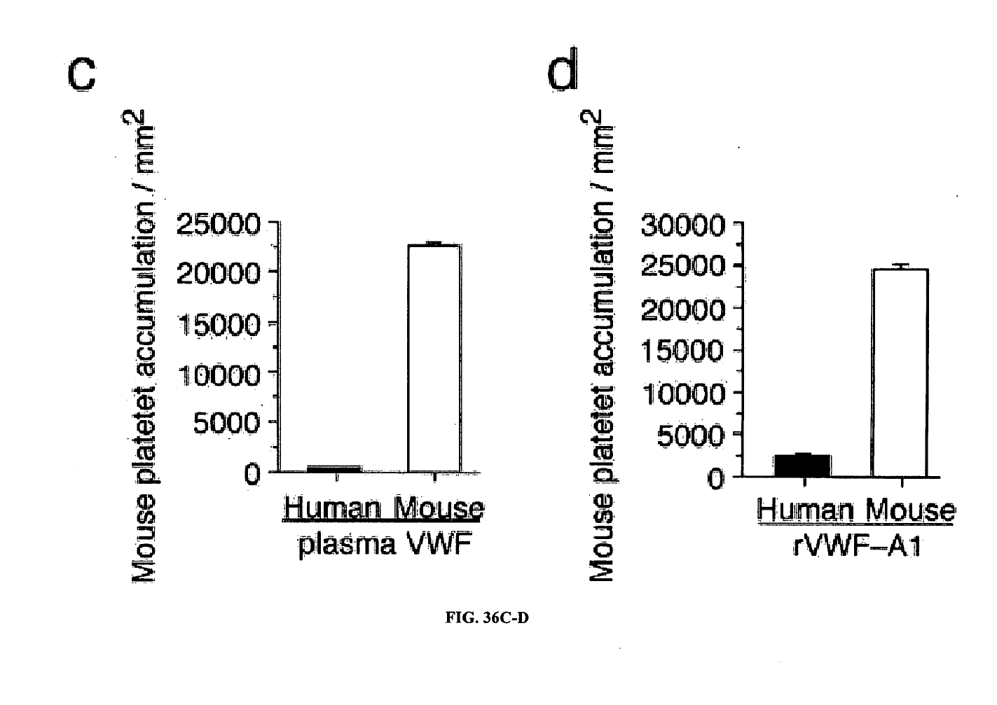

Because the interaction between GPIb α and VWF-A1 is a prerequisite for effective thrombus formation in the arterial circulation, the ex vivo ability of surface-bound murine plasma VWF or its recombinant A1 domain (rVWF-A1) to support human platelet adhesion under physiologically relevant flow conditions was first tested using a parallel-plate flow chamber at a shear rate exceeding 1000 s⁻¹ (Ruggeri, Z. M. et al., 2006). The adhesive properties of VWF are tightly regulated such that it preferentially binds to platelets only when immobilized to sites of vascular injury and under hydrodynamic conditions encountered on the arterial side of the circulation (Sakariassen et al., 1979, Ruggeri, Z. M. et al., 2006). Perfusion of human whole blood over murine plasma VWF or rVWF-A1 resulted in limited platelet deposition (10 to 25%, respectively) as compared with same-species controls (FIG. 36A-B). Similarly, human VWF proteins had a diminished capacity to support murine platelet accumulation under identical conditions (FIGS. 36C-D). This interspecies incompatibility would seem to preclude the study of human platelet behavior in a mouse model of arterial thrombosis.

In order to gain insight into the structural origins of this species incompatibility, models of murine-murine and human-murine GPIb α-VWF-A1 complexes were built based on the crystal structures of the human complex (Fukuda, K., et al., 2005, Dumas, J. J. et al., 2004, Huizing a, E. G. et al., 2002) and human and murine VWF-A1 (Fukuda, K., et al., 2005) (FIG. 37A-E; see Methods).

The A1 domain comprises a Rossmann-like fold with a central, mostly parallel β-sheet flanked on both sides by α-helices (Fukuda, K., et al., 2005). Human and murine VWF-A1 share considerable sequence (86% identity) and structure homology; in fact, the β-sheets of both species are identical within experimental error (a root mean square difference of 0.33 Å for Cα-atoms). The only major difference is the location of helix a 4 (nomenclature as previously described in Dumas, J. J. et al., 2004), which is shifted 2-3 Å away from the GPIb α binding site in the mouse, owing to a difference in a buried hydrophobic residue (FIG. 37 A-B). Although neither the structure of murine GPIb α nor its complex with the VWF-A1 domain are known, the high sequence similarity of the murine and human proteins (including the complex interface), as well as the rigid architecture of the leucine-rich repeats (LRR) of GPIb α, provide high confidence that their 3D structures will be highly homologous.

In the complexes, the major contact region involves the “β-switch” region (residues 227 to 241 in the C-terminal flank of GPIb α), which forms a β-hairpin that augments the β-sheet of the VWF-A1 domain. On its other side, this region of GPIb α packs tightly against the concave face of the LRR, which highly constrains it movement. Residues in mouse and human are mostly invariant on both sides of this interface. Notable exceptions are at position 1326 in VWF-A1, which is histidine (H) in humans versus an arginine (R) in mouse, and at position 238 in GPIb α, which is alanine (A) in humans versus an aspartic acid (D) in mouse (FIGS. 37C and 37D). A model of the murine complex suggests that these changes are complementary, since D238 can form an intermolecular salt-bridge with R1326; D238 in murine GPIb α also shields the positively charged flanking lysine (K) at position 231 (a conserved residue in both species) from unfavorable interactions with R1326 in murine VWF-A1. This salt-bridge cannot form in the human complex due to the presence of a histidine at 1326. However, an intermolecular salt-bridge can occur between R1395 and E225 located at the top of the human complex, which may compensate for this loss (FIG. 37D). No such interaction can occur in the murine complex (FIG. 37C).

In the human GPIb α-murine VWF-A1 interspecies complex, it is believed that the two positively charged residues (GPIb α K231 and VWF-A1 R1326) create an electrostatic clash that impedes binding, owing to the lack of a negatively charged group at position 238 (FIG. 37E). In the murine GPIb α-human VWF-A1 interspecies complex, however, no such electrostatic clash occurs despite the absence of the salt-bridge. There is, however, an overall change in net charge in the binding interface compared with the murine GPIb α-murine VWF-A1 complex (FIG. 37F). This, together with the loss of critical salt-bridges, most likely accounts for the reduced interaction between mouse platelets and human VWF (See Tables 5 and 6).

TABLE 5
Predicted effect of species differences in residues on the human GPIb
α-murine VWF-A1 interspecies complex.

| mVWF- | hGPIb-α | hGPIb α- | |
|---|---|---|---|
| A1 | partner | mVWF-A1 | Reason |
| R1326 | A238 | (−) | Permits electrostatic clash of R1326 |
| | | | with K231 in GPIb α |
| E1330 | K237 | (+) | New salt-bridge E1330-K237 |
| G1370 | none | 0 | No interactions |
| R1395 | E225 | (−) | Loss of salt-bridge (shifts position) |

(+) = net positive,
(−) = net negative,
0 = minimal effect compared with syngeneic complexes.

TABLE 6
Predicted effect of species differences in residues on the murine GPIb
α-human VWF-A1 interspecies complex.

| hVWF- | mGPIb-α | hGPIb α- | |
|---|---|---|---|
| A1 | partner | mVWF-A1 | Reason |
| H1326 | D238 | (−) | Loss of R1326-D238 salt-bridge |
| G1330 | K237 | (−) | Loss of E1330-K237 salt-bridge |
| S1370 | none | 0 | No interactions |
| .R1395 | N225 | (+) | New polar interactions with (shifts |
| | | | position) R1395 |

(+) = net positive,
(−) = net negative,
0 = minimal effect compared with syngeneic complexes.

To explore the importance of the electrostatic mismatches in destabilizing the interspecies complexes, human residues were substituted into murine rVWF-A1 at positions 1326 (R>H), 1330 (E>G), and 1370 (S>G), and the ability of the mutant proteins to support human platelet accumulation under flow was analyzed. As expected, amino acid substitutions at positions 1330 (predicted to remove a salt-bridge) and 1370 (predicted to have no effect) failed to promote the interaction between murine rVWF-A1 and human GPIb α. However, the 1326R>H mutation, which eliminates the electrostatic clash with K231, rendered murine A1 capable of supporting interactions at a level comparable to its wild-type (WT) human counterpart (FIG. 37G). Similarly, conversion of 1326H>R in the human rVWF-A1 protein promoted the binding of mouse platelets, while the reverse substitution in its murine counterpart reduced adhesion by 75%. That a single residue change is sufficient for shifting the binding preferences across species supports the notion that this contact region is a “hot-spot” in the protein interface (Bogan, A. A., et al., 1998).

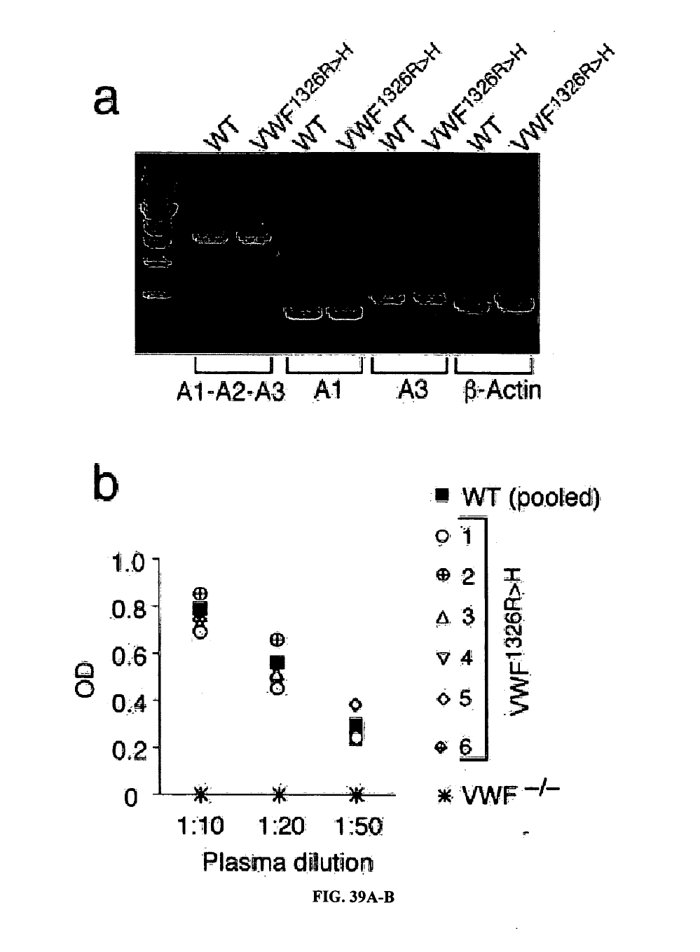

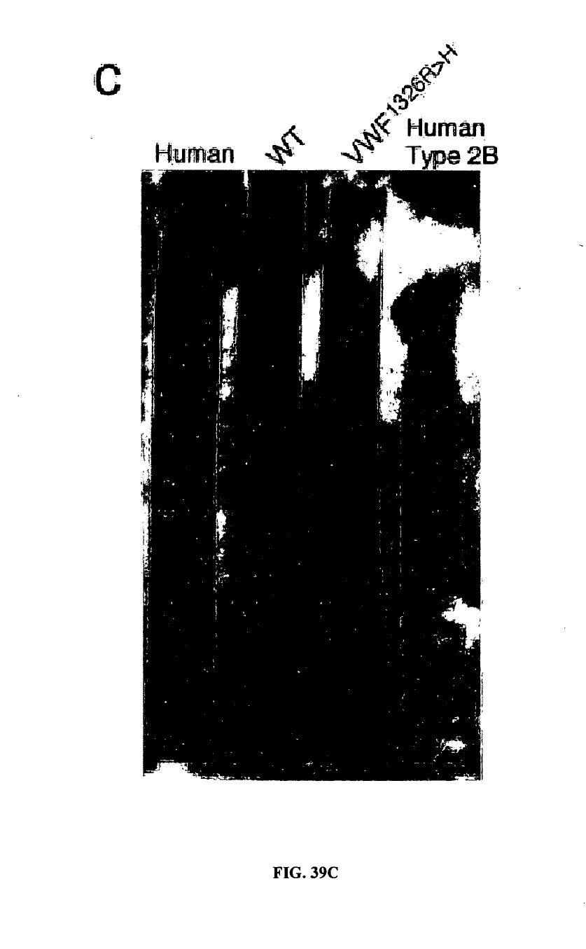

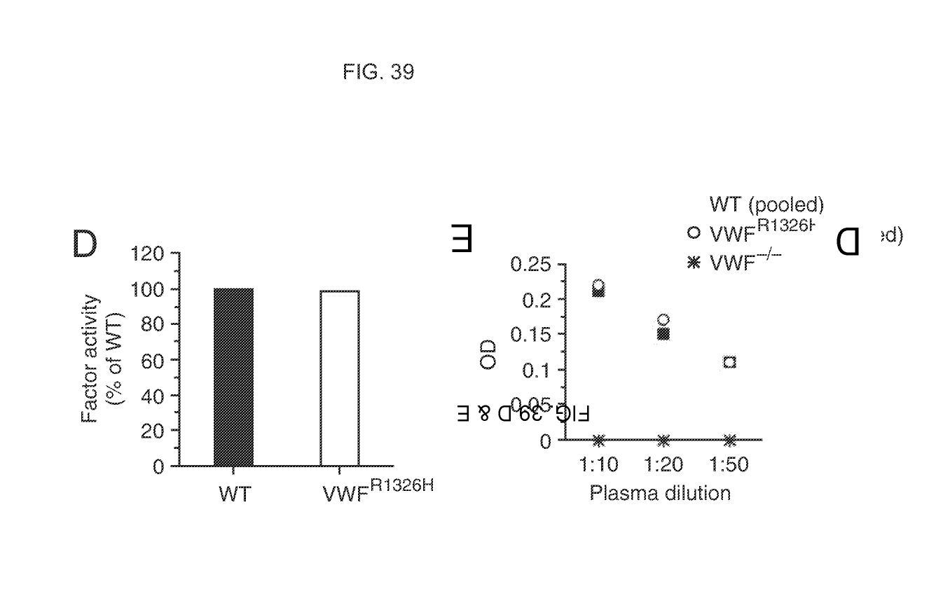

In order to determine the ability of full-length murine VWF containing the 1326R>H mutation (VWF^{1326R>H}) to support human platelet interactions and ultimately thrombus formation in vivo, mice were genetically modified to express VWF^{1326R>H} (FIG. 38B-C). Both homozygous and heterozygous animals were viable, fertile, born at the expected Mendelian ratio, and had platelet counts comparable to WT littermate controls. Moreover, reverse transcription-PCR (RT-PCR) of lung tissue from mutant mice with primers specific for the A1, A2, and/or A3 domains of VWF amplified cDNAs of the correct size and of similar intensity as compared to WT littermate controls (FIG. 39A). VWF gene transcription, antigen levels, VWF multimer pattern, Factor VIII function, and collagen binding in homozygous mutant plasma were found to be equivalent to WT controls (FIG. 39B-E). These results indicate that VWF gene translation, transcription, and posttranslational modifications were not perturbed by the targeting strategy. The ability of plasma VWF to bind to collagen was also not affected by the introduction of the point mutation.

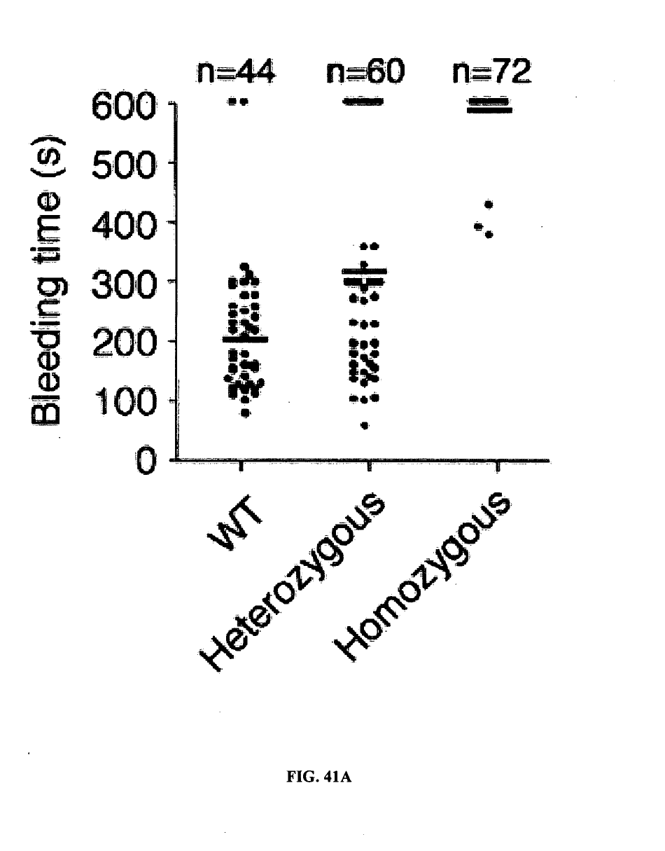

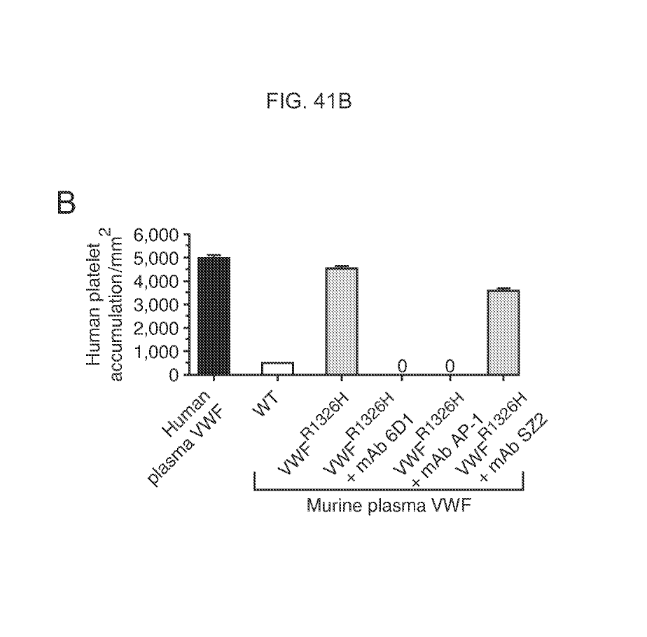

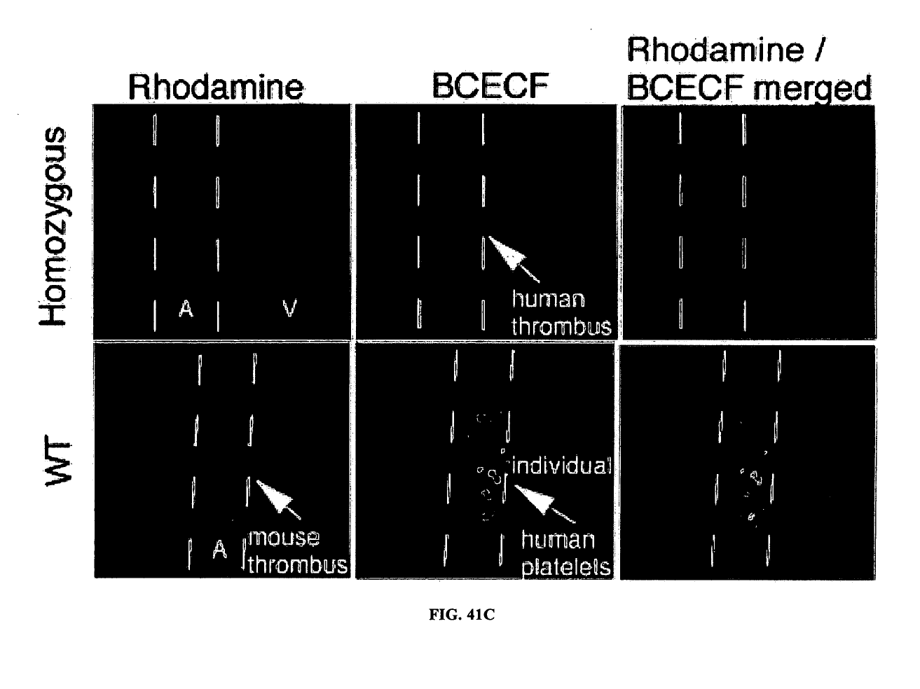

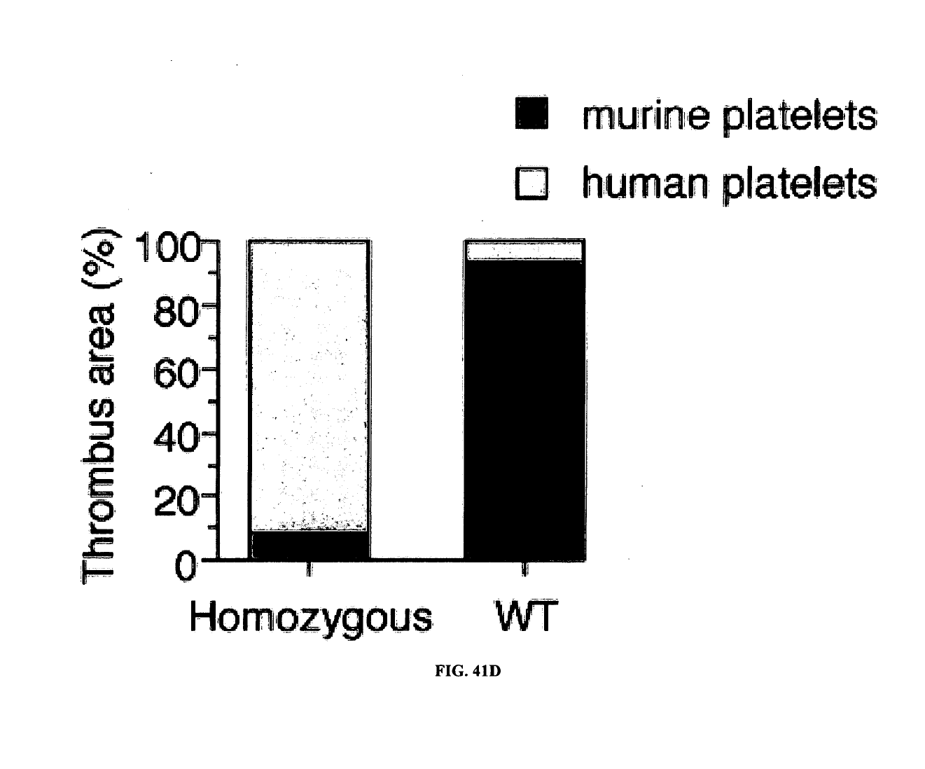

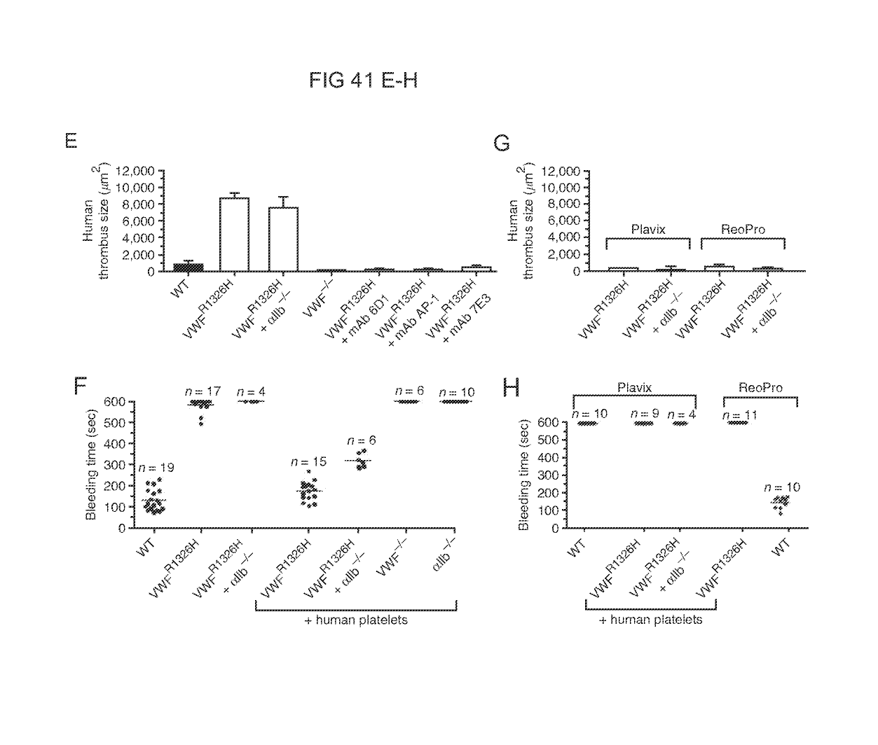

Hemostasis relies on platelet adhesion and activation at sites of vascular injury, which ultimately results in the formation of a hemostatic plug. To demonstrate the importance of VWF-A1 in this process, bleeding times for mice possessing the 1326R>H mutation were measured by removing 1 cm of distal tail (FIG. 41A). In contrast to their WT counterparts, the vast majority of homozygous VWF^{1326R>H} mice were incapable of forming an effective hemostatic plug, as they continued to bleed profusely throughout the duration of the experiment (10 minutes). Moreover, a smaller but statistically significant increase in bleeding time was noted for heterozygous animals (1.9-fold compared with WT; P=0.0055). Thus, disruption of a single salt bridge between murine VWF-A1 and GPIb α is sufficient to impair hemostasis.

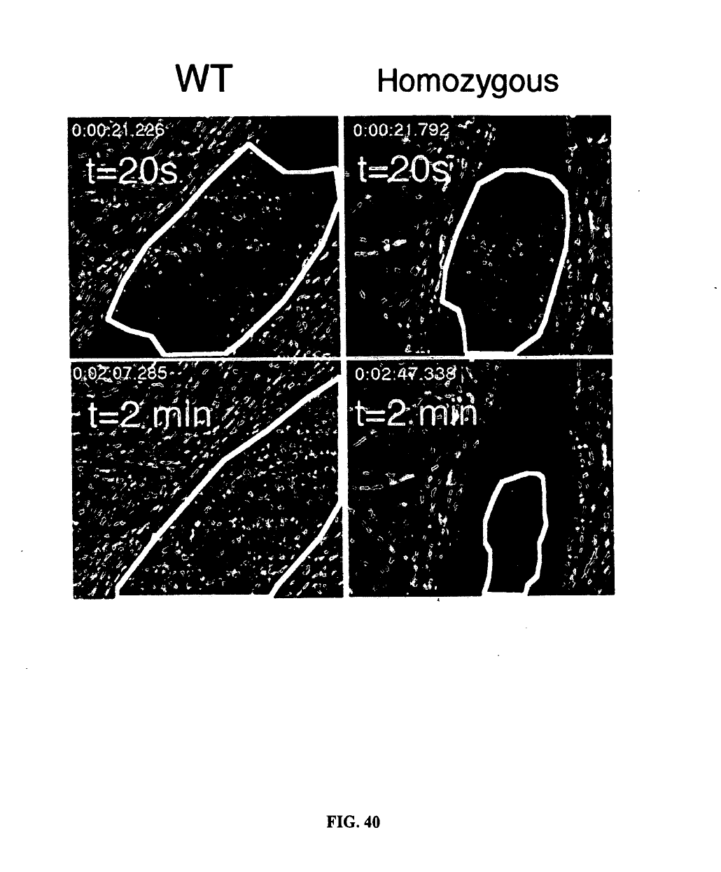

To gain insight into how the 1326R>H mutation alters hemostasis, murine platelet adhesion at sites of vascular damage was evaluated in vivo. A laser-induced vascular injury model was utilized to initiate platelet deposition in arterioles located in the microcirculation of the cremaster muscle of WT and homozygous mutant mice (Furie, B. et al., 2005). Although VWF^{1326R>H} animals can initially form thrombi that fill the vessel lumen, they rapidly dissipated under the prevailing hydrodynamic conditions (FIG. 40). By contrast, thrombi in WT mice continue to enlarge and eventually occlude blood flow under identical conditions.

To demonstrate that the removal of the electrostatic clash between residues 1326 and 231 in murine VWF-A1 and human GPIb α, respectively, promotes substantial interactions between this chimeric receptor-ligand pair, human whole blood was perfused over surface-immobilized plasma VWF obtained from mice homozygous for VWF^{1326R>H}. Remarkably, mutant murine VWF bound human platelets at levels comparable with its human counterpart (FIG. 41B). Moreover, a function-blocking antibody 6D1, which binds exclusively to human GPIb α at the fourth LRR (Shen, Y. et al., 2000), inhibited platelet adhesion, demonstrating a key role for the platelet receptor in adhesion to VWF^{1326R>H}. Another GPIb α function-blocking antibody AP-1, also abrogated interaction between mutant murine VWF and human platelets

By contrast, the antibody SZ2 that recognizes the anionic-sulfated tyrosine sequence of GPIb α (residues 276 to 282) had a minimal affect on platelet accumulation, results consistent with a previous report (Fredrickson et al., 1998). The A1 domain also possesses the ability to support the movement of attached platelets in the direction of the prevailing hydrodynamic force owing to rapid and reversible interactions with GPIb α (Savage et al., 1996, Doggett, T. A. et al., 2002). Translocation velocities of human platelets on either human or mutant murine VWF were therefore compared. Translocation velocities of human platelets on either homozygous mutant murine or native human plasma VWF were also similar (3.5±0.1 μm/sec vs. 3.2±0.1 μm/sec, respectively; mean±s.e.m, n=4), demonstrating that VWF^{1326R->H} functions in a manner indistinguishable from its human counterpart.

The ability of murine VWF^{1326R>H} to support human platelet adhesion in vivo was then tested. The ability of human platelets to preferentially support thrombus formation was monitored simultaneously by labeling purified human cells with BCECF ex-vivo, and mouse platelets with rhodamine 6G by intravenous administration. Fluorescently-labeled human platelets were infused continuously via a catheter inserted into the femoral artery, resulting in a high local concentration of these cells within the microcirculation of the cremaster muscle. Their behavior in response to laser-induced vascular injury was monitored in real-time using confocal intravital microscopy (Furie, B., et al., 2005). Upon induction of laser damage to the vessel wall of arterioles in mice homozygous for VWF^{1326R>H} human platelets rapidly adhered to the site of injury, forming large thrombi composed mainly of human cells (91.7±1.2%; mean±s.e.m) (FIG. 41C-D); average thrombus size was 8,950±1,620 μm² (mean±s.e.m.). In WT mice, by contrast, human platelets had only a limited capacity to bind to the damaged vessel wall, accounting for only 5.7±0.4% of total thrombus area (mean±s.e.m.) (FIG. 41C-D).

Consistent with the critical role of platelet GPIb α in mediating interactions with VWF-A1, pre-treatment of human platelets with mAb 6D1 or with mAb AP-1 greatly reduced thrombus size in the vasculature of VWF^{1326R>H} mice (265±125 μm² and 198±103 μm², respectively; mean±s.e.m.) (FIG. 41E). This was also validated by the observations that human platelet deposition at sites of arterial injury is limited in VWF-deficient mice (185±35 μm²; mean±s.e.m.), demonstrating that the A1 domain of this plasma protein serves as the major ligand for GPIb α in the humanized animal model of thrombosis.

Although GPIb α initiates platelet deposition at arterial shear rates, it is ultimately the platelet integrin αIIb β3 that supports thrombus growth by promoting platelet-platelet interactions. The contribution of human αIIb β3 in this process is demonstrated by the ability of the function blocking antibody 7E3 to also limit thrombus size (529±150 μm²) (FIG. 41E). Confirmation that the interaction formed between human platelets and murine VWF^{1326R>H} is sufficient to promote effective hemostasis is provided by the ability of infused human cells to restore bleeding times in mutant mice to a level observed for their WT counterparts (182±14.5 s vs. 131.5±11.2 s, respectively; mean±s.e.m.) (FIG. 41F). Importantly, human platelet-induced hemostatic clot formation can be completely disrupted in these animals by the pre-administration of either clopidogrel (PLAVIX), an inhibitor of ADP-induced platelet activation, or abciximab (ReoPro™), a Fab fragment of the chimeric human-mouse monoclonal antibody 7E3, which blocks the function of αIIb β3 on human but not murine platelets (Hankey, G. J. et al., Bennett, J. S. et al., 2001) (FIGS. 41G and 41H).

In summary, these studies demonstrate how one can effectively utilize atomic models of interspecies complexes to identify a binding hot spot where a disproportionate amount of the binding free energy is localized, such that a single amino acid substitution significantly affects the interaction (Bogan, A. A., et al., 1998), and in this case switches species specificity. Moreover, a subtle and localized change of this nature limits the possibility of inducing structural perturbations that impact on the function of other domains contained within VWF. These data show that human platelet adhesion to VWF^{1326R>H} is dependent on GPIb α binding to VWF-A1, with other potential ligands for this receptor playing a subservient role in this process (Bergmeier, W. et al., 2006). The reliance of human thrombus formation on the integrin αIIb β3, as well as the ability of the FDA approved drugs PLAVIX and ReoPro™ to impair human platelet-mediated hemostasis indicate that downstream adhesive and activation events known to be critical for clot formation and stability are intact in the mutant VWF animals. Thus, the VWF^{1326R>H} knock-in mice will prove useful in the preclinical evaluation of new antithrombotic therapeutics designed ultimately for human use. These results also have implications for advancing both knowledge of human platelet biology and in preclinical testing of antithrombotic therapies in vivo.

### Example 4

### Genetically Modified VWF-A1 Mice

### General Procedures and Assays

Kinetic evaluation of mutations associated with type 2B and platelet-type VWF suggests that the intrinsic properties of the GPIb α-VWF-A1 tether bond contribute to the regulation of platelet interactions with VWF. This is also supported by preliminary studies investigating the impact of botrocetin on the biophysical properties of this receptor-ligand pair. Thus, by using the information obtained in Example 2, mutations can be incorporated into the murine A1 domain of the VWF gene that increase or decrease the intrinsic on- and off-rates by varying degrees in order to truly understand the importance of these kinetic parameters in controlling platelet adhesion. Moreover, the role of the minor binding site, where the majority of type 2B mutations have been identified, can be further delineated by combining such mutations with those that significantly shorten the lifetime of interaction between GPIb α and VWF-A1. Results indicate that substitution of the murine residue Arg at position 1326 for His at the same location in the human A1 domain results in a diminished on-rate as manifested by the increased requirement for shear flow to promote attachment and a significant increase in k_{off} (shortening of bond lifetime). Subsequent incorporation of the type 2B mutation 1309I>V into this mutant domain significantly reverses the functional defect in adhesion and returns the off-rate closer to that observed for the WT domain. Similar results have been obtained with murine VWF-A1 in which Arg was replaced by His at residue 1326. Thus, introduction of these two mutations separately and then together into the mouse VWF gene will be the initial focus.

Generation of mice that incorporate mutations into their A1 domain that significantly shorten the bond lifetime will be present with prolonged bleeding times and will be resistant to thrombus formation, while the additional incorporation of a type 2B mutation will correct these abnormalities by prolonging the tether bond lifetime to that observed for the WT domain. This should allow for sufficient time to form multiple bonds between platelets and VWF deposited at sites of vascular injury.

By performing a detailed kinetic analysis of mutant VWF-A1 domains prior to the generation of animals with the identical substitutions in amino acids, the likelihood of altering the interaction between platelets and VWF in a similar manner is greatly increased. The role of the intrinsic properties of the bonds formed between this receptor-ligand pair under complex hemodynamic conditions (i.e. in vivo) may be studied.

### Generation and Characterization of Mice Expressing a Mutant VWF-A1 Domain.

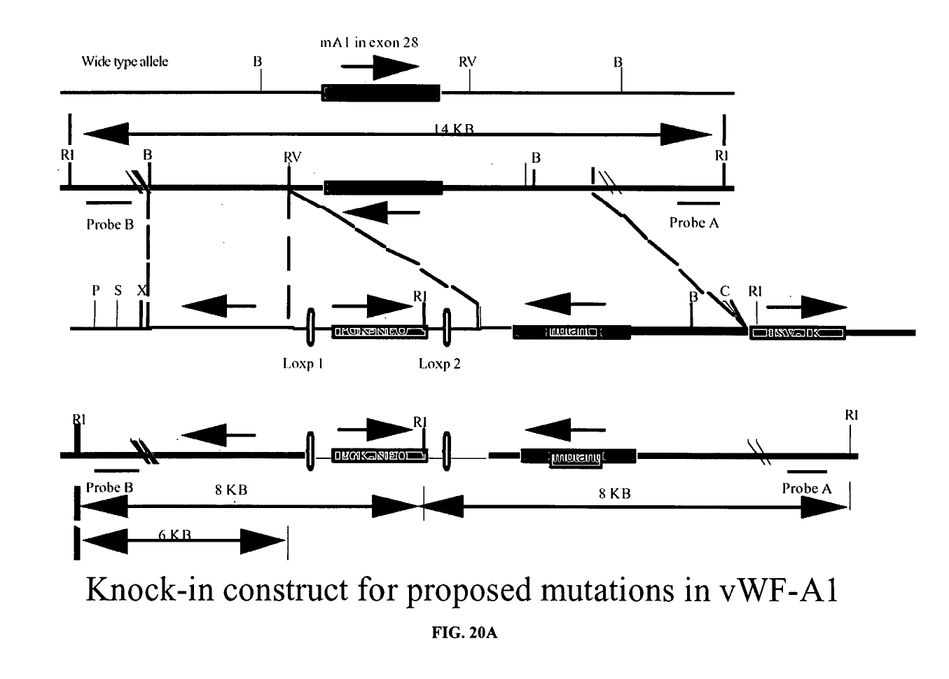

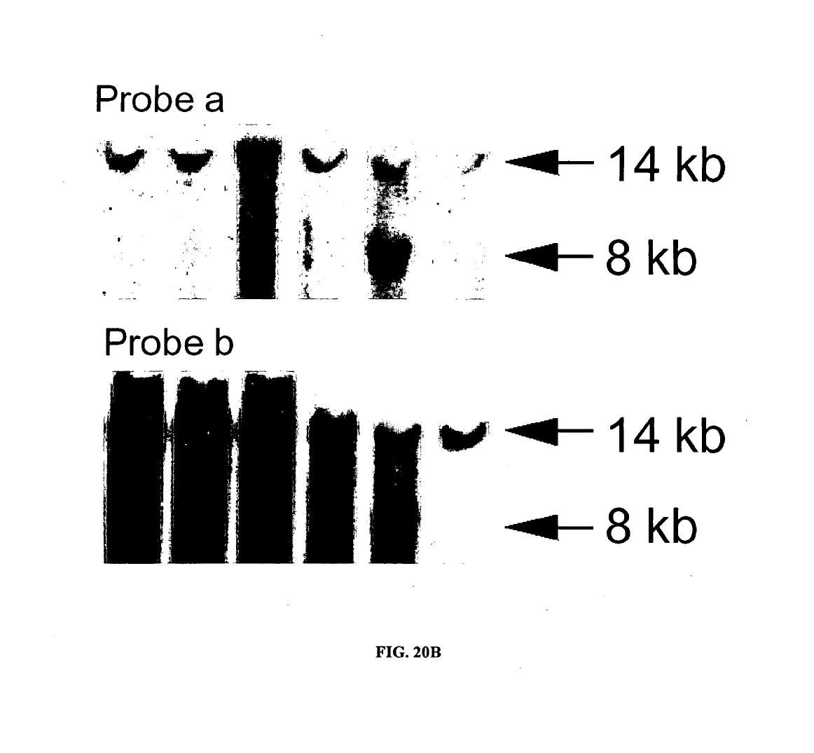

1309I>V single mutant or 1309I>V and 1326R>H double mutant mice were generated as follows. A 100-kb P1 clone containing the majority of the VWF gene (Genomic Systems, St. Louis, Mo.) was obtained. Digestion with Bam HI resulted in a about 5.3 kb fragment containing part of intron 28 (including the splice sites), all of exon 28 and part of intron 29 which was the inserted into the pSP72 vector (Promega Corp., Madison, Wis.). This was subsequently digested with Bam HI and Eco R5 to yield a 2.9 kb (including exon 28) and a 2.4 kb fragment, designated Arm 1 and 2, respectively, both of which were subcloned back into pSP72 vector. This facilitated site-directed mutagenesis of the A1 domain contained within Arm 1. In addition, the 3′ end of Arm 1 was extended 2 kb by PCR. Subsequently, Arms 1 and 2 were inserted into a lox P-targeting vector as shown below (FIG. 20). The fidelity of three constructs containing either the 1309I>V substitution or both 1309I>V and 1326R>H mutations was confirmed by sequence analysis.

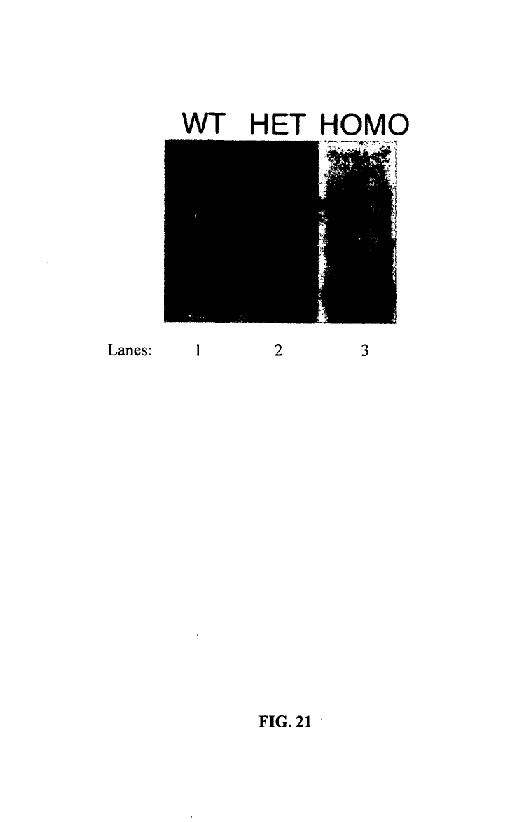

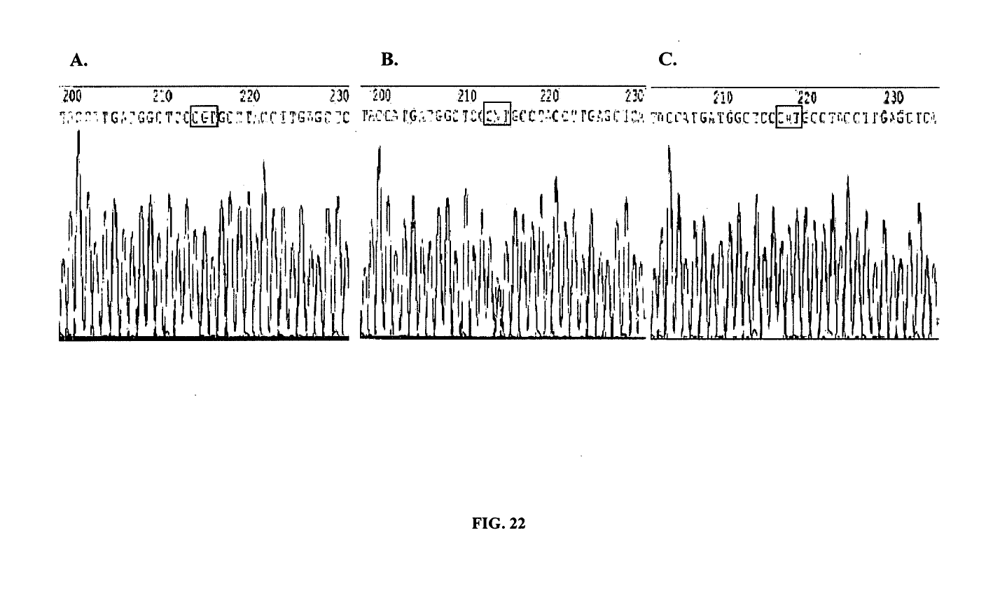

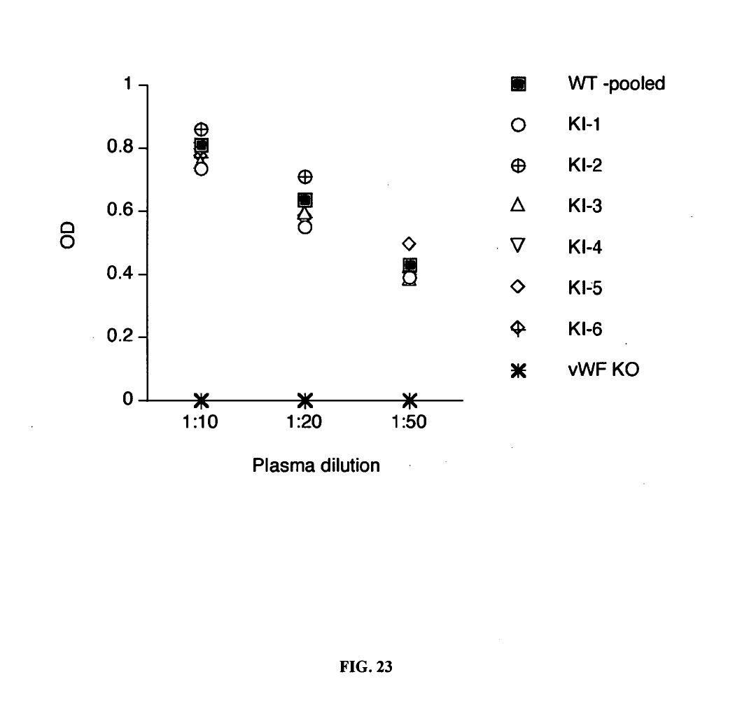

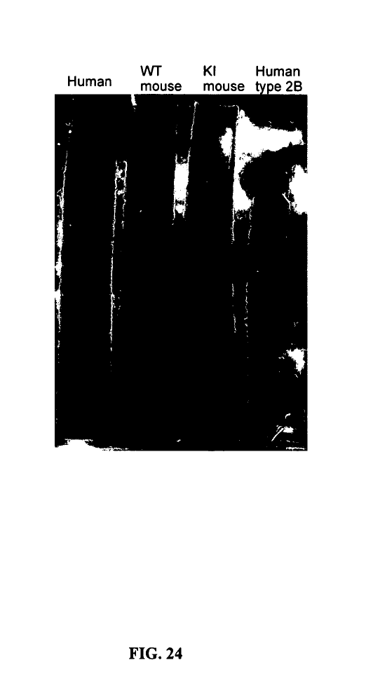

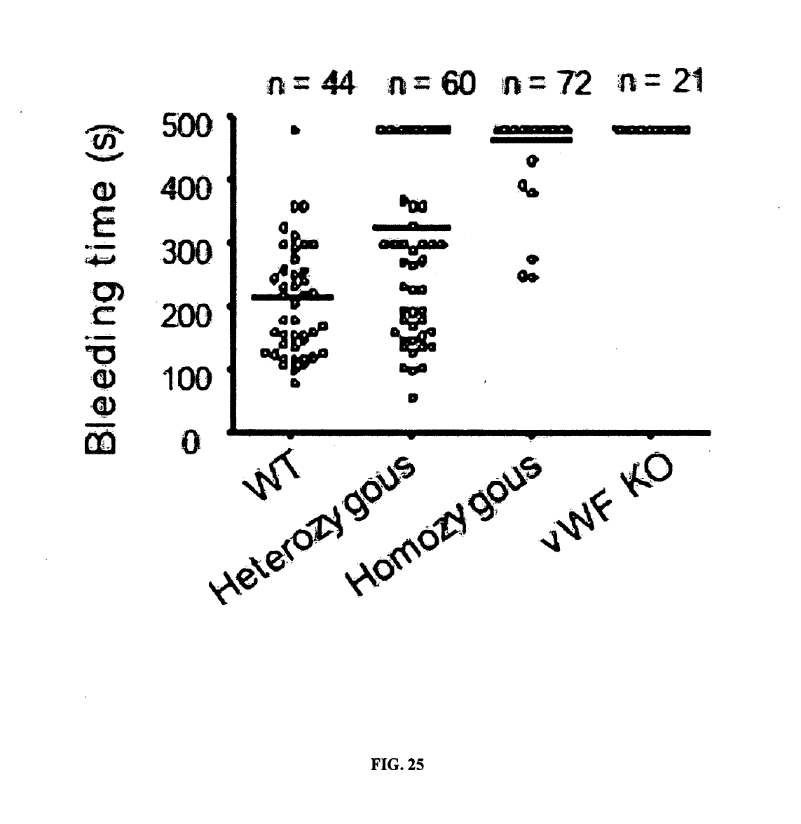

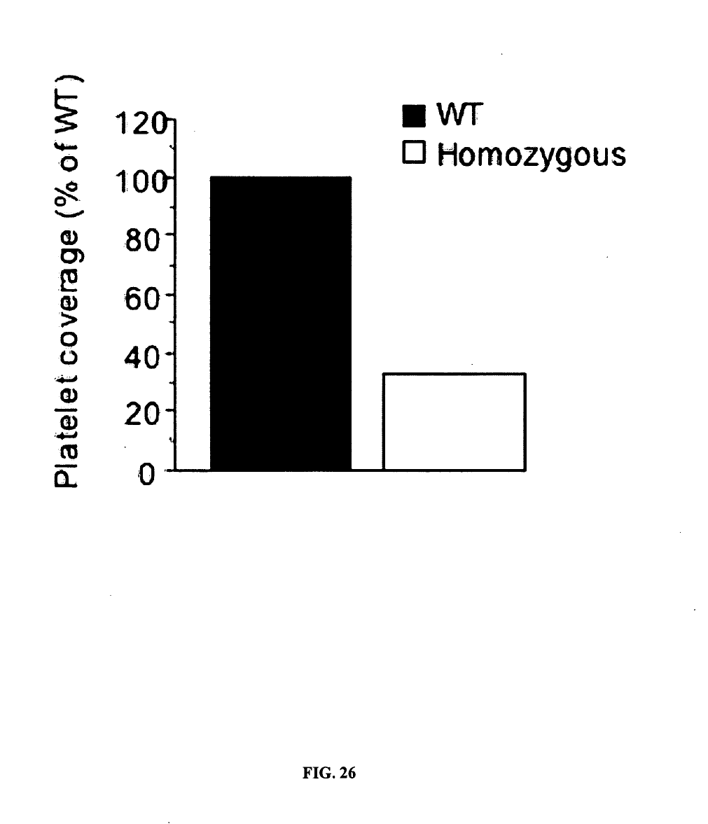

R1 embryonic stem cells derived from a 129/Sv X 129/Sv-CP F1 3.5-day blastocysts were electroporated with 25 μg of linearized targeting construct and selected in both G418 (26 μmol/L) and gancyclovir (0.2 μmol/L). Genomic DNA from resistant clones were digested with EcoRI or KpnI, and analyzed by Southern blot hybridization with probe “a” or “b”, respectively, to determine if the construct was appropriately targeted (FIG. 20B). Targeting both the type 2B (Ile1309Val or 1309I>V) mutation and the Arg1326His (1326R>H) mutant constructs, have been successful. In a second step, embryonic stem cell clones that had undergone homologous recombination were transfected with 25 μg of Cre-recombinase-expressing plasmid and selected for G418. Clones in which the neo-cassette was deleted were identified by PCR and injected into C57BL/6 blastocysts (The Siteman Cancer Center Core Facility, Washington University). Male chimeric mice were bred to C57BL/6 Cre-recombinase (+) females to obtain heterozygous animals. Heterozygous mice lacking the neocassette, but containing the 1326R>H mutation, were interbred to obtain wild-type, heterozygous, and homozygous animals. Animals were identified by both Southern analysis (FIG. 21) and by PCR of the A1 domain (FIG. 22; boxed area denotes the conversion of Arg to His).

Other mutant mice may be generated using any of the vector targeting strategies disclosed herein.

### Determination of the Multimeric Composition of Murine VWF.

For platelet counts, whole blood will be collected into heparinized tubes and 100 μl volumes will be analyzed on a Hemavet (CBC Tech, Oxford, Conn., USA) Coulter Counter. The multimeric structure of murine VWF will be assayed by using the Pharmacia Phast Gel System (Pharmacia LKB Biotechnology). Briefly, samples diluted in 10 mmol/L Tris/HCl, 1 mmol/L EDTA, 2% SDS, 8 mol/L urea, and 0.05% bromophenol blue, pH 8.0, will be applied to a 1.7% agarose gel (LE, Seakem, FMC Bioproducts) in 0.5 mol/L Tris/HCl, pH 8.8, and 0.1% SDS with a stacking gel consisting of 0.8% agarose (HGT, Seakem) in 0.125 mol/L Tris/HCl, pH 6.8, and 0.1% SDS. After electrophoresis the protein will be transferred to a polyvinylidene fluoride membrane (Immobilon P, Millipore) by diffusion blotting for 1 hour at 60° C. The membrane will be blocked with 5% nonfat dry milk protein solution for 1 hour at room temperature. After washing with PBS/T, pH 7.4, the blot will be incubated with a polyclonal antibody raised in rabbits against murine VWF at a dilution of 1:500, washed, and incubated with a goat anti-rabbit horseradish peroxidase (Sigma) diluted 1:2000 in PBS/T. After three washes with PBS/T, the membrane will be incubated with the substrate solution (25 mg 3,3′-diaminobenzidine tetrahydrochloride (Sigma) in 50 mL PBS with 10 μL 30% H₂O₂). The enzyme reaction will be stopped by washing the membrane with distilled water.

### Bleeding Time for Human Platelet-Induced Hemostasis.

This assay provides an indirect measure of the ability of platelets and VWF to support hemostasis by interacting with the injured vessel wall. It also indirectly determines the function of multiple receptors and ligands on platelets that are required to form a hemostatic plug. That said, it provides direct evidence that the bleeding defect in the animals can be corrected by the administration of human platelets (FIG. 12). It is performed by immersing the severed tip (10 mm) of the animal's tail in isotonic saline at 37° C. and monitoring the length of time required for bleeding to cease. Homozygous mutant mice will be infused with an equal volume of either saline or purified human platelets. Platelet specific antibodies or drugs will be administered as described above and their ability to prolong bleeding time evaluated. All experiments will be stopped at 10 minutes by cauterizing the tail (Denis et al., 1998).

### Platelet Adhesion Studies in Mice Expressing a Mutant VWF-A1 Domain.

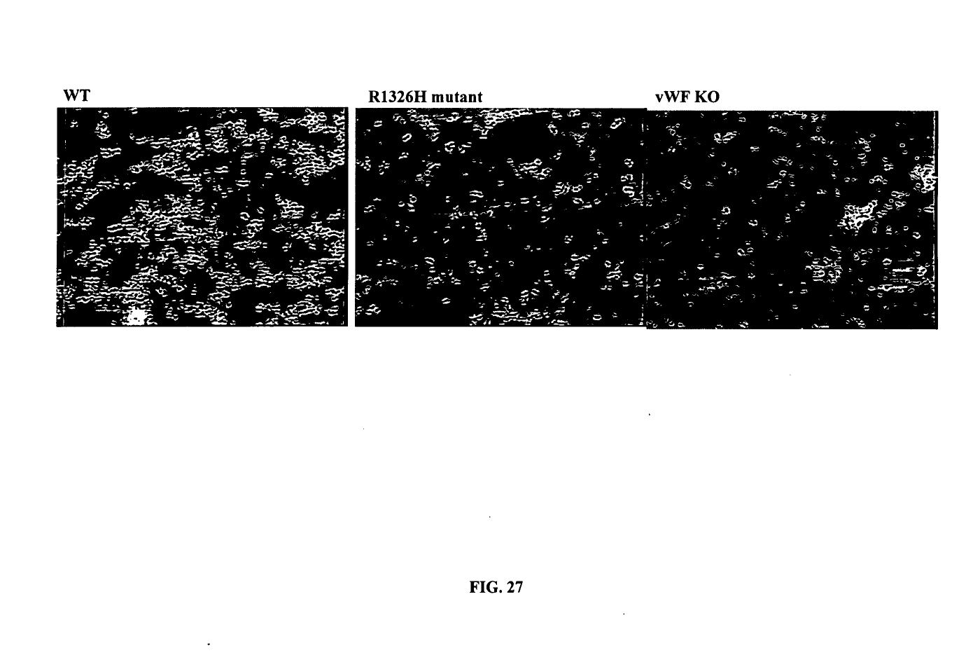

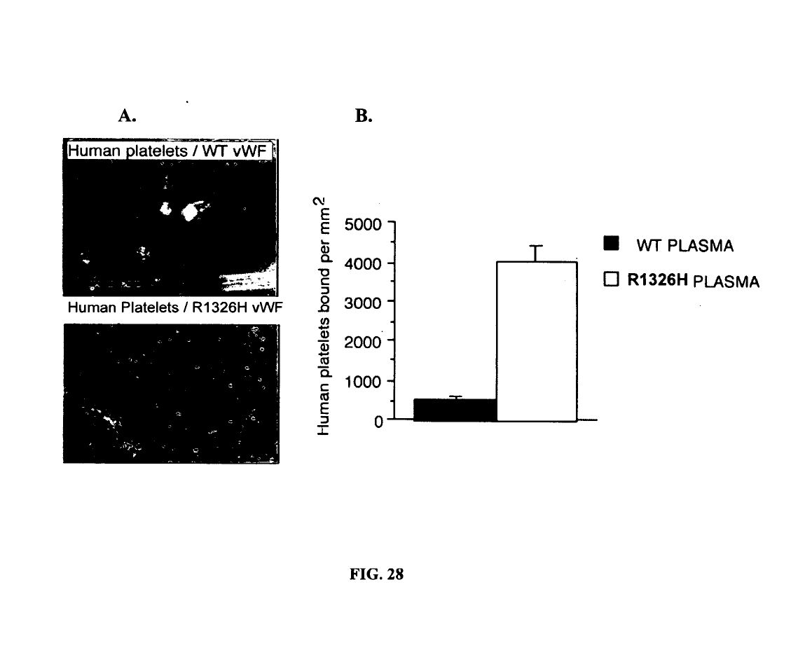

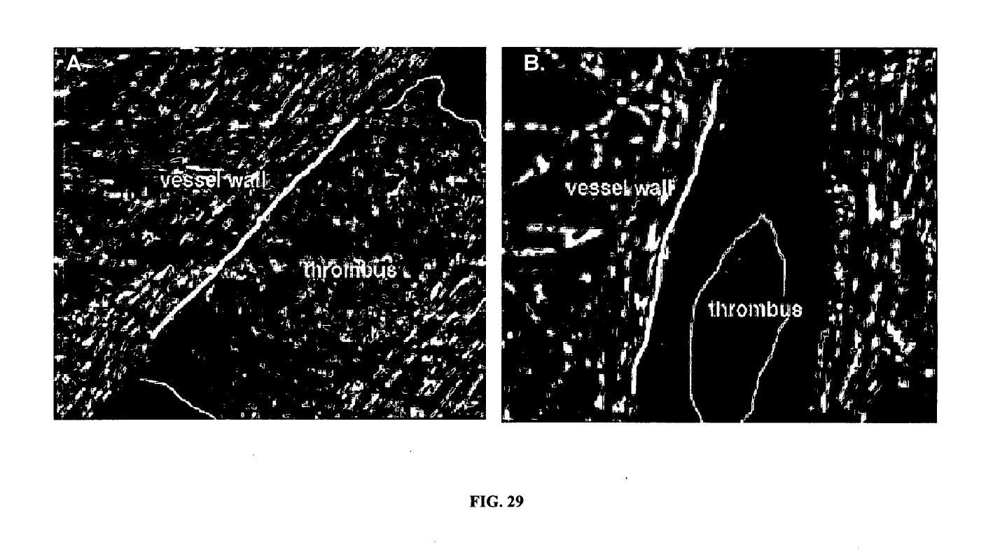

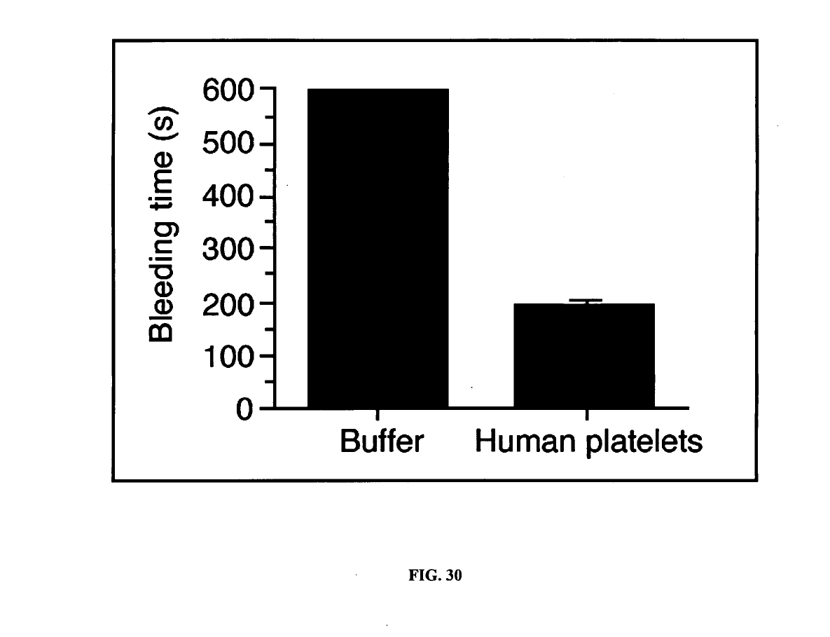

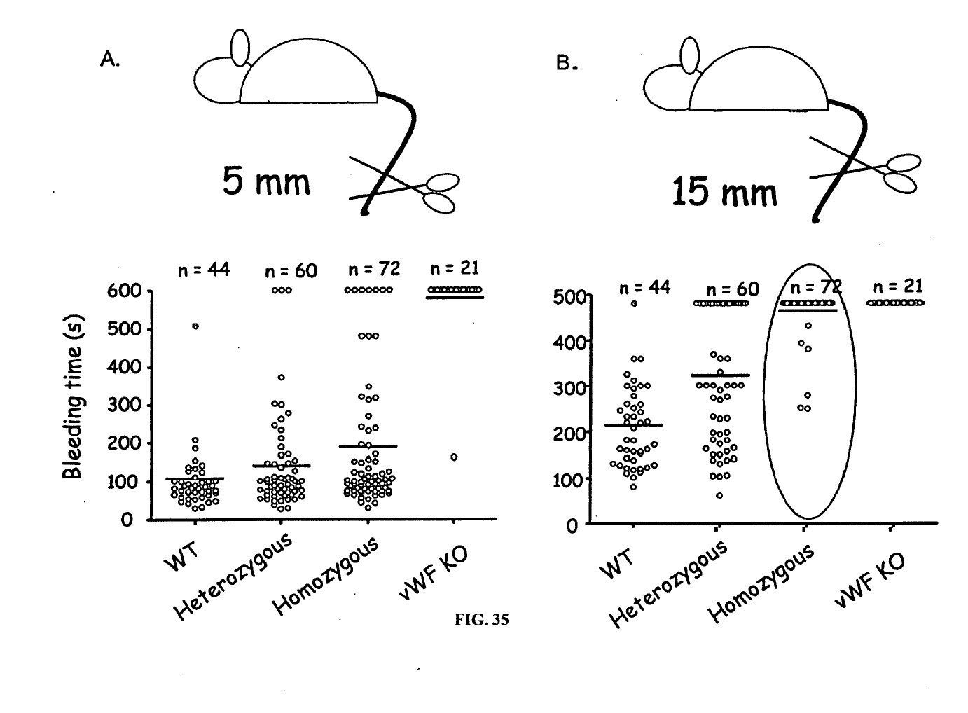

One goal of the work is to generate mice with mutant A1 domains that alter the kinetics of its interactions with GPIb α on mouse platelets. The first mutation introduced was the substitution of histidine for arginine at position 1326. This mutation was chosen based on the crystal structure analysis of the mouse and human A1 domains, which suggested that the location of this amino acid is central to GPIb α binding. Mice bearing this mutation are viable and demonstrate a bleeding phenotype, albeit not as severe as those lacking VWF (VWF KO) (FIG. 35). This was not unexpected as VWF is still present, but has a reduced ability to interact with platelets at high shear rates (>1600 s⁻¹). FIG. 27 demonstrates reduced thrombus formation that occurs when whole blood from these knock-in animals is perfused over collagen-coated cover slips at a shear rate of 1600 s⁻¹. Results thus far indicate a 70% reduction in thrombi formed on collagen as compared to WT controls.

### Evaluation of Platelet-VWF Behavior in Flow.

Blood will be collected by cardiac puncture from anesthetized mice and thrombin-mediated activation prevented by the addition of hirudin (160 U/ml, Sigma) (Andre, et al., 2002). Platelet adhesion to a glass cover slip coated with 100 μg/ml of equine tendon collagen (Helena Laboratories, Beaumont, Tex.) will be assessed in a parallel-plate flow chamber apparatus. Whole blood will be infused through the chamber at a wall shear rate of 1600 s⁻¹ for 3 minutes. As platelet adhesion under these homodynamic conditions requires VWF deposition and subsequent interactions between its A1 domain and GPIb α, the extent of platelet coverage should provide a gross estimate of the degree of impairment between this receptor-ligand pair. In addition, plasma VWF will be purified from these animals to evaluate platelet attachment to this immobilized substrate in flow. The surface area covered by adherent platelets at the end of each experiment will be determined (Image Pro Plus software) and expressed as a percentage of platelet coverage using blood from WT littermates. To better isolate GPIb α-VWF A1 interactions, identical experiments can be performed using platelets isolated from αIIb β3 deficient animals and reconstituting them in platelet poor plasma from the mutant A1 knock-in mice.

### Evaluation of Platelet-VWF Behavior In Vivo.

In addition to the in vitro work, platelet-VWF interactions in vivo will also be studied using intravital microscopy (Falati et al., 2002). This is accomplished by using a murine model of thrombosis that involves laser-induced injury to micro-vessels contained within the mouse cremaster muscle. The surgical preparation of animals, insertion of lines for administration of cells and anesthesia, will be performed as previously described (Coxon et al., 1996). Human platelets will be collected and prepared, fluorescently labeled, perfused into a mouse model (such as the transgenic mouse of the current invention) via an intravenous injection (Pozgajova et al., 2006).

### Surgical Preparation of Animals.

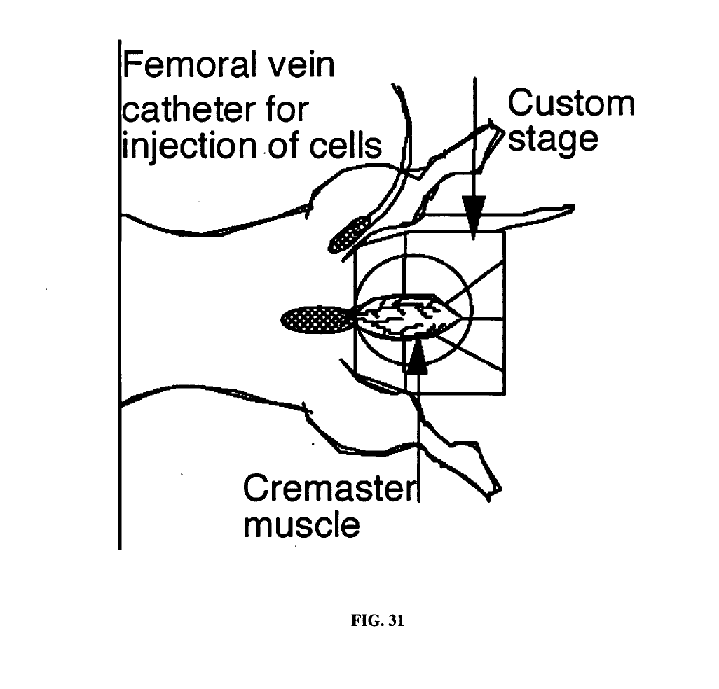

Insertion of lines for administration of cells and anesthesia. Briefly, the skin covering the scrotum will be incised and the intact cremaster muscle dissected free from the connections to the subcutis. The mouse will be placed on a custom-built plexiglass board, and the exposed muscle positioned on a heated circular glass coverslip (25 mm) for viewing. The muscle will be slit along the ventral surface (using a thermal cautery), the testis excised, and the muscle spread across the coverslip with attached sutures (6/0 silk) (FIG. 31). The cremaster muscle will be kept continuously moistened by superfusion throughout the experiment with sterile, bicarbonate-buffered (pH 7.4), saline solution (37° C.) that is pregassed with a 5% CO₂, 95% N₂ mixture for O₂ depletion. All parts of the setup in contact with the superfusion buffer will be presoaked with 1% Etoxaclean (Sigma Chemical Co., St. Louis, Mo.) overnight followed by extensive rinsing in 70% ethanol and endotoxin-free distilled water. The number of mice used for these experiments will be kept to the minimum necessary to establish statistically significant observations. Anesthetized animals will be euthanized after each experiment by CO₂ inhalation.

### Vascular Trauma.

The segment of an arteriole will be visualized and recorded as “pre-injury”. Subsequently, endothelial damage will be induced via a pulsed nitrogen dye laser at 440 nm applied through the microscope objective using the Micropoint laser system (Photonics Instruments, St. Charles, Ill.). The duration of exposure of the endothelium to the laser light will be varied to produce either a mild injury that supports the formation of a platelet monolayer or significant injury resulting in thrombus formation. The region of interest will then be videotaped and analyzed as described below.

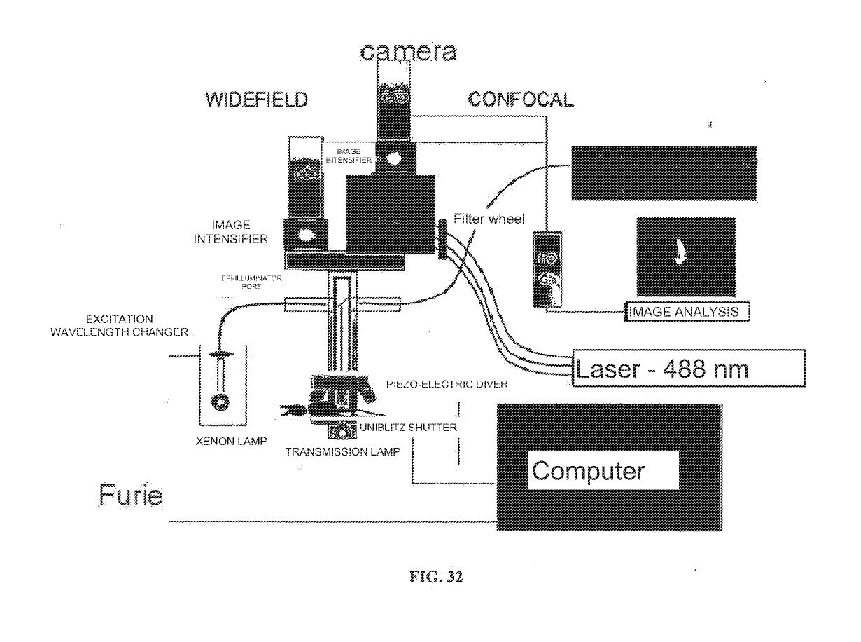

For example, vascular damage can subsequently be induced in arterioles contained within the cremaster muscle of mice by either 1) a pulsed nitrogen dye laser applied through the objective of an intravital microscope (FIG. 32) or 2) standard application of a ferric chloride solution (Furie et al., 2005). The latter method has the advantage of exposing significantly more subendothelial collagen, which will be beneficial for testing the role of the collagen receptors α2 β1 in thrombus formation.

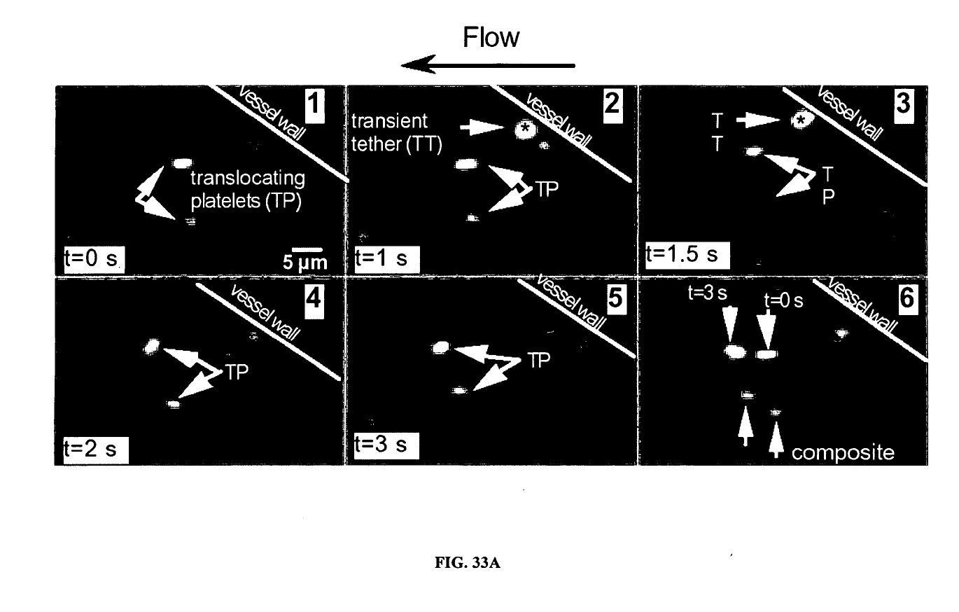

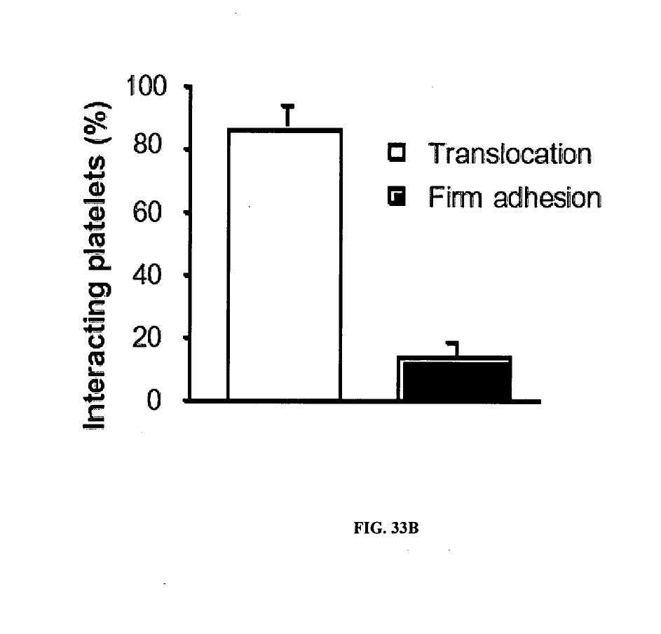

For studies analyzing the dynamic interactions between individual platelets and the injured vessel wall (attachment, translocation, and sticking), cells purified from genetically altered mice will be labeled ex-vivo with a derivative of carboxyfluorescein (BCECF, Molecular Probes) (Diacovo, et al., 1996). A human thrombus generated in the mutant mouse can also be visualized by this technique, thus allowing one to distinguish human platelets from endogenous circulating mouse platelets upon illumination with an appropriate laser light source (see FIG. 40). Cells (1×10⁷/g of BWT) will be subsequently injected intravenously into mice bearing WT mouse (control) or the “humanized” A1 domains and their behavior visualized in the microcirculation using an intravital microscope (Zeiss, Axiotech Vario; IV500, Mikron Instruments, San Diego, Calif.; and the like) equipped with an iXON EM camera or a silicon-intensified camera (VE1000SIT; Dage mti, Michigan City, Ind.), a Yokogawa CSU22 confocal head, and a 488 nm laser line (Andor Technology, Revolution series). A Xenon arc stroboscope (Chadwick Helmuth, El Monte, Calif.) will serve as the light source and fluorescent cells will be viewed through 60× or 100× water immersion objectives (Acroplan, Carl Zeiss Inc.). A tethered platelet will be defined as a cell establishing initial contact with the vessel wall (FIG. 33A, panel 2-3; FIG. 33B). The translocating fraction will be defined as the number of tethered platelets that move at a velocity significantly lower than the centerline velocity for >1 second. The sticking fraction will be defined as the number of translocating cells that become stationary for >30 seconds post-tethering. Second order arterioles (up to 50 μm in diameter) will be evaluated for platelet interactions before and after the injury. Evaluation of platelet circulation in larger arterioles may be less accurate secondary to hemoglobin-mediated quenching of fluorescence emitted from platelets traveling in an area of the blood stream distal to the focal plane of the objective. Epi-illumination will only be used during video recordings to minimize possible phototoxic effects on tissue.

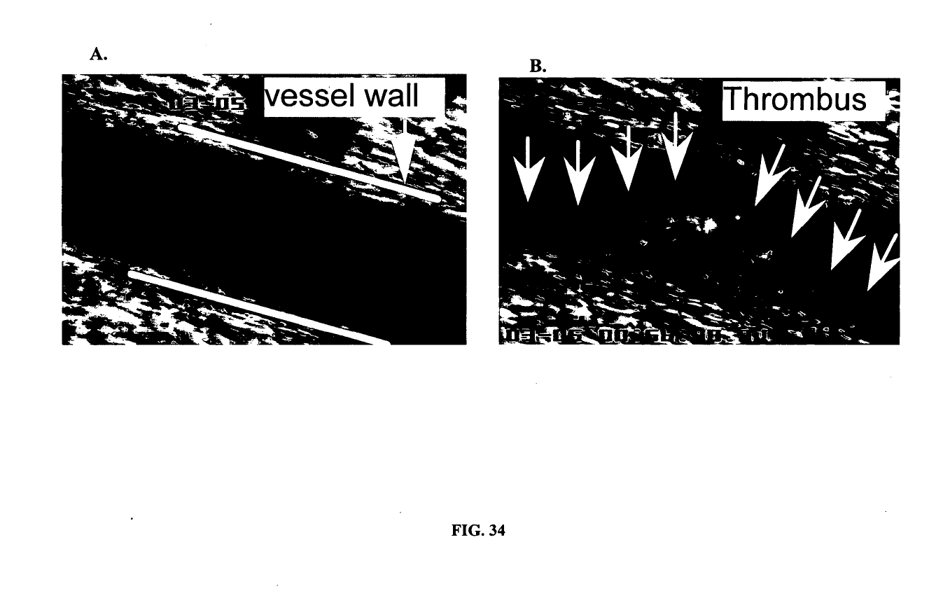

A role for GPIb α as well as the collagen (α2 β1) and the fibrinogen (αIIb β3) receptors can be evaluated by using function-blocking antibodies to these proteins. Moreover, FDA approved anti-thrombotics (such as clopidogrel and tirofiban) can be used to examine whether the drugs inhibit human platelets from forming a thrombus in vivo, validating the mouse model for use in pre-clinical screening. The effect that antibodies and drugs have on altering the interaction between GPIb α-VWF-A1 interaction is determined by evaluating whether thrombus formation in the proposed mice is reduced or augmented upon arteriolar injury (FIG. 34).

For all experiments, the centerline erythrocyte velocity (Vrbc) is measured using an optical doppler velocimeter (Microcirculation Research Institute, Texas A&M College of Medicine, College Station, Tex.) prior to and after inducing the injury. Shear rate (SR) is then calculated based on Poiseulle's law for a Newtonian fluid: SR=8(Vmean/Dv), where Dv is the diameter of the vessel and Vmean is estimated from the measured Vrbc (Vmean=Vrbc/1.6).

### Characterization of Thrombus Formation.

Thrombus formation can be characterized as follows: (1) Early individual platelet interactions with the damaged vessel wall (number of fluorescently labeled human platelets that attach during the first minute post-injury); (2) time required for thrombus generation of >20 μm diameter; (3) the ability of thrombi to remain at the initial site of vascular injury and not break free (measure of stability); (4) time until vessel occlusion; and (5) site of vessel occlusion, that is, at the site of injury or downstream from it. Platelet-vessel wall interactions can be viewed through 40× or 60× water immersion objectives. To standardize in vivo conditions, the velocity of flowing blood (shear rate) pre-injury is determined by measuring the centerline erythrocyte velocity (Vrbc) using an optical doppler velocimeter. Shear rate (SR) can then be calculated based on Poiseulle's law for a Newtonian fluid: SR=8×(Vmean/Dv), where Dv is the diameter of the vessel and Vmean is estimated from the measured Vrbc (Vmean=Vrbc/1.6). Vessel and thrombus diameters are measured using imaging software (ImagePro Plus).

### Administration of Antibodies.

Function-blocking monoclonal antibodies 6D1 (anti-human GPIb α), 6F1 (anti-human α2 β1) and 7E3 (anti-human αIIb. β3) have been generously provided by Dr. Barry Cotler (Rockefeller University, NY). All antibodies are converted to F(ab′)₂ fragments to limit Fc receptor interactions in vivo. An intravenous dose of 10 μg/g body weight is given approximately 10 minutes after the injection of human platelets but 30 minutes prior to inducing vascular injury. Non-function blocking antibodies to these receptors are used as negative controls and administered under identical conditions. To ensure optimal ligand availability for the collagen and fibrinogen receptors on human platelets, mice possessing the A1 domain mutation have been bred with animals genetically deficient in α2 β1 or αIIb β3. Thus, endogenous platelets in these animals not only have a reduced ability to interact with the VWF-A1 domain, but also are incapable of binding to collagen or fibrinogen, respectively. Although human platelets have been shown to circulate in mice for a maximum of 24 hours, we can ensure that an equivalent percentage of human platelets are present at the time of vascular injury under each experimental condition (Xu et al., 2006). Thus, 50 μl is obtained from an inserted venous catheter and flow cytometric analysis will be performed to determine the percentage of circulating fluorescently-labeled human platelets.

### Administration of Drugs.

In comparison to aspirin, clopidogrel (Plavix) is the second most commonly used anti-thrombotic drug that targets one of the ADP receptors (P2Y12) on platelets, causing irreversible inhibition (Hankey, et al., 2003). ADP is a potent mediator of platelet activation and aggregate formation, and thus considerable effort and funds have been devoted to inhibiting this activation pathway in platelets. Clopidogrel was approved by the FDA in 1997 for clinical use and was found to be of benefit in the secondary prevention of major vascular events in patients with a history of cerebrovascular and coronary artery diseases and major cardiac events post coronary artery stent placement (Gachet et al., 2005). Disadvantages of this drug are: 1) It must be metabolized in the liver to generate an active metabolite, thus limiting its effectiveness in acute settings, and 2) irreversible inhibition that results in a marked prolongation of bleeding time.

Clopidogrel has been shown to reduce thrombus size and delay its formation in mice with a maximal effective dose of 50 mg/kg given the day before and 2 hours prior to experimentation (Lenain, et al., 2003). This drug will be obtained from the hospital pharmacy and tablets will be dissolved in sterile water for oral administration. Control animals will receive water in lieu of drug. The effectiveness of this treatment regime will be confirmed by first measuring the responsiveness of platelets isolated from drug-treated WT animals to ADP-induced aggregation using an optical aggregometer (Chrono-Log Corp.) as previously described (Leon et al., 1999). Because the mutant VWF-A1 domain mice also have a defect in platelet aggregation, these animals cannot be used for the purpose of testing to ADP-induced aggregation ex-vivo. However, this additional phenotype will be advantageous because it limits potential competition between human and mouse platelets for binding to ligands exposed at sites of vascular injury. Human platelets will be administered 30 minutes prior to vascular injury and 50 μl of blood drawn to determine the percentage of circulating cells as described above. Platelet rich plasma will also be purified from control and drug treated animals that receive human platelets to evaluate the effectiveness of clopidogrel on preventing ADP-induced aggregation of these cells ex-vivo.

Tirofiban (Aggrastat) is a non-peptide inhibitor of the fibrinogen receptor αIIb β3 that limits the ability of platelets to form aggregates, an event required for thrombus progression. It has a plasma half-life of approximately 2 hours but only remains bound to platelets for seconds, thus necessitating continuous intravenous administration. It is currently approved for short-term treatment of patients with acute coronary syndrome that require interventional catheterization. Thus, the animals will be dosed based on that given for interventional procedures such as angioplasty, which consists of a 25 μg/kg bolus over 3 minutes followed by a continuous maintenance infusion of 0.15 μg/kg/min until the completion of the experiment (Valgimigli et al., 2005). Human platelets will be administered 30 minutes prior to vascular injury and 50 μl of blood drawn to determine the percentage of circulating cells as described above.

### Platelet Donors.

Mice are used as platelet donors. A means to evaluate murine platelet interactions with wild type and mutant VWF-A1 proteins is via in vitro flow chamber assays. Blood from about 10 mice are required to purify adequate numbers of platelets per assay. Blood from donor animals is obtained from the retro-orbital plexus using a heparinized glass pipette. Mice will be anesthetized with Ketamine and Xylazine prior to the procedure and are euthanized by CO₂ inhalation upon completion.

### Bleeding Time for Human Platelet Induced Hemostasis.

This assay is carried out as disclosed above.

### Solution-Phase Binding Assay.

For type 2B mutant VWF, its capacity to bind to platelet GPIb α in solution can be determined. Plasma is harvested from these mice and VWF purified. Various concentrations of the plasma glycoprotein will be indirectly labeled using a non-function blocking, ¹²⁵I-labeled mAb to its A1 domain as previously described (Ribba et al., 1992). After a 30 minutes incubation, a quantity of this mixture will be incubated with platelets purified from β3 deficient mice so to prevent integrin-mediated binding to VWF. After an incubation period of 1 hour, an aliquot of this mixture will be added to a sucrose gradient and centrifuged to pellet the platelets. Radioactivity associated with the pellet vs. supernatant will be determined in a γ-scintillation counter, and the binding estimated as the percent of total radioactivity.

### Example 5

### Generation of Mice Bearing the Majority of the Human von Willebrand Factor A1 Domain

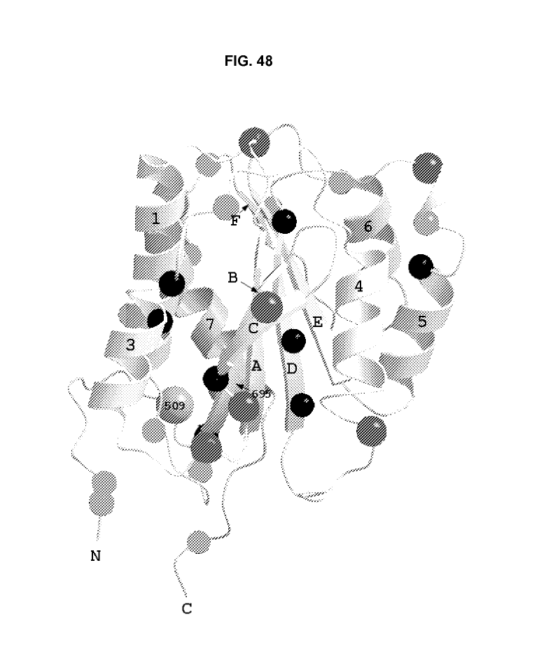

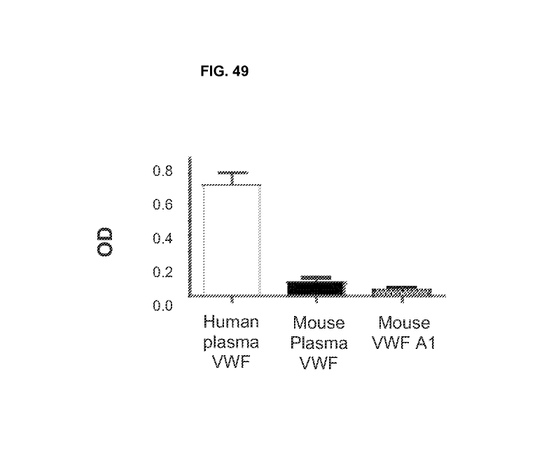

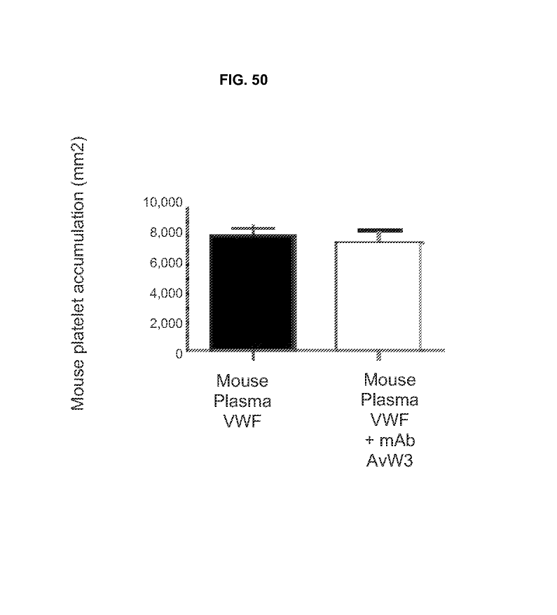

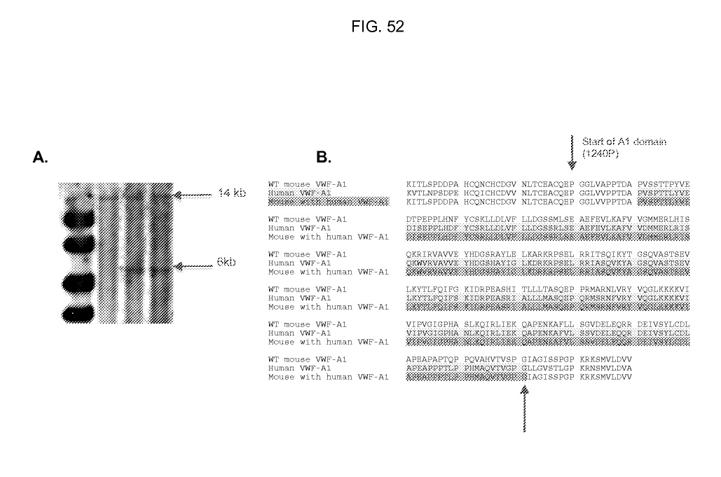

To better simulate human platelet mediated thrombosis in mice, an animal in which the majority of the A1 domain of von Willebrand factor (VWF) has been replaced with its human counterpart (amino acids 1240P through 1481G of the human VWF) was generated. The rationale for generating this animal is the ability to rapidly develop and determine the preclinical efficacy of therapies that specifically target the A1 domain of human VWF. There are 34 amino acid differences between the human and murine A1 domains of VWF (FIGS. 48 and 52). Consequently, an antibody known to bind to the A1 domain of HUMAN VWF (e.g. mAb AvW3) does not interact significantly with surface immobilized mouse plasma VWF or the recombinant mouse VWF A1 domain, as determined by a standard protein ELISA (FIG. 49). Because this antibody is also known to block the interaction between human VWF and human platelets, the lack of binding to mouse VWF or its isolated A1 domain suggests that it would not inhibit interactions between these mouse proteins and mouse platelets. This is confirmed by the inability of mAb AvW3 to reduce mouse platelet binding to surface immobilized mouse plasma VWF in a parallel plate flow chamber assay, as disclosed in Example 1, at a wall shear rate of 1,600 s⁻¹ (FIG. 50).

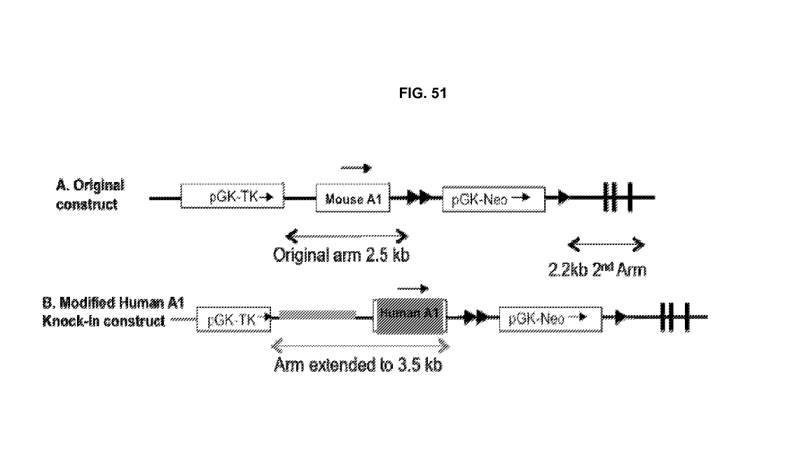

Generating a mouse bearing the majority of the human VWF A1 domain required a targeting approach different from the VWF mutant mice disclosed in Examples 3 and 4, because the use of the original targeting vector did not result in transgenic animals. To generate a transgenic mouse containing a majority of the human VWF A1 domain, Arm 1 of the construct was extended to a total length of about 3.5 kb and >85% of the human A1 domain sequence was substituted for its murine counterpart. The targeting construct is shown in SEQ ID NO:11. Additional modifications from the process disclosed in Examples 3 and 4 required for the generation of the human VWF A1 domain bearing animal included: (i) splitting ES cells 1:6 (vol:vol) instead 1:3; (ii) harvesting ES cells at 50% confluence rather than over 70% confluence; (iii) replacing media for the ES cells 4 hours before harvesting for electroporation; (iv) devising a screening method (see below for details) to identify correctly targeted clones, which included long-range PCR to detect homologous recombination of the targeting arms. This permitted the use of primers external to the 5′ and 3′ regions of the targeting vector. Other modifications include (v) enriching for viable ES cell clones, which required plating cells on feeders and incubating at 37° C. for 20 minutes instead of the typical 45 minutes. Unattached cells were then aspirated away. The loosely attached ES cells were then washed off for use in microinjection. A further modification from the method used in Examples 3 and 4 included (vi) increasing the number of targeted ES cells for injection into blastocysts. 30 correctly targeted ES cells instead of the typical 15 ES cells were used. This permitted more ES cells to have contact with the inner cell mass in order to increase the percentage of ES cells that become incorporated into germ line cells. An additional modification from the method used in Examples 3 and 4 was (vii) the development of new Southern probes (SEQ ID NOs:19 and 20) to detect correctly targeted construct in transgenic mice, as well as (viii) a new PCR strategy to rapidly screen mice that possess the desired construct (for details, see below). The combination of these critical modifications was essential for the generation of a transgenic mouse bearing the majority of the human VWF A1 domain (amino acids 1240P to 1481G). The final vector used for the generation of the mice is shown in (FIG. 51).

Identification of mice bearing the human VWF A1 domain was achieved by performing Southern blot analyses (FIG. 52A) and sequencing of genomic DNA by PCR (FIG. 52B).

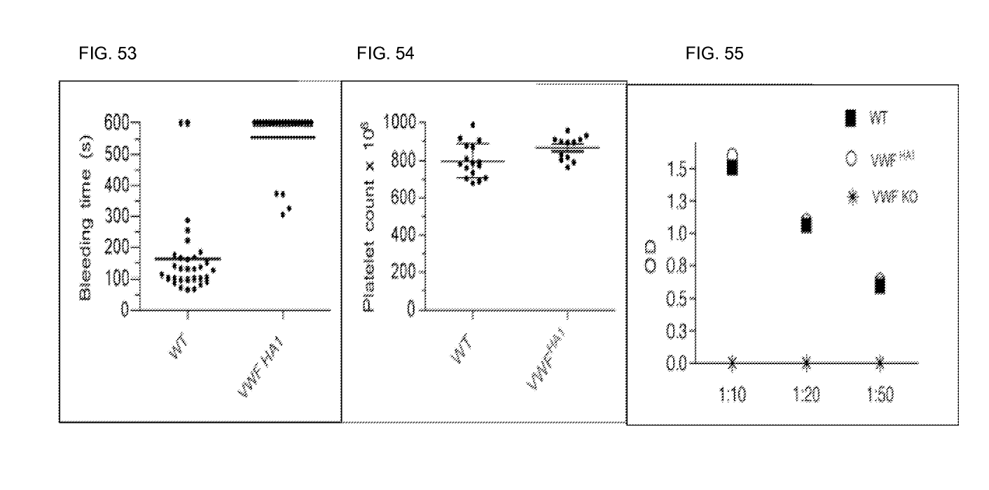

Evaluation of mice homozygous for the VWF-HA1 substitution revealed evidence of a significant bleeding diathesis as manifested by tail bleeding times of greater than 10 minutes versus a mean bleeding time of 180 seconds for WT controls (FIG. 53). This was determined by cutting 1 cm of distal tail tip, immersing it in 37° C. saline, and measuring the time required for hemostasis to occur in anesthetized mice. Importantly, whole blood platelet counts (Hemavet 950FS, Drew Scientific, Dallas, Tex.) and plasma levels of VWF (using standard ELISA) where similar to WT littermate controls (FIGS. 54 and 55, respectively).

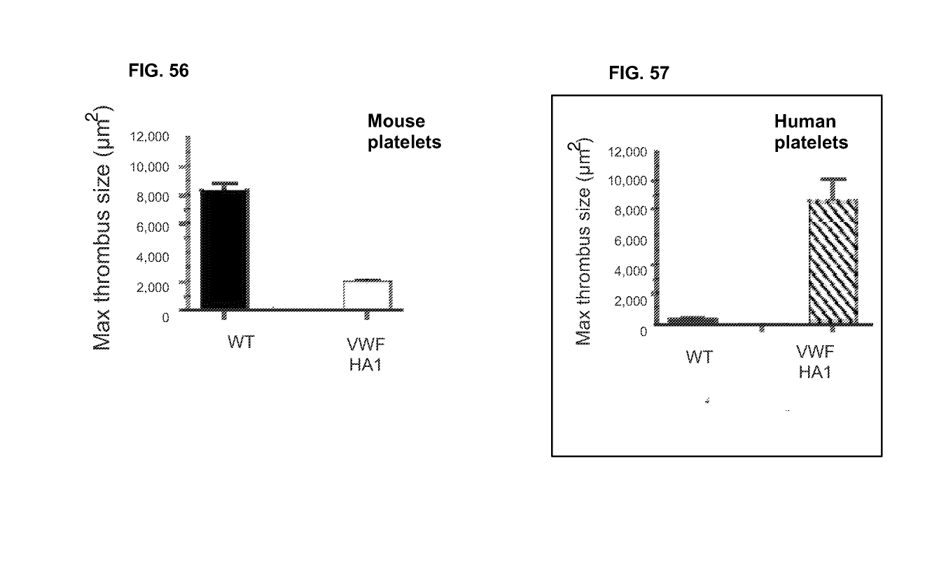

To demonstrate that the defect in hemostasis was due to the inability of mouse platelets to interact with the humanized VWF, intravital studies that assessed the ability of thrombi to form at sites of laser induced injury in the microcirculation of the cremaster muscle in mice were performed. Endogenous circulating mouse platelets were unable to participate in thrombus formation in injured arterioles of VWF HA1 mice (FIG. 56). In contrast, human platelets administered to these animals could support this process (FIG. 57).

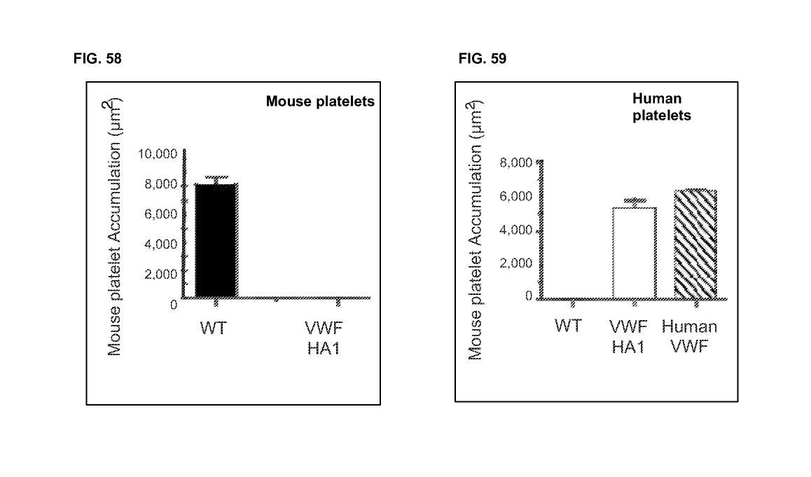

To further demonstrate that mouse VWF containing the majority of the human A1 domain cannot support interactions with mouse platelets, plasma VWF from these animals was surfaced immobilized onto a glass cover slip and incorporated into a parallel plate flow system. WT mouse blood was then infused over the immobilized substrate. GPIbα on mouse platelets could not support any significant interactions with plasma VWF containing the human A1 domain (FIG. 58). By contrast, human platelets could interact at levels observed for human plasma VWF (FIG. 59).

### Long-Range PCR to Detect Homologous Recombination of the Targeting Arms.

Table 7 below shows the reagents used for PCR. Other than the primers and the template DNA, the reagents (Roche extraLong PCR kit 11-732-650-001) were purchased from Roche (Nutley, N.J.).

TABLE 7

| | One Reaction |
|---|---|
| | (μL) |
| 10X Roche Buffer | 2.5 |
| 25 mM MgCl₂ | 0 |
| 2.5 mM dNTPs | 1 |
| primers | |
| for downstream screen: Neo-Forward | 1 for each primer |
| primer (SEQ ID NO: 28) & VWF-Ex-Rev | |
| primer (SEQ ID NO: 29) | |
| for upstream screen: VWF-Ext-F (SEQ ID | 1 for each primer |
| NO: 30) & Neo-Rev (SEQ ID NO: 31) | |
| Roche DNA Pol. | 0.25 |
| ES genomic DNA (about 100 ng) | 1 |
| Water | 18.25 |
| Total volume | 25 |

The PCR program was set at 94° C. for 5 minutes to start; followed by 30 cycles at 94° C. for 1 minutes, 64° C. for 1 minute, and 68° C. for 4 minutes; and finally 68° C. for 8 minutes. The samples were stored at 4° C. The PCR products were then analyzed by gel eletrophoresis. The desired product is a 3.5 KB band for the downstream screen and an approximately 4 KB band for the upstream screen.

PCR Strategy to Rapidly Screen Mice that Possess the Desired Construct.

The reagents, other than the primers and the template DNA, were purchased from Fisher Scientific (Waltham, Mass.).

TABLE 8

| | One Reaction (μL) |
|---|---|
| 10X Assay buffer B | 2.5 |
| 25 mM MgCl₂ | 1.5 |
| 2.5 mM dNTPs | 0.5 |
| Primers | |
| For screening pre-swapped strain, PCR | 0.25 for each primer |
| primer m9677F (SEQ ID NO: 21) & PCR | |
| primer m477R (SEQ ID NO: 22) | |
| For screening swapped strain, PCR | 0.25 for each primer |
| primer H3628R(SEQ ID NO: 23) & PCR | |
| primer H585F (SEQ ID NO: 34) | |
| Taq | 0.25 |
| Tail or ear genome DNA | 2 |
| Water | 17.25 |
| Total volume | 25 |

The PCR program was set at 95° C. for 5 minutes to start; followed by 25 cycles at 95° C. for 1 minute, 60° C. for 1 minute, and 72° C. for 2 minutes; and finally 72° C. for 8 minutes. The samples were stored at 4° C. The PCR products were then analyzed by gel eletrophoresis. The desired product from reactions using primers m9677F and m447R is a 550 by band, and the desired product from reactions using primers m3628R and H585F is a 1100 bp band.

### Example 6

### Use of “Humanized” VWF-A1 Animal for Developing Technologies to Image Sites of Occult Bleeding or Thrombus Formation in Humans

### Perfluorocarbon Nanoparticle Based Imaging Platform.

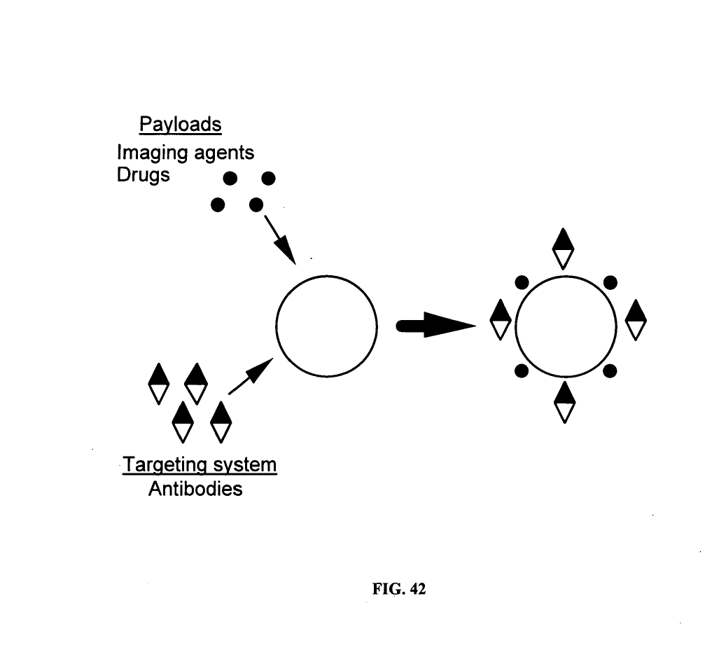

The ability of a VWF-A1 mutant animal, such as the 1326R>H mutant mouse, or the mouse bearing the majority of the human VWF A1 domain (amino acids 1240 through 1481 of VWF), to generate thrombi composed of human platelets at sites of vascular injury in vivo, provides a means for developing imaging technologies designed to detect sites of occult bleeding or thrombus formation in humans. For example, such technologies may prove useful in expediting the discovery of sites of internal bleeding in humans as a result of injuries obtained form a motor vehicle accident. Similarly, it may be useful in detecting injuries obtained in a military battle. Suitable probes include antibodies, small molecules, peptides that recognize molecules expressed on human platelets or the various domains of VWF. However, coupling contrast agents directly to antibodies is cumbersome and insufficient for detection of such complexes in the body by various imaging modalities (i.e. MRI) due to low signal to noise output. Thus, an ideal candidate for detection would not only preserve the specificity associated with monoclonal antibodies, small molecules, or peptides but also have the following properties: 1) high signal-to-noise ratio, 2) long circulating half-life, 3) acceptable toxicity profile, 4) ease of use and production, and 5) compatibility with standard commercially available imaging modalities. Perfluorocarbon Nanoparticle (PNP) may provide the answer. This proposal will take advantage of a novel nanoparticle contrast agent that can be imaged by ultrasound, magnetic resonance, and nuclear imaging (Lanza et al., 2000, Lanza et al., 1997, Yu et al., 2000). This agent is a small (about 150-250 nanometer diameter), lipid encapsulated, perfluorocarbon emulsion that can be administered by vein. Importantly, monoclonal antibodies as well as small molecules and peptides that recognize platelets and/or VWF can be covalently coupled to PNPs. Moreover, PNPs can also be potentially used for targeted drug delivery (FIG. 42).

PNPs have been shown to remain stable in the circulation with a half-life of >1 hour, which permits rapid binding and local contrast enhancement sufficient for diagnostic imaging within 30-60 minutes. PNPs are cleared by the liver and spleen, and are similar to “artificial blood” formulations used to enhance oxygen, which have acceptable safety profiles for clinical use at 10 times greater dose than would be required for targeted contrast enhancement. In addition, perfluorocarbon to be used in this study (perfluorooctylbromide) has an extensive track record for human safety in clinical trials (i.e. Oxygent, Alliance Pharmaceuticals). Thus, this nanoparticle platform provides an ideal opportunity to prove that contrast agents can be targeted specifically to sites of human thrombus formation.

### Preparation of Fluorescently-Labeled Antibody Targeted Nanoparticles.

The basic method for formulating perfluorocarbon nanoparticles comprised of perfluorooctyl bromide (40% w/v), a surfactant co-mixture (2.0%, w/v) and glycerin 9(1.7%, w/v) has been well described (Lanza et al., 2000, Lanza et al., 1997).

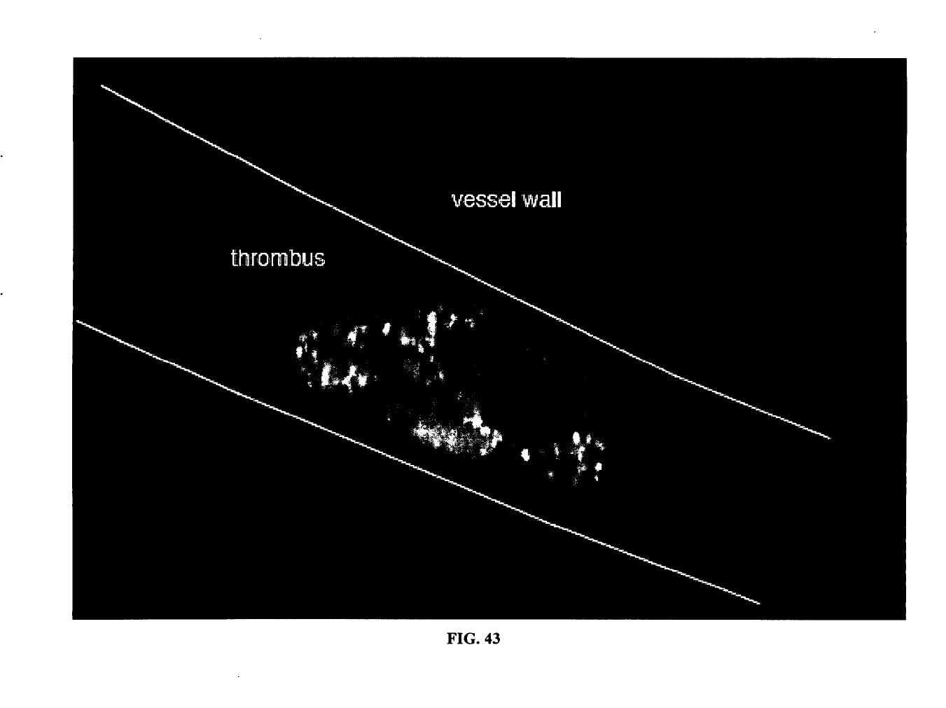

Briefly, the surfactant co-mixture is dissolved in chloroform/methanol, evaporated under reduced pressure, dried in a 50° C. vacuum oven, and finally dispersed into water by sonication. The suspension is combined with perfluorocarbon and then emulsified at 20,000 PSI. Fluorescent nanoparticles are manufactured by including in the lipid mixture 0.1 mole ° A) Fluorescence-FITC or PE prior to the emulsification step. Coupling of monoclonal antibodies involves the introduction of a sulflhydrl group onto the protein by modification of amines with N-succinimidyl S-acetylthioacetate (SATA), which then is reacted with nanoparticles containing activated maleimide. We coupled an antibody that recognizes the human, but not the mouse, platelet receptor allb β₃ and determined the ability of FITC-labeled PNPs to detect a thrombus composed of human platelets at a site of laser-induced vascular injury in the cremaster muscle of a mouse homozygous for the 1326R>H mutation. These antibody-coupled PNPs rapidly and selectively accumulated at the site of the developing human thrombus (FIG. 43).

### Example 7

### Identification of Small Molecules that Mitigate Binding Between GPIb α and the VWF-A1 Domain

Small molecules, often with molecular weights of 500 or below, have proven to be extremely important to researchers for exploring function at the molecular, cellular, and in vivo level. Such compounds have also been proven to be valuable as drugs to treat diseases, and most medicines marketed today are from this class (i.e. Aggrastat—see above). As the interaction between GPIb α and VWF-A1 is essential for the platelet deposition in damaged arterioles, it is a reasonable to assume that disruption of this adhesive event will inhibit or ameliorate thrombus formation. Moreover, it is believed that only partial inhibition is required to achieve this goal based on the phenotype of the mutant A1 domain mice, the inability to form stable thrombi in vivo.

### Computational Design Based on the Structure of the Binary Complex.

Traditional approaches to small molecule discovery typically rely on a step-wise synthesis and screening program for large numbers of compounds to optimize activity profiles. Over the past decade, scientists have used computer models to aid in the development of new chemical agonists or antagonists as well as to better define activity profiles and binding affinities of such compounds. In particular, these tools are being successfully used, in conjunction with traditional research techniques, to examine the structural properties of existing compounds in order to predict their ability to alter the function of biologically relevant proteins. For this approach to be successful, one must have high quality crystal structures of the biological molecule(s) in order to generate an accurate 3-dimensional model so that it can then be used to identify binding regions for small molecules.

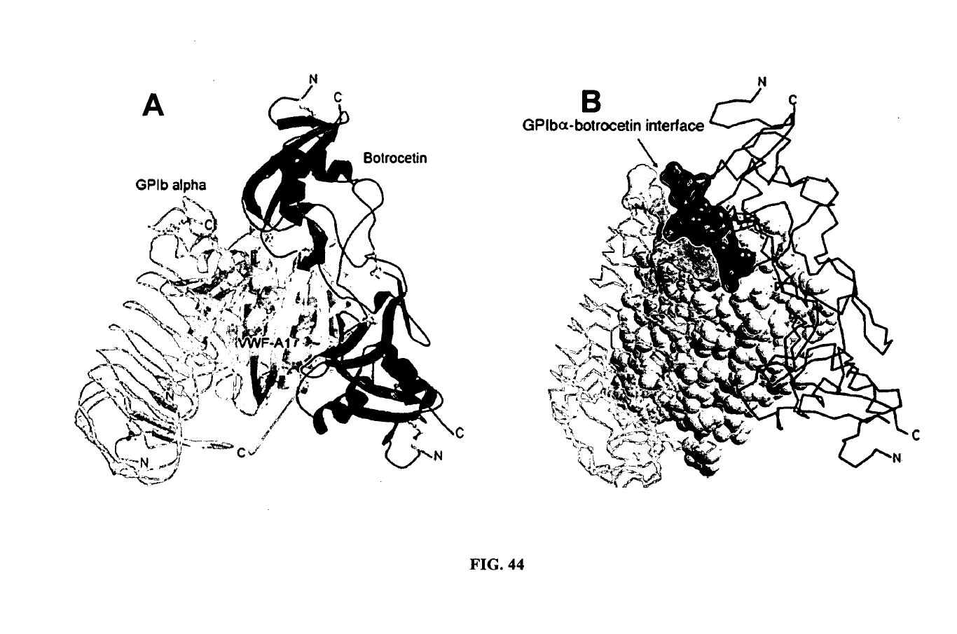

The structure of the binary complex formed when GPIb α binds to the A1 domain of VWF can be determined using such methods. For example, a mechanism by which the snake venom protein botrocetin enhances the interaction between GPIbα and the VWF-A1 in order to promote spontaneous platelet aggregation, resulting in death has been elucidated. Botrocetin was known to bind with high affinity to the A1 domain (crystallization data summary available from PDB access no. 1AUQ and Emsley et al., 1998, see also U.S. Patent Publication No. 2009/0202429, which is incorporated herein in its entirety, for the atomic coordinate data), but was not thought to interact directly with GPIb a. This snake venom has the capacity to form a small, but distinct interface with this platelet receptor so as to prevent its release from the A1 domain, thus facilitating platelet aggregation (FIG. 44). In a sense, nature has created a molecule that modifies the behavior of a known biological interaction, suggesting that one may be able to target man-made structures to this domain as well.

To demonstrate the feasibility of identifying potential small molecule inhibitors in silico, computational modeling software was utilized in conjunction with high-resolution crystal structure results to screen databases for existing compounds that would bind to the A1 domain where it interfaces with botrocetin (exogenous ligand binding site). Several small molecules predicted to bind with sub-micromolar IC₅₀s (concentration of drug required to inhibit the activity by 50%) and that could also severely disrupt binding of this snake venom protein were identified. Thus, potential candidate small molecules can be identified that may interfere with the interaction between GPIb α and the A1 domain of VWF.

### Screening Small Molecule Library for Inhibitors.

Although the use of computational modeling is a state-of-the-art method for identifying lead compounds, it is not without its limitations. Thus, an actual library of 20,000 small molecules manufactured by the Chembridge Corporation (San Diego, Calif.) will also be screened. The library consists of handcrafted drug-like organic molecules with molecular weights in a range of 25-550, which are soluble in DMSO at concentrations ranging from 10-20 mM. The structure and purity (>95%) of these compounds have been validated by NMR. The library is formatted in a 96 well plate for high throughput screening using instrumentation made available through the OCCC (under supervision of the Landry laboratory) and includes a robot plate reader (FLexStation II 384, Molecular Devices, Sunnyvale, Calif.), an 8-tip robotic pipettor (Multiprobe II Plus, Perkin Elmer, Shelton, Conn.), a 96-tip robotic pipettor (Mintrak, Perkin Elmer), and an automated 96 well plate washer (Perkin Elmer).

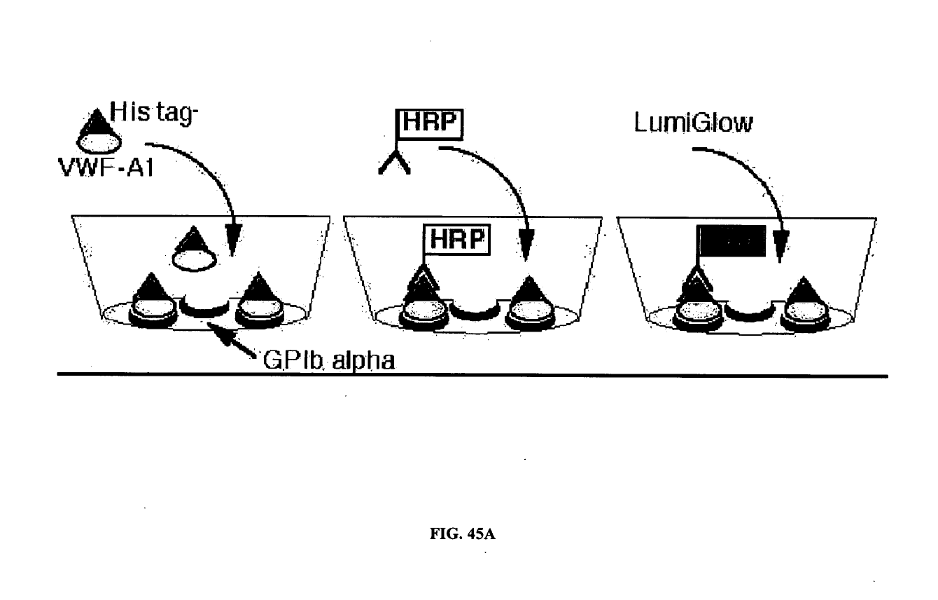

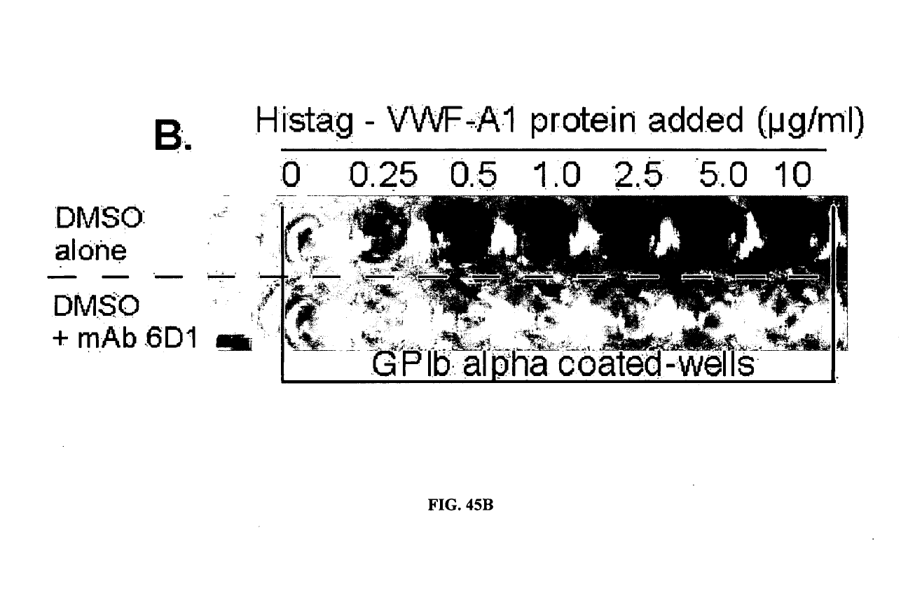

An ELISA based system will be used to screen for compounds that may inhibit the interaction between GPIb α and the VWF-A1 domain. Enzyme-Linked Immunosorbent Assay (ELISA) methods are immunoassay techniques used for detection or quantification of a substance. An example of this assay is demonstrated in FIG. 45A, where an antibody conjugated with horseradish peroxidase (HRP) was used to identify the presence of VWF. Depending on the substrate added, HRP enzyme activity can be detected by either a change in color (chromogenic product) or fluorescence (most sensitive indicator). Schematic representation of the proposed assay system to be used for screening is shown in FIG. 45A.

Assay system: Recombinant GPIb α and VWF-A1 proteins will be generated and purified as disclosed, with the latter containing a 6×His tag. Purified GPIb α will be absorbed overnight (4° C.) to PRO-BIND polystyrene 96-well assay plates (Falcon) at 10 μg/ml per well. Plates will be washed and non-specific binding sites blocked by the addition of TENTC buffer (50 mM Tris, 1 mM EDTA, 0.15 M NaCl, 0.2% casein, 0.05% Tween 20, pH 8.0) for 1 hour at room temperature. Subsequently, plates will be washed with and resuspended in TBS buffer (50 mM Tris, 150 mM NaCl, pH 8.0) and 1 test compound per well added at a final concentration of 10 μM (final DMSO concentration 0.5%). After 30 minutes, recombinant His tagged VWF-A1 protein will be added at a 1:1 Molar ratio to that of GPIb α and left to incubate for 1 hour before washing with TBS buffer. VWF-A1 bound to surface-immobilized GPIb α will be determined by the addition of HRP-conjugated anti-His tag antibody and the A1-antibody conjugate detected by the addition of LumiGlow reagent (KPL, Gaithersburg, Md.). The resulting fluorescence will be quantified by the number of luminescence emissions per second using a FLexStation II 384 plate reader. A sample will be considered positive when the luminescence (in counts per second) is more than 2 standard deviations above the mean value for negative-controls.

Negative controls: Addition of mAb 6D1 to certain wells to prevent VWF-A1 binding to GPIb α or no addition of VWF-A1 protein (FIG. 45B). In either case, no significant fluorescence should be detected. Once compounds of interest have been identified, solubility of these molecules will be confirmed to rule out precipitation as the etiology for blocking interactions between GPIb α and VWF-A1. In addition, a dose effect curve will also be generated (1 nM to 100 μM) to obtain preliminary information regarding the IC₅₀ of the inhibitor. Lead molecules will then be tested for their ability to limit human platelet interactions with plasma VWF in aggregometry and flow chamber assays as described in preliminary results. Ultimately, the most promising compound will be tested in the humanized mouse model of thrombosis.

### Example 8

### Effect of Plavix or ReoPro on Human Platelet-Induced Hemostasis in Homozygous VWF^{1326R>H} Mice

To demonstrate the feasibility of the VWF^{1326R>H} mice to identify anti-thrombotic drugs capable of perturbing human platelet function in vivo, the ability of 2 FDA approved drugs, Plavix and ReoPro, to prevent human platelet-induced hemostasis was tested. As noted above, Clopidogrel has been shown to reduce thrombus size and delay its formation in mice with a maximal effective dose of 50 mg/kg given the day before and 2 hours prior to experimentation. Homozygous VWF^{1326R>H} mice that received this dosing schema, were unable to produce a hemostatic clot when administered human platelets in contrast to homozygous VWF^{1326R>H} mice that received saline in lieu of drug.

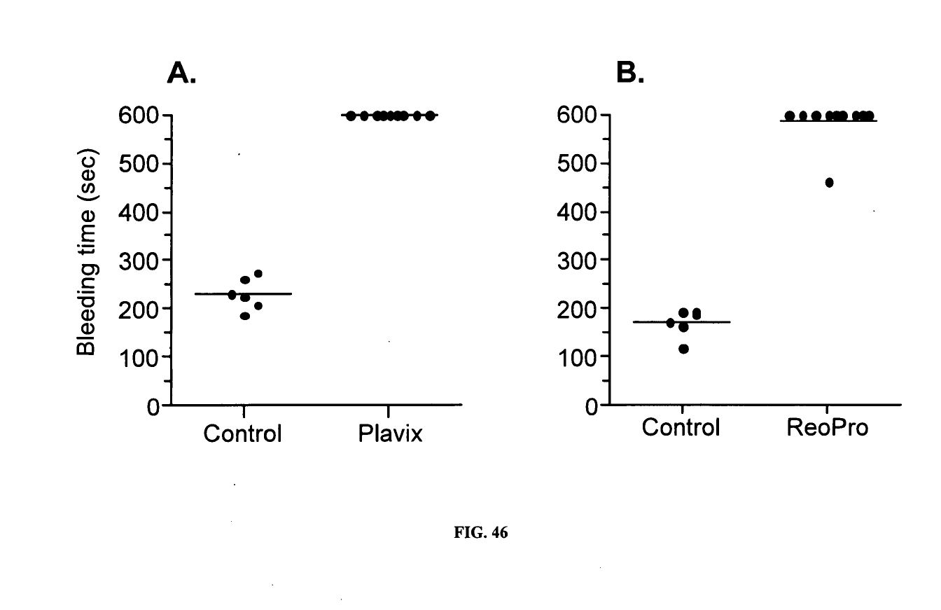

Because Plavix can also block the function of the ADP receptor on murine platelets (see FIG. 46A), the ability of ReoPro to prevent the formation of a hemostatic plug in homozygous VWF^{1326R>H} mice was also tested. ReoPro is currently approved for short-term treatment of patients with acute coronary syndrome that require interventional catheterization. It is administered by intravenously bolus (0.25 mg/kg), followed by an infusion of 0.125 μg/kg/min. This results in >80% αIIb β3 occupancy, and disrupts platelet function for 24-36 hours. It does not bind or disrupt the function of murine αIIb β3. Administration of ReoPro to homozygous VWF^{1326R>H} mice 5 minutes after the infusion of human platelets, prevented the formation of a hemostatic plug (mean bleeding time 579 seconds) (FIG. 46B). By contrast, animals that received a non-function blocking antibody to human αIIb β3 were able to form a hemostatic plug (mean bleeding 175 seconds).

### Example 9

### Determining the Efficacy of Anti-Platelet Drugs Administered to Patients by Studying the Ability of Platelets Harvested from Patients on Therapies in the VWF^{1326R>H} Mouse

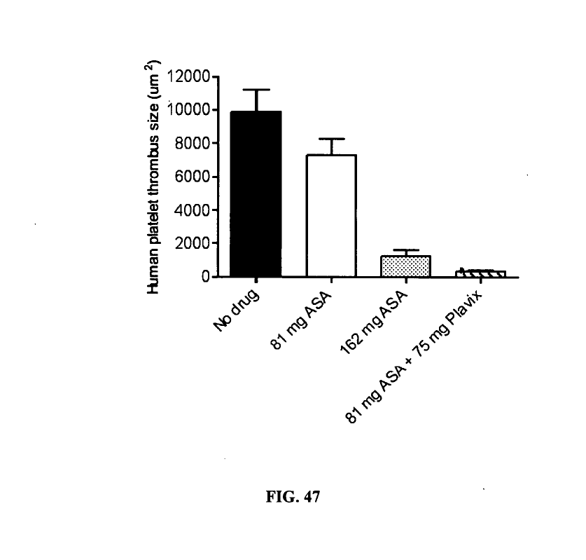

Intravital microscopic study was carried out to evaluate the ability of the VWF^{1326R>H} mouse to determine the efficacy of anti-platelet therapies given to patients at risk or with active cardiovascular disease. The typical prophylactic dose of aspirin (ASA) of 81 mg did not prevent laser-injury induced human platelet thrombus formation in the genetically modified animal while increasing the daily dose to 162 mg was preventative (FIG. 47). Similarly, platelets administered from a patient on 81 mg of ASA and 75 mg Plavix also prevented thrombus formation.

### Example 10

### Differential Effects of Ristocetin on Human Platelet Aggregration in VWF^{R1326H} and VWF^{HA1} Mouse Plasma

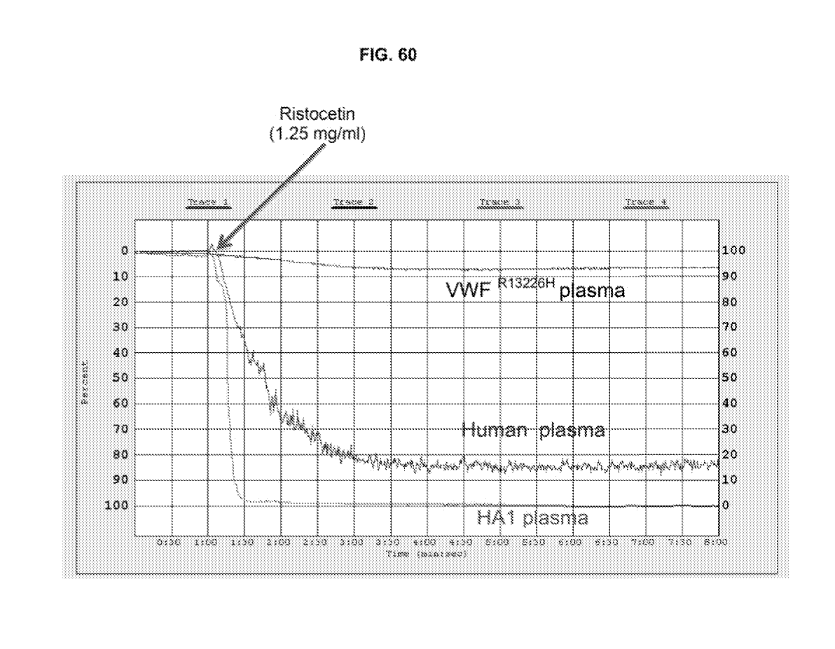

Although the R1326H mutation in murine VWF can support human platelet mediated hemostasis and thrombosis, there still exists a major difference between the human and mouse A1 domains. Specifically, only the human form can be activated to bind to GPIb alpha on human platelets. Ristocetin is an antibiotic that was taken off the market due to its ability to cause von Willebrand factor to bind the platelet receptor GPIb alpha, so when ristocetin is added to normal blood, it causes platelet clumping. This is demonstrated in the following experiment in which human platelets were resuspended in platelet poor plasma from either VWF^{R1326H} or VWF^{HA1} mice, and the ability of ristocetin to induce aggregation was determined by optical aggregometry. Whereas ristocetin could activate plasma from VWF^{HA1} mice so that it could cause human platelet aggregation comparable to its human counterpart, such was not the case with plasma obtained from VWF^{R1326H} animals (FIG. 60). Thus, murine VWF bearing the majority of the human A1 domain functions in manner more closely to that in humans, making it a better biological platform for studying human platelet function and the effects of antiplatelet agents for use in human beings.

### DOCUMENTS

Alon R, Chen S, Fuhlbrigge R, Puri K D, Springer T A. The kinetics and shear threshold of transient and rolling interactions of L-selectin with its ligand on leukocytes. Proc Natl Acad Sci USA. 1998; 95:11631-1166.
Andre P, Prasad K S, Denis C V, He M, Papalia J M, Hynes R O, Phillips D R, Wagner D D. CD40L stabilizes arterial thrombi by a beta3 integrin-dependent mechanism. Nat. Med. 2002 March; 8(3):247-52.
Andrews R K, Booth W J, Gorman J J, Castaldi P A, Berndt M C. Purification of botrocetin from Bothrops jararaca venom. Analysis of the botrocetin-mediated interaction between von Willebrand factor and the human platelet membrane glycoprotein Ib-IX complex. Biochemistry. 1989; 28:8317-8326.
Azzam K, Garfinkel L I, Bal dit Sollier C et al. Antithrombotic effect of a recombinant von Willebrand factor, VCL, on nitrogen laser-induced thrombus formation in guinea pig mesenteric arteries. Thromb Haemost 1995: 73:318-323.
Bennett J S. Novel platelet inhibitors. Annu Rev Med. 2001; 52:161-84.
Bergmeier W, Piffath C L, Goerge T, Cifuni S M, Ruggeri Z M, Ware J, Wagner D D. The role of platelet adhesion receptor GPIb alpha far exceeds that of its main ligand, von Willebrand factor, in arterial thrombosis. Proc Natl Aced Sci USA. 2006 Nov. 7; 103(45):16900-5.
Bogan A A, Thorn K S. Anatomy of hot spots in protein interfaces. J Mol. Biol. 1998 Jul. 3; 280(1):1-9.
Bonnefoy et al., von Willebrand factor A1 domain can adequately substitute for A3 domain in recruitment of flowing platelets to collagen. Journal of thrombosis and haemostasis JTH (2006) Volume: 4, Issue: 10, Pages: 2151-2161.
Bonthron D T, Handin R I, Kaufman R J et al. Structure of pre-pro-von Willebrand factor and its expression in heterologous cells. Nature 1986; 324:270-273.
Celikel R, Ruggeri Z M, and Varughese K I. Von Willebrand factor conformation and adhesive function is modulated by an internalized water molecule. Nat Struct Biol. 2000; 7:881-884.
Chen S and Springer T A. An automatic braking system that stabilizes leukocyte rolling by an increase in selectin bond number with shear. 1999. J. Cell Biol. 144:185-200.
Chen S, Springer T A. Selectin receptor-ligand bonds: formation limited by shear rate and dissociation governed by the bell model. Proc natl acad sci USA. 2001; 98:950-955.
Chesla S E, Selvaraj P, Zhu C. Measuring two-dimensional receptor-ligand binding kinetics by micropipette. Biophys J. 1998; 75:1553-1572.
Collaborative Computational Project, Number 4. The CCP4 suite: programs for protein crystallography. Acta Crystallogr D Biol Crystallogr. 1994 Sep. 1; 50(Pt 5):760-3.
Cooney K A, Ginsburg D. Comparative analysis of type 2b von Willebrand disease mutations: implications for the mechanism of von Willebrand factor binding to platelets. Blood. 1996; 87:2322-2328.
Cooney, K. A., and D. Ginsburg. 1996. Comparative analysis of type 2B von Willebrand disease mutations: implications for the mechanism of von Willebrand factor binding to platelets. Blood 87:2322-2328.
Coxon, A., P. Rieu, F. J. Barkalow, S. Askari, A. H. Sharpe, U. H. von Andrian, M. A. Arnaout, and T. N. Mayadas. 1996. A novel role for the beta 2 integrin CD11b/CD18 in neutrophil apoptosis: a homeostatic mechanism in inflammation. Immunity. 5:653-666.
Cruz et al., (2000) J. Biol. Chem. 275, 19098-19105
Cruz M A, Diacovo T G, Emsley J, Liddington R, Handin R I. 2000. Mapping the GPib binding site in the von Willebrand factor A1 domain. J Biol. Chem. 275, 19098-19105.
Cruz M A, Yuan H, Lee J R, et al. Interaction of von Willebrand factor (VWF) with collagen. 1995. J Biol. Chem. 270, 10822-10827.
Cruz M A, Handin R I, Wise R J. The interaction of the von Willebrand factor-A1 domain with platelet glycoprotein Ib/IX. The role of glycosylation and disulfide bonding in a monomeric recombinant A1 domain protein. J Biol. Chem. 1993 Oct. 5; 268(28):21238-45.
Cruz, M. A., T. G. Diacovo, J. Emsley, R. Liddington, and R. I. Handin. 2000. Mapping the glycoprotein Ib-binding site in the von willebrand factor A1 domain. J Biol. Chem. 275:19098-19105.
Denis C, Methia N, Frenette P S, et al. A mouse model of severe von Willebrand disease: defects in hemostasis and thrombosis. Proc Natl Acad Sci, USA. 1998; 95:9524-9529.
Diacovo T G, Puri K D, Warnock R A, Springer T A, von Andrian U H. Platelet-mediated lymphocyte delivery to high endothelial venules. Science. 1996 Jul. 12; 273(5272):252-5.
Doggett T A, Girdhar G, Lawshe A, Schmidtke D W, Laurenzi I J, Diamond S L, Diacovo T G. Selectin-like kinetics and biomechanics promote rapid platelet adhesion in flow: the GPIb(alpha)-VWF tether bond. Biophys J. 2002 July; 83(1):194-205.
Dumas J J, Kumar R, McDonagh T, Sullivan F, Stahl M L, Somers W S, Mosyak L. Crystal structure of the wild-type von Willebrand factor A1-glycoprotein Ibalpha complex reveals conformation differences with a complex bearing von Willebrand disease mutations. J Biol Chem. 2004 May 28; 279(22):23327-34.
Emsley et al., Crystal Structure of the von Willebrand Factor A1 Domain and Implications for the Binding of Platelet Glycoprotein Ib (1998) The Journal of Biological Chemistry, 273, 10396-10401.
Emsley J, Knight C G, Farndale R W, Barnes M J, and Liddington R C. Structural basis of collagen recognition by integrin alpha2 beta1. Cell 2000; 101:47-56.
Esnouf R M. An extensively modified version of MolScript that includes greatly enhanced coloring capabilities. J Mol Graph Model. 1997 April; 15(2):132-4, 112-3.
Evans E, Berk D, Leung A. Detachment of agglutinin-bonded red blood cells. I. Forces to rupture molecular-point attachments, Biophys J. 1991 April; 59(4):838-48.
Evans E, Leung A, Hammer D, Simon S. Chemically distinct transition states govern rapid dissociation of single L-selectin bonds under force. Proc Natl Acad Sci USA. 2001 Mar. 27; 98(7):3784-9. Epub 2001 Mar. 13.
Ewenstein B M. von Willebrand's disease. 1997. Ann Rev Med 48, 525-542.
Falati S, Gross P, Merrill-Skoloff G, Furie B C, Furie B. Real-time in vivo imaging of platelets, tissue factor and fibrin during arterial thrombus formation in the mouse. Nat. Med. 2002 October; 8(10):1175-81.
Federici A B, Bader R, Pagani S, Colibretti M L, De Marco L, and Mannucci P M. Binding of von Willebrand factor to glycoproteins Ib and IIb/IIIa complex: affinity is related to multimeric size. Br J. Haematol. 1989; 73:93-99.
Fredrickson B J, Dong J F, McIntire L V, López J A. Shear-dependent rolling on von Willebrand factor of mammalian cells expressing the platelet glycoprotein Ib-IX-V complex. Blood. 1998 Nov. 15; 92(10):3684-93.
Fressinaud E, Baruch, D, Girma J P, et al.: von Willebrand factor-mediate platelet adhesion to collagen involves platelet glycoprotein Ilb/IIIa as well as glycoprotein Ib. 1988. J Lab Clin Med 112, 58-67.
Fukuda K, Doggett T A, Bankston L A, Cruz M A, Diacovo T G, Liddington R C. Structural basis of von Willebrand factor activation by the snake toxin botrocetin. Structure. 2002; 10:943-950.
Fukuda K, Doggett T, Laurenzi I J, Liddington R C, Diacovo T G. The snake venom protein botrocetin acts as a biological brace to promote dysfunctional platelet aggregation. Nat Struct Mol. Biol. 2005 February; 12(2):152-9.
Furie B, Furie B C. Thrombus formation in vivo. J Clin Invest. 2005 December; 115(12):3355-62.
Gachet C, Hechler B. The platelet P2 receptors in thrombosis. Semin Thromb Hemost. 2005 April; 31(2):162-7.
Gillespie, D. T. 1976. A general method for numerically simulating the stochastic time evolution of coupled chemical reactions. J. Comput. Phys. 22:403-434.
Ginsburg, D., and J. E. Sadler. 1993. von Willebrand disease: a database of point mutations, insertions, and deletions. For the Consortium on von Willebrand Factor Mutations and Polymorphisms, and the Subcommittee on von Willebrand Factor of the Scientific and Standardization Committee of the International Society on Thrombosis and Haemostasis. Thromb. Haemost. 69:177-184.
Greenberg A W, Brunk D K, Hammer D A. Cell-free rolling mediated by L-selectin and sialyl Lewis (x) reveals the shear threshold effect. Biophys J. 2002; 79:2391-2402.
Handa M, Titani K, Holland L Z, et al. The von Willebrand factor-binding domain of platelet membrane glycoprotein Ib. Characterization by monoclonal antibodies and partial amino acid sequence analysis of proteolytic fragments. 1986. J Biol. Chem. 26, 12579-12585.
Hankey G J, Eikelboom J W. Antiplatelet drugs. Med J. Aust. 2003 Jun. 2; 178(11):568-74.
Hillery C A, Mancuso D J, Sadler J E, Ponder J W et al. Type 2M von Willebrand disease: F6061 and 1662F mutations in the glycoprotein Ib binding domain selectively impair ristocetin—but not botrocetin-mediated binding of von Willebrand factor to platelets. Blood 1998; 91:1572-15781.
Howard M A, and Firkin B G. Ristocetin—a new tool in the investigation of platelet aggregation. Thromb Diath Haemorrh. 1971; 26:362-369.
Huang et al., A Structural Explanation for the Antithrombotic Activity of ARC1172, a DNA Aptamer that Binds von Willebrand Factor Domain A1 Structure, (2009) Volume 17, Issue 11, 1476-1484.
Huizing a E G, Tsuji S, Romijn R A P, et al. Structures of Glycoprotein Iba and its complex with von Willebrand Factor A1 domain. Science. 2002; 297:1176-1179.
Inbal A, Kornbrot N, Harrison P, Randi A M, Sadler J E. Effect of type IIB von Willebrand disease mutation Arg(545)Cys on platelet glycoprotein Ib binding—studies with recombinant von Willebrand factor. Thromb Haemost. 1993 Dec. 20; 70(6):1058-62.
Italiano J E Jr, Bergmeier W, Tiwari S, Falet H, Hartwig J H, Hoffmeister K M, Andre P, Wagner D D, Shivdasani R A. Mechanisms and implications of platelet discoid shape. Blood. 2003 Jun. 15; 101(12):4789-96. Epub 2003 Feb. 13.
Jaffe E A, Hoyer L W and Nachman R L Synthesis of antihemophilic factor antigen by cultured human endothelial cells. 1973. J Clin Invest. 52, 2757-2764.

Jenkins et al. (1998) “Molecular Modeling of Ligand and Mutation Sites of the Type A Domains of Human von Willebrand Factor and Their Relevance to von Willebrand's Disease” Vol. 91, No. 6, Blood, pp. 2032-2044.
Kalafatis M, Takahashi Y, Girma J P, et al. Localization of the collagen-interactive domain of human von Willebrand factor between amino acid residues Gly 911 and Glu 1365. Blood 1987; 70:1577-1583.
King M R, Hammer D A. Multiparticle adhesive dynamics: hydrodynamic recruitment of rolling leukocytes. Proc Natl Acad Sci USA. 2001 Dec. 18; 98(26):14919-24.
Kohler G, Milstein C: Continuous cultures of fused cells secreting antibody of predefined specificity. Nature 1975; 256:495-497.
Kroner P. A., et al., Expressed Full-Length von Willebrand Factor Containing Missense Mutations Linked to Type IIB von Willebrand Disease shows enhanced binding to platelets. Blood 1992. Vol. 79, pp 2048-2055.
Lankhof H, van Hoeij M, Schiphorst M E, Bracke M et al. A3 domain is essential for interaction of von Willebrand factor with collagen type III. Thromb Haemost. 1996; 75:950-958.
Lanza G M, Abendschein D R, Hall C S, Marsh J N, Scott M J, Scherrer D E, Wickline S A. Molecular imaging of stretch-induced tissue factor expression in carotid arteries with intravascular ultrasound. Invest Radiol. 2000 April; 35(4):227-34.
Lanza G M, Wallace K D, Fischer S E, Christy D H, Scott M J, Trousil R L, Cacheris W P, Miller J G, Gaffney P J, Wickline S A. High-frequency ultrasonic detection of thrombi with a targeted contrast system. Ultrasound Med Biol. 1997; 23(6):863-70.
Lenain N, Freund M, Leon C, Cazenave J P, Gachet C. Inhibition of localized thrombosis in P2Y1-deficient mice and rodents treated with MRS2179, a P2Y1 receptor antagonist. J Thromb Haemost. 2003 June; 1(6):1144-9.
Léon C, Hechler B, Freund M, Eckly A, Vial C, Ohlmann P, Dierich A, LeMeur M,
Cazenave J P, Gachet C. Defective platelet aggregation and increased resistance to thrombosis in purinergic P2Y(1) receptor-null mice. J Clin Invest. 1999 December; 104(12):1731-7.
Mancuso D J, Kroner P A, Christopherson P A et al. Type 2M: Milwaukee-1 von Willebrand disease: an in-frame deletion in the Cys509-Cys695 loop of von Willebrand factor A1 domain causes deficient binding of von Willebrand factor to platelets. Blood 1996; 88: 2559-2568.
Mancuso D J, Tuley E A, Westfield L A et al. Structure of the gene for human von Willebrand factor. J Biol. Chem. 1989; 264:19514-19527.
Marshall B T, Long M, Piper J W, Yago T, McEver R P, Zhu C. Direct observation of catch bonds involving cell-adhesion molecules. Nature. 2003; 423:190-193.
Merkel R, Nassoy P, Leung A, Ritchie K, Evans E. Energy landscapes of receptor-ligand bonds explored with dynamic force spectroscopy. Nature. 1999 Jan. 7; 397(6714):50-3.
Meyer D, Baumgartner H R. Role of von Willebrand factor in platelet adhesion to subendothelium. 1983. Br J. Haematol. 54:1-9.
Meyer D, Fressinaud E, Gaucher C, Layergne J-M, Hilbert L, Ribba A S et al. Gene defects in 150 unrelated French cases with type 2 von Willebrand disease: from patient to gene. Thromb and Haemost. 1997; 78:451-456.
Miura, S., C. Q. Li, Z. Cao, H. Wang, M. R. Wardell, and J. E. Sadler. 2000. Interaction of von Willebrand factor domain A1 with platelet glycoprotein Ib alpha-(1-289). Slow intrinsic binding kinetics mediate rapid platelet adhesion. J. Biol. Chem. 275:7539-7546.
Murata M, Ware J, and Ruggeri Z M. Site-directed mutagenesis of a soluble recombinant fragment of platelet glycoprotein Ib alpha demonstrating negatively charged residues involved in von Willebrand factor binding. 1991. J Biol. Chem. 266, 8149-8155.
Nachman R L., Levine R, Jaffe E A. Synthesis of factor VIII antigen by cultured guinea-pig megakaryocytes 1977. J Clin Invest. 60, 914-921.
Ni H, Ramakrishnan V, Ruggeri Z M, Papalia J M, Phillips D R, Wagner D D. Increased thrombogenesis and embolus formation in mice lacking glycoprotein V Blood. 2001 Jul. 15; 98(2):368-73
Offermanns S. Activation of platelet function through G protein-coupled receptors. Circ Res. 2006 Dec. 8; 99(12):1293-304.
Pareti F T, Fujimura Y, Dent J A, et al. Isolation and characterization of a collagen binding domain in human von Willebrand factor. J Biol. Chem. 1986; 261:15310-15315.
Pareti F T, Niiya K, McPherson J M, et al. Isolation and characterization of two domains of human von Willebrand factor that interact with fibillar collagen types I and III. J Biol. Chem. 1987; 262:13835-13841.
Pietu G, Meulien P, Cherel G, et al. Production in Escherichia coli of a biologically active subfragment of von Willebrand factor corresponding to the platelet glycoprotein Ib, collagen, and heparin binding domains. Biochem. Biophys. Res. Comm. 1989; 164:1339-1347.
Pozgajová M, Sachs U J, Hein L, Nieswandt B. Reduced thrombus stability in mice lacking the alpha2A-adrenergic receptor. Blood. 2006 Jul. 15; 108(2):510-4.
Rabinowitz, I. et al. (1992) Proc. Natl. Acad. Sci. USA 89, 9846-9849
Rathore V, et al., PECAM-1 negatively regulates GPIb/V/IX signaling in murine platelets. Blood 2003; 102:3658-3664.
Read M S, Smith S V, Lamb, M A, and Brinkhous K M. Role of botrocetin in platelet agglutination: formation of an activated complex of botrocetin and von Willebrand factor. Blood 1989; 74:1031-1035
Ribba A S, Voorberg J, Meyer D, Pannekoek H, Pietu G. Related Articles, Links Free Full Text Characterization of recombinant von Willebrand factor corresponding to mutations in type IIA and type IIB von Willebrand disease. J Biol. Chem. 1992; 267:23209-23215.
Roth G J, Titani K, Hoyer L W, et al. Localization of binding sites within human von Willebrand factor for monomeric type III collagen. Biochemistry 1986; 25:8357-8361.
Roth G J. Developing relationships: arterial platelet adhesion, glycoprotein Ib, and leucine-rich glycoproteins. Blood. 1991; 77:5-19.
Ruggeri Z M, and Ware J. The structure and function of von Willebrand factor. Thromb Haemost. 1992; 67:594-599.
Ruggeri, Z. M. et al. Blood. 108, 1903-1910 (2006)
Ruggeri Z M, Zimmerman T S. The complex multimeric composition of factor VIII/von Willebrand factor. Blood. 1981 June; 57(6):1140-3.
Ruggeri, Z. M., F. I. Pareti, P. M. Mannucci, N., Ciavarella, and T. S. Zimmerman. 1980. Heightened interaction between platelets and factor VIII/von Willebrand factor in a new subtype of von Willebrand's disease. N. Engl. J. Med. 302:1047-1051.
Sadler J E, Matsushita T, Dong Z, Tuley E A, Westfield L A. Molecular mechanism and classification of von Willebrand disease. 1995. Thromb and Haemost 74, 161-166.
Sadler J E, Shelton-Inloes B B, Sorace J M, Harlan J et al. Cloning and characterization of two cDNAs coding for human von Willebrand factor. Proc Natl Acad Sci USA 1985; 82:6394-6398.
Sadler, J. E. et al. (2006) J. Thromb. Haemost. 4, 2103-2114
Sakariassen K S, Bolhuis P A and Sixma J J. human platelet adhesion to artery subendothelium is mediated by Factor VIII-von Willebrand factor bound to the subendothelium. 1979. Nature 279:636-638.
Sauer and Henderson (1988) Proc. Natl. Acad. Sci. USA 85:5166.
Savage B, Saldivar E, Ruggeri Z M. 1996. Initiation of platelet adhesion by arrest onto fibrinogen or translocation on von Willebrand factor. Cell. 84, 289-297.
Sen U, Vasudevan S, Subbarao G, McClintock R A, Celikel R, Ruggeri Z M, Varughese K I. Crystal structure of the von Willebrand factor modulator botrocetin. Biochemistry. 2001; 40:345-352.
Shelton-Inloes B B, Titani K, Sadler E. cDNA sequences for human von Willebrand factor reveal five types of repeated domains and five possible protein sequence polymorphisms. Biochemistry 1986; 25:3164-3171.
Shen Y, Romo G M, Dong J F, Schade A, McIntire L V, Kenny D, Whisstock J C, Berndt M C, Lopez J A, Andrews R K. Requirement of leucine-rich repeats of glycoprotein (GP) Ibalpha for shear-dependent and static binding of von Willebrand factor to the platelet membrane GP Ib-IX-V complex. Blood. 2000 Feb. 1; 95(3):903-10.
Siedlecki C A, Lestini B J, Kottke-Marchant K K, Eppell S J, Wilson D L, and Marchant R E. Shear-dependent changes in the three-dimensional structure of human von Willebrand factor. Blood 1996; 88:2939-2950.
Simson D A, Ziemann F, Strigl M, Merkel R. Micropipet-based pico force transducer: in depth analysis and experimental verification. Biophys J. 1998 April; 74(4):2080-8.
Sixma et al., (1991) Eur J. Biochem. 196:369-75
Smith C, Estavillo D, Easley J, Bankston L A, Liddington R C, Cruz M A. Mapping the collagen-binding site in the I domain of the glycoprotein Ia/IIa (integrin alpha(2)beta(1)). J Biol Chem. 2000 Feb. 11; 275(6):4205-9.
Sporn L A, Chavin S I, Marder V J and Wagner, D D. Biosynthesis of von Willebrand protein by human megakaryocytes. 1985. J Clin Invest. 76, 1102-1106.
Sugimoto M, Ricca G, Hrinda M E, Schreiber A B, Searfoss G H, Bottini E, Ruggeri Z M. Functional modulation of the isolated glycoprotein Ib binding domain of von Willebrand factor expressed in Escherichia coli. Biochemistry. 1991 May 28; 30(21):5202-9.
Sugimoto et al., (1993) J Biol. Chem. 268:12185-92
Tees, D. F. J., and H. L. Goldsmith. 1996. Kinetics and locus of failure of receptor-ligand-mediated adhesion between latex spheres. I. Protein-carbohydrate bond. Biophys. J. 71:1102-1114.
Valgimigli M, Percoco G, Malagutti P, Campo G, Ferrari F, Barbieri D, Cicchitelli G, McFadden E P, Merlini F, Ansani L, Guardigli G, Bettini A, Parrinello G, Boersma E, Ferrari R; STRATEGY Investigators. Tirofiban and sirolimus-eluting stent vs abciximab and bare-metal stent for acute myocardial infarction: a randomized trial. JAMA. 2005 May 4; 293(17):2109-17.
Verweij, C L, Diergaarde P J, Hart, M, et al. Full length von Willebrand factor (VWF) cDNA encodes a highly repetitive protein considerably larger than the mature VWF subunit. EMBO J. 1986; 5:1839-1863.
Ware J, Russell S, Ruggeri Z M. Generation and rescue of a murine model of platelet dysfunction: the Bernard-Soulier syndrome. Proc Natl Acad Sci USA. 2000 Mar. 14; 97(6):2803-8.
Weiss H J, Sussman I I, Hoyer L W. Stabilization of factor VIII in plasma by the von Willebrand factor. 1977. J Clin Invest. 60, 390-404.
Whittaker C A, Hynes R O. Distribution and evolution of von Willebrand/integrin A domains: widely dispersed domains with roles in cell adhesion and elsewhere. Mol Biol Cell. 2002 October; 13(10):3369-87.
Xu H, Zhang X, Mannon R B, Kirk A D. Platelet-derived or soluble CD154 induces vascularized allograft rejection independent of cell-bound CD154. J Clin Invest. 2006 March; 116(3):769-74.
Yamamoto H, Vreys I, Stassen J M et al. Antagonism of VWF inhibits both injury induced arterial and venous thrombosis in the hamster. Thromb Haemost 1998: 79:202-210.
Yoon, B. J., and S. Kim. 1990. A boundary collocation method for the motion of two spheroids in Stokes flow: Hydrodynamic and colloidal interactions. Int. J. Multiphase Flow. 16:639-649.
Yoshida E, Fujimura Y, Miura S, Sugimoto M, Fukui H, Narita N, Usami Y, Suzuki M, Titani K. Alboaggregin-B and botrocetin, two snake venom proteins with highly homologous amino acid sequences but totally distinct functions on von Willebrand factor binding to platelets. Biochem Biophys Res Commun. 1993; 19: 1386-1392.
Yu X, Song S K, Chen J, Scott M J, Fuhrhop R J, Hall C S, Gaffney P J, Wickline S A, Lanza G M. High-resolution MRI characterization of human thrombus using a novel fibrin-targeted paramagnetic nanoparticle contrast agent. Magn Reson Med. 2000 December; 44(6):867-72.

All documents cited in this application are hereby incorporated by reference as if recited in full herein.

Although illustrative embodiments of the present invention have been described herein, it should be understood that the invention is not limited to those described, and that various other changes or modifications may be made by one skilled in the art without departing from the scope or spirit of the invention.

What is claimed is:

1. A transgenic non-human animal comprising in its genome a polynucleotide encoding a von Willebrand factor (VWF) polypeptide, wherein the transgenic non-human animal expresses the VWF and forms a thrombus when in the presence of human platelets.

2. The transgenic non-human animal according to claim 1, wherein the VWF polypeptide comprises amino acids 1240P through 1481G of SEQ ID NO:6.

3. The transgenic non-human animal according to claim 1, wherein the VWF polypeptide is at least 90% identical to the amino acid sequence depicted in SEQ ID NO:25.

4. The transgenic non-human animal according to claim 1, wherein the polynucleotide encodes a VWF to which AvW3 specifically binds.

5. The transgenic non-human animal according to claim 1, wherein the animal is selected from the group consisting of mouse, rat, hamster, guinea pig, rabbit, dog, goat, horse, and monkey.

6. The transgenic non-human animal according to claim 1, wherein the animal is a mouse.

7. A transgenic mouse comprising in its genome a polynucleotide encoding a von Willebrand factor (VWF) polypeptide, wherein the transgenic mouse expresses the VWF and forms a thrombus when in the presence of human platelets.

8. The transgenic mouse according to claim 7, wherein the VWF polypeptide comprises amino acids 1240P through 1481G of SEQ ID NO:6.

9. The transgenic mouse according to claim 7, wherein the VWF polypeptide is at least 90% identical to the amino acid sequence depicted in SEQ ID NO:25.

10. The transgenic mouse according to claim 7, wherein the polynucleotide encodes a VWF to which monoclonal antibody AvW3 specifically binds.

11. A nucleic acid sequence comprising SEQ ID NO:13.

12. A vector comprising the nucleic acid sequence of claim 11.

13. A mouse-human chimeric polypeptide sequence comprising the amino acid sequence of SEQ ID NO:25.

14. A method for identifying a candidate agent that modulates human platelet mediated thrombosis comprising:
(a) providing a candidate agent;
(b) providing a non-human transgenic animal according to claim 1;
(c) administering the candidate agent to the non-human transgenic animal or to VWF produced by the non-human transgenic animal; and
(d) evaluating an effect, if any, of the candidate agent on human platelet mediated thrombosis in the non-human transgenic animal or the VWF produced by the non-human transgenic animal by detecting an alteration in interactions between the VWF and human platelets.

15. The method according to claim 14, wherein the VWF polypeptide comprises amino acids 1240P through 1481G of SEQ ID NO:6.

16. The method according to claim 14, wherein the VWF polypeptide is at least 90% identical to amino acid sequence depicted in SEQ ID NO:25.

17. The method according to claim 14, wherein the polynucleotide encodes a VWF to which AvW3 specifically binds.

18. The method according to claim 14, wherein the animal is selected from the group consisting of mouse, rat, hamster, guinea pig, rabbit, dog, goat, horse, and monkey.

19. The method according to claim 14, wherein the animal is a mouse.

20. A method for identifying a candidate agent that modulates human platelet mediated thrombosis comprising:
(a) providing a candidate agent;
(b) providing a transgenic mouse according to claim 7;
(c) administering the candidate agent to the transgenic mouse or to VWF produced by the transgenic mouse; and
(d) evaluating an effect, if any, of the candidate agent on human platelet mediated thrombosis in the transgenic mouse or the VWF produced by the transgenic mouse by detecting an alteration in interactions between the VWF and human platelets.

21. The method according to claim 20, wherein the VWF polypeptide comprises amino acids 1240P through 1481G of SEQ ID NO:6.

22. The method according to claim 20, wherein the VWF polypeptide is at least 90% identical to amino acid sequence depicted in SEQ ID NO:25.

23. The method according to claim 20, wherein the polynucleotide encodes a VWF to which AvW3 specifically binds.

24. The method according to claim 20, wherein the evaluating step comprises the use of a diagnostic assay for determining GPIb-alpha-VWF-A1 protein interaction.

25. The method of claim 24, wherein the diagnostic assay comprises perfusing platelets into a flow chamber at a shear flow rate of at least 100 s⁻¹, wherein the VWF protein is immobilized on a bottom surface of the chamber.

26. The method of claim 24, wherein the diagnostic assay comprises perfusing platelets into the transgenic mouse.

27. The method of claim 25, wherein the perfusion of platelets occurs prior to administration of the agent.

28. The method of claim 25, wherein the platelets are human platelets.

29. The method of claim 25, wherein the platelets are not murine platelets.

30. The method of claim 25, wherein the administration of the candidate agent and the perfusion of the platelets occur sequentially.

31. The method of claim 26, wherein the perfusion of platelets occurs prior to administration of the agent.

32. The method of claim 26, wherein the platelets are human platelets.

33. The method of claim 26, wherein the platelets are not murine platelets.

34. The method of claim 26, wherein perfusion of platelets is followed by perfusion of a labeled agent.

35. The method of claim 20, wherein the evaluating step comprises detecting an increase or decrease in the dissociation rate between the VWF produced by the transgenic mouse and GPIb-alpha protein by at least two-fold.

36. The method of claim 20, wherein the evaluating step comprises detecting an increase or decrease of platelet adhesion to a surface expressing VWF produced by the transgenic mouse.

37. The method of claim 20, wherein the evaluating step comprises detecting an increase or decrease in a stabilization of an interaction between VWF-A1 protein and GPIb-alpha protein.

38. The method of claim 20, wherein the evaluating step comprises detecting thrombosis formation.

39. The method of claim 20, wherein the evaluating step comprises identifying an occurrence of an abnormal thrombotic event in the transgenic mouse.

40. The method of claim 39, wherein the abnormal thrombotic event comprises abnormal bleeding, abnormal clotting, death, or a combination thereof.

41. The method of claim 20, wherein the evaluating step comprises dynamic force microscopy, a coagulation factor assay, a platelet adhesion assay, thrombus imaging, a bleeding time assay, aggregometry, review of real-time video of blood flow, a Doppler ultrasound vessel occlusion assay, or a combination thereof.

42. The method of claim 25, wherein perfusion platelets is followed by perfusion of a labeled agent.

43. The method of any one of claims 34 or 42, wherein the labeled agent comprises one or more of a nanoparticle, a fluorophore, a quantum dot, a microcrystal, a radiolabel, a dye, a gold biolabel, an antibody, or a small molecule ligand.

44. The method of any one of claims 34 or 42, wherein the labeled agent targets a platelet receptor, a VWF protein, or a portion thereof.

45. A method for determining whether platelet function or morphology in a subject is abnormal, the method comprising:
a) affixing a protein comprising a VWF-A1 domain obtained from the transgenic non-human animal of claim 1 to a surface of a flow chamber;
b) perfusing through the flow chamber a volume of blood or plasma from a subject at a shear flow rate of at least about 100 s⁻¹;
c) perfusing a targeted molecular imaging agent into the flow chamber; and
d) comparing the flow rate of the blood or plasma from the subject as compared to a normal flow rate, so as to determine whether the subject's platelet function or morphology is abnormal.

46. The method of claim 45, wherein the affixing comprises:
(i) coating a surface of the chamber with an antibody that specifically binds VWF-A1 domain, and
(ii) perfusing the VWF-A1 protein produced by the transgenic mouse in the flow chamber at a shear flow rate of at least 100 s⁻¹.

47. The method of claim 45, wherein the targeted molecular imaging agent comprises a nanoparticle, a fluorophore, a quantum dot, a microcrystal, a radiolabel, a dye, a gold biolabel, an antibody, a peptide, a small molecule ligand, or a combination thereof.

48. The method of claim 45, wherein the targeted molecular imaging agent binds to a platelet receptor, a platelet ligand, or any region of a VWF protein or a portion thereof.

49. The method of claim 45, wherein the targeted molecular imaging agent comprises horseradish peroxidase (HRP) coupled to an antibody that specifically binds to VWF-A1 or a fragment thereof.

50. The method of claim 45, wherein the comparing step comprises a platelet adhesion assay, fluorescence imaging, a chromogenic indicator assay, a microscopy morphology analysis, or any combination thereof.

51. The method of claim 45, wherein platelets bound to VWF-A1 are less than about 500 cells/mm².

52. The method of claim 45, wherein the platelets are substantially spherical.

53. The method of claim 45, wherein the subject is selected from the group consisting of a human, a canine, a feline, a murine, a porcine, an equine, or a bovine.

54. The method of claim 45, wherein the protein comprising the VWF-A1 is affixed to the chamber with an agent selected from the group consisting of an antibody, a peptide, and a Fab fragment that specifically binds to a VWF polypeptide or a portion thereof.

55. A method for producing chimeric von Willebrand Factor A1 protein that specifically binds to human platelets, the method comprising:
(a) providing a non-human animal expressing a chimeric von Willebrand Factor A1 protein, wherein the chimeric protein causes the platelet binding specificity of the non-human animal von Willebrand Factor A1 protein to change to be specific for human platelets; and
(b) harvesting the chimeric von Willebrand Factor A1 from the non-human animal, which specifically binds human platelets.

56. A method for calibrating an aggregometry device or a device for measuring clot formation or retraction, the method comprising:
a) providing hematologic data obtained from a subject, wherein blood or platelets from the subject is assessed by the device;
b) determining whether or not a thrombotic event occurs in the transgenic non-human animal of claim 1, wherein the animal is perfused with a sample of blood or platelets from the subject; and
c) correlating data obtained from step (b) with the data obtained in step (a) so as to calibrate the device, wherein a certain data obtained from the device is indicative of the corresponding thrombotic outcome determined in the transgenic non-human animal of claim 1.

57. The method of claim 56, wherein the thrombotic event comprises blood clotting, abnormal bleeding, abnormal clotting, death, or a combination thereof.
